(19) United States
(12) Patent Application Publication
(10) Pub. No.: US 2024/0166660 A1
(43) Pub. Date: May 23, 2024

(54) KRAS G12C INHIBITORS

(71) Applicant: BeiGene Switzerland GmbH, Basel (CH)

(72) Inventors: Guoliang ZHANG, Beijing (CN); Jianzhuang MIAO, Beijing (CN); Ce WANG, Beijing (CN); Zhikun NI, Beijing (CN); Huaguo LI, Beijing (CN)

(21) Appl. No.: 18/261,629

(22) PCT Filed: Jan. 14, 2022
(86) PCT No.: PCT/CN2022/071943

§ 371 Date: Jul. 14, 2023

(30) Foreign Application Priority Data
Jan. 15, 2021 (WO) PCT/CN2021/072190

(51) Int. Cl. C07D 491/052; C07D 491/048; C07D 519/00

(52) U.S. Cl. CPC C07D 491/052; C07D 491/048; C07D 519/00

(57) ABSTRACT

Disclosed herein are compounds or a stereoisomer thereof, or a pharmaceutically acceptable salt thereof useful as G12C inhibitors, and a pharmaceutical composition comprising the same.

### FIELD OF THE INVENTION

Disclosed herein are compounds or a stereoisomer thereof, or a pharmaceutically acceptable salt thereof useful as G12C inhibitors, and a pharmaceutical composition comprising the same.

### BACKGROUND OF THE INVENTION

RAS is one of the most well-known oncogenes. In human beings, three RAS genes (HRAS, KRAS and NRAS) encode four highly homologous RAS proteins (HRAS, KRAS-4A, KRAS-4B and NRAS). RAS proteins are small GTPases, they function as binary molecular switches that involved in transduction of extracellular growth and differentiation signaling.

RAS generally cycles between a GDP-bound “off” state and a GTP-bound “on” state. This cycle is regulated by several factors. Guanine nucleotide exchange factors (GEFs), including SOS1 and SOS2 facilitate the exchange and formation of GTP-bound RAS. While, GTPase-activating proteins (GAPs), for example, NF-1 promote the hydrolysis of GTP and therefore turn RAS back to GDP-bound inactivate state (Kessler et al, PNAS, 2019, 116 (32): 15823-15829). Once bound to GTP, RAS initiates conformational changes in two specific regions Switch 1 and Switch 2, which allows engagement and activation of downstream effector proteins to initiate a cascade of intracellular signaling pathways. These effectors include RAF-MEK-ERK and PI3K-AKT-mTOR pathways, both of which have crucial roles in regulating cell proliferation, differentiation, and survival (Cox et al., Nature Reviews Drug Discovery, 2014, 13:828-851).

RAS mutations have been identified in around 30% of human tumors. These mutations occur frequently as single-base missense mutations in codons 12, 13 or 61, resulting in stabilization of the activated GTP-bound RAS form and constitutive activation of RAS downstream signaling pathways. KRAS is the most frequently mutated RAS in cancer, account for 85% of all RAS-driven cancers, followed by NRAS (12%) and HRAS (3%). KRAS mutation has been detected in around 95% of pancreatic ductal adenocarcinoma, 50% of colorectal adenocarcinoma and 30% of lung adenocarcinoma. The majority of KRAS mutations occur at residue 12, and the mutation type varied in different cancers. In colon cancer and pancreatic cancer, the predominant KRAS mutation is G12D (glycine to lysine), while in non-small cell lung cancer (NSCLC), nearly half of KRAS mutations are G12C (glycine to cysteine) (Cox et al., Nature Reviews Drug Discovery, 2014, 13:828-851).

Based on the critical role of RAS in cell proliferation and its high mutation rate in human cancers, RAS has long been considered as a therapeutic target for many cancers. However, despite several decades of research effort, no anti-RAS small molecular has been clinically approved. The main reason is that druggable pockets on the surface of RAS are lacking (Papke et al., Science, 2017, 355: 1158-1163). Recently, more and more studies suggested that RAS might be able to be drugged with small molecules. Several inhibitors that directly target KRAS G12C are under the investigation (Patricelli et al, Cancer Discovery, 2016, 6(3); 316-29) (Fell et al, ACS Med. Chem. Lett. 2018, 9, 12, 1230-1234).

Small molecule selectively inhibitors of KRAS are being developed to prevent or treat diseases, for example, WO2015/054572A1 provides compounds having activity as inhibitors of G12C mutant RAS protein. WO2016/164675A1 and WO2017/015562A1 disclose substituted quinazoline compounds as KRAS G12C inhibitors. Compounds with KRAS G12C inhibitory activity are further reported by WO2014/152588A1, WO2016/049524A1, WO2016/168540A1, WO2017/058728A1, WO2017/058792A1, WO2017/058805A1, WO2017/058915A1, WO2017/087528A1, WO2018/064510A1, WO2018/068017A1, WO2018/119183A2, WO2018/206539A1, WO2018/218069A1, WO2019/051291A1, WO2019/055540A1, WO2019/137985A1, WO2019/141250A1, WO2019/150305A1, WO2019/155399A1, WO2019/213516 A1, WO2019/213526A1, WO2019/215203A1, WO20192/17307A1, WO2019/217691A1, WO2019/232419A1, WO2020/028706A1, WO2020/027084A1, WO2020/027083A1, WO2020/035031A1, WO2020/047192A1, and WO2020/101736A1.

Thus, new inhibitors that selectively target mutant KRAS with high efficacy and safety are still highly desirable. Continued efforts on developing KRAS G12C inhibitors will arise a new therapeutic way for KRAS G12C driven cancers.

### SUMMARY OF THE INVENTION

Disclosed herein are bridged compounds of Formula (I), and the methods of use. The bridged compounds disclosed herein inhibit KRAS G12C activity and are useful in the treatment of various diseases including cancer. The first embodiment comprises the following aspects:

Aspect 1: A compound of Formula (I):

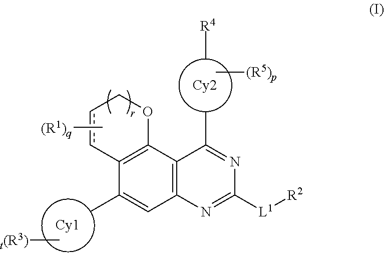

or a pharmaceutically acceptable salt thereof, or a stereoisomer thereof, wherein
[symbol] is a single bond or a double bond;
Cy1 is selected from —C₅₋₁₂cycloalkyl, 5- to 12-membered heterocyclyl, —C₆₋₁₂aryl or 5- to 12-membered heteroaryl;
Cy2 is selected from 4- to 12-membered cycloalkyl or 4- to 12-membered heterocyclyl;
L¹ is selected from a single bond, —CO—NR^{a}—, —NR^{a}—CO—, —O—, —NR^{a}—, —NR^{a}(CH₂)ₘ—, —S—, —(CH₂)ₘ—, —O—(CH₂)ₘ—, —O—CH(R^{a})—, —CH(R^{a})—, —CH(R^{a})(CH₂)ₘ—, —(CH₂)ₘ—O—, —CO—, —SO₂—, cycloalkylene, oxetandiyl, tetrahydrofurandiyl, tetrahydropyrandiyl, azetidindiyl, pyrrilidindiyl, piperidindiyl, or piperizindiyl;
R¹ is selected from —H, —C₁₋₈alkyl, —C₂₋₈alkenyl, —C₂₋₈alkynyl, —NR^{b}R^{c}, —C₃₋₁₂cycloalkyl, —C₃₋₁₂heterocyclyl, —C₆₋₁₂aryl, or —C₅₋₁₂heteroaryl, each of said —C₁₋₈alkyl, —C₂₋₈alkenyl, —C₂₋₈alkynyl, —C₃₋₁₂cycloalkyl, —C₃₋₁₂heterocyclyl, —C₆₋₁₂aryl, or —C₅₋₁₂heteroaryl is optionally substituted with at least one R⁶;
R² is selected from —H, —C₁₋₈alkyl, —NR^{b}R^{c}, —C₃₋₁₂cycloalkyl, 3- to 12-membered heterocyclyl, —C₆₋₁₂aryl, or 5- to 12-membered heteroaryl, wherein each of said —C₃₋₁₂cycloalkyl, 3- to 12-membered heterocyclyl, —C₆₋₁₂aryl, or 5- to 12-membered heteroaryl is optionally substituted with at least one R⁶;
R⁶, at each of its occurrences, is independently —C₁₋₈alkyl, halogen, hydroxy, oxo, —C₁₋₈alkoxy, —NR^{b}R^{c}, —C₃₋₈cycloalkyl, 3- to 8-membered heterocyclyl, —C₆₋₁₂aryl, or 5- to 12-membered heteroaryl, wherein said —C₁₋₈alkyl, —C₁₋₈alkoxy, —C₃₋₈cycloalkyl, 3- to 8-membered heterocyclyl, —C₆₋₁₂aryl, or 5- to 12-membered heteroaryl is optionally substituted with at least one halogen, hydroxy, amino, —C₁₋₈alkoxy, —NR^{b}R^{c}, —CN, cycloalkyl, heterocyclyl, aryl or heteroaryl;
R³ is selected from hydrogen, halogen, oxo, —C₁₋₈alkyl, —C₂₋₈alkenyl, —C₂₋₈alkynyl, —C₃₋₈cycloalkyl, 3- to 8-membered heterocyclyl, —C₆₋₁₂aryl, 5- to 8-membered heteroaryl, —OR^{a}, —NR^{a}R^{b} or —CN, wherein each of said —C₁₋₈alkyl, —C₂₋₈alkenyl, —C₂₋₈alkynyl, —C₃₋₈cycloalkyl, 3- to 8-membered heterocyclyl, —C₆₋₁₂aryl, or 5- to 12-membered heteroaryl is optionally substituted with at least one halogen, hydroxy, —C₁₋₈alkoxy, —C₃₋₈cycloalkyl, 3- to 8-membered heterocyclyl, —C₆₋₁₂aryl, or 5- to 12-membered heteroaryl; or
two R³ together with the atom(s) to which they are attached, form a 3- to 8-membered ring, said ring comprising 0, 1 or 2 additional heteroatom(s) independently selected from nitrogen, oxygen or optionally oxidized sulfur as ring member(s), said ring is optionally substituted with at least one halogen, hydroxy, amino, —C₁₋₈alkyl, —C₁₋₈alkoxy, —NR^{b}R^{c}, —CN, cycloalkyl, heterocyclyl, aryl or heteroaryl;
R⁵ is selected from —C₁₋₈alkyl, —C₂₋₈alkenyl, —C₂₋₈alkynyl, oxo, —NR^{b}R^{c}, —CO—NR^{b}R^{c}, cycloalkyl, heterocyclyl, aryl, or heteroaryl, —(CH₂)ₘ—CN, or hydrogen;
R⁴ is selected from

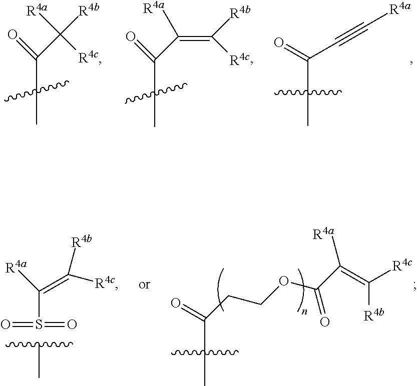

wherein each R^{4a}, R^{4b} and R^{4c} are independently hydrogen, deuterium (D), cyano (CN), halogen, —OR^{a}, —C₁₋₈alkyl, —C₃₋₁₂cycloalkyl, —C₃₋₁₂heterocyclyl, —C₆₋₁₂aryl, —C₅₋₁₂heteroaryl, —NR^{b}R^{c}, —CONR^{b}R^{c}, each of said —C₁₋₈alkyl, —C₃₋₁₂cycloalkyl, —C₃₋₁₂heterocyclyl, —C₆₋₁₂aryl or —C₅₋₁₂heteroaryl is optionally substituted with at least one R⁶;
wherein each R^{a}, R^{b} and R^{c} are independently hydrogen, deuterium (D), cyano (CN), halogen, hydroxy, —C₁₋₈alkoxy, —C₁₋₈alkyl, —C₂₋₈alkenyl, —C₂₋₈alkynyl, —C₃₋₈cycloalkyl, 3- to 12-membered heterocyclyl, aryl, heteroaryl, —NR^{d}R^{e}, or —CO—NR^{d}R^{e}, each of said —C₁₋₈alkyl, —C₂₋₈alkenyl, —C₂₋₈alkynyl, cycloalkyl, heterocyclyl, aryl, or heteroaryl is optionally substituted with R^{f}; or
optionally (R^{a} and R^{b}), or (R^{a} and R^{c}) together with the atom(s) to which they are attached, form a 4- to 6 membered ring, said ring is optionally substituted with at least one R^{g};
each R^{f} is selected from halogen, hydroxy, oxo, —C₁₋₈alkoxy, —NR^{d}R^{e}, —CO—NR^{d}R^{e}, —NR^{d}—CO—R^{e}, cycloalkyl, heterocyclyl, aryl, or heteroaryl, wherein each said —C₁₋₈alkoxy, cycloalkyl, heterocyclyl, aryl, or heteroaryl is optionally substituted with halogen, hydroxy or —C₁₋₄alkyl;
R^{a}, R^{e} and R^{g} are each independently hydrogen, deuterium (D), halogen, —C₁₋₈alkyl; each said —C₁₋₈alkyl is optionally substituted with at least one halogen, oxo, CF₃ or —COCH₃;
p, q and t are independently selected from 0, 1, 2, 3 or 4;
r is selected from 0, 1 or 2;
each m and n are independently 0, 1, 2, 3, 4, 5 or 6.

Aspect 2: The compound according to Aspect 1, wherein Cy1 is selected from 6-, 7-, 8-, 9-, 10-membered ring, the said ring is saturated or unsaturated ring comprising 0, 1 or 2 heteroatom(s) independently selected from nitrogen, oxygen or optionally oxidized sulfur as ring member(s), preferred Cy1 is selected from single ring, spiral ring, fused ring, bridged ring.

Aspect 3: The compound according to Aspect 1, wherein Cy1 is selected from naphthalene, benzene, benzopyrazole, pyridine, benzocyclopentane, benzothiazole, benzimidazole, indole, quinoline, isoquinoline, benzotetrahydrofuran, tetrahydroquinoline, tetrahydroisoquinoline, dihydroindole, 1,2-dihydronaphthalene, 1,4-dihydronaphthalene, or tetralin.

Aspect 4: The compound according to any one of Aspects 1-3, wherein R³ is selected from hydrogen, F, Cl, Br, I, oxo, methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole, —OH, —OCH₃, —OC₂H₅, —OC₃H₇, —OC₄H₉, —OC₅H₁₁, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉ or —CN, each of said methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole is optionally substituted with at least one F, Cl, Br, I, OH, —OCH₃, —OC₂H₅, —OC₃H₇, —OC₄H₉, —OC₅H₁₁, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole.

Aspect 5: The compound according to Aspect 4, wherein R³ is selected from hydrogen, F, Cl, Br, I, oxo, —CH₃, —C₂H₅, —C₃H₇, —C₄H₉, —CF₃, cyclopropyl, cyclobutyl, cyclopentyl, —OH, —OCH₃, —OC₂H₅, —OC₃H₇, —OC₄H₉, —OC₅H₁₁, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉ or —CN.

Aspect 6: The compound according to any one of Aspects 1 to 3, wherein two R³ together with the atom(s) to which they are attached, form a 3-, 4-, 5-, 6-, 7- or 8-membered ring, said ring comprising 0, 1 or 2 additional heteroatom(s) independently selected from nitrogen or oxygen as ring member(s), said ring is optionally substituted with at least one F, Cl, Br, I, OH, NH₂, —CH₃, —C₂H₅, —C₃H₇, —C₄H₉, —C₅H₁₁, —OCH₃, —OC₂H₅, —OC₃H₇, —OC₄H₉, —OC₅H₁₁, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CN, cyclopropyl, cyclobutyl, cyclopentyl, cyclohexyl, cycloheptyl, cyclooctyl, azacyclopropyl, azacyclobutyl, tetrahydropyrrole, piperidine, piperazine, morpholine, propylene oxide, oxacyclobutane, oxacyclopentane, oxacyclohexane, phenyl, pyrrole, imidazole, pyrazole, thiazole, thiophene, oxazole, pyridine, indole, quinoline, isoquinoline, benzimidazole, benzothiazole, benzopyrazole.

Aspect 7: The compound according to Aspect 6, wherein two R³ together with the atom(s) to which they are attached, form a ring, said ring is selected from phenyl, pyrrole, imidazole, pyrazole, thiazole, thiophene, oxazole, pyridine, indole, quinoline, isoquinoline, benzimidazole, benzothiazole or benzopyrazole, each of said phenyl, pyrrole, imidazole, pyrazole, thiazole, thiophene, oxazole, pyridine, indole, quinoline, isoquinoline, benzimidazole, benzothiazole or benzopyrazole is optionally substituted with at least one F, Cl, Br, I, OH, NH₂, —CH₃, —C₂H₅, —C₃H₇, —C₄H₉, —C₅H₁₁, —OCH₃, —OC₂H₅, —OC₃H₇, —OC₄H₉, —OC₅H₁₁, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CN.

Aspect 8: The compound according to any one of Aspects 1-7, wherein Cy1 is selected from

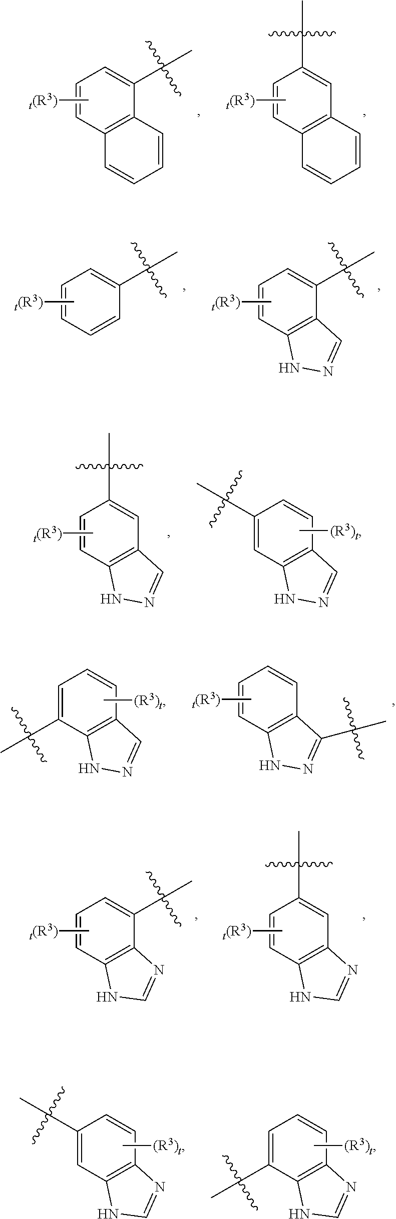

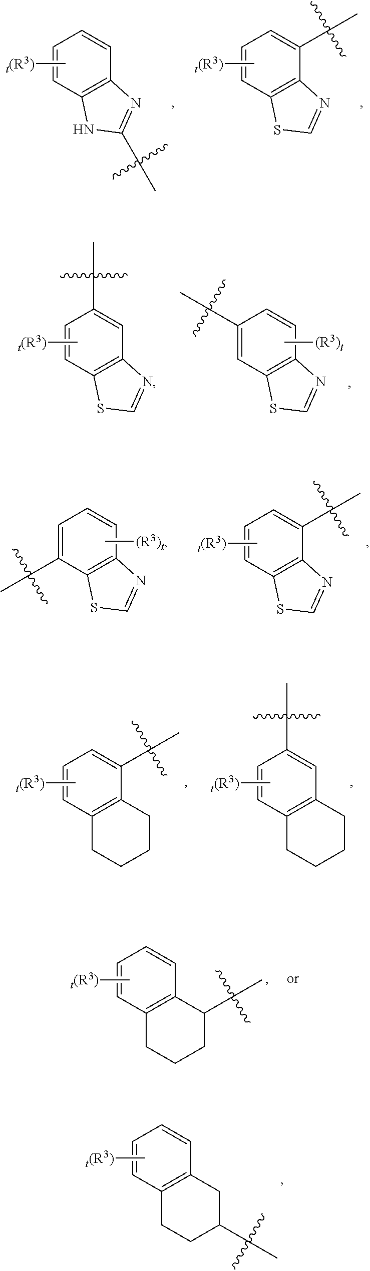

wherein R³ and t are defined as for formula (I), preferably t is 0, 1 or 2, and R³, at each of its occurrences, is independently selected from halogen, —C₁₋₈alkyl, —C₃₋₈cycloalkyl, -haloC₁₋₈alkyl, —CN, —OR^{a}, or —NR^{a}R^{b}, wherein R^{a} and R^{b} are independently hydrogen, —C₁₋₈alkyl or —C₃₋₈cycloalkyl; or two germinal R³ together with the atom to which they are attached, form a C₃₋₈cycloalkyl ring; more preferably R³, at each of its occurrences, is independently selected from F, Cl, Br, methyl, —CF₃, NH₂, CN, cyclopropyl, or hydroxy.

Aspect 9: The compound according to Aspect 1, wherein

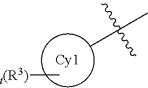

moiety is selected from

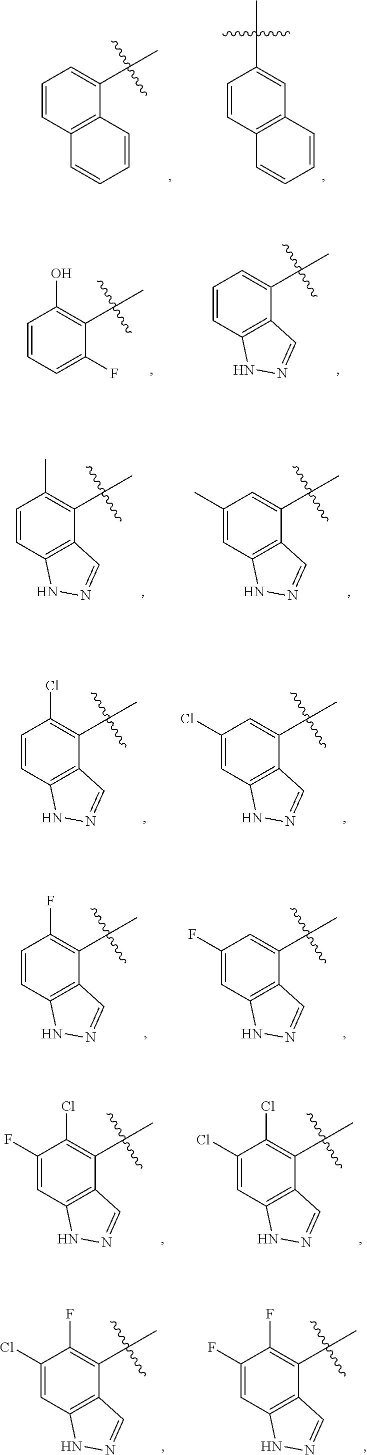

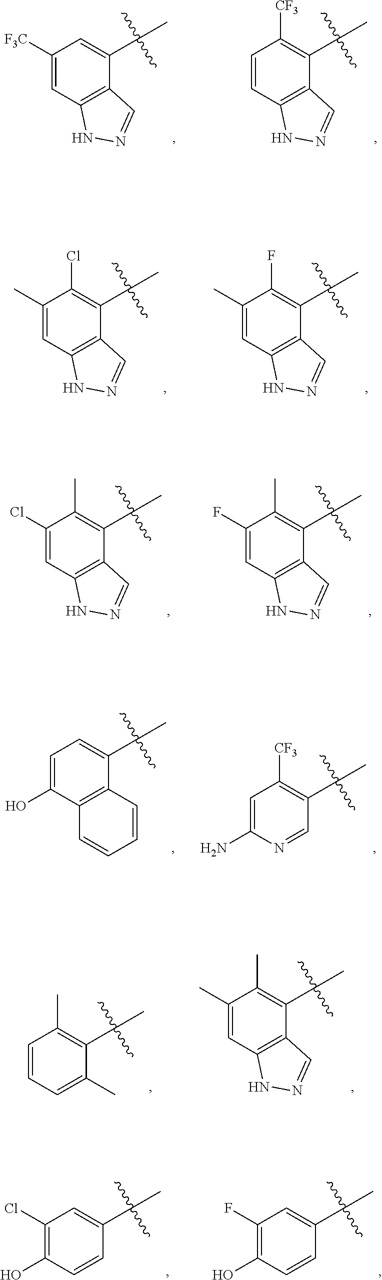

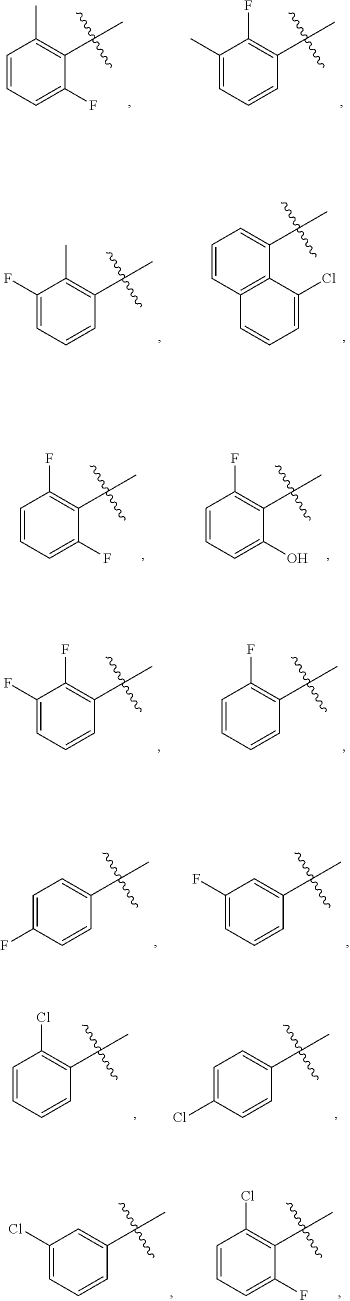

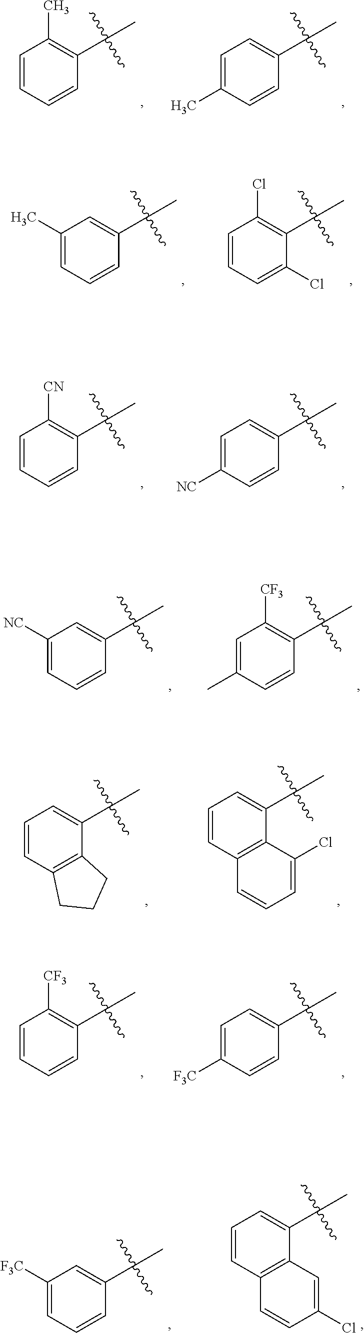

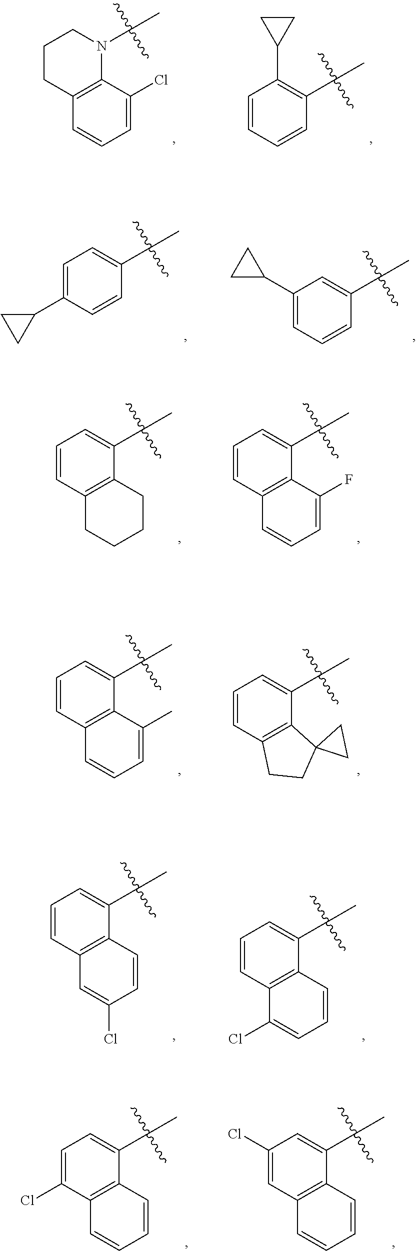

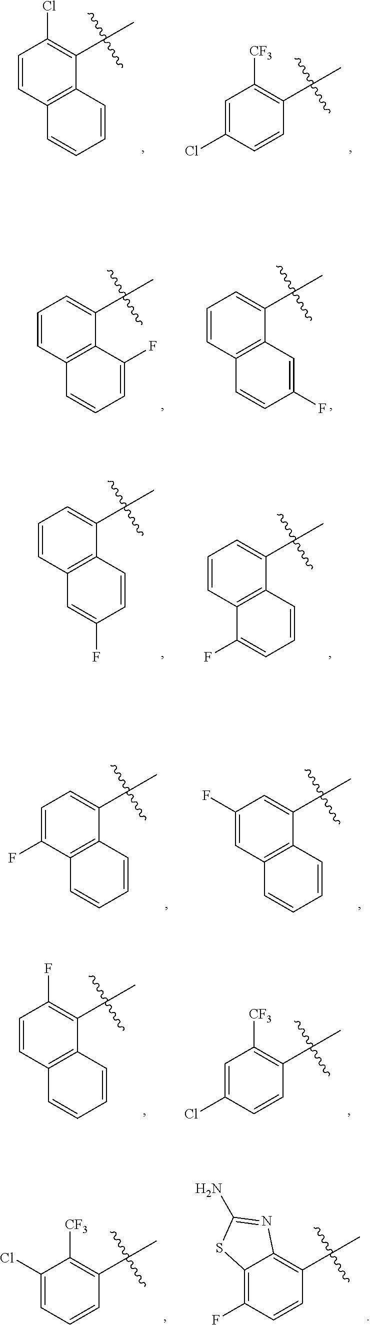

Aspect 10: The compound according to any one of Aspects 1-8, wherein Cy2 is selected from 6-, 7-, 8-, 9-, 10-membered ring, the said ring is saturated or unsaturated ring comprising 0, 1 or 2 additional heteroatom(s) independently selected from nitrogen, oxygen or optionally oxidized sulfur as ring member(s); preferably, Cy2 is a 5-, 6-, or 7-membered monocyclic heterocyclic ring comprising one or two nitrogen as ring member(s), a 7- to 12-membered bicyclic fused heterocyclic ring comprising one or two nitrogen as ring member(s), or a 7- to 12-membered bicyclic spiro heterocyclic ring comprising one or two nitrogen as ring member(s).

Aspect 11: The compound according to Aspect 10, wherein Cy2 is selected from

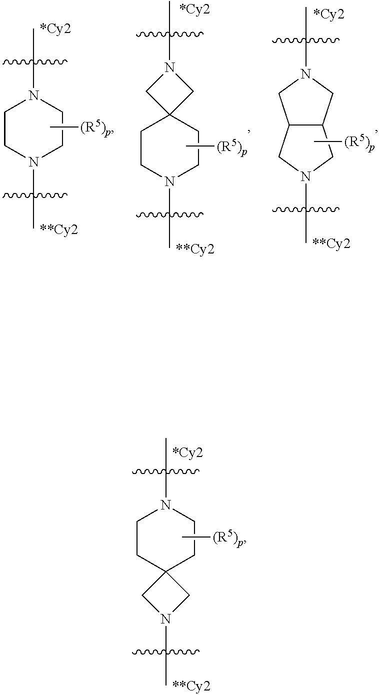

wherein *^{Cy2} refers to the position attached to R⁴ moiety, and **^{Cy2} refers to the position attached to the

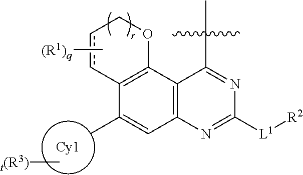

moiety.

Aspect 12: The compound according to Aspect 11, wherein R⁵ is selected from —H, methyl, ethyl, propyl, butyl, amyl, hexyl, heptyl, octyl, cyclopropyl, cyclobutyl, cyclopentyl, cyclohexyl, cycloheptyl, cyclooctyl, phenyl, oxo, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CO—N(CH₃)₂, —CO—N(C₂H₅)₂, —CO—N(C₃H₇)₂, —CO—N(C₄H₉)₂, —CO—N(CH₃)C₂H₅, —CO—N(CH₃)C₃H₇, —CO—N(CH₃)C₄H₉, —CO—N(C₂H₅)C₃H₇, —CO—N(C₂H₅)C₄H₉, —CO—N(C₃H₇)C₄H₉, —CH₂—CN, —(CH₂)₂—CN, —(CH₂)₃—CN; preferably methyl or —CH₂—CN.

Aspect 13 The compound according to any one of Aspects 1-12, wherein R⁴ is selected from

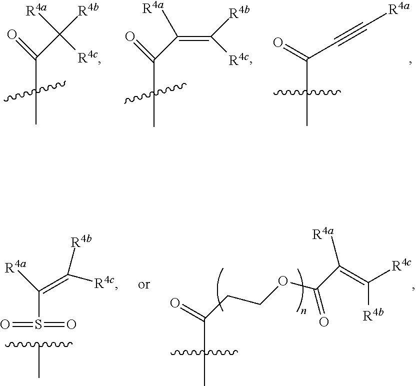

wherein each R^{4a}, R^{4b} and R^{4c} are independently hydrogen, deuterium (D), cyano (CN), F, Cl, Br, I, hydroxy, —OCH₃, —OC₂H₅, —OC₃H₇, —OC₄H₉, —OC₅H₁₁, methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CO—NH₂, —CO—N(CH₃)₂, —CO—N(C₂H₅)₂, —CO—N(C₃H₇)₂, —CO—N(C₄H₉)₂, —CO—N(CH₃)C₂H₅, —CO—N(CH₃)C₃H₇, —CO—N(CH₃)C₄H₉, —CO—N(C₂H₅)C₃H₇, —CO—N(C₂H₅)C₄H₉, —CO—N(C₃H₇)C₄H₉, each of said methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole is optionally substituted with R^{f}; each R^{f} is selected from F, Cl, Br, I, oxo, hydroxy, —OCH₃, —OC₂H₅, —OC₃H₇, —OC₄H₉, —OC₅H₁₁, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CO—NH₂, —CO—N(CH₃)₂, —CO—N(C₂H₅)₂, —CO—N(C₃H₇)₂, —CO—N(C₄H₉)₂, —CO—N(CH₃)C₂H₅, —CO—N(CH₃)C₃H₇, —CO—N(CH₃)C₄H₉, —CO—N(C₂H₅)C₃H₇, —CO—N(C₂H₅)C₄H₉, —CO—N(C₃H₇)C₄H₉, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, or benzopyrazole.

Aspect 14: The compound according to Aspect 13, wherein R⁴ is selected from

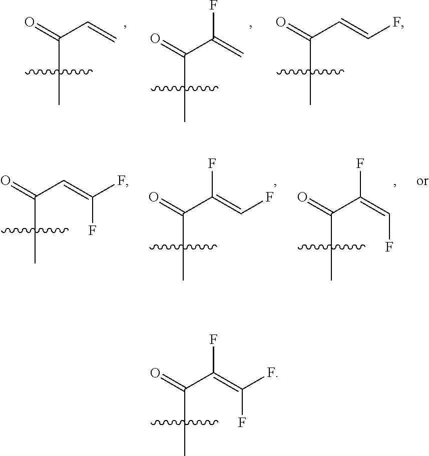

Aspect 15: The compound according to Aspect 1, wherein the

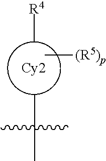

moiety is selected from

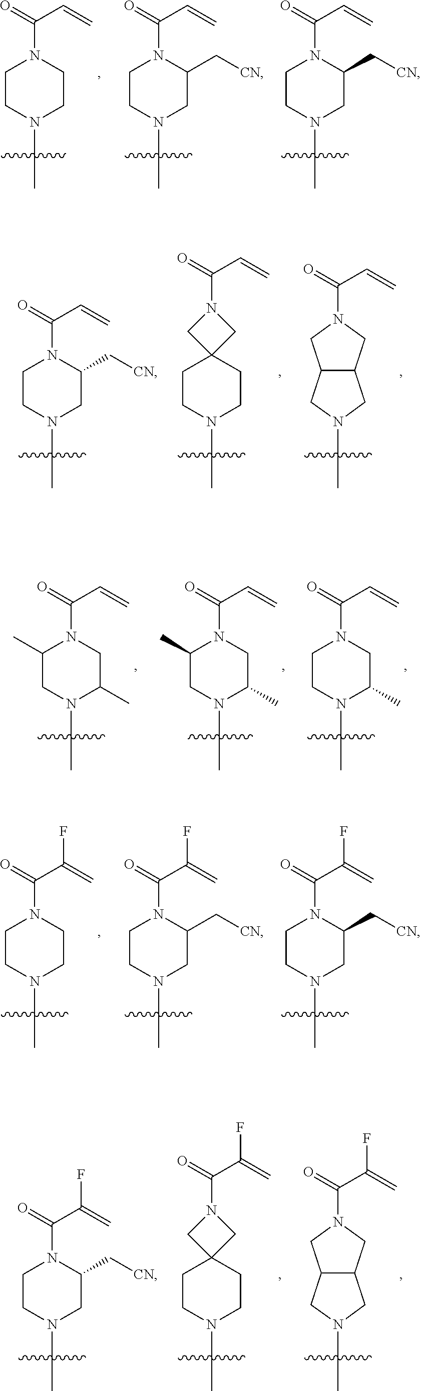

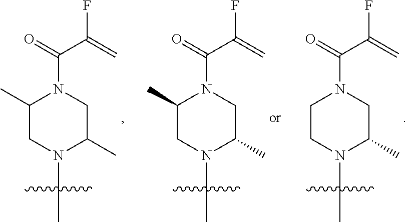

Aspect 16: The compound according to any one of Aspects 1-15, wherein R¹ is selected from —H, methyl, ethyl, propyl, butyl, amyl, hexyl, heptyl, octyl, cyclopropyl, cyclobutyl, cyclopentyl, cyclohexyl, cycloheptyl, cyclooctyl or phenyl, each of said methyl, ethyl, propyl, butyl, amyl, hexyl, heptyl, octyl, cyclopropyl, cyclobutyl, cyclopentyl, cyclohexyl, cycloheptyl, cyclooctyl or phenyl is optionally substituted with at least one R⁶.

Aspect 17: The compound according to Aspect 16, wherein R¹ is selected from —H, methyl, ethyl, propyl, butyl, amyl, hexyl, heptyl, octyl, cyclopropyl, cyclobutyl, cyclopentyl, cyclohexyl, cycloheptyl, cyclooctyl or phenyl.

Aspect 18: The compound according to any one of Aspects 1-17, wherein R² is selected from —H, methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, each of said methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉ is optionally substituted with at least one R⁶.

Aspect 19: The compound according to Aspect 18, wherein R² is selected from H, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉,

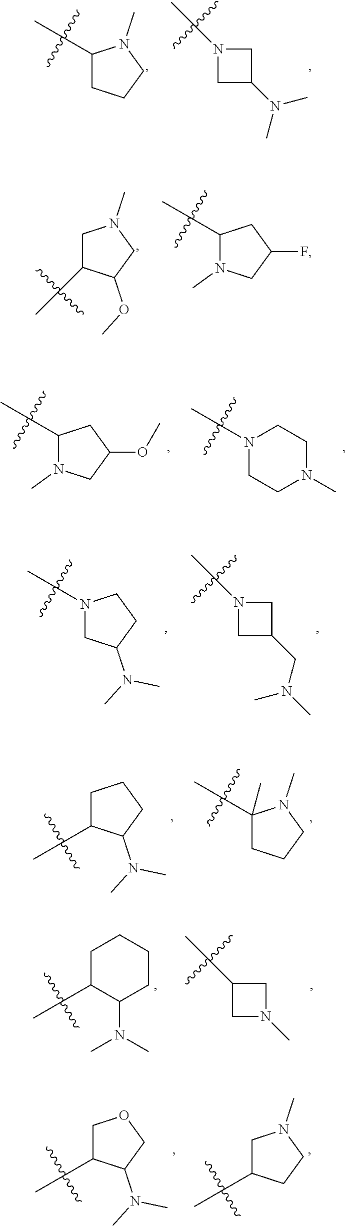

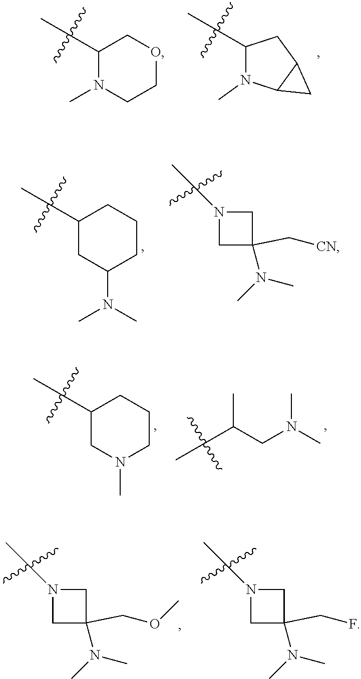

Aspect 20: The compound according to any one of Aspects 1-19, wherein the

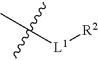

moiety is selected from —H,

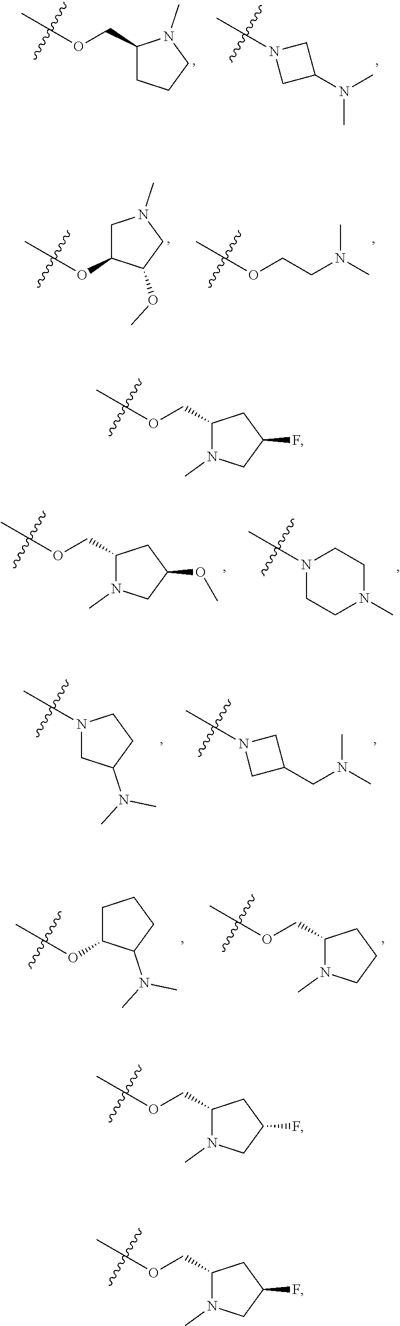

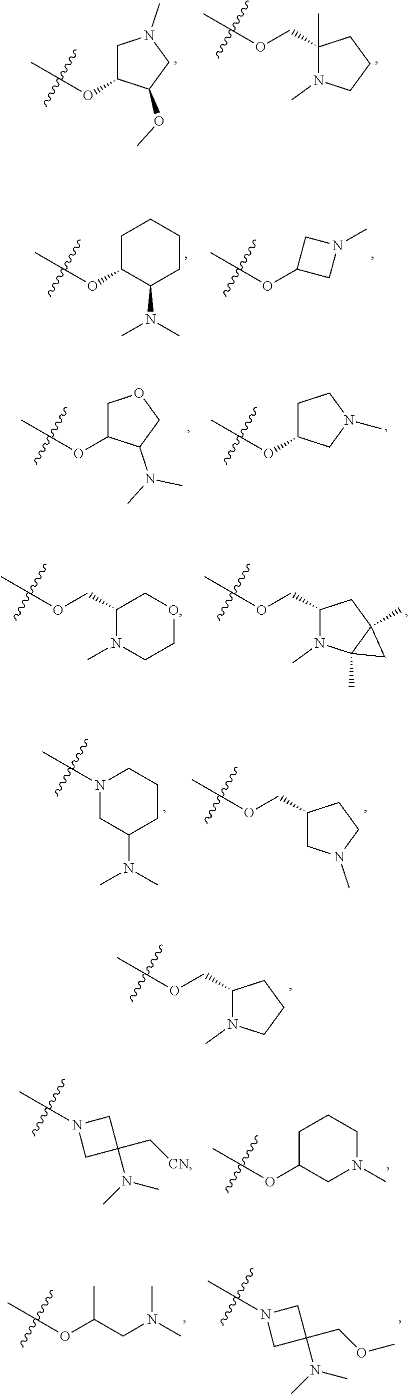

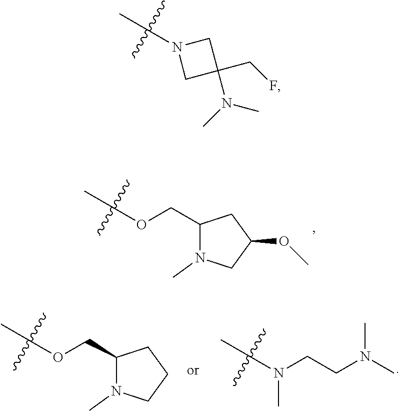

Aspect 21: The compound according to any one of aspects 1-20, wherein r is 0, and [symbol] is a single bond or a double bond; or r is 1, and [symbol] is a single bond or a double bond.

Aspect 22: The compound according to Aspect 1, wherein the compound is selected from

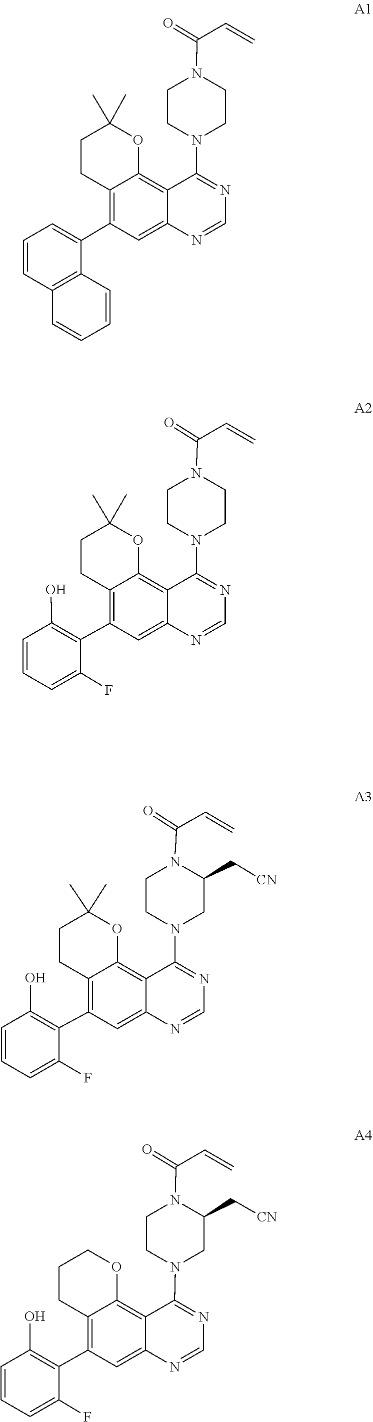 [SMILES: C=CC(=O)N1CCN(c2ncnc3cc(-c4cccc5ccccc45)c4c(c23)OC(C)(C)CC4)CC1 ; C=CC(=O)N1CCN(c2ncnc3cc(-c4c(O)cccc4F)c4c(c23)OC(C)(C)CC4)CC1 ; C=CC(=O)N1CCN(c2ncnc3cc(-c4c(O)cccc4F)c4c(c23)OC(C)(C)CC4)C[C@@H]1CC#N ; C=CC(=O)N1CCN(c2ncnc3cc(-c4c(O)cccc4F)c4c(c23)OCCC4)C[C@@H]1CC#N]

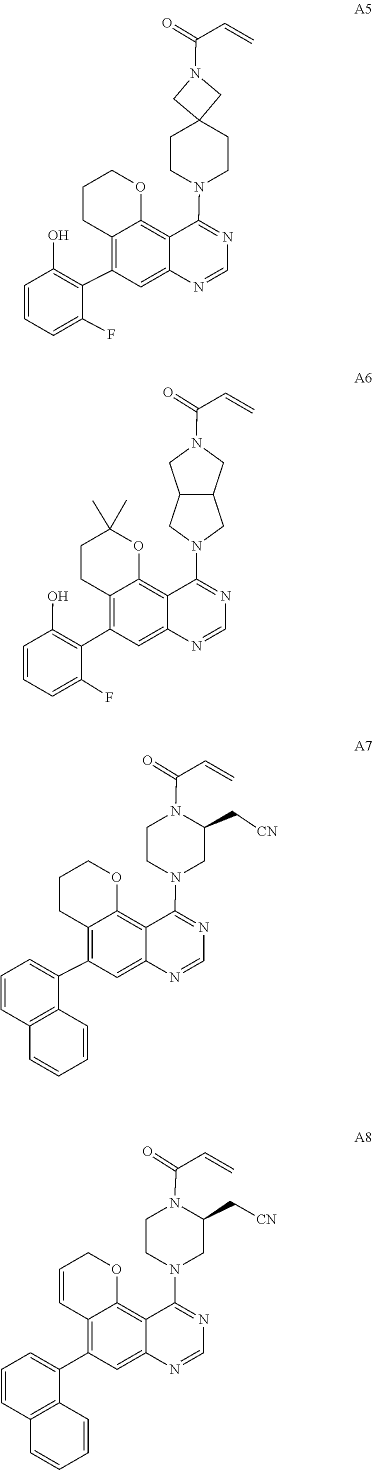 [SMILES: C=CC(=O)N1CC2(CCN(c3ncnc4cc(-c5c(O)cccc5F)c5c(c34)OCCC5)CC2)C1 ; C=CC(=O)N1CC2CN(c3ncnc4cc(-c5c(O)cccc5F)c5c(c34)OC(C)(C)CC5)CC2C1 ; C=CC(=O)N1CCN(c2ncnc3cc(-c4cccc5ccccc45)c4c(c23)OCCC4)C[C@@H]1CC#N ; C=CC(=O)N1CCN(c2ncnc3cc(-c4cccc5ccccc45)c4c(c23)OCC=C4)C[C@@H]1CC#N]

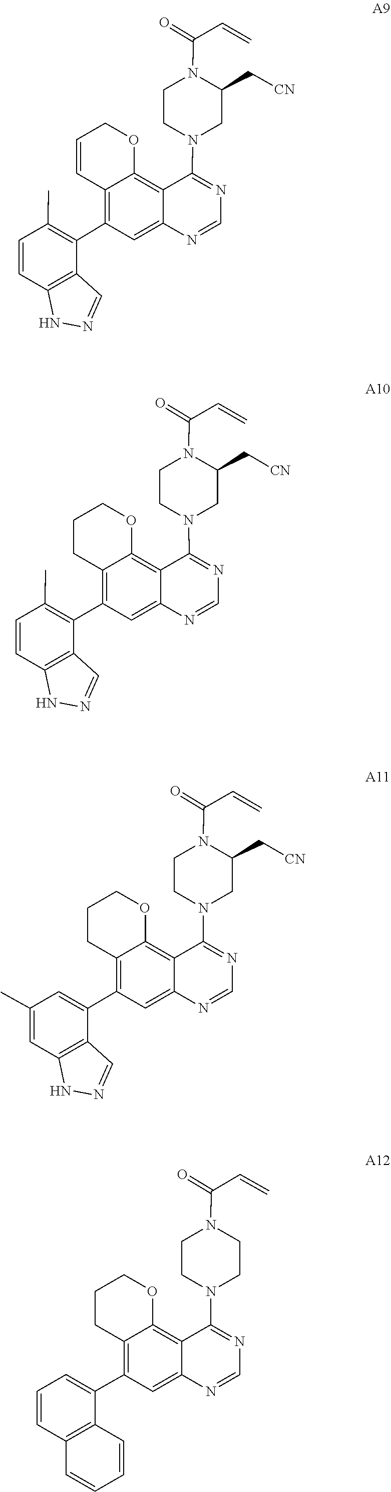 [SMILES: C=CC(=O)N1CCN(c2ncnc3cc(-c4c(C)ccc5[nH]ncc45)c4c(c23)OCC=C4)C[C@@H]1CC#N ; C=CC(=O)N1CCN(c2ncnc3cc(-c4c(C)ccc5[nH]ncc45)c4c(c23)OCCC4)C[C@@H]1CC#N ; C=CC(=O)N1CCN(c2ncnc3cc(-c4cc(C)cc5[nH]ncc45)c4c(c23)OCCC4)C[C@@H]1CC#N ; C=CC(=O)N1CCN(c2ncnc3cc(-c4cccc5ccccc45)c4c(c23)OCCC4)CC1]

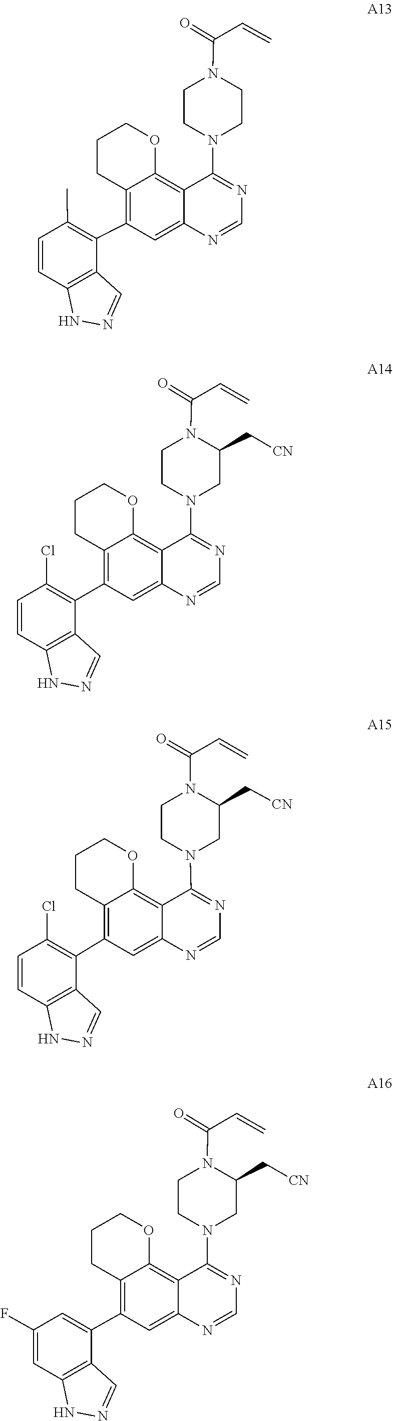 [SMILES: C=CC(=O)N1CCN(c2ncnc3cc(-c4c(C)ccc5[nH]ncc45)c4c(c23)OCCC4)CC1 ; C=CC(=O)N1CCN(c2ncnc3cc(-c4c(Cl)ccc5[nH]ncc45)c4c(c23)OCCC4)C[C@@H]1CC#N ; C=CC(=O)N1CCN(c2ncnc3cc(-c4c(Cl)ccc5[nH]ncc45)c4c(c23)OCCC4)C[C@@H]1CC#N ; C=CC(=O)N1CCN(c2ncnc3cc(-c4cc(F)cc5[nH]ncc45)c4c(c23)OCCC4)C[C@@H]1CC#N]

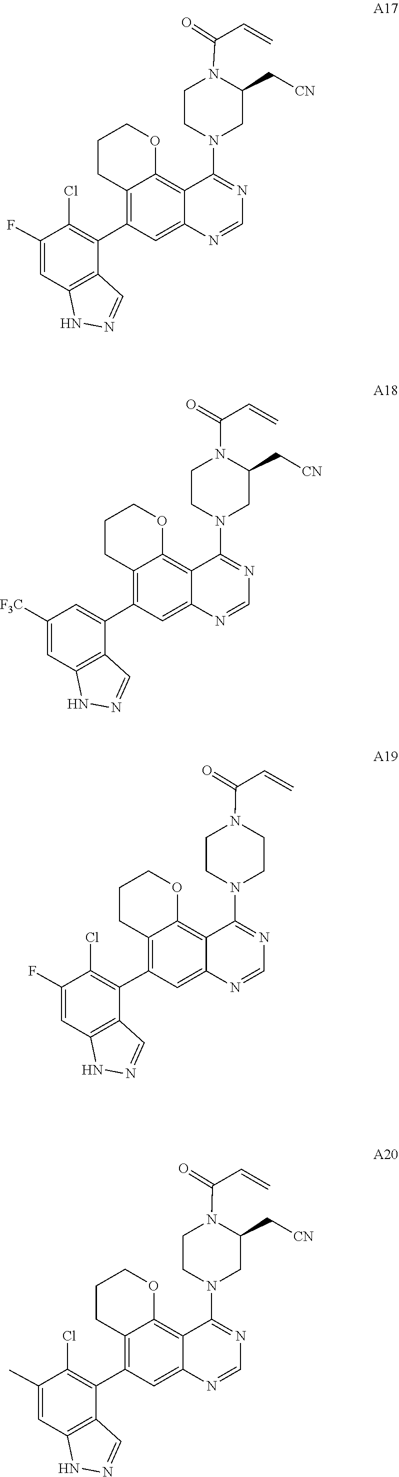

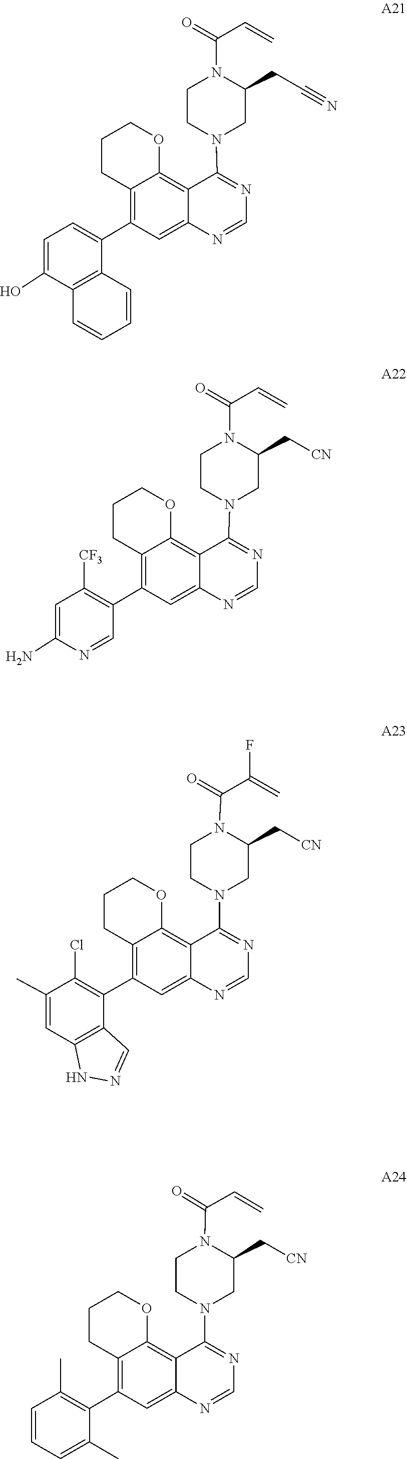 [SMILES: C=CC(=O)N1CCN(c2ncnc3cc(-c4ccc(O)c5ccccc45)c4c(c23)OCCC4)C[C@@H]1CC#N ; C=CC(=O)N1CCN(c2ncnc3cc(-c4cnc(N)cc4C(F)(F)F)c4c(c23)OCCC4)C[C@@H]1CC#N ; C=C(F)C(=O)N1CCN(c2ncnc3cc(-c4c(Cl)c(C)cc5[nH]ncc45)c4c(c23)OCCC4)C[C@@H]1CC#N ; C=CC(=O)N1CCN(c2ncnc3cc(-c4c(C)cccc4C)c4c(c23)OCCC4)C[C@@H]1CC#N]

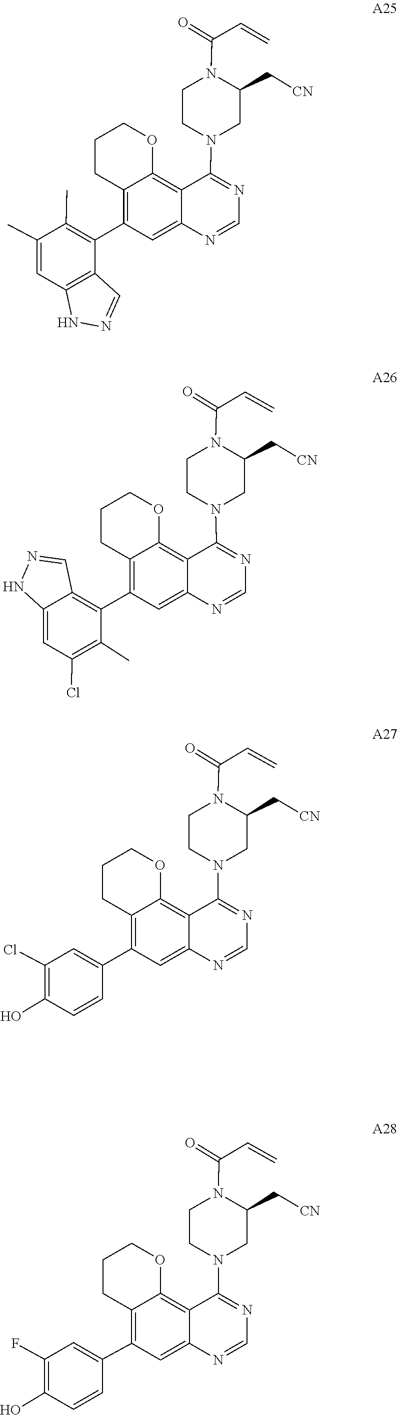 [SMILES: C=CC(=O)N1CCN(c2ncnc3cc(-c4c(C)c(C)cc5[nH]ncc45)c4c(c23)OCCC4)C[C@@H]1CC#N ; C=CC(=O)N1CCN(c2ncnc3cc(-c4c(C)c(Cl)cc5[nH]ncc45)c4c(c23)OCCC4)C[C@@H]1CC#N ; C=CC(=O)N1CCN(c2ncnc3cc(-c4ccc(O)c(Cl)c4)c4c(c23)OCCC4)C[C@@H]1CC#N ; C=CC(=O)N1CCN(c2ncnc3cc(-c4ccc(O)c(F)c4)c4c(c23)OCCC4)C[C@@H]1CC#N]

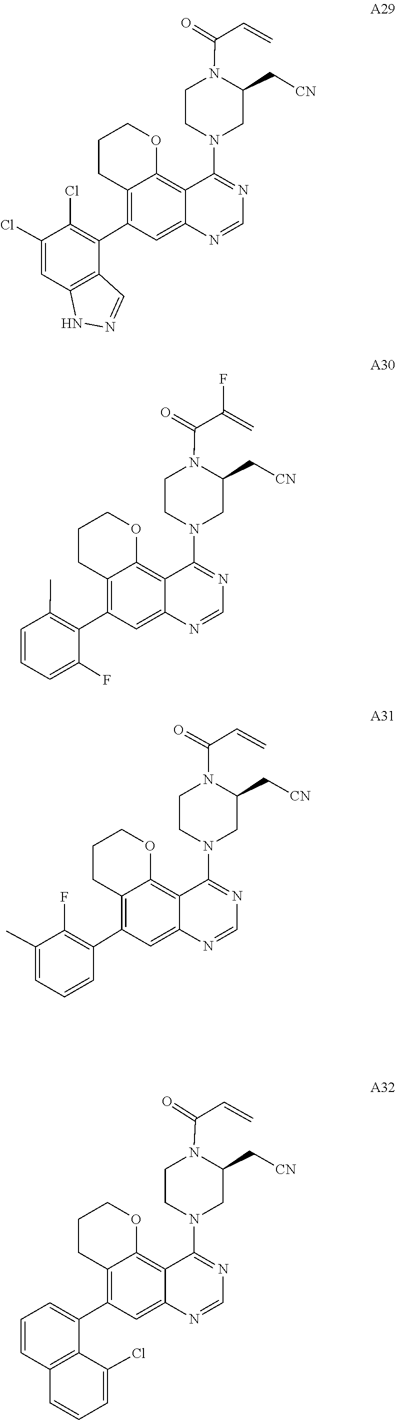 [SMILES: C=CC(=O)N1CCN(c2ncnc3cc(-c4c(Cl)c(Cl)cc5[nH]ncc45)c4c(c23)OCCC4)C[C@@H]1CC#N ; C=C(F)C(=O)N1CCN(c2ncnc3cc(-c4c(C)cccc4F)c4c(c23)OCCC4)C[C@@H]1CC#N ; C=CC(=O)N1CCN(c2ncnc3cc(-c4cccc(C)c4F)c4c(c23)OCCC4)C[C@@H]1CC#N ; C=CC(=O)N1CCN(c2ncnc3cc(-c4cccc5cccc(Cl)c45)c4c(c23)OCCC4)C[C@@H]1CC#N]

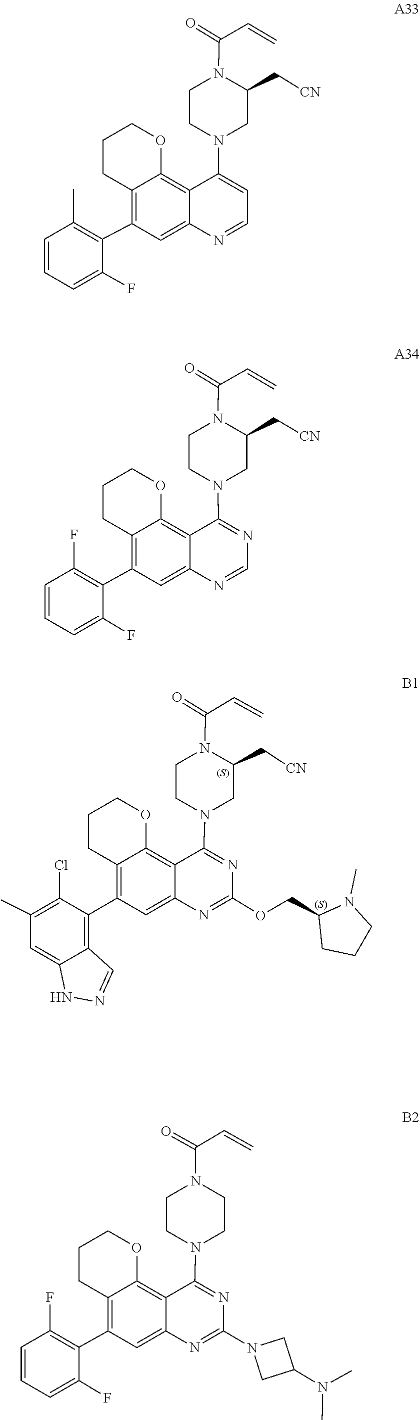

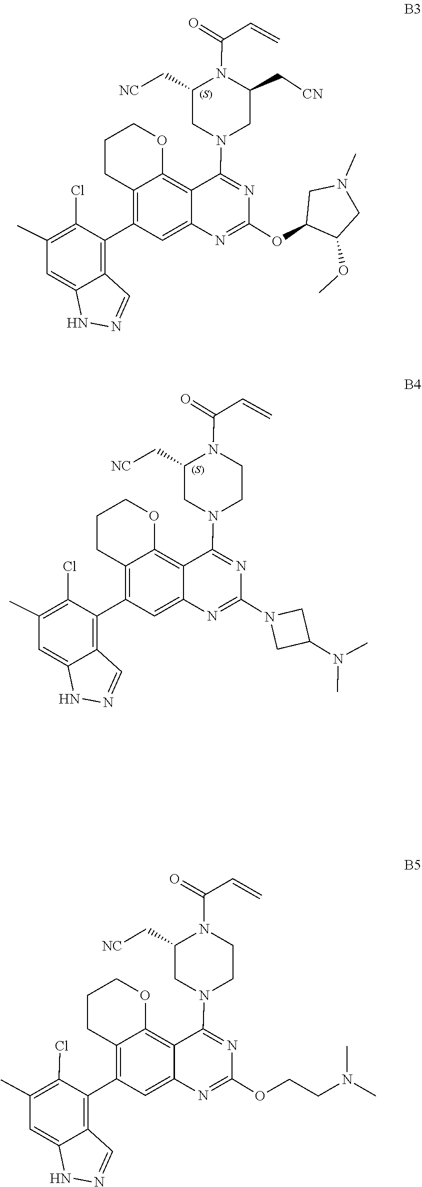

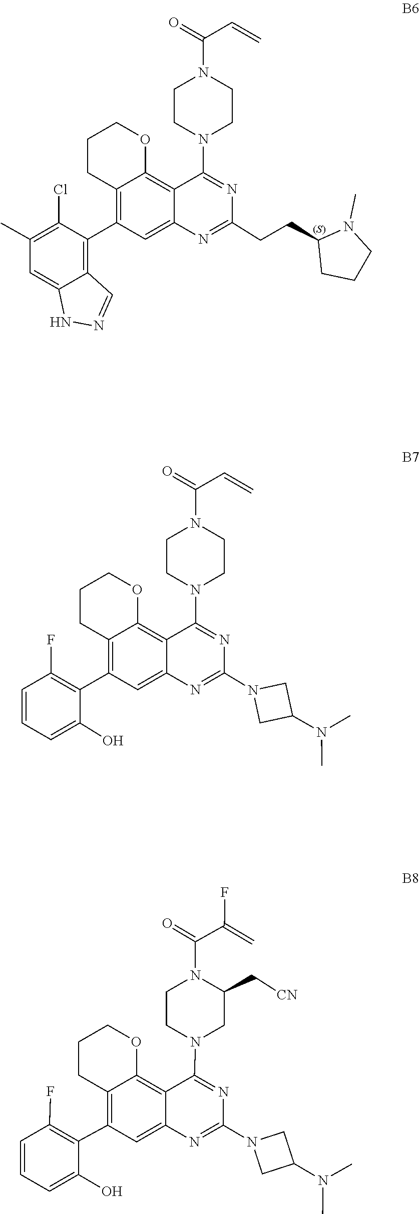

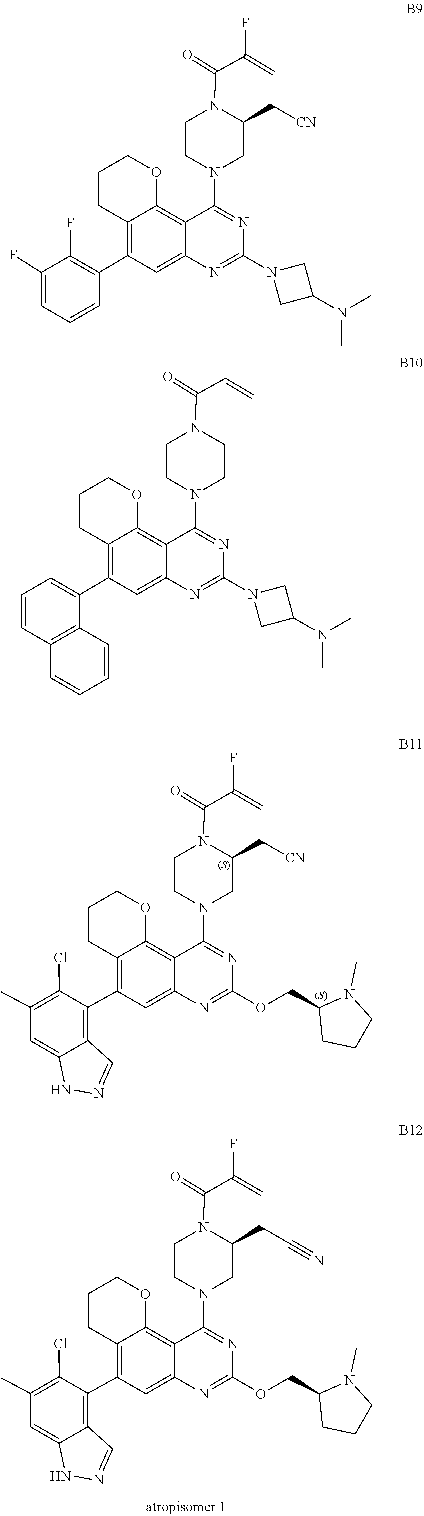

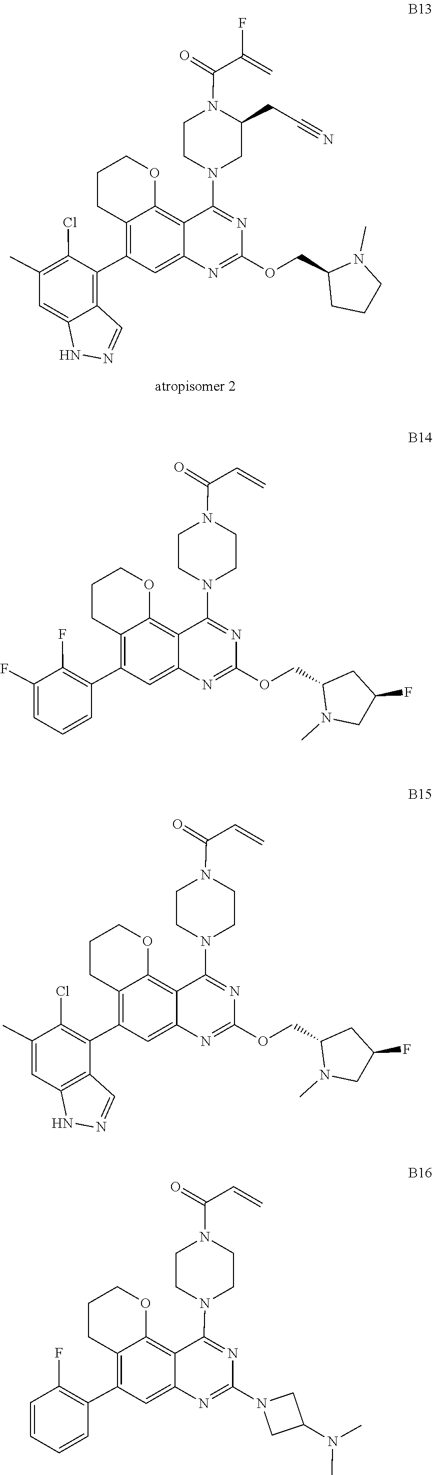 [SMILES: C=C(F)C(=O)N1CCN(c2nc(OC[C@@H]3CCCN3C)nc3cc(-c4c(Cl)c(C)cc5[nH]ncc45)c4c(c23)OCCC4)C[C@@H]1CC#N ; C=CC(=O)N1CCN(c2nc(OC[C@@H]3C[C@@H](F)CN3C)nc3cc(-c4cccc(F)c4F)c4c(c23)OCCC4)CC1 ; C=CC(=O)N1CCN(c2nc(OC[C@@H]3C[C@@H](F)CN3C)nc3cc(-c4c(Cl)c(C)cc5[nH]ncc45)c4c(c23)OCCC4)CC1 ; C=CC(=O)N1CCN(c2nc(N3CC(N(C)C)C3)nc3cc(-c4ccccc4F)c4c(c23)OCCC4)CC1]

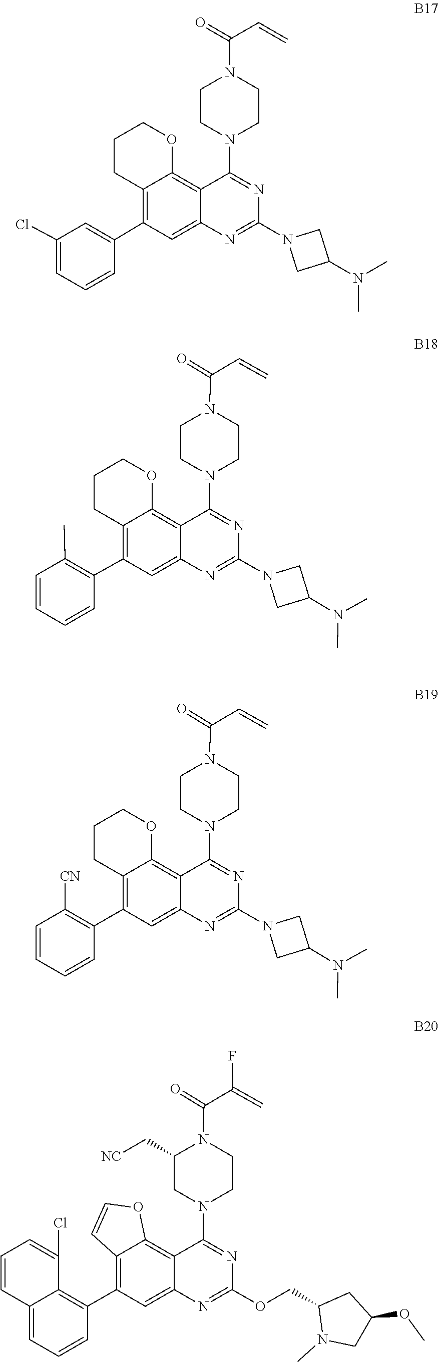 [SMILES: C=CC(=O)N1CCN(c2nc(N3CC(N(C)C)C3)nc3cc(-c4cccc(Cl)c4)c4c(c23)OCCC4)CC1 ; C=CC(=O)N1CCN(c2nc(N3CC(N(C)C)C3)nc3cc(-c4ccccc4C)c4c(c23)OCCC4)CC1 ; C=CC(=O)N1CCN(c2nc(N3CC(N(C)C)C3)nc3cc(-c4ccccc4C#N)c4c(c23)OCCC4)CC1 ; C=C(F)C(=O)N1CCN(c2nc(OC[C@@H]3C[C@@H](OC)CN3C)nc3cc(-c4cccc5cccc(Cl)c45)c4ccoc4c23)C[C@@H]1CC#N]

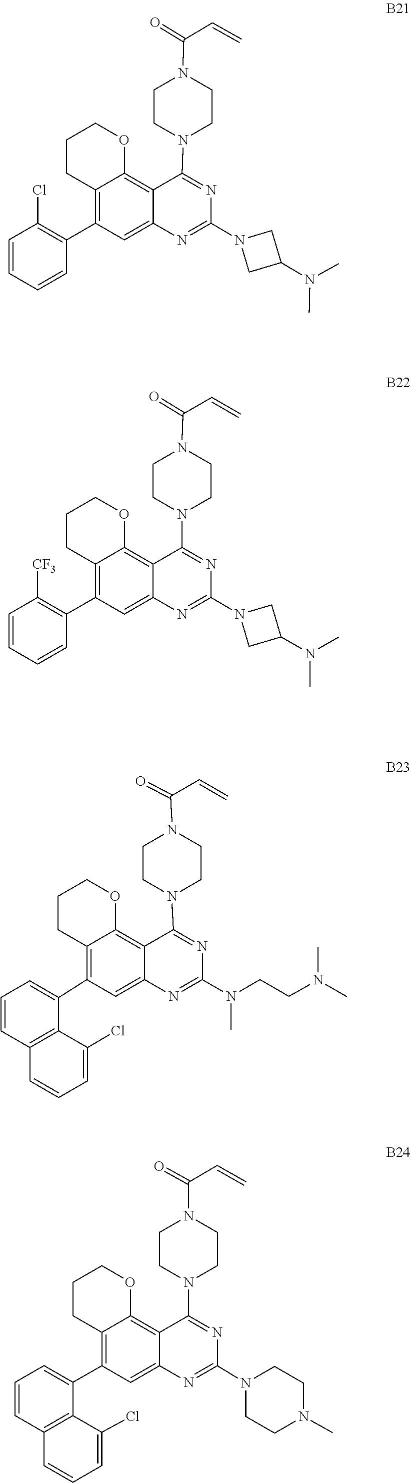 [SMILES: C=CC(=O)N1CCN(c2nc(N3CC(N(C)C)C3)nc3cc(-c4ccccc4Cl)c4c(c23)OCCC4)CC1 ; C=CC(=O)N1CCN(c2nc(N3CC(N(C)C)C3)nc3cc(-c4ccccc4C(F)(F)F)c4c(c23)OCCC4)CC1 ; C=CC(=O)N1CCN(c2nc(N(C)CCN(C)C)nc3cc(-c4cccc5cccc(Cl)c45)c4c(c23)OCCC4)CC1 ; C=CC(=O)N1CCN(c2nc(N3CCN(C)CC3)nc3cc(-c4cccc5cccc(Cl)c45)c4c(c23)OCCC4)CC1]

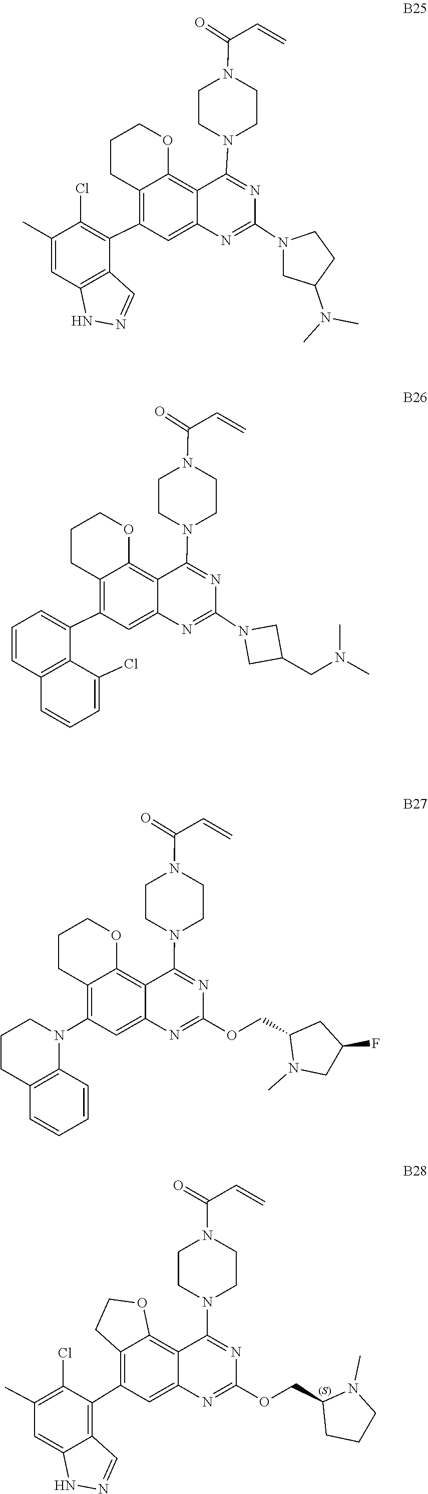

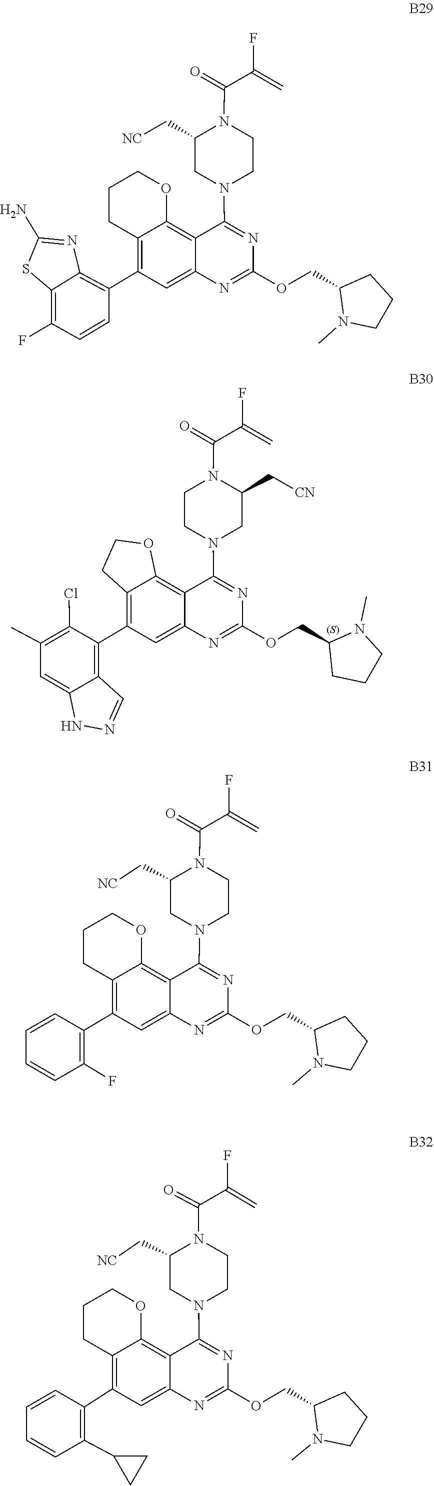

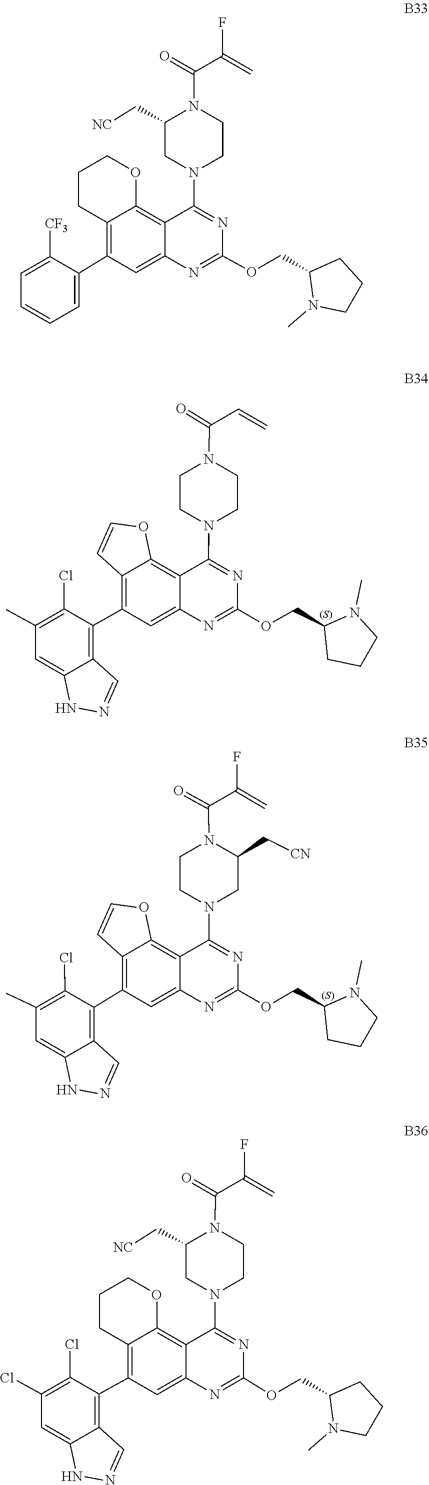

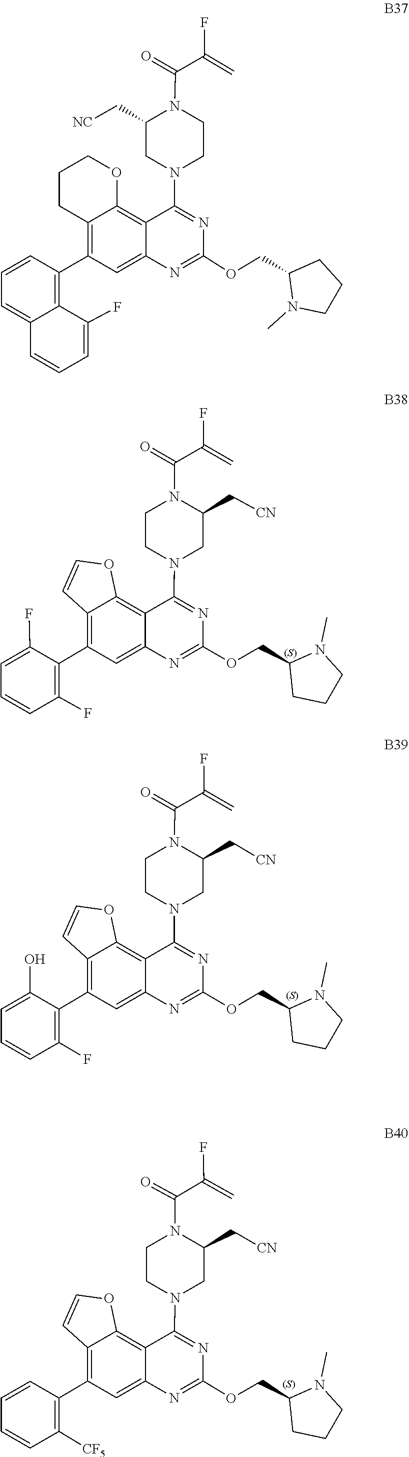

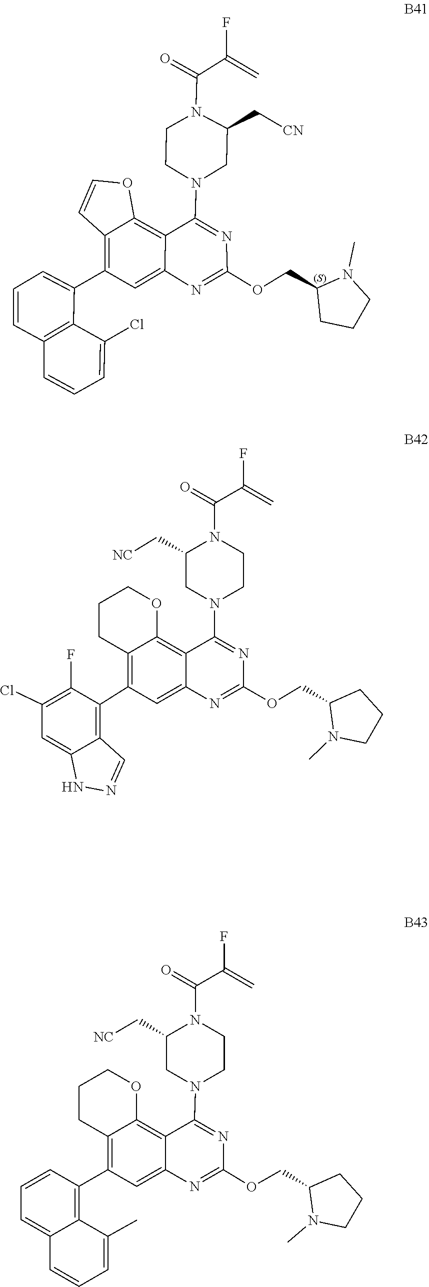

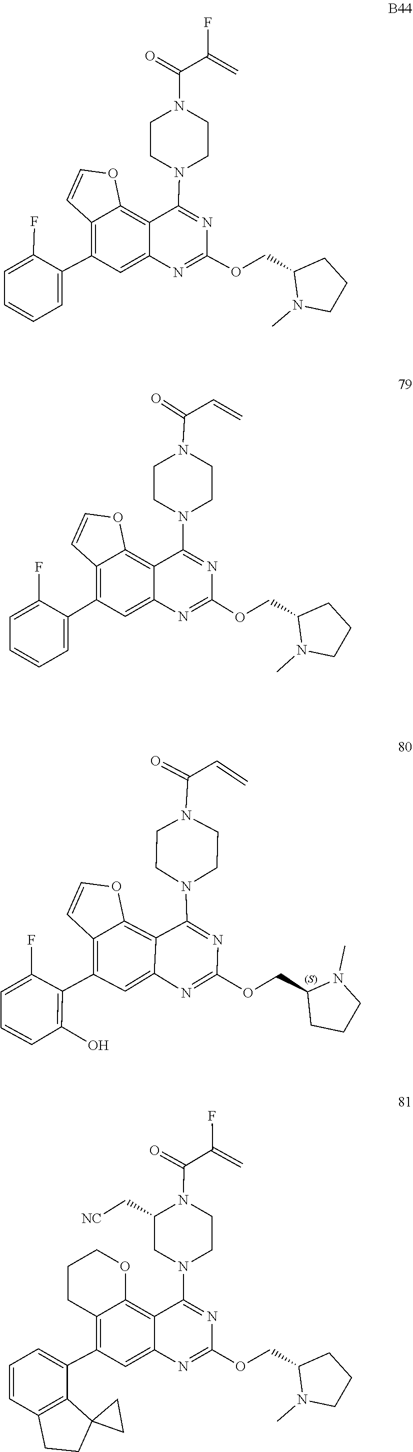

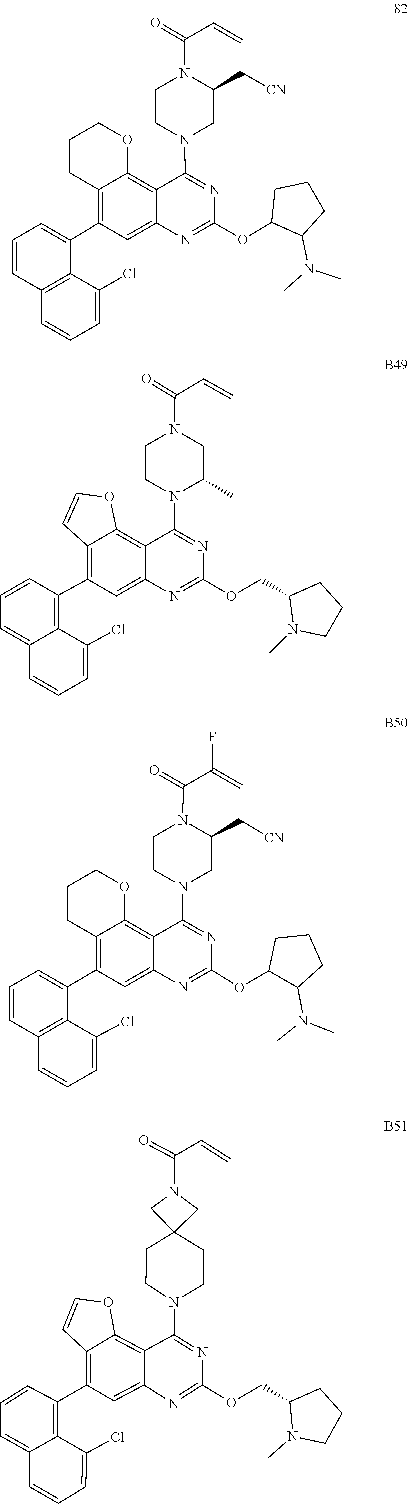 [SMILES: C=CC(=O)N1CCN(c2nc(OC3CCCC3N(C)C)nc3cc(-c4cccc5cccc(Cl)c45)c4c(c23)OCCC4)C[C@@H]1CC#N ; C=CC(=O)N1CCN(c2nc(OC[C@@H]3CCCN3C)nc3cc(-c4cccc5cccc(Cl)c45)c4ccoc4c23)[C@@H](C)C1 ; C=C(F)C(=O)N1CCN(c2nc(OC3CCCC3N(C)C)nc3cc(-c4cccc5cccc(Cl)c45)c4c(c23)OCCC4)C[C@@H]1CC#N ; C=CC(=O)N1CC2(CCN(c3nc(OC[C@@H]4CCCN4C)nc4cc(-c5cccc6cccc(Cl)c56)c5ccoc5c34)CC2)C1]

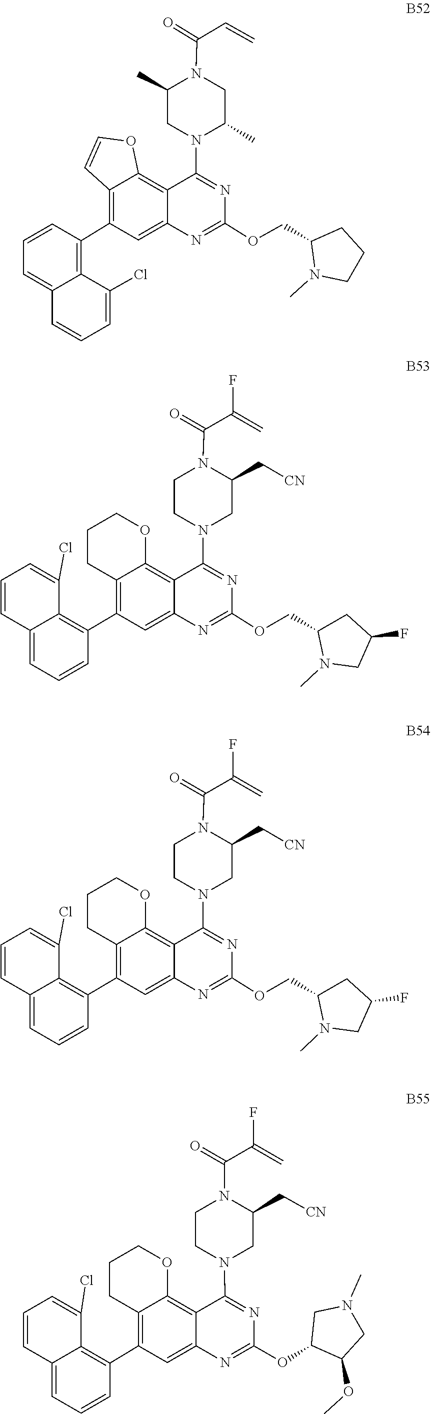 [SMILES: C=CC(=O)N1C[C@H](C)N(c2nc(OC[C@@H]3CCCN3C)nc3cc(-c4cccc5cccc(Cl)c45)c4ccoc4c23)C[C@H]1C ; C=C(F)C(=O)N1CCN(c2nc(OC[C@@H]3C[C@@H](F)CN3C)nc3cc(-c4cccc5cccc(Cl)c45)c4c(c23)OCCC4)C[C@@H]1CC#N ; C=C(F)C(=O)N1CCN(c2nc(OC[C@@H]3C[C@H](F)CN3C)nc3cc(-c4cccc5cccc(Cl)c45)c4c(c23)OCCC4)C[C@@H]1CC#N ; C=C(F)C(=O)N1CCN(c2nc(O[C@@H]3CN(C)C[C@H]3OC)nc3cc(-c4cccc5cccc(Cl)c45)c4c(c23)OCCC4)C[C@@H]1CC#N]

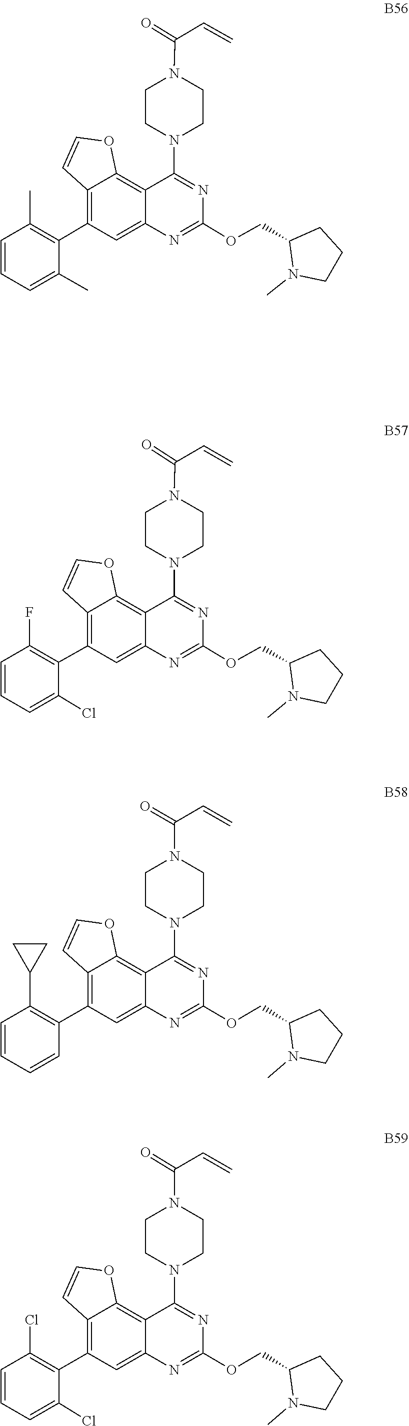 [SMILES: C=CC(=O)N1CCN(c2nc(OC[C@@H]3CCCN3C)nc3cc(-c4c(C)cccc4C)c4ccoc4c23)CC1 ; C=CC(=O)N1CCN(c2nc(OC[C@@H]3CCCN3C)nc3cc(-c4c(F)cccc4Cl)c4ccoc4c23)CC1 ; C=CC(=O)N1CCN(c2nc(OC[C@@H]3CCCN3C)nc3cc(-c4ccccc4C4CC4)c4ccoc4c23)CC1 ; C=CC(=O)N1CCN(c2nc(OC[C@@H]3CCCN3C)nc3cc(-c4c(Cl)cccc4Cl)c4ccoc4c23)CC1]

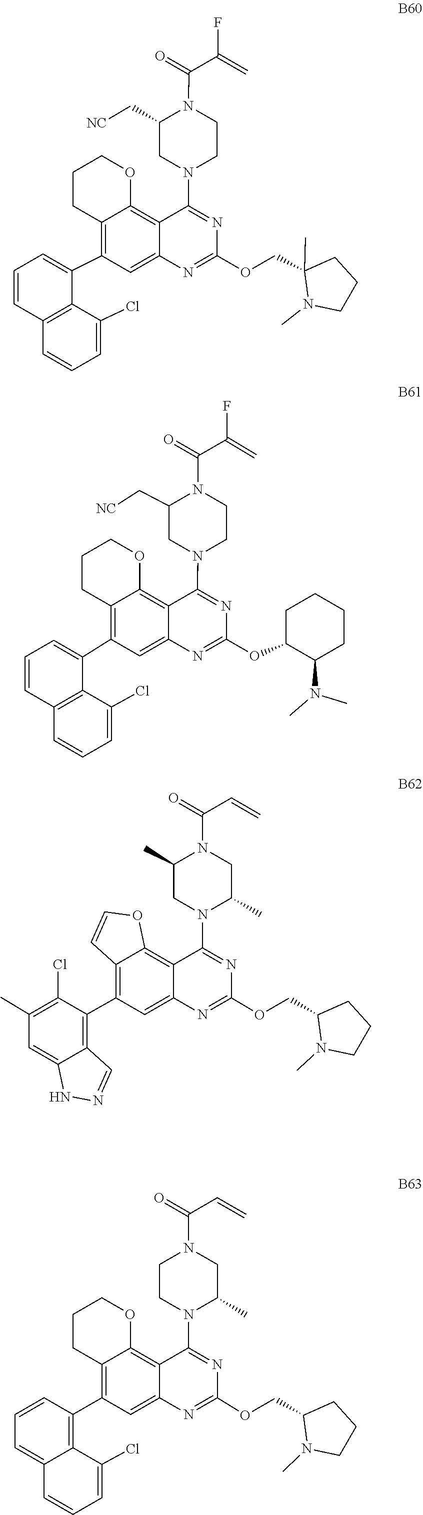

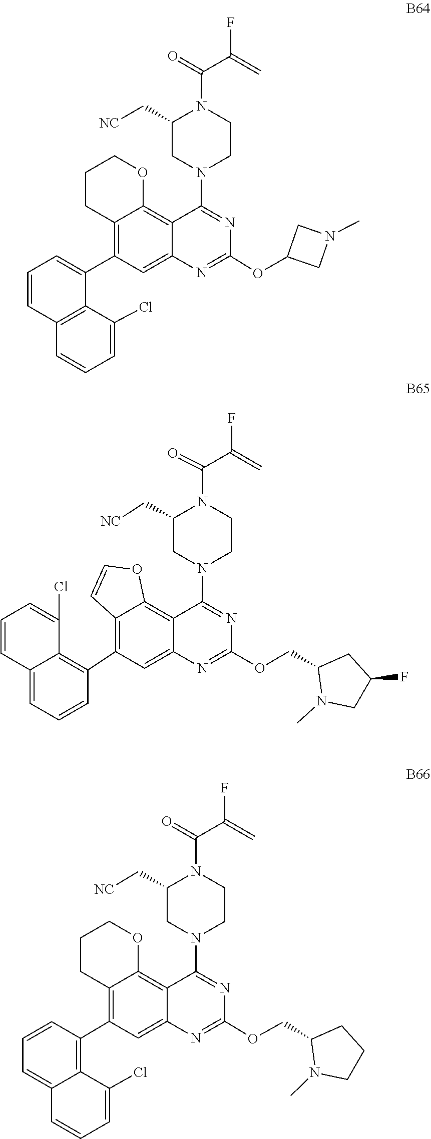 [SMILES: C=C(F)C(=O)N1CCN(c2nc(OC3CN(C)C3)nc3cc(-c4cccc5cccc(Cl)c45)c4c(c23)OCCC4)C[C@@H]1CC#N ; C=C(F)C(=O)N1CCN(c2nc(OC[C@@H]3C[C@@H](F)CN3C)nc3cc(-c4cccc5cccc(Cl)c45)c4ccoc4c23)C[C@@H]1CC#N ; C=C(F)C(=O)N1CCN(c2nc(OC[C@@H]3CCCN3C)nc3cc(-c4cccc5cccc(Cl)c45)c4c(c23)OCCC4)C[C@@H]1CC#N]

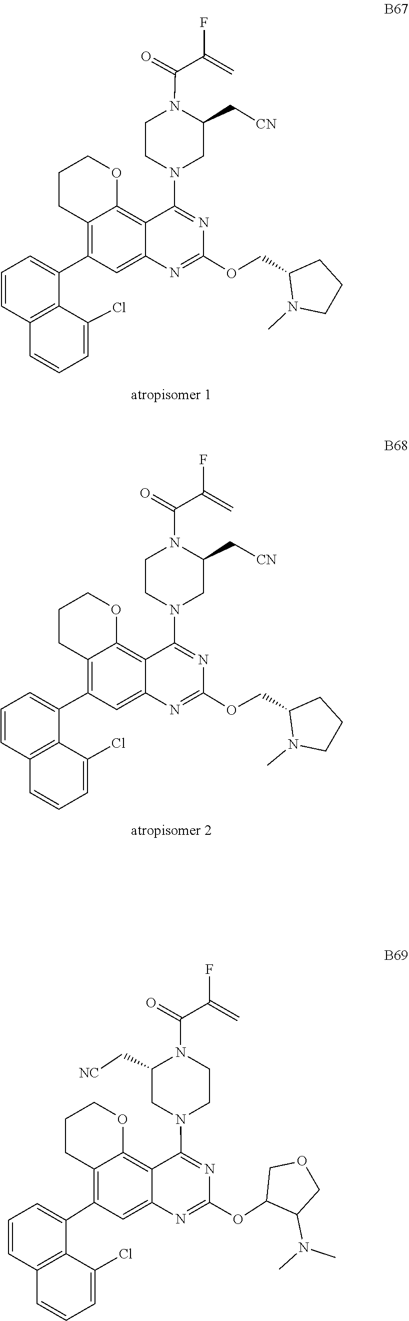 [SMILES: C=C(F)C(=O)N1CCN(c2nc(OC[C@@H]3CCCN3C)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc5cccc(Cl)c45)c4c(c23)OCCC4)C[C@@H]1CC#N ; C=C(F)C(=O)N1CCN(c2nc(OC3COCC3N(C)C)nc3cc(-c4cccc5cccc(Cl)c45)c4c(c23)OCCC4)C[C@@H]1CC#N]

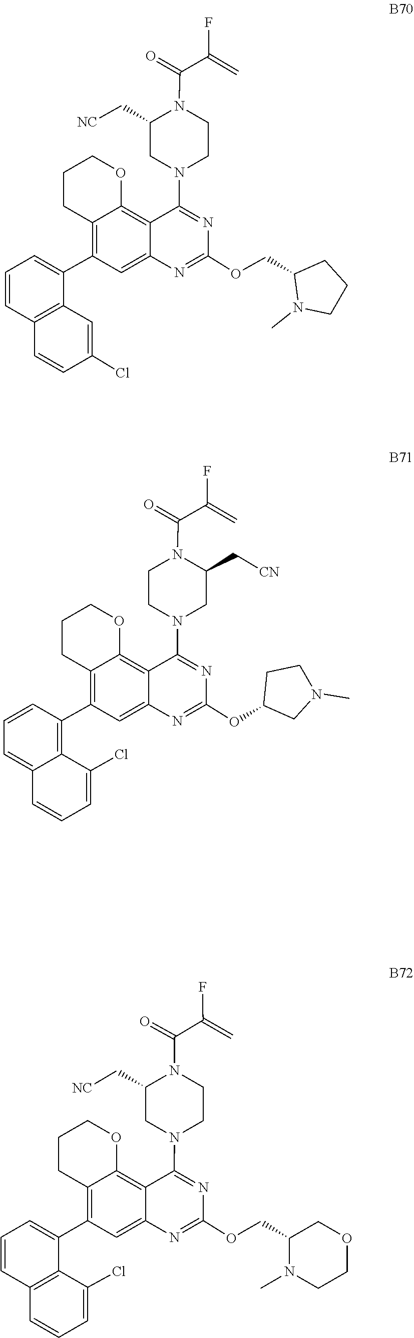 [SMILES: C=C(F)C(=O)N1CCN(c2nc(OC[C@@H]3CCCN3C)nc3cc(-c4cccc5ccc(Cl)cc45)c4c(c23)OCCC4)C[C@@H]1CC#N ; C=C(F)C(=O)N1CCN(c2nc(O[C@@H]3CCN(C)C3)nc3cc(-c4cccc5cccc(Cl)c45)c4c(c23)OCCC4)C[C@@H]1CC#N ; C=C(F)C(=O)N1CCN(c2nc(OC[C@@H]3COCCN3C)nc3cc(-c4cccc5cccc(Cl)c45)c4c(c23)OCCC4)C[C@@H]1CC#N]

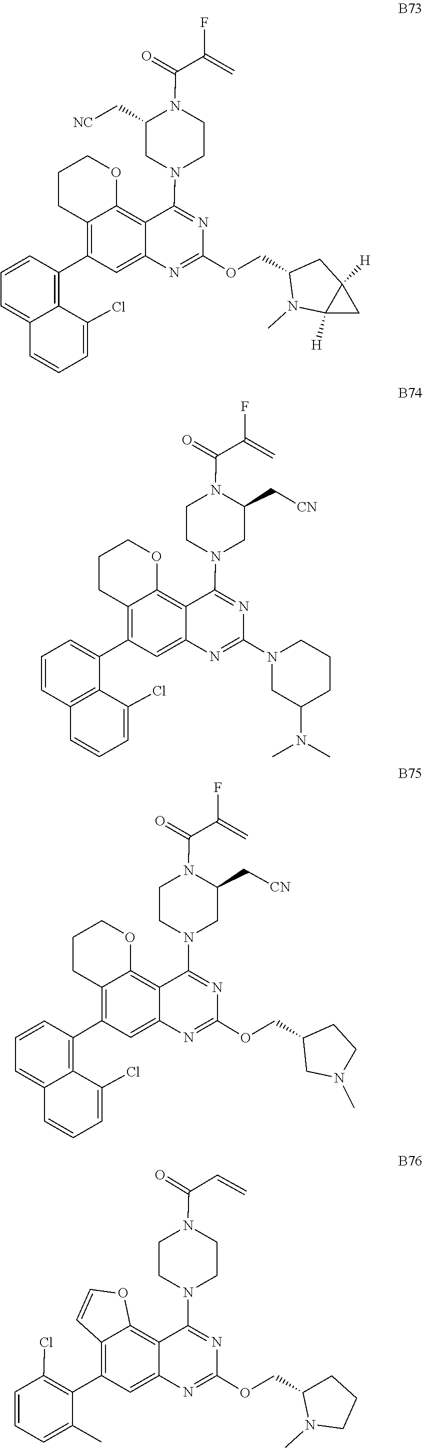 [SMILES: C=C(F)C(=O)N1CCN(c2nc(OC[C@@H]3C[C@H]4C[C@H]4N3C)nc3cc(-c4cccc5cccc(Cl)c45)c4c(c23)OCCC4)C[C@@H]1CC#N ; C=C(F)C(=O)N1CCN(c2nc(N3CCCC(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCN(C)C3)nc3cc(-c4cccc5cccc(Cl)c45)c4c(c23)OCCC4)C[C@@H]1CC#N ; C=CC(=O)N1CCN(c2nc(OC[C@@H]3CCCN3C)nc3cc(-c4c(C)cccc4Cl)c4ccoc4c23)CC1]

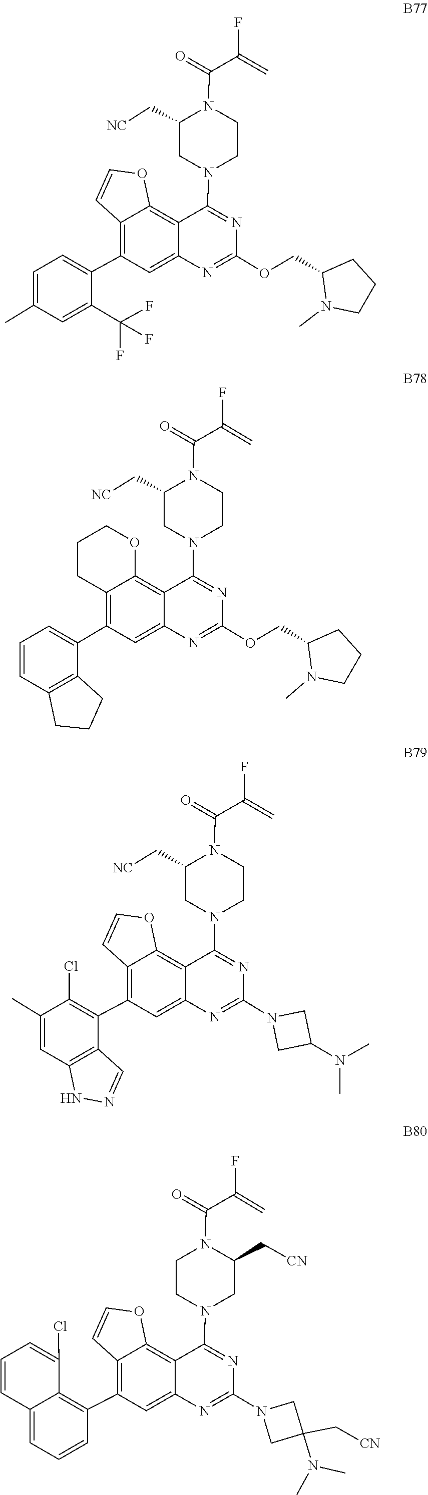

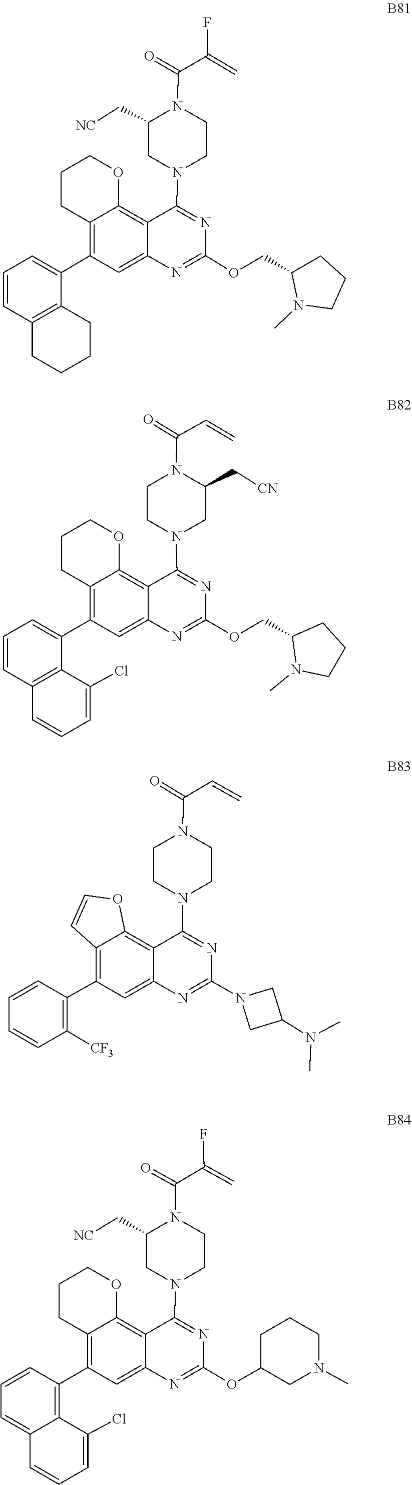 [SMILES: C=C(F)C(=O)N1CCN(c2nc(OC[C@@H]3CCCN3C)nc3cc(-c4cccc5c4CCCC5)c4c(c23)OCCC4)C[C@@H]1CC#N ; C=CC(=O)N1CCN(c2nc(OC[C@@H]3CCCN3C)nc3cc(-c4cccc5cccc(Cl)c45)c4c(c23)OCCC4)C[C@@H]1CC#N ; C=CC(=O)N1CCN(c2nc(N3CC(N(C)C)C3)nc3cc(-c4ccccc4C(F)(F)F)c4ccoc4c23)CC1 ; C=C(F)C(=O)N1CCN(c2nc(OC3CCCN(C)C3)nc3cc(-c4cccc5cccc(Cl)c45)c4c(c23)OCCC4)C[C@@H]1CC#N]

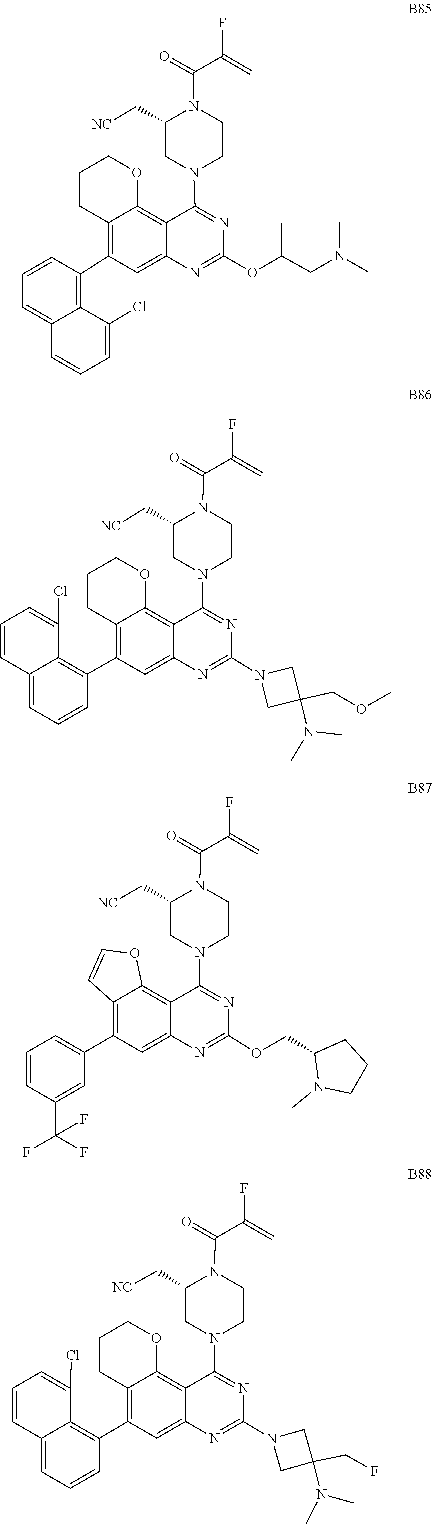 [SMILES: C=C(F)C(=O)N1CCN(c2nc(OC(C)CN(C)C)nc3cc(-c4cccc5cccc(Cl)c45)c4c(c23)OCCC4)C[C@@H]1CC#N ; C=C(F)C(=O)N1CCN(c2nc(N3CC(COC)(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc(C(F)(F)F)c4)c4ccoc4c23)C[C@@H]1CC#N ; C=C(F)C(=O)N1CCN(c2nc(N3CC(CF)(N(C)C)C3)nc3cc(-c4cccc5cccc(Cl)c45)c4c(c23)OCCC4)C[C@@H]1CC#N]

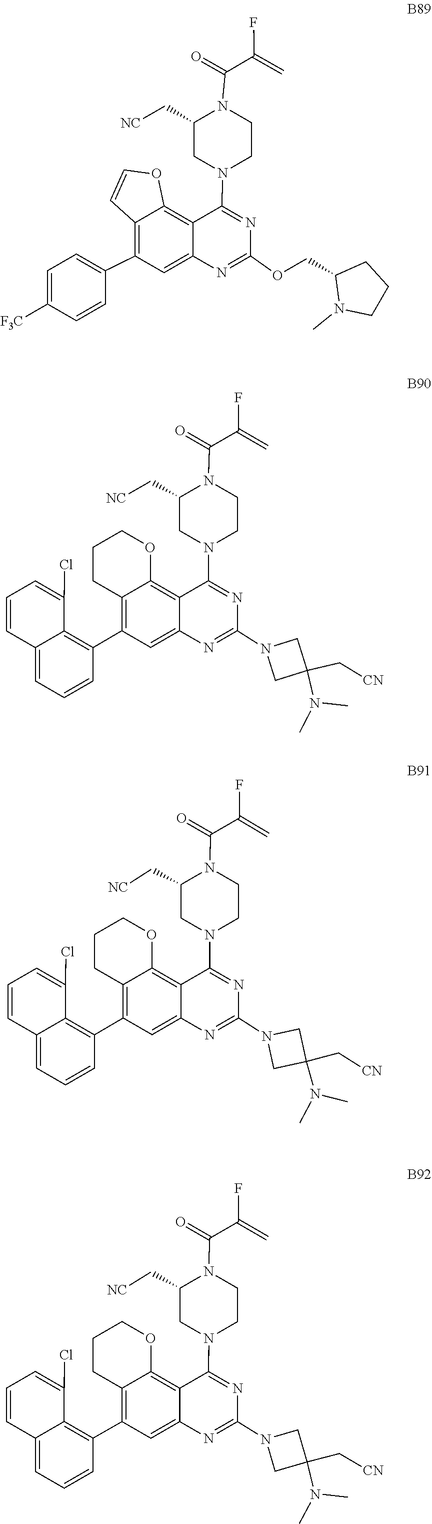 [SMILES: C=C(F)C(=O)N1CCN(c2nc(OC[C@@H]3CCCN3C)nc3cc(-c4ccc(C(F)(F)F)cc4)c4ccoc4c23)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N]

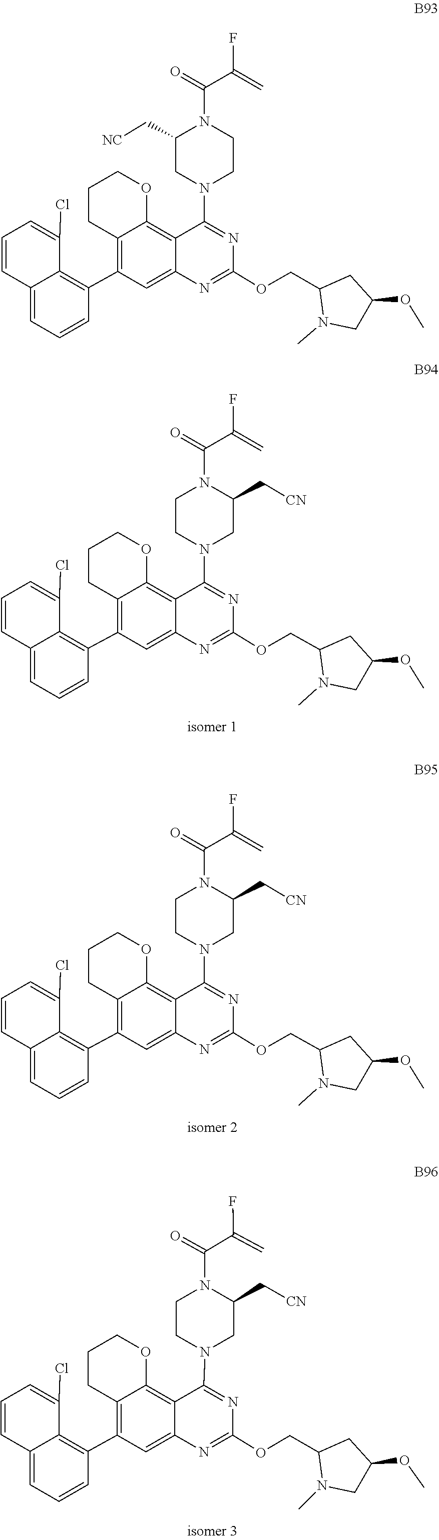 [SMILES: C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N]

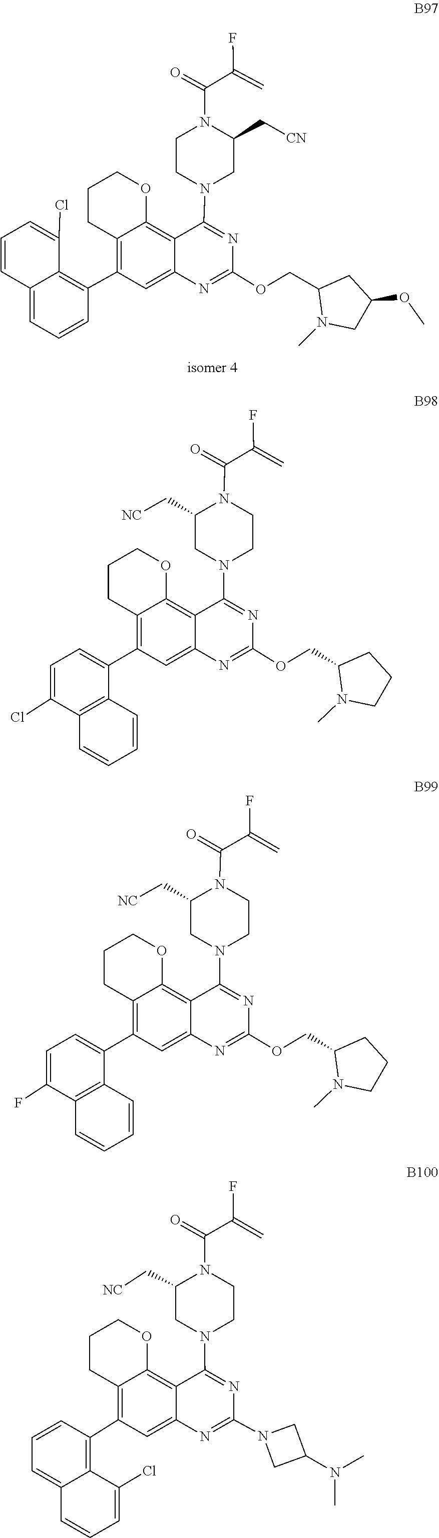 [SMILES: C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4ccc(Cl)c5ccccc45)c4c(c23)OCCC4)C[C@@H]1CC#N ; C=C(F)C(=O)N1CCN(c2nc(OC[C@@H]3CCCN3C)nc3cc(-c4ccc(F)c5ccccc45)c4c(c23)OCCC4)C[C@@H]1CC#N ; C=C(F)C(=O)N1CCN(c2nc(N3CC(N(C)C)C3)nc3cc(-c4cccc5cccc(Cl)c45)c4c(c23)OCCC4)C[C@@H]1CC#N]

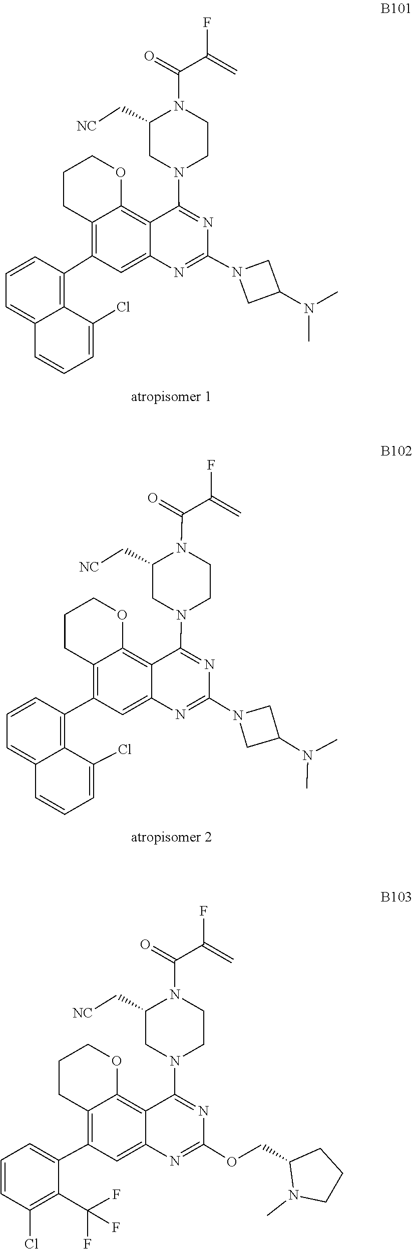 [SMILES: C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc(Cl)c4C(F)(F)F)c4c(c23)OCCC4)C[C@@H]1CC#N]

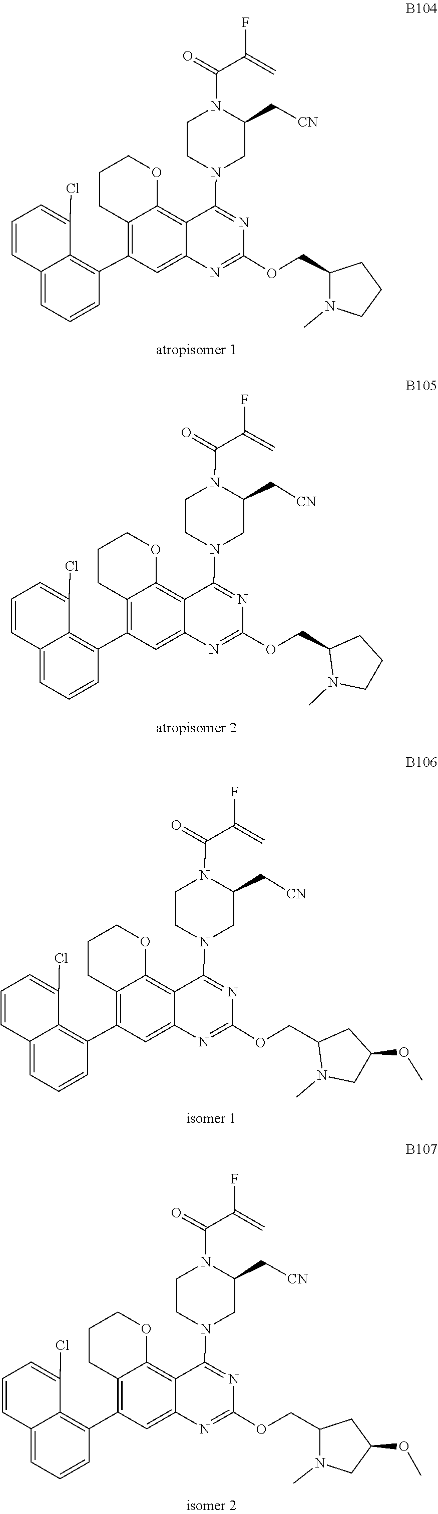 [SMILES: C=C(F)C(=O)N1CCN(c2nc(OC[C@H]3CCCN3C)nc3cc(-c4cccc5cccc(Cl)c45)c4c(c23)OCCC4)C[C@@H]1CC#N ; C=C(F)C(=O)N1CCN(c2nc(OC[C@H]3CCCN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N]

Aspect 23: A pharmaceutical composition comprising the compound disclosed herein or a stereoisomer thereof, or a pharmaceutically acceptable salt thereof and a pharmaceutically acceptable excipient.

Aspect 24: A method of treating cancer, comprising administering a subject in need thereof the compound disclosed herein or a stereoisomer thereof, or a pharmaceutically acceptable salt thereof.

### DETAILED DESCRIPTION OF THE INVENTION

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl groups. An alkyl group defined herein is optionally deuterated or tritiated.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CHCl₂, —CF₃, and the like.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1, 3-dienyl, hex-1-enyl, hex-2-enyl, hex-3-enyl, hex-4-enyl, and hexa-1, 3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from a monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4, 4], [4, 5], [5, 5], [5, 6] and [6, 6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5, 6] and [6, 6] ring systems.

The term “spiro cycloalkyl” refers to a cyclic structure which contains carbon atoms and is formed by at least two rings sharing one atom. The term “7 to 12 membered spiro cycloalkyl” refers to a cyclic structure which contains 7 to 12 carbon atoms and is formed by at least two rings sharing one atom.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 10 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl, preferably cyclohexenyl.

The term “fused cycloalkenyl” refers to a bicyclic cycloalkyl group as defined herein which contains at least one double bond and is formed by two or more rings sharing two adjacent atoms.

The term “cycloalkynyl” refers to non-aromatic cycloalkyl groups of from 5 to 10 carbon atoms having single or multiple rings and having at least one triple bond.

The term “fused cycloalkynyl” refers to a bicyclic cycloalkyl group as defined herein which contains at least one triple bond and is formed by two or more rings sharing two adjacent atoms.

The term “benzo fused cycloalkyl” is a bicyclic fused cycloalkyl in which a 4- to 8-membered monocyclic cycloalkyl ring fused to a benzene ring. For example, a benzo fused cycloalkyl is

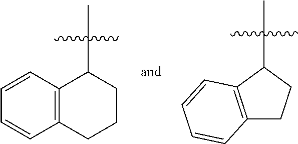 [SMILES: *C1CCCc2ccccc21 ; *C1CCc2ccccc21]

wherein the wavy lines indicate the points of attachment.

The term a “benzo fused cycloalkenyl” is a bicyclic fused cycloalkenyl in which a 4- to 8-membered monocyclic cycloalkenyl ring fused to a benzene ring.

The term a “benzo fused cycloalkynyl” is a bicyclic fused cycloalkynyl in which a 4- to 8-membered monocyclic cycloalkynyl ring fused to a benzene ring.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆cycloalkenyl, 2, 3-dihydro-1H-indenyl, 1H-indenyl, 1, 2, 3, 4-tetralyl, dihydroindenyl (e.g., 2,3-dihydro-1H-indene), dihydronaphthyl (e.g., 1, 4-dihydronaphthyl), dihydroacenaphthylenyl, etc. Preferred embodiments are a 8 to 9 membered fused ring, which refer to cyclic structures containing 8 to 9 ring atoms within the above examples.

The term “aryl” used alone or in combination with other terms refers to a group selected from:
a) 5- and 6-membered carbocyclic aromatic rings, e.g., phenyl;
b) bicyclic ring systems such as 7- to 12-membered bicyclic ring systems, wherein at least one ring is carbocyclic and aromatic, e.g., naphthyl and indanyl; and,
c) tricyclic ring systems such as 10- to 15-membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” refers to a bicyclic aryl ring as defined herein. The typical bicyclic fused aryl is naphthalene.

The term “heteroaryl” refers to a group selected from:
a) 5-, 6-, 7- or 8-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
b) 7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
c) 11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring (s) of the heteroaryl group can be oxidized to form N-oxides.

Specifically, the term “bicyclic fused heteroaryl” refers to a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

Representative examples of bicyclic fused heteroaryl include, but not limited to, the following groups benzisoxazolyl, benzodiazolyl, benzofuranyl, benzofurazanyl, benzofuryl, benzoimidazolyl, benzoisothiazolyl, benzothiadiazolyl, benzothiazolyl, benzothienyl, benzothiophenyl, benzotriazolyl, benzoxadiazolyl, benzoxazolyl, furopyridinyl, furopyrrolyl, imidazopyridinyl, imidazopyridyl, imidazothiazolyl, indazolyl, indolizinyl, indolyl, isobenzofuryl, isoindolyl, isoquinolinyl (or isoquinolyl), naphthyridinyl, phthalazinyl, pteridinyl, purinyl, pyrazinopyridazinyl, pyrazolopyridinyl, pyrazolopyrimidinyl, pyrazolopyridyl, pyrazolotriazinyl, pyridazolopyridyl, pyrrolopyridinyl, quinazolinyl, quinolinyl (or quinolyl), quinoxalinyl, thiazolopyridyl, thienopyrazinyl, thienopyrazolyl, thienopyridyl, thienopyrrolyl, thienothienyl, or triazolopyridyl.

The term a “benzo fused heteroaryl” is a bicyclic fused heteroaryl in which a 5- to 7-membered (preferably, 5- or 6-membered) monocyclic heteroaryl ring as defined herein fused to a benzene ring.

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2, 4-pyrimidinyl, 3, 5-pyrimidinyl, 2, 4-imidazolyl, imidazopyridinyl, isoxazolyl, oxazolyl, thiazolyl, isothiazolyl, thiadiazolyl (such as 1, 2, 3-thiadiazolyl, 1, 2, 4-thiadiazolyl, or 1, 3, 4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1, 2, 3-oxadiazolyl, 1, 2, 4-oxadiazolyl, or 1, 3, 4-oxadiazolyl), phthalazinyl, pyrazinyl, pyridazinyl, pyrrolyl, triazolyl (such as 1, 2, 3-triazolyl, 1, 2, 4-triazolyl, or 1, 3, 4-triazolyl), quinolinyl, isoquinolinyl, pyrazolyl, pyrrolopyridinyl (such as 1H-pyrrolo[2, 3-b]pyridin-5-yl), pyrazolopyridinyl (such as 1H-pyrazolo[3, 4-b]pyridin-5-yl), benzoxazolyl (such as benzo[d]oxazol-6-yl), pteridinyl, purinyl, 1-oxa-2, 3-diazolyl, 1-oxa-2, 4-diazolyl, 1-oxa-2, 5-diazolyl, 1-oxa-3, 4-diazolyl, 1-thia-2, 3-diazolyl, 1-thia-2, 4-diazolyl, 1-thia-2, 5-diazolyl, 1-thia-3, 4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl), or carbazolyl (9H-carbazolyl).

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “optionally oxidized sulfur” used herein refer to S, SO or SO₂.

The term “monocyclic heterocyclyl” refers to monocyclic groups in which at least one ring member (e.g., 1-3 heteroatoms, 1 or 2 heteroatoms) is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2, 5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1, 2-dithietanyl, 1, 3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1, 4-oxathianyl, 1, 4-dioxepanyl, 1, 4-oxathiepanyl, 1, 4-oxaazepanyl, 1, 4-dithiepanyl, 1, 4-thiazepanyl and 1, 4-diazepanyl, 1, 4-dithianyl, 1, 4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1, 4-dioxanyl, 1, 3-dioxolanyl, pyrazolinyl, pyrazolidinyl, dithianyl, dithiolanyl, pyrazolidinyl, imidazolinyl, pyrimidinonyl, or 1, 1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/3-membered, 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include, but not limited to the following groups: 2, 3-dihydrospiro[indene-1, 2′-pyrrolidine] (e.g., 2, 3-dihydrospiro[indene-1, 2′-pyrrolidine]-1′-yl), 1, 3-dihydrospiro[indene-2, 2′-pyrrolidine] (e.g., 1, 3-dihydrospiro[indene-2, 2′-pyrrolidine]-1′-yl), azaspiro[2.4]heptane (e.g., 5-azaspiro[2.4]heptane-5-yl), 2-oxa-6-azaspiro[3.3]heptane (e.g., 2-oxa-6-azaspiro[3.3]heptan-6-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1, 7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl is divided into bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered, 6-membered/6-membered, or 6-membered/7-membered bicyclic fused heterocyclyl. Representative examples of (bicyclic) fused heterocycles include, but not limited to, the following groups octahydrocyclopenta[c]pyrrole, octahydropyrrolo[3, 4-c]pyrrolyl, octahydroisoindolyl, isoindolinyl, octahydro-benzo[b][1, 4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl (e.g., 2,3-dihydrobenzofuran or 1,3-dihydrobenzofuran), dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxezinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, tetrahydropyrazolopyrimidinyl (e.g., 4, 5, 6, 7-tetrahydropyrazolo[1, 5-a]pyrimidin-3-yl), or benzoisoquinolinyl (e.g., 2,3-dihydro-1H-benzo[de]isoquinolinyl).

The term “benzo fused heterocyclyl” is a bicyclic fused heterocyclyl in which a monocyclic 4 to 9-membered heterocyclyl as defined herein (preferably 5- or 6-membered) fused to a benzene ring.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

The term “at least one substituents” disclosed herein includes, for example, from 1 to 4, such as from 1 to 3, further as 1 or 2, substituents, provided the theory of valence is met. For example, “at least one substituents R^{6d}” disclosed herein includes from 1 to 4, such as from 1 to 3, further as 1 or 2, substituents selected from the list of R^{6d} as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer (s). In some embodiments, the term “substantially pure” means that the target stereoisomer contains no more than 10%, for example, no more than 5%, such as no more than 1%, by weight of any other stereoisomer (s).

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller C. H., et al. “Chromatographic resolution of enantiomers: Selective review.” J. Chromatogr, 113 (3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer Irving W, Ed. Drug Stereochemistry: Analytical Methods and Pharmacology. New York: Marcel Dekker Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

The term “drop” in the synthesis method refers to 0.01 ml-0.1 ml, further such as 0.02 ml, 0.03 ml, 0.04 ml, 0.05 ml, 0.06 ml, 0.07 ml, 0.08 ml, 0.09 ml. In some embodiments, the term “drop” refers to 0.04 ml, 0.05 ml, 0.06 ml.

The term “a little amount” in the synthesis method refers the catalytic amount. In some embodiments, the term “a little amount” refers to 3%, 5%, 10%, 15% weight ratio of the main reactant.

The term “substantially pure” as used herein means that the target stereoisomer contains no more than 35%, such as no more than 30%, further such as no more than 25%, even further such as no more than 20%, by weight of any other stereoisomer (s). In some embodiments, the term “substantially pure” means that the target stereoisomer contains no more than 10%, for example, no more than 5%, such as no more than 1%, by weight of any other stereoisomer (s).

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

Preparative HPLC was conducted on any commercially available column (e.g., 150×21.2 mm ID, 5 pm, Gemini NXC 18, Waters Xselect CSH C18, or Waters Xbridge C18) at a flow rate of 5-20 ml/min, injection volume of 1-2 ml, at room temperature and UV Detection at 214 nm and 254 nm. Mobile phase A is ACN (optionally with 0.1% FA); Mobile phase B is water (optionally with 0.1% FA or 0.03% NH₃·H₂O). Gradient Table: Mobile Phase A (20%-90%, 30%-90%, 40%-90% or 50%-90%), Time (min): 0-15 min, 0-17 min, or 0-20 min.

Chiral analytic HPLC was used for the retention time analysis of different chiral examples, the conditions were divided into the methods as below according to the column, mobile phase, solvent ration used.

Preparative TLC was conducted on any commercially preparative TLC plate. Mobile phase A is PE or DCM (optionally with 0.1% FA); Mobile phase B is EA or MeOH. Gradient Table: Mobile Phase A (0-100%, 20%-90%, 30%-90%, 40%-90% or 50%-90%).

The compounds disclosed herein, and/or the pharmaceutically acceptable salts thereof, can be synthesized from commercially available starting materials taken together with the disclosure herein. The following scheme illustrates methods for preparation of some of the compounds disclosed herein.

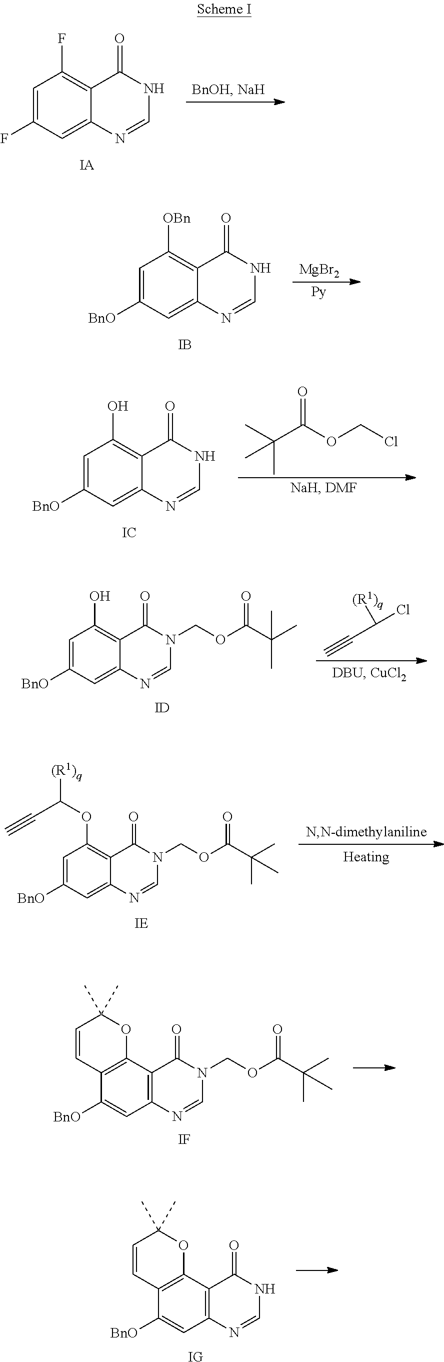

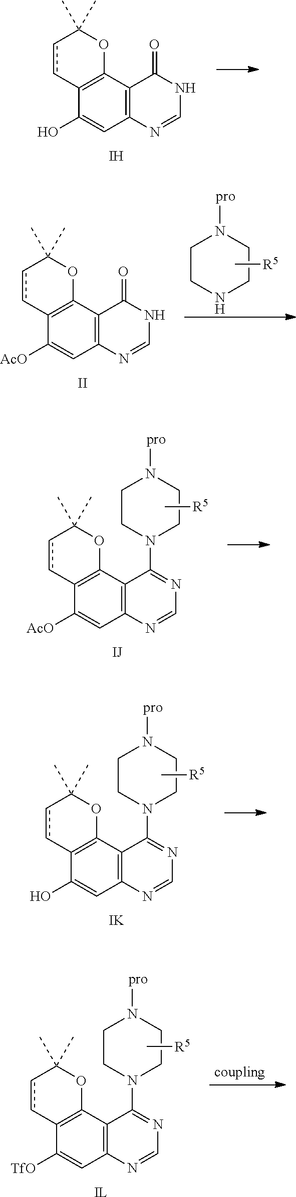

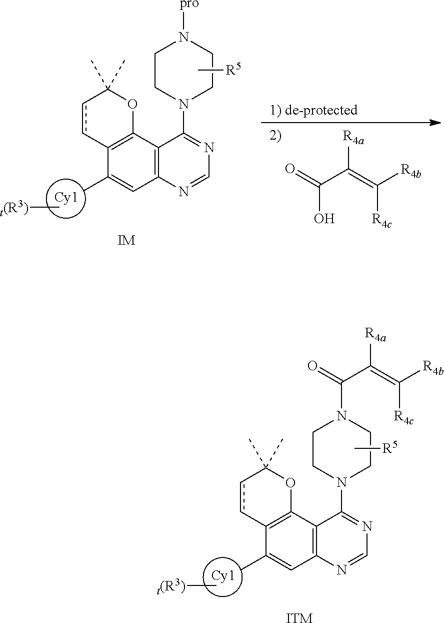

wherein R^{4a}, R^{4b}, R^{4c}, (R¹) q, Cy1 and t(R³) are as defined for the compound of Formula (I).

Compounds of general formula of ITM can be prepared according to the scheme I. Two fluorine atoms of the Formula IA are replaced by benzyloxyl group in the presence of a base to give Formula IB. One of the benzyl group of formula IB is removed selectively using magnesium bromide and pyridine to give Formula IC and then the amine group is protected with methyl pivalate to form formula ID, which is reacted with Substituted Alkynes and CuCl₂ or CuCl under basic conditions to give Formula IE and then heated in the presence of N,N-dimethylaniline to give formula IF. the protected group of the amine group is removed to form formula IG. Formula IG is reduced to form formula IH. Using the protection-deprotection sequence formula II is obtained, which is then react with different substituted piperazine in the presence of pyBOP and base to form formula IJ. II also can be chloridized using phosphoryl trichloride or sulfurous dichloride and reacted with different substituted piperazine with or without base to form IJ. The protected group of the hydroxyl group such as the actyl group is removed to form IK. Then form the key formula triflate IL in the presence of 1,1,1-trifluoro-N-phenyl-N-((trifluoromethyl)sulfonyl)methanesulfonamide and base. The formula IL can react with different Arylboric acid or Arylborates in the metal catalyzed coupling reaction conditions to form IM. Then remove the protective group and react with different substituted acryloyl chloride with base or react with different substituted acryloyl acid in presence of T3P with base to form target formula ITM.

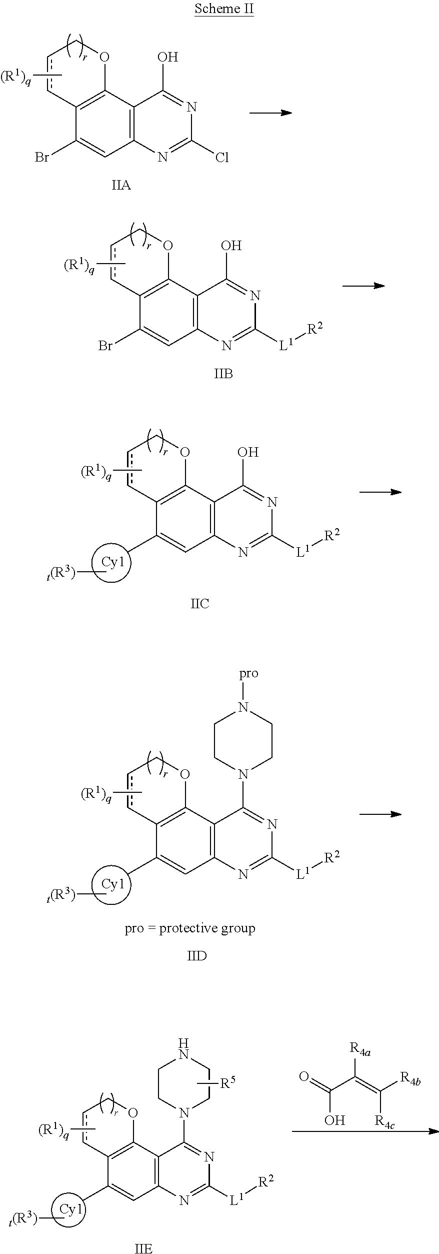

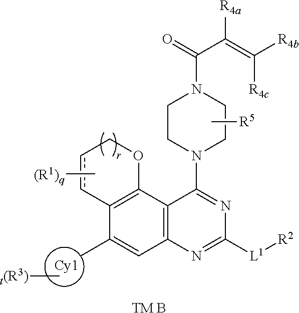

wherein R^{4a}, R^{4b}, R^{4c}, (R¹) q, r, L¹, R², R⁵, Cy1 and t(R³) are as defined for the compound of Formula (I).

Compounds of general formula of TM B can be prepared according to the scheme II. The formula IIA is formed as in the scheme IV or V. The Chlorine atom of the formula IIA is replaced by R²L1H in the presence of a base to give Formula IIB. The formula IIB can react with different Arylboric acid or Arylborates in the metal catalyzed coupling reaction conditions to form IIC, which is then react with different substituted piperazine in the presence of pyBOP and base to form formula IID. IIC also can be chloridized using phosphoryl trichloride or sulfurous dichloride and reacted with different substituted piperazine with or without base to form IID. Then remove the protective group of formula IID and react with different substituted acryloyl chloride with base or react with different substituted acryloyl acid in presence of T3P with base to form target formula TM B.

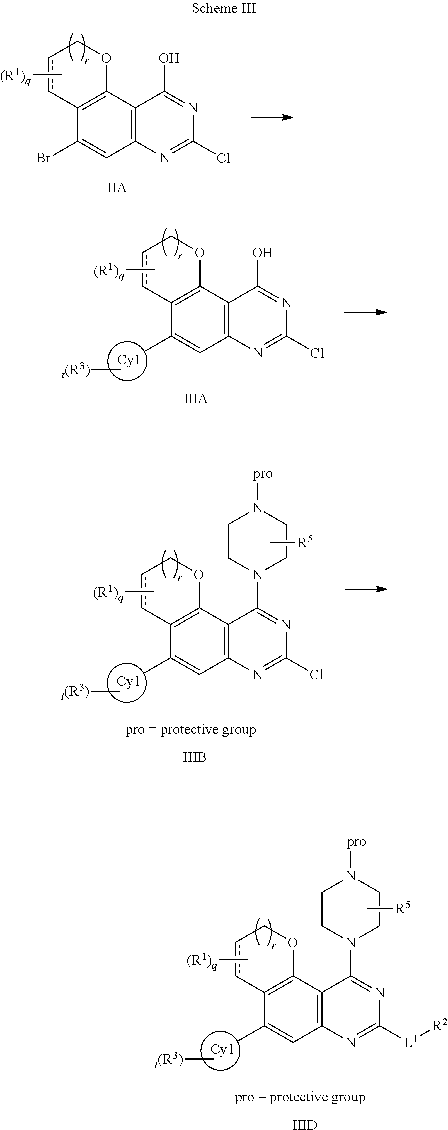

wherein (R¹) q, r, L¹, R², R⁵, Cy1 and t(R³) are as defined for the compound of Formula (I).

The formula IID also can be prepared according to the scheme III, The formula IIA is formed as in the scheme IV or V. The formula IIA can react with different Arylboric acid or Arylborates in the metal catalyzed coupling reaction conditions to form IIIA, which is then react with different substituted piperazine in the presence of pyBOP and base to form formula IIIB, which is replaced by R²L1H in the presence of a base to give Formula IID.

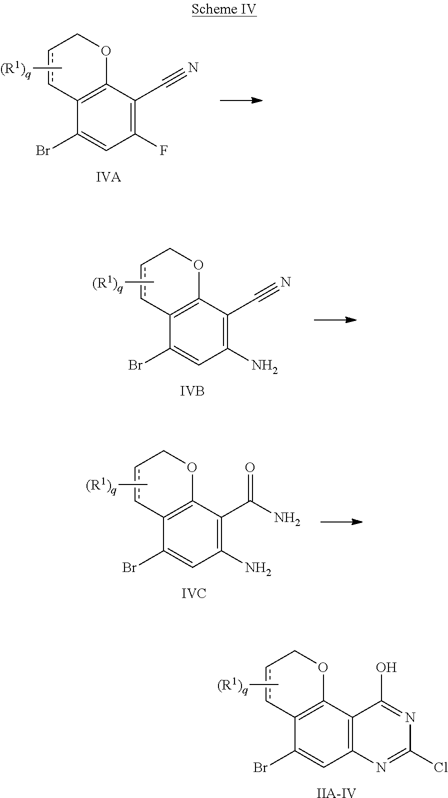

wherein r is 1, (R¹) q are as defined for the compound of Formula (I).

The formula IIA-IV can be prepared according to the scheme IV. Formula IVA is replaced by NH2 to form formula IVB, which is oxidized to afford the formula IVC. The IVC is converted to the formula IIA-IV in the presence of thiophosgene.

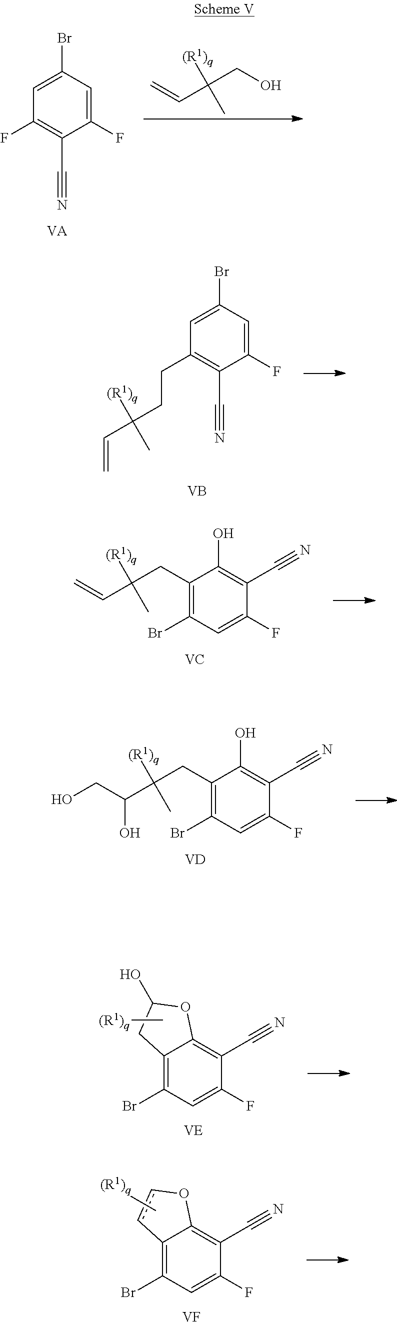

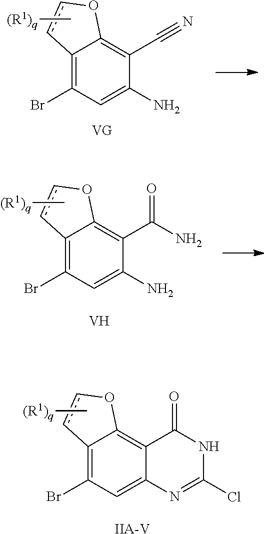

wherein r is 0, (R¹) q are as defined for the compound of Formula (I).

The formula IIA-V can be prepared according to the scheme V. VA is replaced by different Allyl alcohol to afford the formula VB, which is treated with lewis acid to form the formula VC. VC is oxidized to form the formula VD, and further to oxidize to convert formula VE, which is dehydrated or reduced to form VF. VF is replaced by NH2 to form VG, which is oxidized to afford the formula VH. The formula VH is converted to formula IIA-V in the presence of thiophosgene.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄. ¹H NMR spectra are recorded on Bruck-400 nuclear magnetic resonance spectrometer with TMS (tetramethylsilane) as the internal standard. LC/MS data are recorded by using Agilent1100 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw@.

In the following examples, the following abbreviations are used:
AcOH Acetic acid
Aq. Aqueous
BINAP 2,2′-bis(diphenylphosphino)-1,1′-binaphthalene
Brine Saturated aqueous sodium chloride solution
Bn Benzyl
BnBr Benzyl Bromide
Boc Tert-butoxycarbonyl
CH₂Cl₂ or DCM Dichloromethane
DIAD Diisopropyl azodicarboxylate
DMF N,N-Dimethylformamide
Dppf 1,1′-bis(diphenylphosphino)ferrocene
DBU 1,8-diazabicyclo[5.4.0]undec-7-ene
DHP 3,4-Dihydro-2H-pyran
DIEA or DIPEA N,N-diisopropylethylamine
DMAP 4-N,N-dimethylaminopyridine
DMB (2,4-dimethoxyphenyl)methanamine
DMF N,N-dimethylformamide
DMSO Dimethyl sulfoxide
EtOAc or EA Ethyl acetate
EtOH Ethanol
Et₂O or ether Diethyl ether
g Grams
h or hr Hour
HATU O-(7-Azabenzotriazol-1-yl)-N,N,N′,N′-tetramethyluronium hexafluorophosphate
Hex Hexane
HCl Hydrochloric acid
HMDS Hexamethyldisilazane
HPLC High-performance liquid chromatography
i-PrOH Isopropyl alcohol
LCMS Liquid chromatography-mass spectrometry
mg Milligrams
mL Milliliters
mmol Millimole
MeCN Acetonitrile
MeOH Methanol
Min Minutes
ms or MS Mass spectrum
MTBE methyl tert-butyl ether
Na₂SO₄ Sodium sulfate
NBS N-Bromosuccinimide
NMO 4-Methylmorpholine N-oxide
NMP N-Methyl Pyrrolidone
PE petroleum ether
PMB (4-methoxyphenyl)methanamine
POCl3 phosphorous oxychloride
PyBOP Benzotriazol-1-yl-oxytripyrrolidinophosphonium hexafluorophosphate
PddppfCl2 [1,1′-Bis(diphenylphosphino)ferrocene]dichloropalladium(II)
Prep Preparative
PTSA 4-Methylbenzenesulfonic acid
Rt or rt Room temperature
sat. Saturated
TBSCI tert-Butyldimethylsilyl chloride
t-BuOK Potassium tert-butoxide
T3P n-Propylphosphonic cyclic anhydride
TFA Trifluoroacetic acid
THF Tetrahydrofuran
TLC thin layer chromatography
μL Microliters

### EXAMPLES

### Compound A1: 1-(4-(2,2-dimethyl-5-(naphthalen-1-yl)-3,4-dihydro-2H-pyrano[2,3-f]quinazolin-10-yl)piperazin-1-yl)prop-2-en-1-one

### Step A: (Z)—N-(3,5-difluorophenyl)-2-(hydroxyimino)acetamide

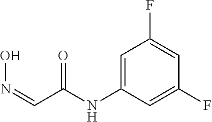 [SMILES: O=C(/C=N\O)Nc1cc(F)cc(F)c1]

A solution of 2,2,2-trichloroacetaldehyde (391 g, 2.65 mol) was added dropwise a solution of sodium sulfate (652 g, 4.59 mol) dissolved in water (1.95 L) at 50° C. over one hour. In another vessel 3,5-difluoroaniline (250 g, 1.94 mol) and concentrated hydrogen chloride (170 mL) was heated at 60° C. till completely dissolved. Then this salt solution was added to the original reaction mixture at 50° C. over 0.5 hour. A solution of hydroxylamine hydrochloride (431 g, 6.2 mol) in water (975 mL) was added over one hour. The resulting suspension was stirred at 55° C. for 5 hours. The reaction was cooled to 10° C. and filtered. The solid was washed with cold water (500 mL) and dried to give the title compound (320 g, 82%). ¹H-NMR (400 MHz, DMSO-d₆) δ 12.34 (s, 1H), 10.60 (s, 1H), 7.64 (s, 1H), 7.49-7.43 (m, 2H), 6.97-6.92 (m, 1H) ppm. MS: M/e 201 (M+1)⁺.

### Step B: 4,6-difluoroindoline-2,3-dione

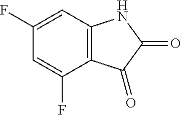 [SMILES: O=C1Nc2cc(F)cc(F)c2C1=O]

50% H₂SO₄ (w/w, 200 g) was added to con.H₂SO₄ (1 L) at 0˜5° C. The solution was heated to 60° C. before adding the product of step A (230 g, 1.25 mol) in several portions over 2 hours, maintaining the temperature between 58˜62° C. The reaction was completed. The reaction was cooled to 10° C. and put into cold water (3.0 L) about 5° C. After 2 hours the mixture was filtered and the cake was collected and dried to give the target compound (135 g, 76%). ¹H NMR (400 MHz, DMSO-d6) δ 11.41 (s, 1H), 6.96-6.83 (m, 1H), 6.62 (dd, J=8.8, 2.0 Hz, 1H) ppm. MS: M/e 184 (M+1)⁺

### Step C: 2-amino-4,6-difluorobenzoic acid

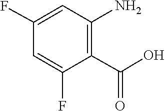 [SMILES: Nc1cc(F)cc(F)c1C(=O)O]

A solution of NaOH (284 g, 7.1 mol) in 500 mL H₂O was added to a suspension of the product of step B (130 g, 0.71 mol) in H₂O (800 mL) at room temperature over 20 min. The mixture was heated to 63° C. before adding H₂O₂ (35%, 250 mL) over an hour, maintaining the temperature at 63-67° C. After the addition, the mixture was stirred at this temperature for 2 hours. The mixture was allowed cool to room temperature and acidified to pH=5-6 with con. HCl. The mixture was filtered and the cake was collected, dried to give the target compound (100 g, 81%). ¹H NMR (400 MHz, DMSO-d6) δ 6.35 (td, J=11.6, 2.4, 1.6 Hz, 1H), 6.24 (td, J=12.0, 9.4, 2.4 Hz, 1H) ppm. MS: M/e: 174 (M+1)⁺

### Step D: 5,7-difluoroquinazolin-4(3H)-one

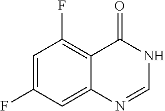 [SMILES: O=c1[nH]cnc2cc(F)cc(F)c12]

The product of step C (96 g, 0.55 mol) was dissolved in 2-methoxyethanol (700 mL) and formamidine acetate (114.2 g, 1.1 mol) was added. After the addition, the reaction mixture was stirred at 108° C. for 4 hours with the condenser pipe open in air. Most of solvent was removed to give the residue, which was treated with H₂O (1.5 L) and filtered. The cake was collected, dried to give target compound (170 g, 83%). ¹H NMR (400 MHz, DMSO-d6) δ 11.37 (s, 1H), 8.12 (s, 1H), 7.41-7.26 (m, 2H) ppm. MS: M/e 183 (M+1)⁺.

### Step E: 5,7-bis(benzyloxy)quinazolin-4(3H)-one

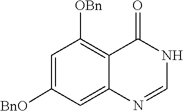 [SMILES: O=c1[nH]cnc2cc(OCc3ccccc3)cc(OCc3ccccc3)c12]

To a suspension of NaH (60%, 17.6 g, 0.44 mol) in DMA (200 mL) was added dropwise phenylmethanol (40 g, 0.37 mol) at 0˜10° C. After then, the reaction was stirred at room temperature for half an hour. The reaction mixture was cooled to 0° C. and the product of step D (20 g, 0.11 mol) was added portion wise. After the addition, the reaction was stirred at 90° C. overnight. The reaction mixture was cooled to room temperature and poured into H₂O (200 mL). After stirred for 10 min, the mixture was filtered and the cake was collected, dried to give target compound (34 g, 83%). ¹H NMR (400 MHz, DMSO-d6) δ 11.75 (s, 1H), 7.94 (s, 1H), 7.66-7.25 (m, 10H), 6.76 (d, J=15.8 Hz, 2H), 5.22 (s, 6H) ppm. MS: M/e 359 (M+1)⁺.

### Step F: 7-(benzyloxy)-5-hydroxyquinazolin-4(3H)-one

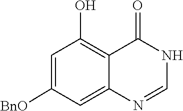 [SMILES: O=c1[nH]cnc2cc(OCc3ccccc3)cc(O)c12]

A mixture of the product of Step E (34 g, 95 mmol) and MgBr₂ (26.2 g, 142 mmol) in pyridine (150 mL) was stirred at 120° C. for 8 hours. The reaction mixture was concentrated to give the residue, which was treated with H₂O (500 mL), then acidified to pH=4-5 with AcOH. The mixture was filtered and the cake was collected, dried to give target compound (20 g, 78.5%). ¹H NMR (400 MHz, DMSO-d6) δ 12.49 (s, 1H), 11.88 (s, 1H), 8.07 (s, 1H), 7.52-7.28 (m, 5H), 6.71 (s, 1H), 6.52 (d, J=2.4 Hz, 1H), 5.23 (s, 2H) ppm. MS: M/e 269 (M+1)⁺.

### Step G: (7-(benzyloxy)-5-hydroxy-4-oxoquinazolin-3(4H)-yl)methyl pivalate

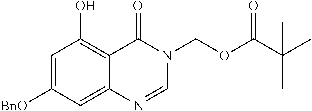 [SMILES: CC(C)(C)C(=O)OCn1cnc2cc(OCc3ccccc3)cc(O)c2c1=O]

NaH (1.6 g, 40.9 mmol) was suspended in dry DMF (30 mL), then the product of step F (5 g, 18.6 mmol) was added portionwise at 0° C. Then the mixture was stirred for an hour at room temperature. The reaction was cooled to 0° C. and chloromethyl pivalate (8.1 g, 54.1 mmol) was added dropwise at 0° C. After the addition, the mixture was stirred for an hour at 0° C. The reaction was quenched with H₂O/AcOH (100 mL/10 mL) and filtered, the cake was collected and recrystallized from EtOH (10 mL) to give target compound (4.0 g, 56.3%). ¹H NMR (400 MHz, CDCl₃) δ 11.33 (s, 1H), 8.16 (s, 1H), 7.54-7.32 (m, 5H), 6.76 (d, J=2.4 Hz, 1H), 6.59 (d, J=2.4 Hz, 1H), 5.87 (s, 2H), 5.15 (s, 2H), 1.21 (s, 9H) ppm. MS: M/e 383 (M+1)⁺.

### Step H: (7-(benzyloxy)-5-((2-methylbut-3-yn-2-yl)oxy)-4-oxoquinazolin-3(4H)-yl)methyl pivalate

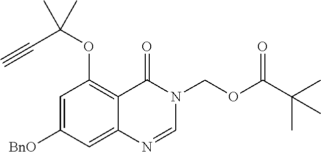 [SMILES: C#CC(C)(C)Oc1cc(OCc2ccccc2)cc2ncn(COC(=O)C(C)(C)C)c(=O)c12]

CuI (50 mg, 0.26 mmol) was added to a mixture of product of step G (5.0 g, 13.1 mmol), K₂CO₃ (3.6 g, 26.2 mmol), KI (3.7 g, 22.3 mol) in DMF (50 mL) under N₂, followed by 3-chloro-3-methylbut-1-yne (2.7 g, 26.12 mmol). After the addition, the reaction mixture was stirred at 60° C. for an hour. The reaction mixture was filtered and the filtrate was concentrated to give the crude product, which was directly used to the next step. ¹H NMR (400 MHz, CDCl₃) δ 8.21 (s, 1H), 7.48-7.29 (m, 6H), 6.92 (d, J=2.0 Hz, 1H), 5.87 (s, 2H), 5.16 (s, 2H), 2.59 (s, 1H), 1.76 (s, 6H), 1.19 (s, 9H) ppm. MS: M/e 449 (M+1)⁺.

### Step I: (5-(benzyloxy)-2,2-dimethyl-10-oxo-2H-pyrano[2,3-f]quinazolin-9(10H)-yl)methyl pivalate

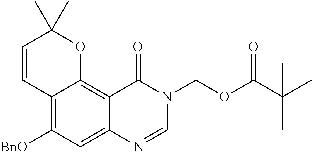 [SMILES: CC1(C)C=Cc2c(OCc3ccccc3)cc3ncn(COC(=O)C(C)(C)C)c(=O)c3c2O1]

A mixture of crude product of H (crude from step H) in dimethyl phenyl amine (20 mL) was stirred refluxed for 20 min. The reaction was concentrated to give the residue, which was purified by column chromatography (petroleum ether/EtOAc/CH₂Cl₂=10:1:1-3:1:1) quickly to give the crude product, which was recrystallized from EtOAc to give the target compound (2.5 g, 50%). ¹H NMR (400 MHz, CDCl₃) δ 8.13 (s, 1H), 7.43-7.40 (m, 5H), 6.73 (t, J=5.0 Hz, 2H), 5.88 (s, 2H), 5.63 (d, J=10.0 Hz, 1H), 5.17 (s, 2H), 2.97 (d, J=14.2 Hz, 1H), 1.55 (s, 6H), 1.20 (s, 9H) ppm. MS: M/e 449 (M+1)⁺.

### Step J: 5-(benzyloxy)-2,2-dimethyl-2H-pyrano[2,3-f]quinazolin-10(9H)-one

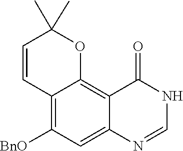 [SMILES: CC1(C)C=Cc2c(OCc3ccccc3)cc3nc[nH]c(=O)c3c2O1]

The product of Step I (5.2 g, 11.6 mmol) was dissolved in NH₃ (g)/MeOH (10.0 M, 100 mL) and the mixture was stirred for a weekend. The reaction was concentrated to give the residue, which was directly used to the next step without further purification. MS: M/e 335 (M+1)⁺.

### Step K: 5-hydroxy-2,2-dimethyl-3,4-dihydro-2H-pyrano[2,3-f]quinazolin-10(9H)-one

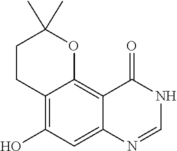 [SMILES: CC1(C)CCc2c(O)cc3nc[nH]c(=O)c3c2O1]

A mixture of product J (11.6 mol) and Pd/C (0.5 g) in MeOH (50 mL) was stirred overnight under H₂ (1 atm). The reaction mixture was filtered and the filtrate was concentrated, which was recrystallized from EtOAc to give the target compound (2.0 g, 70%). ¹H NMR (400 MHz, DMSO-d6) δ 11.41 (s, 1H), 10.36 (s, 1H), 7.75 (s, 1H), 6.51 (s, 1H), 2.57 (t, J=6.8 Hz, 2H), 1.74 (t, J=6.8 Hz, 2H), 1.29 (s, 6H) ppm. MS: M/e: 247 (M+1)⁺.

### Step L: 2,2-dimethyl-10-oxo-3,4,9,10-tetrahydro-2H-pyrano[2,3-f]quinazolin-5-yl acetate

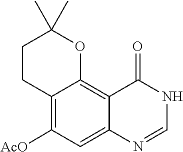 [SMILES: CC(=O)Oc1cc2nc[nH]c(=O)c2c2c1CCC(C)(C)O2]

A mixture of the product of Step K (1.6 g, 6.5 mmol) and pyridine (1 mL) in acetic anhydride (20 mL) was stirred at 70° C. for 0.5 hour. TLC indicated the reaction was completed. The reaction was concentrated to dryness. The residue was taken up in water (30 mL) and the resulting mixture stirred at 80° C. for 30 min. The solid was collected by filtration and dried to give the title compound (1.8 g, 96%). ¹H-NMR (400 MHz, CDCl₃) δ 7.94 (s, 1H), 6.98 (s, 1H), 2.66 (t, J=6.8 Hz, 2H), 2.36 (s, 3H), 1.86 (t, J=6.8 Hz, 2H), 1.46 (s, 6H) ppm. MS: M/e 289 (M+1)⁺.

### Step M: tert-butyl 4-(5-acetoxy-2,2-dimethyl-3,4-dihydro-2H-pyrano[2,3-f]quinazolin-10-yl)piperazine-1-carboxylate

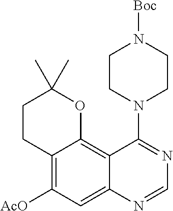 [SMILES: CC(=O)Oc1cc2ncnc(N3CCN(C(=O)OC(C)(C)C)CC3)c2c2c1CCC(C)(C)O2]

To a stirred suspension of 2,2-dimethyl-10-oxo-3,4,9,10-tetrahydro-2H-pyrano[2,3-f]quinazolin-5-yl acetate (500 mg, 1.68 mmol) in DCE (10 mL) was added DIPEA (865 mg, 6.71 mol), followed by POCl₃ (512 mg, 3.35 mmol). After the addition, the reaction was stirred at 85° C. for an hour. The mixture was concentrated to give the residue, which was dissolved in DCE (10 mL) and tert-butyl piperazine-1-carboxylate (312 mg, 1.68 mmol) was added. Then the mixture was stirred at 80° C. for another hour. The mixture was concentrated and purified by column chromatography (PE:EA=3:1, then change into CH₂Cl₂:MeOH=100:1) to give the target compound (310 mg, 39.6%). MS: M/e 457 (M+1)⁺.

### Step N: tert-butyl 4-(5-hydroxy-2,2-dimethyl-3,4-dihydro-2H-pyrano[2,3-f]quinazolin-10-yl)piperazine-1-carboxylate

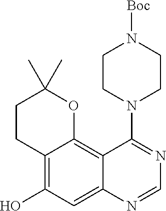 [SMILES: CC(C)(C)OC(=O)N1CCN(c2ncnc3cc(O)c4c(c23)OC(C)(C)CC4)CC1]

A mixture of the product of step M (310 mg, 0.66 mmol) in MeOH (NH₃ 7.0 M, 5 mL) was stirred for half an hour. The reaction mixture was concentrated to give the target compound (290 mg, 100%). MS: M/e 415 (M+1)⁺.

### Step O: tert-butyl 4-(2,2-dimethyl-5-(((trifluoromethyl)sulfonyl)oxy)-3,4-dihydro-2H-pyrano[2,3-f]quinazolin-10-yl)piperazine-1-carboxylate

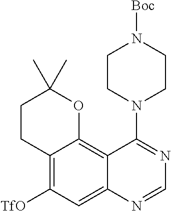 [SMILES: CC(C)(C)OC(=O)N1CCN(c2ncnc3cc(OS(=O)(=O)C(F)(F)F)c4c(c23)OC(C)(C)CC4)CC1]

To a stirred solution of the product of step N (41.4 mg, 0.1 mmol) in dry THF (5 mL) was added K₂CO₃ (27.6 mg, 0.2 mmol), then 1,1,1-trifluoro-N-phenyl-N-((trifluoromethyl)sulfonyl)methanesulfonamide (42.8 mg, 0.12 mmol) was added under N₂. After the addition, the reaction mixture was stirred overnight. EtOAc (10 mL) was added and the mixture was washed with H₂O (10 mL), brine, dried over Na₂SO₄, concentrated and purified by column chromatography (petroleum ether/EA=5:1-3:1) to give the target compound (31 mg, 54.9%). MS: M/e 547 (M+1)⁺.

### Step P: tert-butyl 4-(2,2-dimethyl-5-(naphthalen-1-yl)-3,4-dihydro-2H-pyrano[2,3-f]quinazolin-10-yl)piperazine-1-carboxylate

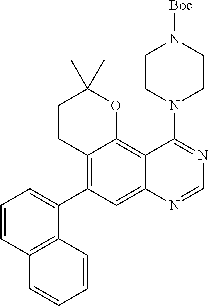 [SMILES: CC(C)(C)OC(=O)N1CCN(c2ncnc3cc(-c4cccc5ccccc45)c4c(c23)OC(C)(C)CC4)CC1]

PddppfCl₂ (4.5 mg, 0.055 mmol) was added to a stirred solution of the product of step 0 (30 mg, 0.055 mmol), naphthalen-1-ylboronic acid (10.4 mg, 0.06 mmol) and K₂CO₃ (14 mg, 0.11 mmol) in dioxane/H₂O (5 mL/1 mL) under N₂. After the addition, the reaction was stirred at 100° C. for 5 hours. The reaction was treated with EA (15 mL) and washed with H₂O (10 mL), brine, dried over Na₂SO₄, concentrated and purified by prep-TLC (petroleum ether/EA=1:1) to give the target compound (18 mg, 62.4%). MS: M/e 525 (M+1)⁺.

### Step Q: 1-(4-(2,2-dimethyl-5-(naphthalen-1-yl)-3,4-dihydro-2H-pyrano[2,3-f]quinazolin-10-yl)piperazin-1-yl)prop-2-en-1-one

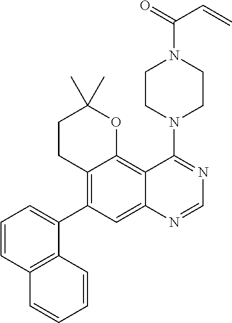 [SMILES: C=CC(=O)N1CCN(c2ncnc3cc(-c4cccc5ccccc45)c4c(c23)OC(C)(C)CC4)CC1]

To a stirred solution of the product of step P (18 mg, 0.034 mmol) in CH₂Cl₂ (5 mL) was added HCl/dioxane (4.0 M, 2 mL) and the mixture was stirred overnight. The reaction mixture was concentrated and directly stirred in CH₃CN/H₂O (5 mL/1 mL), and aq.NaHCO₃ (0.5 mL) was added. Then a solution of acryloyl chloride (3.4 mg, 0.0374 mmol) in CH₂Cl₂ (0.5 mL) was added dropwise. The reaction mixture was diluted with EA (10 mL) and washed with brine, dried over Na₂SO₄, concentrated, purified by Prep-TLC to give the target compound (10 mg)¹H NMR (400 MHz, DMSO-d₆) δ 8.53 (s, 1H), 8.04 (dd, J=8.2, 4.0 Hz, 2H), 7.70-7.60 (m, 1H), 7.59-7.54 (m, 1H), 7.51-7.46 (m, 2H), 7.41 (d, J=8.4 Hz, 1H), 7.15 (s, 1H), 6.86 (dd, J=16.8, 10.4 Hz, 1H), 6.17 (dd, J=16.8, 2.3 Hz, 1H), 5.74 (dd, J=10.4, 2.3 Hz, 1H), 3.76 (s, 8H), 2.38-2.13 (m, 2H), 1.72 (t, J=6.4 Hz, 2H), 1.40 (s, 3H), 1.38 (s, 3H) ppm. MS: M/e 479 (M+1)⁺.

### Compound A2: 1-(4-(5-(2-fluoro-6-hydroxyphenyl)-2,2-dimethyl-3,4-dihydro-2H-pyrano[2,3-f]quinazolin-10-yl)piperazin-1-yl)prop-2-en-1-one

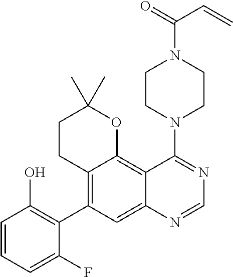 [SMILES: C=CC(=O)N1CCN(c2ncnc3cc(-c4c(O)cccc4F)c4c(c23)OC(C)(C)CC4)CC1]

The compound A2 was synthesized starting from the corresponding starting materials according the similar procedures described as those of Compound A1 (1 mg). ¹H NMR (400 MHz, CD₃OD) δ 8.41 (s, 1H), 7.25 (dd, J=15.2, 8.4 Hz, 1H), 7.14 (s, 1H), 6.87-6.62 (m, 3H), 6.25 (dd, J=16.8, 1.8 Hz, 1H), 5.78 (dd, J=10.6, 1.8 Hz, 1H), 3.86-3.75 (m, 8H), 2.73-2.44 (m, 2H), 1.88 (t, J=6.8 Hz, 2H), 1.43 (s, 3H), 1.40 (s, 3H) ppm. MS: M/e 463 (M+1)⁺.

### Compound A3: 2-((2S)-1-acryloyl-4-(5-(2-fluoro-6-hydroxyphenyl)-2,2-dimethyl-3,4-dihydro-2H-pyrano[2,3-f]quinazolin-10-yl)piperazin-2-yl)acetonitrile

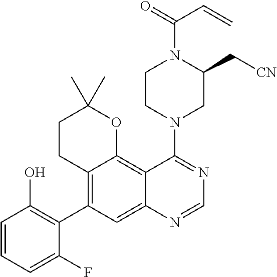 [SMILES: C=CC(=O)N1CCN(c2ncnc3cc(-c4c(O)cccc4F)c4c(c23)OC(C)(C)CC4)C[C@@H]1CC#N]

The compound A3 was synthesized starting from the corresponding starting materials according the similar procedures described as those of Compound A1 to give the target compound (2.14 mg). ¹H NMR (400 MHz, CD₃OD) δ 8.44 (s, 1H), 7.28-7.22 (m, 1H), 7.17 (s, 1H), 6.92-6.66 (m, 4H), 6.28 (d, J=16.8 Hz, 1H), 5.82 (d, J=10 Hz, 1H), 4.60-4.44 (m, 2H), 4.24-4.04 (m, 2H), 3.71-3.48 (m, 2H), 2.88 (s, 2H), 2.73-2.45 (m, 2H), 1.96-1.81 (m, 2H), 1.47-1.41 (m, 6H) ppm. MS: M/e 502 (M+1)⁺.

### Compound A4: 2-((2S)-1-acryloyl-4-(5-(2-fluoro-6-hydroxyphenyl)-3,4-dihydro-2H-pyrano[2,3-f]quinazolin-10-yl)piperazin-2-yl)acetonitrile

### Step A: (7-(benzyloxy)-4-oxo-5-(prop-2-yn-1-yloxy)quinazolin-3(4H)-yl)methyl pivalate

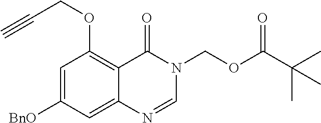 [SMILES: C#CCOc1cc(OCc2ccccc2)cc2ncn(COC(=O)C(C)(C)C)c(=O)c12]

A mixture of (7-(benzyloxy)-5-hydroxy-4-oxoquinazolin-3(4H)-yl)methyl pivalate (1.17 g, 7.8 mmol), 3-bromoprop-1-yne, K₂CO₃ (1.0 g, 7.8 mmol) and DMF (10 mL) was stirred for 2 hours at 110° C. in a sealed tube. The reaction mixture was filtered and the filtrate was concentrated to give the residue, which was purified by column chromatography (petroleum ether/EtOAc=3:1-2:1) to give the target compound (1.0 g, 99%). H NMR (400 MHz, CDCl₃) δ 8.23 (s, 1H), 7.52-7.30 (m, 5H), 6.87 (d, J=2.0 Hz, 1H), 6.75 (d, J=2.1 Hz, 1H), 5.88 (s, 2H), 5.17 (s, 2H), 4.85 (d, J=2.2 Hz, 2H), 2.55 (s, 1H), 1.19 (s, 9H). ppm. MS: M/e 421 (M+1)⁺

### Step B: (5-(benzyloxy)-10-oxo-2H-pyrano[2,3-f]quinazolin-9(10H)-yl)methyl pivalate

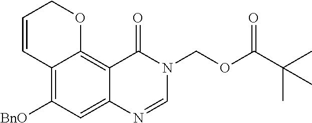 [SMILES: CC(C)(C)C(=O)OCn1cnc2cc(OCc3ccccc3)c3c(c2c1=O)OCC=C3]

A mixture of the product of Step A (2.6 mmol) in N,N-dimethylaniline (10 mL) was refluxed for 10 minutes under N₂. Most solvent was removed to give the residue, which was purified by column chromatography (petroleum ether/EtOAc=6:1-2:1) to give target compound (400 mg, 36%). ¹H NMR (400 MHz, CDCl₃) δ 8.17 (s, 1H), 7.45-7.35 (m, 5H), 6.91-6.67 (m, 2H), 5.87 (s, 2H), 5.80 (d, J=10.0 Hz, 1H), 5.17 (s, 2H), 5.02 (dd, J=3.6, 1.7 Hz, 2H), 1.19 (s, 9H). MS: M/e: 421 (M+1)⁺

### Step C: 5-(benzyloxy)-2H-pyrano[2,3-f]quinazolin-10(9H)-one

A mixture of the product of Step B (400 mg, 0.94 mmol) in NH₃/MeOH (7M, 5 mL) was stirred overnight at room temperature. The reaction mixture was concentrated to give target compound, which was directly used to the next step. MS: M/e 307 (M+1)⁺.

### Step D: 5-hydroxy-3,4-dihydro-2H-pyrano[2,3-f]quinazolin-10(9H)-one

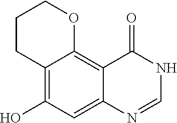 [SMILES: O=c1[nH]cnc2cc(O)c3c(c12)OCCC3]

A mixture of the product of step C (100 mg, 0.32 mmol) and Pd/C (100 mg) in MeOH (10 mL) was stirred overnight under H₂ (1 atm). The reaction mixture was filtered. The filtrate was concentrated to give the residue, which was washed with EtOAc/Petroleum ether (2 mL/5 mL) to give the target compound (70 mg, 100%). ¹H NMR (400 MHz, CD₃OD) δ 7.77 (s, 1H), 6.49 (s, 1H), 4.33-4.08 (m, 2H), 2.61 (t, J=6.6 Hz, 2H), 2.04-1.80 (m, 2H). ppm. MS: M/e 219 (M+1)⁺.

Step E: 10-oxo-3,4,9,10-tetrahydro-2H-pyrano[2,3-f]quinazolin-5-yl acetate

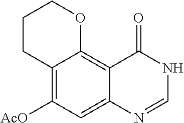 [SMILES: CC(=O)Oc1cc2nc[nH]c(=O)c2c2c1CCCO2]

A mixture of the product of Step D (70 mg, 0.32 mmol) and pyridine (1 drop) in Ac₂O (10 mL) was stirred at 80° C. for half an hour. The reaction mixture was concentrated to give the residue, which was treated with H₂O (10 mL) and stirred at 80° C. for an hour. The reaction mixture was extracted with EtOAc (10 mL×5). The combined organic layers were washed with brine, dried over Na₂SO₄ and concentrated to give the target compound, which was directly used to the next step. MS: M/e 261 (M+1)⁺.

### Step F: benzyl (S)-4-(5-acetoxy-3,4-dihydro-2H-pyrano[2,3-f]quinazolin-10-yl)-2-(cyanomethyl)piperazine-1-carboxylate

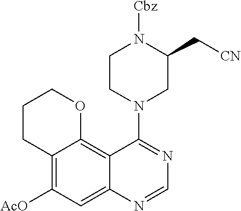 [SMILES: CC(=O)Oc1cc2ncnc(N3CCN(C(=O)OCc4ccccc4)[C@@H](CC#N)C3)c2c2c1CCCO2]

To a stirred solution of 10-oxo-3,4,9,10-tetrahydro-2H-pyrano[2,3-f]quinazolin-5-yl acetate (271 mg, 1.04 mmol) in DCE (20 mL) was added DIPEA (516 mg, 4 mmol), followed by POCl₃ (318 mg, 2.08 mmol). Then the mixture was stirred at 90° C. for 1.5 hours. The mixture became clear and concentrated to give the residue, which was dissolved in DCE (20 mL) again, and benzyl (S)-2-(cyanomethyl)piperazine-1-carboxylate (404 mg, 1.56 mmol). After then, the mixture was stirred at 90° C. for an hour. The mixture was concentrated to give the crude product (100%), which was directly used to the next step. MS: M/e 502 (M+1)⁺.

### Step G: benzyl (S)-2-(cyanomethyl)-4-(5-hydroxy-3,4-dihydro-2H-pyrano[2,3-f]quinazolin-10-yl)piperazine-1-carboxylate

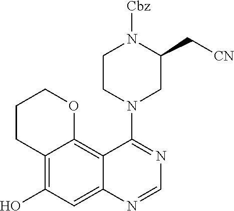 [SMILES: N#CC[C@H]1CN(c2ncnc3cc(O)c4c(c23)OCCC4)CCN1C(=O)OCc1ccccc1]

A mixture of the product of step F(1.04 mmol) in NH3/MeOH(g) (7.0 M, 10 mL) was stirred for half an hour. The reaction mixture was concentrated to give the target compound (crude, 100%), which was directly used to the next step. MS: M/e 460 (M+1)⁺.

### Step H: benzyl (S)-2-(cyanomethyl)-4-(5-(((trifluoromethyl)sulfonyl)oxy)-3,4-dihydro-2H-pyrano[2,3-f]quinazolin-10-yl)piperazine-1-carboxylate

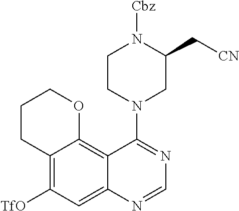 [SMILES: N#CC[C@H]1CN(c2ncnc3cc(OS(=O)(=O)C(F)(F)F)c4c(c23)OCCC4)CCN1C(=O)OCc1ccccc1]

To a stirred solution of the product of step G (1.04 mmol) in dry THF (10 mL) was added K₂CO₃ (287 mg, 2.08 mmol), then 1,1,1-trifluoro-N-phenyl-N-((trifluoromethyl)sulfonyl)methanesulfonamide (445 mg, 1.25 mmol) was added under N₂. After the addition, the reaction mixture was stirred overnight. EtOAc (20 mL) was added and the mixture was washed with H₂O (20 mL), brine, dried over Na₂SO₄, concentrated and purified by column chromatography (petroleum ether/EtOAc=5:1-2:1) to give the target compound (235 mg, 38.3%). MS: M/e 592 (M+1)⁺.

### Step I: benzyl (2S)-4-(5-(2-(benzyloxy)-6-fluorophenyl)-3,4-dihydro-2H-pyrano[2,3-f]quinazolin-10-yl)-2-(cyanomethyl)piperazine-1-carboxylate

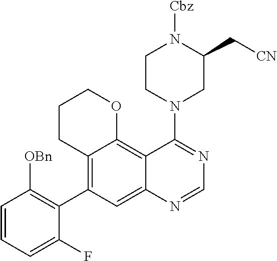 [SMILES: N#CC[C@H]1CN(c2ncnc3cc(-c4c(F)cccc4OCc4ccccc4)c4c(c23)OCCC4)CCN1C(=O)OCc1ccccc1]

PddppfCl₂ (12.3 mg, 0.0169 mmol) was added to a stirred solution of the product of step H (100 mg, 0.169 mmol), (2-(benzyloxy)-6-fluorophenyl)boronic acid (50 mg, 0.2 mmol) and aq. sat. Na₂CO₃ (0.5 mL) in dioxane (5 mL) under N₂. After the addition, the reaction was stirred at 90° C. overnight. The reaction was treated with EtOAc (15 mL) and washed with H₂O (10 mL), brine, dried over Na₂SO₄, concentrated and purified by Pre-TLC (petroleum ether/EtOAc=2:1) to give the target compound (65 mg, 59.8%). MS: M/e 644 (M+1)⁺.

### Step J: 2-((2S)-4-(5-(2-fluoro-6-hydroxyphenyl)-3,4-dihydro-2H-pyrano[2,3-f]quinazolin-10-yl)piperazin-2-yl)acetonitrile

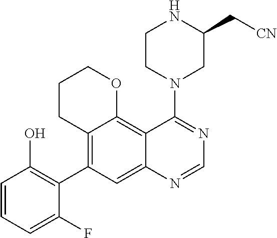 [SMILES: N#CC[C@H]1CN(c2ncnc3cc(-c4c(O)cccc4F)c4c(c23)OCCC4)CCN1]

To a stirred solution of the product of step I (65 mg, 0.101 mmol) in EtOH/THF (5 mL/5 mL) was added Pd/C (50 mg), and the mixture was stirred overnight under H₂ (1 atm). The reaction mixture was filtered and the filtrate was concentrated to give the target compound (crude, 100%), which was directly used to the next step. MS: M/e 420 (M+1)⁺.

### Step K: 2-((2S)-1-acryloyl-4-(5-(2-fluoro-6-hydroxyphenyl)-3,4-dihydro-2H-pyrano[2,3-f]quinazolin-10-yl)piperazin-2-yl)acetonitrile

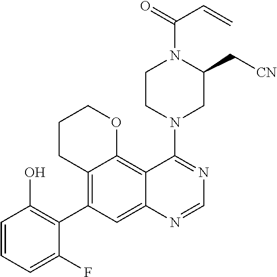 [SMILES: C=CC(=O)N1CCN(c2ncnc3cc(-c4c(O)cccc4F)c4c(c23)OCCC4)C[C@@H]1CC#N]

To a stirred solution of the product of step J (0.101 mmol) in CH₃CN (5 mL) was added sat.aq.NaHCO₃ (0.5 mL), and a solution of acryloyl chloride (27 mg, 0.303 mmol) in CH₃CN (1 mL) was added. After stirred for an hour, the reaction was treated with H₂O (5 mL) and extracted with EtOAc (5 mL×2). The combined organic layers were washed with brine, dried over Na₂SO₄, concentrated and purified by Pre-TLC (EtOAc100%) to give the intermediate, which was dissolved in THF/H₂O (4 mL/2 mL) and a little amount of LiOH (2 mg) was added and stirred overnight. The reaction mixture was acidified to pH=4˜5 with citric acid, extracted with EtOAc (5 mL×2). The combined organic layers were washed with brine, dried over Na₂SO₄, concentrated and purified again by Prep-TLC (EtOAc100%) to give the target compound (6 mg). ¹H NMR (400 MHz, CD₃OD) δ 8.45 (s, 1H), 7.24 (dd, J=15.2, 8.4 Hz, 1H), 7.19 (d, J=1.2 Hz, 1H), 6.97-6.66 (m, 3H), 6.29 (d, J=16.4 Hz, 1H), 5.83 (d, J=10.2 Hz, 1H), 5.20 (s, 0.5H), 4.59 (s, 0.5H), 4.51-4.40 (m, 2H), 4.39-4.28 (m, 1H), 4.15-4.03 (m, 2H), 3.43-3.33 (m, 1H), 3.25-3.07 (m, 2H), 2.99-2.86 (m, 2H), 2.73-2.48 (m, 2H), 2.11-2.01 (m, 2H) ppm. MS: M/e 474 (M+1)⁺.

### Compound A5: 1-(7-(5-(2-fluoro-6-hydroxyphenyl)-3,4-dihydro-2H-pyrano[2,3-f]quinazolin-10-yl)-2,7-diazaspiro[3.5]nonan-2-yl)prop-2-en-1-one

### Step A: (5-hydroxy-10-oxo-2H-pyrano[2,3-f]quinazolin-9(10H)-yl)methyl pivalate

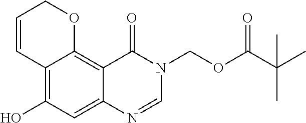 [SMILES: CC(C)(C)C(=O)OCn1cnc2cc(O)c3c(c2c1=O)OCC=C3]

A mixture of (5-(benzyloxy)-10-oxo-2H-pyrano[2,3-f]quinazolin-9(10H)-yl)methyl pivalate (1 g, 2.38 mmol) in TFA (10 mL) was stirred at refluxed overnight. The reaction mixture was concentrated to give the residue, which was poured into H₂O (30 mL) and extracted with EA (20 mL×3). The combined organic layers were washed with brine, dried over Na₂SO₄, concentrated and purified by column chromatography (petroleum ether/EA=5:1˜1:1) to give the target compound (480 mg, 61.1%). MS: M/e 331 (M+1)⁺.

### Step B: (10-oxo-5-(((trifluoromethyl)sulfonyl)oxy)-2H-pyrano[2,3-f]quinazolin-9(10H)-yl)methyl pivalate

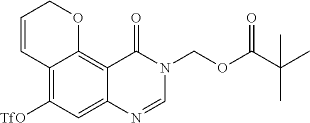 [SMILES: CC(C)(C)C(=O)OCn1cnc2cc(OS(=O)(=O)C(F)(F)F)c3c(c2c1=O)OCC=C3]

The product of step A (480 mg, 1.45 mmol) was dissolved in THF (20 mL), K₂CO₃ (400 mg, 2.9 mmol) was added, followed by 1,1,1-trifluoro-N-phenyl-N-((trifluoromethyl)sulfonyl)methanesulfonamide (621 mg, 1.74 mmol) under N₂. After the addition, the reaction mixture was stirred overnight. The reaction was treated with EA (30 mL), washed with H₂O (20 mL), brine, dried over Na₂SO₄, concentrated and purified by column chromatography (petroleum ether/EA=10:1˜2:1) to give the target compound (426 mg, 63.5%). MS: M/e 463 (M+1)⁺.

### Step C: (5-(2-(benzyloxy)-6-fluorophenyl)-10-oxo-2H-pyrano[2,3-f]quinazolin-9(10H)-yl)methyl pivalate

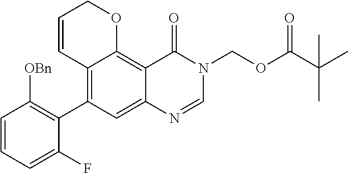 [SMILES: CC(C)(C)C(=O)OCn1cnc2cc(-c3c(F)cccc3OCc3ccccc3)c3c(c2c1=O)OCC=C3]

PddppfCl₂ (63.3 mg, 0.0865 mmol) was added to a stirred mixture of the product of step B (400 mg, 0.864 mmol), (2-(benzyloxy)-6-fluorophenyl)boronic acid (255 mg, 1.04 mmol) and sat.aq.Na₂CO₃ (2 mL) in dioxane (20 mL) under N₂. After the addition, the reaction was stirred at 100° C. for an hour. The reaction mixture was treated with EA (30 mL), washed with brine, dried over Na₂SO₄, concentrated and purified by column chromatography (petroleum ether/EA=7:1-2:1) to give the target compound (402 mg, 90.5%). MS: M/e 515 (M+1)⁺.

### Step D: 5-(2-(benzyloxy)-6-fluorophenyl)-2,9-dihydro-10H-pyrano[2,3-f]quinazolin-10-one

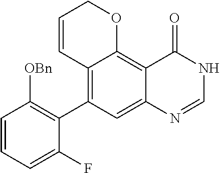 [SMILES: O=c1[nH]cnc2cc(-c3c(F)cccc3OCc3ccccc3)c3c(c12)OCC=C3]

A mixture of the product of step C (372 mg, 0.72 mmol) in NH₃/MeOH (g) (10.0 M, 10 mL) was stirred overnight. The reaction mixture was concentrated to give the target compound (300 mg, 100%). MS: M/e 401 (M+1)⁺.

### Step E: tert-butyl 7-(5-(2-(benzyloxy)-6-fluorophenyl)-2H-pyrano[2,3-f]quinazolin-10-yl)-2,7-diazaspiro[3.5]nonane-2-carboxylate

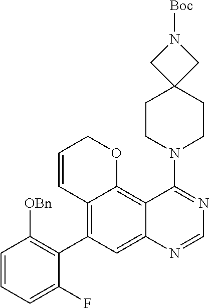 [SMILES: CC(C)(C)OC(=O)N1CC2(CCN(c3ncnc4cc(-c5c(F)cccc5OCc5ccccc5)c5c(c34)OCC=C5)CC2)C1]

To a stirred solution of the product of step D (60 mg, 0.15 mmol) in DCE (5 mL) was added DIPEA (77.4 mg, 0.6 mmol), followed by POCl₃ (46 mg, 0.3 mmol). After the addition, the reaction was stirred for 2 hours. The reaction mixture was concentrated to give the residue, which was dissolved in DCE (5 mL), and tert-butyl 2,7-diazaspiro[3.5]nonane-2-carboxylate (51 mg, 0.225 mmol) was added. Then stirred at 90° C. for 2 hours. The reaction mixture was concentrated to give the residue, which was purified by Prep-TLC (petroleum ether/EA=2:1) to give the target compound (26 mg, 26%). MS: M/e 609 (M+1)⁺.

### Step F: tert-butyl 7-(5-(2-fluoro-6-hydroxyphenyl)-3,4-dihydro-2H-pyrano[2,3-f]quinazolin-10-yl)-2,7-diazaspiro[3.5]nonane-2-carboxylate

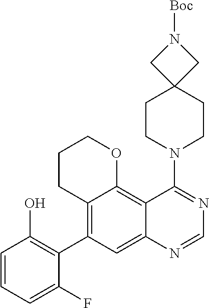 [SMILES: CC(C)(C)OC(=O)N1CC2(CCN(c3ncnc4cc(-c5c(O)cccc5F)c5c(c34)OCCC5)CC2)C1]

Pd/C (20 mg) was added to a stirred solution of the product of step E (26 mg, 0.043 mmol) in EtOH/THF (2 mL/2 mL). Then the mixture was stirred overnight under H₂ (1 atm). The reaction mixture was filtered and the filtrate was concentrated to give the target compound (21 mg, 94.2%), which was directly used to the next step. MS: M/e 521 (M+1)⁺.

### Step G: 2-(10-(2,7-diazaspiro[3.5]nonan-7-yl)-3,4-dihydro-2H-pyrano[2,3-f]quinazolin-5-yl)-3-fluorophenol

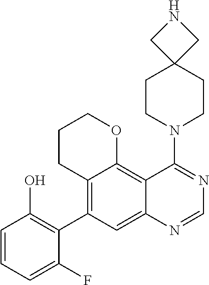 [SMILES: Oc1cccc(F)c1-c1cc2ncnc(N3CCC4(CC3)CNC4)c2c2c1CCCO2]

To a stirred solution of the product of step F (21 mg, 0.043 mmol) in CH₂Cl₂ (5 mL) was added HCl/dioxane (4.0 M, 1 mL). After the addition, the reaction was stirred for 2 hours. The reaction mixture was concentrated to give the target compound, which was directly used to the next step without further purification. MS: M/e 421 (M+1)⁺.

### Step H: 1-(7-(5-(2-fluoro-6-hydroxyphenyl)-3,4-dihydro-2H-pyrano[2,3-f]quinazolin-10-yl)-2,7-diazaspiro[3.5]nonan-2-yl)prop-2-en-1-one

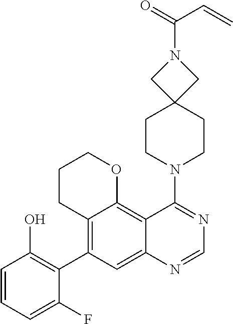 [SMILES: C=CC(=O)N1CC2(CCN(c3ncnc4cc(-c5c(O)cccc5F)c5c(c34)OCCC5)CC2)C1]

To a stirred solution of the product of step G (0.043 mmol) in CH₃CN (3 mL) was added sat. aq.NaHCO₃ (0.5 mL), then a solution of acryloyl chloride (7.3 mg, 0.086 mmol) in CH₃CN (1 mL) was added. After the addition, the reaction was stirred for 2 hours. The reaction was treated with EA/H₂O (3 mL/2 mL). The organic layer was separated, dried over Na₂SO₄, concentrated and purified by Prep-TLC (EA/MeOH=10:1) to give the intermediate, which was dissolved in THF/H₂O (3 mL/1 mL) and a little amount of LiOH (2 mg) was added and stirred overnight. The reaction mixture was acidified to pH=3-4 and extracted with EA (3 mL). The organic layer was dried over Na₂SO₄, concentrated, purified by Prep-TLC (EA/MeOH=10:1) to give the target compound (0.45 mg). ¹H NMR (400 MHz, CD₃OD) δ 8.45 (s, 1H), 7.34-7.26 (m, 1H), 7.06 (s, 1H), 6.82-6.70 (m, 1H), 6.41-6.25 (m, 2H), 5.8-5.73 (m, 1H), 5.34 (t, J=4.4 Hz, 1H), 4.48-4.42 (m, 2H), 4.15 (s, 2H), 3.90 (s, 2H), 2.86-2.51 (m, 4H), 2.12-1.92 (m, 6H), 1.66-1.56 (m, 2H) ppm. MS: M/e 475 (M+1)⁺.

### Compound A6: 1-(5-(5-(2-fluoro-6-hydroxyphenyl)-2,2-dimethyl-3,4-dihydro-2H-pyrano[2,3-f]quinazolin-10-yl)hexahydropyrrolo[3,4-c]pyrrol-2(1H)-yl)prop-2-en-1-one

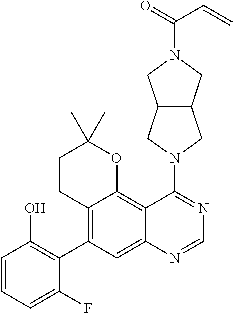 [SMILES: C=CC(=O)N1CC2CN(c3ncnc4cc(-c5c(O)cccc5F)c5c(c34)OC(C)(C)CC5)CC2C1]

The compound A6 was synthesized starting from the corresponding starting materials according the similar procedures described as those of Compound A1 give the target compound (15 mg). ¹H NMR (400 MHz, CD₃OD) δ 8.39 (s, 1H), 7.32-7.22 (m, 1H), 7.03 (s, 1H), 6.78-6.68 (m, 2H), 6.65-6.55 (m, 1H), 6.28 (d, J=16.8 Hz, 1H), 5.76 (d, J=10.4 Hz, 1H), 4.17-4.07 (m, 2H), 4.04-3.95 (m, 2H), 3.95-3.86 (m, 1H), 3.82-3.59 (m, 2H), 3.58-3.46 (m, 1H), 3.23-3.06 (m, 2H), 2.77-2.49 (m, 2H), 1.91 (t, J=6.4 Hz, 2H), 1.39 (s, 6H) ppm. MS: M/e 489 (M+1)⁺.

### Compound A7: (S)-2-(1-acryloyl-4-(5-(naphthalen-1-yl)-3,4-dihydro-2H-pyrano[2,3-f]quinazolin-10-yl)piperazin-2-yl)acetonitrile

### Step A: benzyl (S)-2-(cyanomethyl)-4-(5-hydroxy-3,4-dihydro-2H-pyrano[2,3-f]quinazolin-10-yl)piperazine-1-carboxylate

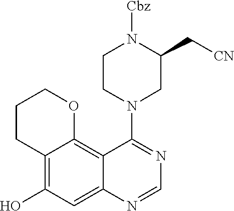 [SMILES: N#CC[C@H]1CN(c2ncnc3cc(O)c4c(c23)OCCC4)CCN1C(=O)OCc1ccccc1]

To a stirred mixture of 10-oxo-3,4,9,10-tetrahydro-2H-pyrano[2,3-f]quinazolin-5-yl acetate (300 mg, 1.15 mmol) in DCE (10 mL) was added DIPEA (593 mg, 4.6 mmol), followed by POCl₃ (352 mg, 2.3 mmol). Then the mixture was stirred for 2.5 hours at 90° C. Benzyl (S)-2-(cyanomethyl)piperazine-1-carboxylate (744 mg, 2.87 mmol) was added and the mixture was stirred for another an hour at 90° C. The reaction mixture was concentrated to give the residue, which was dissolved in NH₃/MeOH (g) (7.0 M, 10 mL) and stirred for half an hour. The reaction mixture was concentrated to give crude product, which was directly used to the next step. MS: M/e 460 (M+1)⁺.

### Step B: benzyl (S)-2-(cyanomethyl)-4-(5-(((trifluoromethyl)sulfonyl)oxy)-3,4-dihydro-2H-pyrano[2,3-f]quinazolin-10-yl)piperazine-1-carboxylate

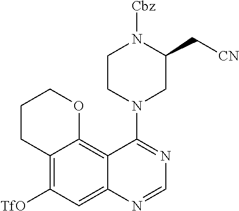 [SMILES: N#CC[C@H]1CN(c2ncnc3cc(OS(=O)(=O)C(F)(F)F)c4c(c23)OCCC4)CCN1C(=O)OCc1ccccc1]

To a stirred solution of the product of step A (1.15 mmol) in THF (20 mL) was added K₂CO₃ (318 mg, 2.3 mmol), then trifluoromethanesulfonic anhydride (488 mg, 1.73 mmol) was added and the mixture was stirred overnight under N₂. The reaction mixture was treated with EA (20 mL), washed with H₂O, brine, dried over Na₂SO₄, concentrated and purified by column chromatography (petroleum ether/EA=10:1˜5:1˜2:1) to give the target compound (150 mg, 22%). MS: M/e 592 (M+1)⁺.

### Step C: benzyl (S)-2-(cyanomethyl)-4-(5-(naphthalen-1-yl)-3,4-dihydro-2H-pyrano[2,3-f]quinazolin-10-yl)piperazine-1-carboxylate

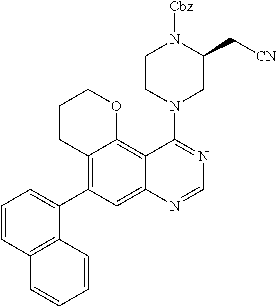 [SMILES: N#CC[C@H]1CN(c2ncnc3cc(-c4cccc5ccccc45)c4c(c23)OCCC4)CCN1C(=O)OCc1ccccc1]

To a stirred solution of the product of step B (85 mg, 0.144 mmol) and naphthalen-1-ylboronic acid (36 mg, 0.173 mmol) in dioxane (5 mL) was added aq.sat. Na₂CO₃ (1 mL), followed by PddppfCl₂ (10.5 mg, 0.0144 mmol) under N₂. After the addition, the reaction mixture was stirred at 90° C. for 3 hours under N₂. The reaction mixture was treated with EA (10 mL), washed with H₂O, brine, dried over Na₂SO₄, concentrated and purified by Prep-TLC (petroleum ether/EA=2:1) to give the target compound (40 mg, 48.9%). MS: M/e 570 (M+1)⁺.

### Step D: (S)-2-(4-(5-(naphthalen-1-yl)-3,4-dihydro-2H-pyrano[2,3-f]quinazolin-10-yl)piperazin-2-yl)acetonitrile

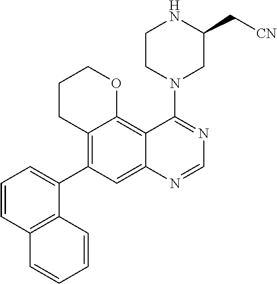 [SMILES: N#CC[C@H]1CN(c2ncnc3cc(-c4cccc5ccccc45)c4c(c23)OCCC4)CCN1]

To a stirred solution of the product of step C (40 mg, 0.069 mmol) in EtOH/THF (3 mL/3 mL) was added Pd/C (40 mg). After then, the mixture was stirred overnight under H₂ (1 atm). The reaction mixture was filtered and the filtrate was concentrated to give the target compound (32 mg, 100%). MS: M/e 436 (M+1)⁺.

### Step E: (S)-2-(1-acryloyl-4-(5-(naphthalen-1-yl)-3,4-dihydro-2H-pyrano[2,3-f]quinazolin-10-yl)piperazin-2-yl)acetonitrile

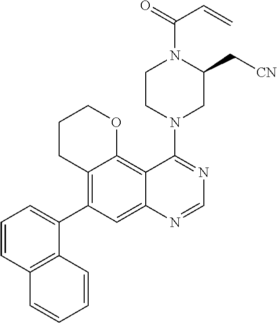 [SMILES: C=CC(=O)N1CCN(c2ncnc3cc(-c4cccc5ccccc45)c4c(c23)OCCC4)C[C@@H]1CC#N]

To a stirred solution of the product of step D (32 mg, 0.073 mmol) in CH₃CN (3 mL) was added aq. sat. NaHCO₃ (0.5 mL), then a solution of acryloyl chloride (8 mg, 0.088 mmol) in CH₃CN (2 mL) was added. After the addition, the reaction mixture was stirred for an hour. The reaction mixture was extracted with EA (10 mL), washed with brine, dried over Na₂SO₄, concentrated and purified by Prep-TLC (EA) to give the target compound (8 mg). ¹H NMR (400 MHz, DMSO-d6) δ 8.57 (s, 1H), 8.09 (s, 2H), 7.73-7.56 (m, 2H), 7.56-7.46 (m, 3H), 7.24 (s, 1H), 6.95 (s, 1H), 6.27 (d, J=16 Hz, 1H), 5.86 (d, J=10 Hz, 1H), 5.22-4.86 (m, 1H), 4.62-4.34 (m, 3H), 4.21-3.96 (m, 1H), 3.29-2.85 (m, 5H), 2.46-2.21 (m, 2H), 1.95 (s, 2H) ppm. MS: M/e 490 (M+1)⁺.

### Compound A8: (S)-2-(1-acryloyl-4-(5-(naphthalen-1-yl)-2H-pyrano[2,3-f]quinazolin-10-yl)piperazin-2-yl)acetonitrile

### Step A: (5-(naphthalen-1-yl)-10-oxo-2H-pyrano[2,3-f]quinazolin-9(10H)-yl)methyl pivalate

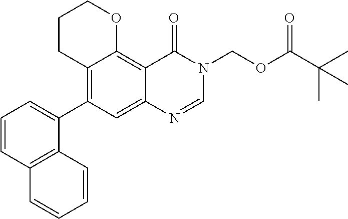 [SMILES: CC(C)(C)C(=O)OCn1cnc2cc(-c3cccc4ccccc34)c3c(c2c1=O)OCCC3]

To a stirred solution of (10-oxo-5-(((trifluoromethyl)sulfonyl)oxy)-2H-pyrano[2,3-f]quinazolin-9(10H)-yl)methyl pivalate (115 mg, 0.25 mmol) and naphthalen-1-ylboronic acid (51.6 mg, 0.3 mmol) in dioxane (5 mL) was added aq.sat. Na₂CO₃ (1 mL, sat.), followed by PddppfCl₂ (18 mg, 0.025 mmol) under N₂. After the addition, the reaction mixture was stirred at 90° C. for an hour under N₂. The reaction mixture was extracted with EA (10 mL), washed with brine, dried over Na₂SO₄, concentrated and purified by Prep-TLC to give the target compound. MS: M/e 441 (M+1)⁺.

### Step B: 5-(naphthalen-1-yl)-2,9-dihydro-10H-pyrano[2,3-f]quinazolin-10-one

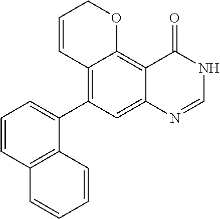 [SMILES: O=c1[nH]cnc2cc(-c3cccc4ccccc34)c3c(c12)OCC=C3]

A mixture of the product of step A (90 mg, 0.2 mmol) in NH₃(g)/MeOH (7.0 M, 5 mL) was stirred overnight at room temperature. The reaction mixture was concentrated to give crude target compound, which was directly used to the next step. MS: M/e 327 (M+1)⁺.

### Step C: tert-butyl (S)-2-(cyanomethyl)-4-(5-(naphthalen-1-yl)-2H-pyrano[2,3-f]quinazolin-10-yl)piperazine-1-carboxylate

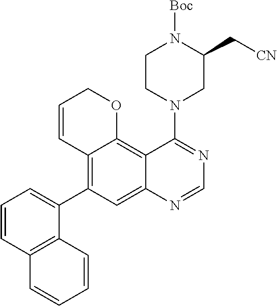 [SMILES: CC(C)(C)OC(=O)N1CCN(c2ncnc3cc(-c4cccc5ccccc45)c4c(c23)OCC=C4)C[C@@H]1CC#N]

The product of step B (0.2 mmol), tert-butyl (S)-2-(cyanomethyl)piperazine-1-carboxylate (67.5 mg, 0.3 mmol) and DBU (60.8 mg, 0.4 mmol) were dissolved in CH₃CN (5 mL), then pyBOP (135 mg, 0.26 mmol) was added and the mixture was stirred for 2 hours. EA (10 mL) was added and washed with H₂O, brine, dried over Na₂SO₄, concentrated and purified by Prep-TLC (petroleum ether/EA=2:1) to give the target compound (53 mg, 50%). MS: M/e 534 (M+1)⁺.

### Step D: (S)-2-(1-acryloyl-4-(5-(naphthalen-1-yl)-2H-pyrano[2,3-f]quinazolin-10-yl)piperazin-2-yl)acetonitrile

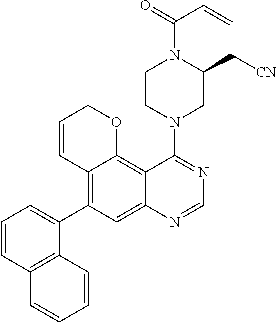 [SMILES: C=CC(=O)N1CCN(c2ncnc3cc(-c4cccc5ccccc45)c4c(c23)OCC=C4)C[C@@H]1CC#N]

To a stirred solution of the product of step C (53 mg, 0.1 mmol) in CH₂Cl₂ (3 mL) was added HCl/EA (4.0 M, 1 mL) and the mixture was stirred for an hour. The reaction mixture was concentrated to give the residue, which was dissolved in CH₃CN/sat. aq.NaHCO₃ (3 mL/0.5 mL), then a solution of acryloyl chloride (13.5 mg, 0.15 mmol) in CH₃CN (1 mL) was added. After the addition, the reaction was stirred for 2 hours. The reaction was treated with EA/H₂O (3 mL/2 mL). The organic layer was separated, dried over Na₂SO₄, concentrated and purified by Prep-TLC (EA) to give the target compound (8 mg). ¹H NMR (400 MHz, DMSO-d6) δ 8.52 (s, 1H), 8.05 (d, J=7.6 Hz, 2H), 7.70-7.54 (m, 2H), 7.53-7.43 (m, 2H), 7.26 (d, J=4.8 Hz, 1H), 6.89 (s, 1H), 6.21 (d, J=17.2 Hz, 1H), 5.79 (s, 3H), 5.15-4.83 (m, 3H), 4.52-4.26 (m, 2H), 4.17-3.87 (m, 2H), 3.22-2.82 (m, 5H) ppm. MS: M/e 488 (M+1)⁺.

### Compound A9: 2-((2S)-1-acryloyl-4-(5-(5-methyl-1H-indazol-4-yl)-2H-pyrano[2,3-f]quinazolin-10-yl)piperazin-2-yl)acetonitrile

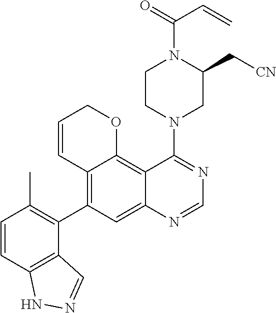 [SMILES: C=CC(=O)N1CCN(c2ncnc3cc(-c4c(C)ccc5[nH]ncc45)c4c(c23)OCC=C4)C[C@@H]1CC#N]

The compound A9 was synthesized starting from the corresponding starting materials according the similar procedures described as those of Compound A8 to give the target compound (8 mg). ¹H NMR (400 MHz, CD₃OD) δ 8.50 (s, 1H), 7.54 (t, J=7.6 Hz, 2H), 7.40 (d, J=8.4 Hz, 1H), 7.27 (s, 1H), 6.98-6.74 (m, 1H), 6.30 (d, J=16.8 Hz, 1H), 5.96 (d, J=10 Hz, 1H), 5.89-5.78 (m, 2H), 5.22-4.96 (m, 2H), 4.68-4.48 (m, 2H), 4.25-4.08 (m, 2H), 3.74-3.46 (m, 2H), 2.96-2.65 (m, 3H), 2.22 (s, 3H) ppm. MS: M/e 492 (M+1)⁺.

### Compound A10: 2-((2S)-1-acryloyl-4-(5-(5-methyl-1H-indazol-4-yl)-3,4-dihydro-2H-pyrano[2,3-f]quinazolin-10-yl)piperazin-2-yl)acetonitrile

### Step A: tert-butyl (S)-4-(5-(benzyloxy)-2H-pyrano[2,3-f]quinazolin-10-yl)-2-(cyanomethyl)piperazine-1-carboxylate

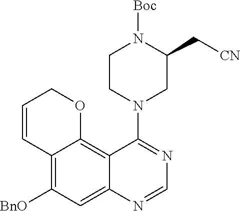 [SMILES: CC(C)(C)OC(=O)N1CCN(c2ncnc3cc(OCc4ccccc4)c4c(c23)OCC=C4)C[C@@H]1CC#N]

To a stirred mixture of 5-(benzyloxy)-2,9-dihydro-10H-pyrano[2,3-f]quinazolin-10-one (728 mg, 2.38 mmol), tert-butyl (S)-2-(cyanomethyl)piperazine-1-carboxylate (0.8 g, 3.57 mmol) in CH₃CN (10 mL) was added DBU (0.72 g, 4.76 mmol), followed by pyBOP (1.6 g, 3.09 mmol). The suspension became clear from suspension, soon became suspension again. The mixture was stirred for another 2 hours and filtered. The filtrate was diluted with EA (10 mL), washed with H₂O, brine, dried over Na₂SO₄, concentrated and purified by column chromatography (petroleum ether/EA=10:1-2:1) to give the target compound (312 mg, 25.5%). MS: M/e 514 (M+1)⁺.

### Step B: tert-butyl (S)-2-(cyanomethyl)-4-(5-hydroxy-3,4-dihydro-2H-pyrano[2,3-f]quinazolin-10-yl)piperazine-1-carboxylate

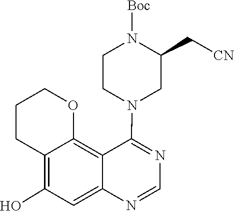 [SMILES: CC(C)(C)OC(=O)N1CCN(c2ncnc3cc(O)c4c(c23)OCCC4)C[C@@H]1CC#N]

To a stirred solution of the product of step A (312 mg, 0.6 mmol) in EtOH/THF (5 mL/5 mL) was added Pd/C (100 mg). After the addition, the reaction was stirred overnight under H₂ (1 atm). The reaction mixture was filtered and the filtrate was concentrated to give the target compound (250 mg, 97.9%). MS: M/e 426 (M+1)⁺.

### Step C: tert-butyl (S)-2-(cyanomethyl)-4-(5-(((trifluoromethyl)sulfonyl)oxy)-3,4-dihydro-2H-pyrano[2,3-f]quinazolin-10-yl)piperazine-1-carboxylate

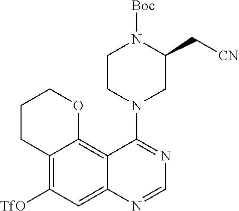 [SMILES: CC(C)(C)OC(=O)N1CCN(c2ncnc3cc(OS(=O)(=O)C(F)(F)F)c4c(c23)OCCC4)C[C@@H]1CC#N]

To a stirred solution of the product of step B (248 mg, 0.58 mmol) in THF (10 mL) was added K₂CO₃ (160 mg, 1.16 mmol), then 1,1,1-trifluoro-N-phenyl-N-((trifluoromethyl)sulfonyl)methanesulfonamide (311 mg, 0.87 mmol) was added and the mixture was stirred overnight. The reaction mixture was treated with EA (10 mL) and washed with H₂O, brine, dried over Na₂SO₄, concentrated and purified by column chromatography (petroleum ether/EA=5:1-2:1) to give the target compound (272 mg, 8.2%). MS: M/e 558 (M+1)⁺.

### Step D: tert-butyl (2S)-2-(cyanomethyl)-4-(5-(5-methyl-1H-indazol-4-yl)-3,4-dihydro-2H-pyrano[2,3-f]quinazolin-10-yl)piperazine-1-carboxylate

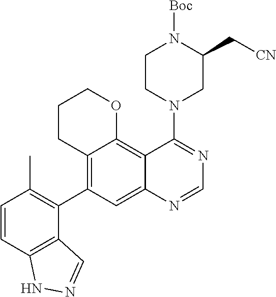 [SMILES: Cc1ccc2[nH]ncc2c1-c1cc2ncnc(N3CCN(C(=O)OC(C)(C)C)[C@@H](CC#N)C3)c2c2c1CCCO2]

To a stirred mixture of the product of C (111.4 mg, 0.2 mmol), (5-methyl-1H-indazol-4-yl)boronic acid (52.8 mg, 0.3 mmol), Pd(dppf)Cl₂ (15 mg, 0.02 mmol) in sat.aq Na₂CO₃ (0.5 mL)/dioxane(2 mL) under N₂. After the addition, the reaction was stirred for 2 hours at 100° C. The reaction was treated with EA (10 mL), washed with H₂O, brine, dried over Na₂SO₄, concentrated and purified by Prep-TLC (petroleum ether/EA=1:1) to give the target compound (30 mg, 27.8%). MS: M/e 540 (M+1)⁺.

### Step E: 2-((2S)-1-acryloyl-4-(5-(5-methyl-1H-indazol-4-yl)-3,4-dihydro-2H-pyrano[2,3-f]quinazolin-10-yl)piperazin-2-yl)acetonitrile

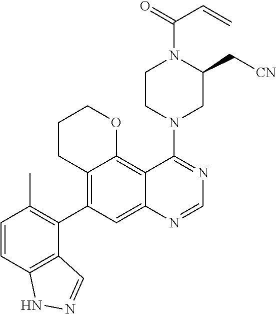 [SMILES: C=CC(=O)N1CCN(c2ncnc3cc(-c4c(C)ccc5[nH]ncc45)c4c(c23)OCCC4)C[C@@H]1CC#N]

To a stirred solution of the product of step D (30 mg, 0.056 mmol) in CH₂Cl₂ (5 mL) was added TFA (1 mL). Then the mixture was stirred for an hour. The reaction mixture was concentrated to give the residue, which was dissolved in EA (10 mL) and washed with aq.sat. Na₂CO₃, brine, dried over Na₂SO₄, concentrated to give the intermediate, which was dissolved in CH₃CN (3 mL) and sat.aq.NaHCO₃ (0.5 mL,) was added, then a solution of acryloyl chloride (5 mg, 0.056 mmol) in CH₃CN (0.5 mL) was added. After the addition, the reaction mixture was stirred for 20 min. The reaction mixture was extracted with EA (10 mL), the organic layer was washed with brine, dried over Na₂SO₄, concentrated and purified by Prep-TLC (EA) to give the target compound (9 mg). ¹H NMR (400 MHz, DMSO-d6) δ 13.14 (s, 1H), 8.58 (s, 1H), 7.55-7.47 (m, 2H), 7.35 (d, J=7.6 Hz, 1H), 7.10 (s, 1H), 6.89 (s, 1H), 6.21 (d, J=16.4 Hz, 1H), 5.80 (d, J=10.4 Hz, 1H), 5.09 (s, 0.5H), 4.89 (s, 0.5H), 4.55-4.28 (m, 3H), 4.15-3.92 (m, 2H), 3.72-3.45 (m, 1H), 3.12-2.82 (m, 4H), 2.27 (s, 2H), 2.14 (d, J=6.4 Hz, 3H), 1.92 (s, 2H) ppm. MS: M/e 494 (M+1)⁺.

### Compound A11: (S)-2-(1-acryloyl-4-(5-(6-methyl-1H-indazol-4-yl)-3,4-dihydro-2H-pyrano[2,3-f]quinazolin-10-yl)piperazin-2-yl)acetonitrile

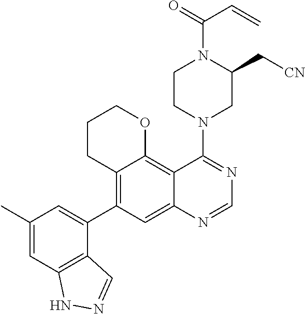 [SMILES: C=CC(=O)N1CCN(c2ncnc3cc(-c4cc(C)cc5[nH]ncc45)c4c(c23)OCCC4)C[C@@H]1CC#N]

The compound A11 was synthesized starting from the corresponding starting materials according the similar procedures described as those of Compound A10

to give the target compound (5 mg). ¹H NMR (400 MHz, DMSO-d6+D2O) δ 8.48 (s, 1H), 7.73 (s, 1H), 7.41 (s, 1H), 7.23 (s, 1H), 6.95 (s, 1H), 6.93-6.79 (m, 1H), 6.21 (d, J=17.2 Hz, 1H), 5.81 (d, J=10.4 Hz, 1H), 5.08 (s, 5.08), 4.86 (s, 1H), 4.55-4.28 (m, 4H), 4.16-3.92 (m, 2H), 3.6-3.43 (m, 1H), 3.33 (d, J=13.6 Hz, 1H), 3.23-2.85 (m, 4H), 2.50 (s, 3H), 1.97-1.83 (m, 2H) ppm. MS: M/e 494 (M+1)⁺.

### Compound A12: 1-(4-(5-(naphthalen-1-yl)-3,4-dihydro-2H-pyrano[2,3-f]quinazolin-10-yl)piperazin-1-yl)prop-2-en-1-one

### Step A: tert-butyl 4-(5-hydroxy-3,4-dihydro-2H-pyrano[2,3-f]quinazolin-10-yl)piperazine-1-carboxylate

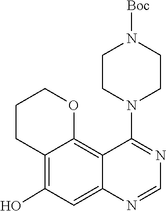 [SMILES: CC(C)(C)OC(=O)N1CCN(c2ncnc3cc(O)c4c(c23)OCCC4)CC1]

To a stirred solution of tert-butyl 4-(5-(benzyloxy)-2H-pyrano[2,3-f]quinazolin-10-yl)piperazine-1-carboxylate (293 mg, 0.616 mmol) in EtOH/THF (10 mL/10 mL) was added Pd/C (100 mg), then the mixture was stirred overnight under H₂ (1 atm) at room temperature. The reaction mixture was filtered and the filtrate was concentrated to give the target compound (210 mg, 88.2%). MS: M/e 387 (M+1)⁺.

### Step B: tert-butyl 4-(5-(((trifluoromethyl)sulfonyl)oxy)-3,4-dihydro-2H-pyrano[2,3-f]quinazolin-10-yl)piperazine-1-carboxylate

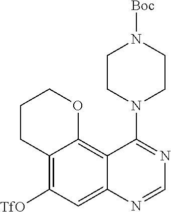 [SMILES: CC(C)(C)OC(=O)N1CCN(c2ncnc3cc(OS(=O)(=O)C(F)(F)F)c4c(c23)OCCC4)CC1]

To a stirred solution of the product of step A (210 mg, 0.54 mmol) in THF (10 mL) was added K₂CO₃ (149 mg, 1.08 mmol), then 1,1,1-trifluoro-N-phenyl-N-((trifluoromethyl)sulfonyl)methanesulfonamide (291 mg, 0.82 mmol) was added. After the addition, the reaction mixture was stirred for 5 hours. The reaction mixture was treated with EA (20 mL), washed with H₂O, brine, dried over Na₂SO₄, concentrated and purified by column chromatography (petroleum ether/EA=5:1-2:1) to give the target compound (247 mg, 88.2%). MS: M/e 519 (M+1)⁺.

### Step C: tert-butyl 4-(5-(naphthalen-1-yl)-3,4-dihydro-2H-pyrano[2,3-f]quinazolin-10-yl)piperazine-1-carboxylate

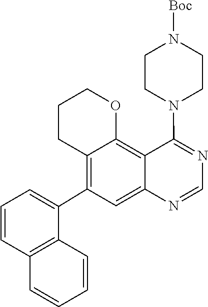 [SMILES: CC(C)(C)OC(=O)N1CCN(c2ncnc3cc(-c4cccc5ccccc45)c4c(c23)OCCC4)CC1]

To a stirred mixture of the product of step B (80 mg, 0.154 mmol), naphthalen-1-ylboronic acid (32 mg, 0.185 mmol) in sat. aq.Na₂CO₃/dioxane (0.5 mL/0.5 mL) was added PddppfCl₂ (11.2 mg, 0.0154 mmol) under N₂. After the addition, the reaction was stirred for an hour at 100° C. The reaction mixture was treated with EA (5 mL), washed with H₂O, brine, dried over Na₂SO₄, concentrated and purified by Prep-TLC (petroleum ether/EA=1:1) to give the target compound (53 mg, 69.4%). MS: M/e 497 (M+1)⁺.

### Step D: 1-(4-(5-(naphthalen-1-yl)-3,4-dihydro-2H-pyrano[2,3-f]quinazolin-10-yl)piperazin-1-yl)prop-2-en-1-one

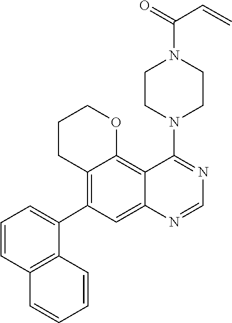 [SMILES: C=CC(=O)N1CCN(c2ncnc3cc(-c4cccc5ccccc45)c4c(c23)OCCC4)CC1]

To a stirred solution of the product of C (53 mg, 0.107 mmol) in CH₂Cl₂ (5 mL) was added TFA (1 mL). After the addition, the reaction was stirred for 3 hours. The reaction mixture was concentrated to give the residue, which was dissolved in EA (10 mL) and washed with aq.sat. Na₂CO₃, brine, dried over Na₂SO₄, concentrated to give the residue, which was dissolved in CH₃CN (5 mL), sat.aq.NaHCO₃ (0.5 mL) was added, then a solution of acryloyl chloride (9.7 mg, 0.107 mmol) in CH₃CN (0.5 mL) was added to the mixture. EA (5 mL) was added and the mixture was washed with brine, dried over Na₂SO₄, concentrated and purified by Prep-TLC (CH₂Cl₂/MeOH=10:1) to give the target compound (18 mg). ¹H NMR (400 MHz, DMSO-d6) δ 8.49 (s, 1H), 8.55-7.98 (m, 2H), 7.65-7.53 (m, 2H), 7.52-7.41 (m, 3H), 7.16 (s, 1H), 6.92-6.79 (m, 1H), 6.17 (d, J=16.0 Hz, 1H), 5.74 (d, J=11.2 Hz, 1H), 4.33 (s, 2H), 3.82-3.60 (m, 8H), 2.37-2.16 (m, 2H), 1.87 (s, 2H) ppm. MS: M/e 451 (M+1)⁺.

### Compound A13: 1-(4-(5-(5-methyl-1H-indazol-4-yl)-3,4-dihydro-2H-pyrano[2,3-f]quinazolin-10-yl)piperazin-1-yl)prop-2-en-1-one

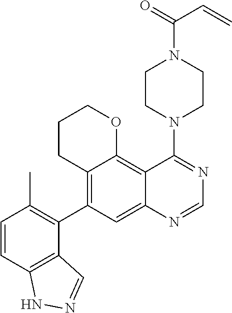 [SMILES: C=CC(=O)N1CCN(c2ncnc3cc(-c4c(C)ccc5[nH]ncc45)c4c(c23)OCCC4)CC1]

The compound A13 was synthesized starting from the corresponding starting materials according the similar procedures described as those of Compound A12 to give the target compound (3 mg). ¹H NMR (400 MHz, DMSO-d6) δ 13.10 (s, 1H), 8.47 (s, 1H), 7.50 (d, J=8.8 Hz, 2H), 7.34 (d, J=8.4 Hz, 1H), 7.08 (s, 1H), 6.86 (dd, J=16.6, 10.4 Hz, 1H), 6.16 (d, J=16.8 Hz, 1H), 5.73 (d, J=12.4 Hz, 1H), 4.34 (s, 2H), 3.86-3.53 (m, 8H), 2.27-2.23 (m, 2H), 2.14 (s, 3H), 1.94-1.84 (m, 2H) ppm. MS: M/e 455 (M+1)⁺.

### Compound A14 and 15: 2-((2S)-1-acryloyl-4-(5-(5-chloro-1H-indazol-4-yl)-3,4-dihydro-2H-pyrano[2,3-f]quinazolin-10-yl)piperazin-2-yl)acetonitrile

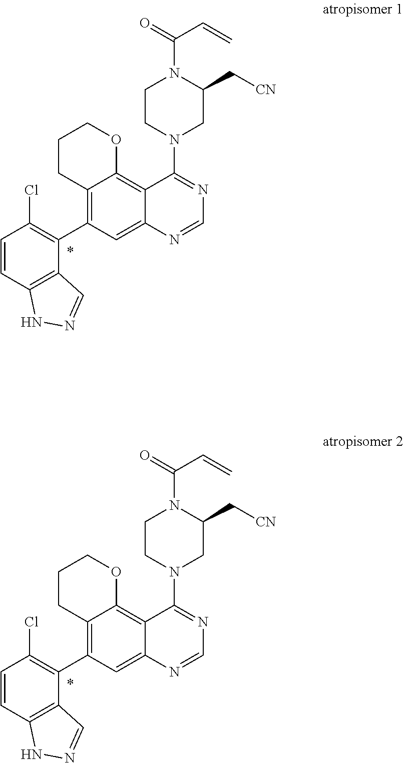 [SMILES: C=CC(=O)N1CCN(c2ncnc3cc(-c4c(Cl)ccc5[nH]ncc45)c4c(c23)OCCC4)C[C@@H]1CC#N ; C=CC(=O)N1CCN(c2ncnc3cc(-c4c(Cl)ccc5[nH]ncc45)c4c(c23)OCCC4)C[C@@H]1CC#N]

The compound A14 was synthesized starting from the corresponding starting materials according the similar procedures described as those of Compound A10 to give two isomers A14 (atropisomer 1, 1.3 mg) and A15 (atropisomer 2, 1.2 mg)

A14: ¹H NMR (400 MHz, DMSO-d6) δ 13.22 (s, 1H), 8.58 (s, 1H), 8.23 (s, 1H), 8.01 (s, 1H), 7.45-7.26 (m, 2H), 6.97 (s, 1H), 6.27 (d, J=16.4 Hz, 1H), 5.86 (d, J=10.4 Hz, 1H), 5.11 (s, 0.5H), 4.88 (s, 0.5), 4.6-4.35 (m, 4H), 4.26-3.93 (m, 2H), 3.27-2.92 (m, 5H), 2.51 (s, 2H), 2.11-2.02 (m, 1H) ppm. MS: M/e 514 (M+1)⁺.

A15: ¹H NMR (400 MHz, DMSO-d6) δ 13.45 (s, 1H), 8.51 (d, J=6.4 Hz, 1H), 7.70-7.60 (m, 2H), 7.56-7.50 (m, 1H), 7.16 (s, 1H), 6.85 (s, 1H), 6.20 (d, J=16.8 Hz, 1H), 5.79 (d, J=10.4 Hz, 1H), 5.11 (s, 0.5H), 4.88 (s, 0.5), 4.55-4.21 (m, 4H), 4.15-3.85 (m, 2H), 3.22-2.82 (m, 5H), 2.45-2.20 (m, 2H), 2.05-1.96 (m, 1H) ppm. MS: M/e 514 (M+1)⁺.

### Compound A16: (S)-2-(1-acryloyl-4-(5-(6-fluoro-1H-indazol-4-yl)-3,4-dihydro-2H-pyrano[2,3-f]quinazolin-10-yl)piperazin-2-yl)acetonitrile

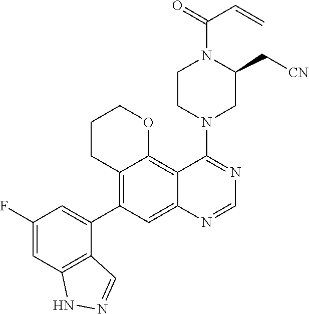 [SMILES: C=CC(=O)N1CCN(c2ncnc3cc(-c4cc(F)cc5[nH]ncc45)c4c(c23)OCCC4)C[C@@H]1CC#N]

The compound A16 was synthesized starting from the corresponding starting materials according the similar procedures described as those of Compound A10 to give the target compound (9 mg). ¹H NMR (400 MHz, DMSO-d6) δ 13.34 (s, 1H), 8.50 (s, 1H), 7.82 (s, 1H), 7.41 (d, J=8.8 Hz, 1H), 7.25 (s, 1H), 7.04 (d, J=9.2 Hz, 1H), 7.00-6.76 (m, 1H), 6.20 (d, J=16.4 Hz, 1H), 5.79 (d, J=10.4 Hz, 1H), 5.09 (s, 0.5H), 4.87 (s, 0.5H), 4.55-4.25 (m, 4H), 4.17-3.86 (m, 2H), 3.26-2.84 (m, 6H), 1.93 (s, 2H) ppm. MS: M/e 498 (M+1)⁺.

### Compound A17: 2-((2S)-1-acryloyl-4-(5-(5-chloro-6-fluoro-1H-indazol-4-yl)-3,4-dihydro-2H-pyrano[2,3-f]quinazolin-10-yl)piperazin-2-yl)acetonitrile

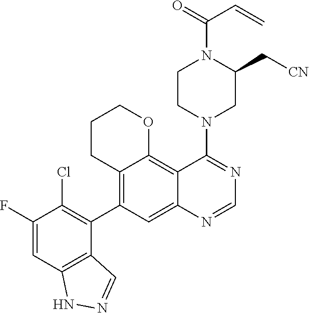 [SMILES: C=CC(=O)N1CCN(c2ncnc3cc(-c4c(Cl)c(F)cc5[nH]ncc45)c4c(c23)OCCC4)C[C@@H]1CC#N]

The compound A17 was synthesized starting from the corresponding starting materials according the similar procedures described as those of Compound A10 to give the target compound (1.5 mg). ¹H NMR (400 MHz, DMSO-d6) δ 13.54 (s, 1H), 8.51 (d, J=6.8 Hz, 1H), 7.77-7.60 (m, 2H), 7.20 (s, 1H), 7.05-6.75 (m, 1H), 6.20 (d, J=16.8 Hz, 1H), 5.79 (d, J=10.4 Hz, 1H), 4.99 (1H), 4.52-4.21 (m, 4H), 4.20-3.82 (m, 2H), 3.25-2.81 (m, 4H), 2.46-2.20 (m, 2H), 2.05-1.93 (m, 2H) ppm. MS: M/e 532 (M+1)⁺.

### Compound A18: (S)-2-(1-acryloyl-4-(5-(6-(trifluoromethyl)-1H-indazol-4-yl)-3,4-dihydro-2H-pyrano[2,3-f]quinazolin-10-yl)piperazin-2-yl)acetonitrile

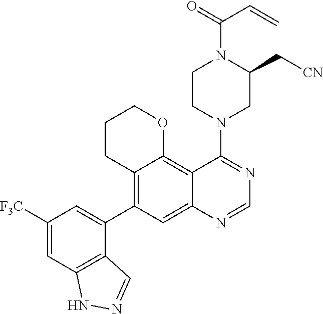 [SMILES: C=CC(=O)N1CCN(c2ncnc3cc(-c4cc(C(F)(F)F)cc5[nH]ncc45)c4c(c23)OCCC4)C[C@@H]1CC#N]

The compound A18 was synthesized starting from the corresponding starting materials according the similar procedures described as those of Compound A10 to give the target compound (5.2 mg). ¹H NMR (400 MHz, DMSO-d6) δ 13.75 (s, 1H), 8.50 (s, 1H), 8.01 (d, J=4.8 Hz, 2H), 7.37 (s, 1H), 7.27 (s, 1H), 7.02-6.77 (m, 1H), 6.20 (d, J=16.8 Hz, 1H), 5.79 (d, J=10.4 Hz, 1H), 4.98 (1H), 4.51-4.27 (m, 4H), 4.16-3.89 (m, 2H), 3.56 (s, 1H), 3.00 (m, 5H), 1.92 (s, 2H) ppm. MS: M/e 548 (M+1)⁺.

### Compound A19: 1-(4-(5-(5-chloro-6-fluoro-1H-indazol-4-yl)-3,4-dihydro-2H-pyrano[2,3-f]quinazolin-10-yl)piperazin-1-yl)prop-2-en-1-one

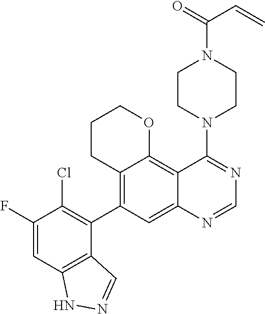 [SMILES: C=CC(=O)N1CCN(c2ncnc3cc(-c4c(Cl)c(F)cc5[nH]ncc45)c4c(c23)OCCC4)CC1]

The compound A19 was synthesized starting from the corresponding starting materials according the similar procedures described as those of Compound A10 to give the target compound (0.5 mg). ¹H NMR (400 MHz, CD₃OD) δ 8.47 (s, 1H), 7.63 (s, 1H), 7.53 (d, J=8.4 Hz, 1H), 7.20 (s, 1H), 6.84 (dd, J=16.8, 10.8 Hz, 1H), 6.27 (d, J=16.8 Hz, 1H), 5.80 (d, J=10.8 Hz, 1H), 5.34 (s, 0.5H), 4.61 (s, 0.5H), 4.45-4.38 (m, 2H), 3.93-3.74 (m, 8H), 2.63-2.52 (m, 1H), 2.42-2.30 (m, 1H), 2.17 (t, 1H) ppm. MS: M/e 493 (M+1)⁺.

### Compound A20: 2-((2S)-1-acryloyl-4-(5-(5-chloro-6-methyl-1H-indazol-4-yl)-3,4-dihydro-2H-pyrano[2,3-f]quinazolin-10-yl)piperazin-2-yl)acetonitrile

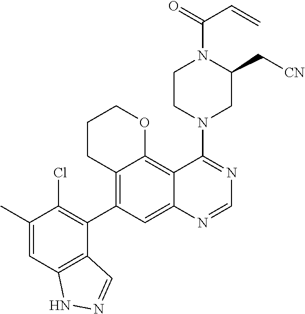 [SMILES: C=CC(=O)N1CCN(c2ncnc3cc(-c4c(Cl)c(C)cc5[nH]ncc45)c4c(c23)OCCC4)C[C@@H]1CC#N]

The compound A20 was synthesized starting from the corresponding starting materials according the similar procedures described as those of Compound A10 to give the target compound (3 mg). ¹H NMR (400 MHz, DMSO-d6) δ 13.32 (s, 1H), 8.55 (s, 1H), 7.63 (s, 1H), 7.54 (s, 1H), 7.14 (s, 1H), 7.01-6.75 (m, 1H), 6.21 (d, J=16.4 Hz, 1H), 5.80 (d, J=10.8 Hz, 1H), 5.11 (s, 0.5H), 4.88 (s, 0.5H), 4.52-4.28 (m, 4H), 4.18-3.87 (m, 2H), 3.21-2.81 (m, 4H), 2.44-2.17 (m, 5H), 1.94 (s, 2H) ppm. MS: M/e 528 (M+1)⁺.

### Compound A21: (S)-2-(1-acryloyl-4-(5-(4-hydroxynaphthalen-1-yl)-3,4-dihydro-2H-pyrano[2,3-f]quinazolin-10-yl)piperazin-2-yl)acetonitrile

### Step A: tert-butyl (S)-2-(cyanomethyl)-4-(5-(4-(pivaloyloxy)naphthalen-1-yl)-3,4-dihydro-2H-pyrano[2,3-f]quinazolin-10-yl)piperazine-1-carboxylate

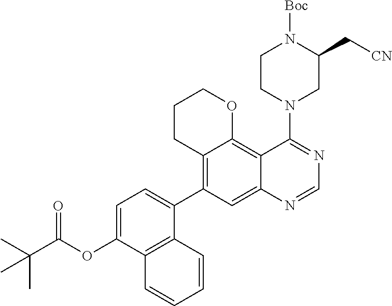 [SMILES: CC(C)(C)OC(=O)N1CCN(c2ncnc3cc(-c4ccc(OC(=O)C(C)(C)C)c5ccccc45)c4c(c23)OCCC4)C[C@@H]1CC#N]

To a stirred solution of tert-butyl (S)-2-(cyanomethyl)-4-(5-(((trifluoromethyl)sulfonyl)oxy)-3,4-dihydro-2H-pyrano[2,3-f]quinazolin-10-yl)piperazine-1-carboxylate (100 mg, 0.179 mmol), 4-(4,4,5,5-tetramethyl-1,3,2-dioxaborolan-2-yl)naphthalen-1-yl pivalate (70 mg, 0.197 mmol) in aq.sat. Na₂CO₃/dioxane (0.5 mL/5 mL) was added PddppfCl₂ (13 mg, 0.0179 mmol) under N₂. After the addition, the reaction was stirred for an hour at 100° C. The reaction mixture was diluted with EA (10 mL), washed with H₂O, brine, dried over Na₂SO₄, concentrated and purified by Prep-TLC (petroleum ether/EA=2:1) to give the target compound (76 mg, 66.7%). MS: M/e 636 (M+1)⁺.

### Step B: (S)-4-(10-(4-acryloyl-3-(cyanomethyl)piperazin-1-yl)-3,4-dihydro-2H-pyrano[2,3-f]quinazolin-5-yl)naphthalen-1-yl pivalate

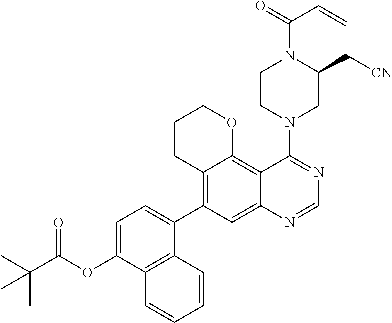 [SMILES: C=CC(=O)N1CCN(c2ncnc3cc(-c4ccc(OC(=O)C(C)(C)C)c5ccccc45)c4c(c23)OCCC4)C[C@@H]1CC#N]

To a stirred solution of the product of A (76 mg, 0.119 mmol) in CH₂Cl₂ (5 mL) was added TFA (0.5 mL). After the addition, the reaction was stirred overnight. The reaction mixture was concentrated to give the residue, which was dissolved in EA (10 mL) and washed with aq.sat. Na₂CO₃, brine, dried over Na₂SO₄, concentrated to give the intermediate, which was dissolved in CH₃CN (5 mL), sat. aq.NaHCO₃ (0.5 mL) was added, then a solution of acryloyl chloride (10.8 mg, 0.12 mmol) in CH₃CN (1 mL) was added. The reaction mixture was stirred for 1 hour. EA (10 mL) was added and the mixture was washed with brine, dried over Na₂SO₄, concentrated and purified by Prep-TLC (CH₂Cl₂/MeOH=10:1) to give the target compound (25 mg, 35.6%). MS: M/e 590 (M+1)⁺.

### Step C: (S)-2-(1-acryloyl-4-(5-(4-hydroxynaphthalen-1-yl)-3,4-dihydro-2H-pyrano[2,3-f]quinazolin-10-yl)piperazin-2-yl)acetonitrile

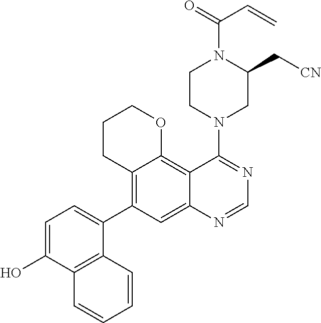 [SMILES: C=CC(=O)N1CCN(c2ncnc3cc(-c4ccc(O)c5ccccc45)c4c(c23)OCCC4)C[C@@H]1CC#N]

To a stirred solution of the product of step B (25 mg, 0.042 mmol) in THF/H₂O (5 mL/1 mL) was added a little amount of LiOH (2 mg). After the addition, the reaction was stirred overnight. The reaction mixture was acidified to pH=4˜5, then extracted with EA (10 mL×2). The combined organic layers were washed with brine, dried over Na₂SO₄, concentrated and purified by Prep-TLC (CH₂Cl₂/MeOH=10:1) to give the target compound (9 mg). ¹H NMR (400 MHz, DMSO-d6) δ 10.36 (s, 1H), 8.49 (s, 1H), 8.23 (d, J=8.4 Hz, 1H), 7.54-7.31 (m, 3H), 7.29-7.12 (m, 2H), 7.02-6.78 (m, 2H), 6.20 (d, J=16.4 Hz, 1H), 5.79 (d, J=10.8 Hz, 1H), 5.09 (s, 0.5H), 4.87 (s, 0.5H), 4.51-4.22 (m, 3H), 4.18-3.87 (m, 1.5H), 4.56 (s, 0.5H), 3.29-2.85 (m, 5H), 2.43-2.17 (m, 2H), 1.87 (s, 2H) ppm. MS: M/e 506 (M+1)⁺.

### Compound A22: (S)-2-(1-acryloyl-4-(5-(6-amino-4-(trifluoromethyl)pyridin-3-yl)-3,4-dihydro-2H-pyrano[2,3-f]quinazolin-10-yl)piperazin-2-yl)acetonitrile

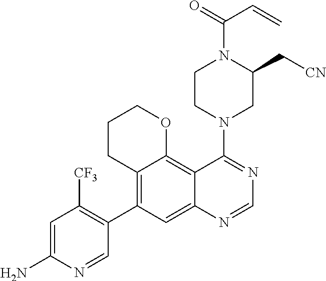 [SMILES: C=CC(=O)N1CCN(c2ncnc3cc(-c4cnc(N)cc4C(F)(F)F)c4c(c23)OCCC4)C[C@@H]1CC#N]

The compound A22 was synthesized starting from the corresponding starting materials according the similar procedures described as those of Compound A10 to give the target compound (8 mg). ¹H NMR (400 MHz, DMSO-d6) δ 8.51 (s, 1H), 7.90 (s, 1H), 7.10 (s, 1H), 6.86 (s, 2H), 6.70 (s, 2H), 6.19 (d, J=16.8 Hz, 1H), 5.79 (d, J=9.6 Hz, 1H), 5.09 (s, 0.5H), 4.87 (s, 0.5H), 4.53-4.02 (m, 5H), 3.83 (s, 1H), 3.22-2.83 (m, 5H), 2.32-2.18 (m, 1H), 2.06-1.82 (m, 2H) ppm. MS: M/e 524 (M+1)⁺.

### Compound A23: 2-((2S)-4-(5-(5-chloro-6-methyl-1H-indazol-4-yl)-3,4-dihydro-2H-pyrano[2,3-f]quinazolin-10-yl)-1-(2-fluoroacryloyl)piperazin-2-yl)acetonitrile

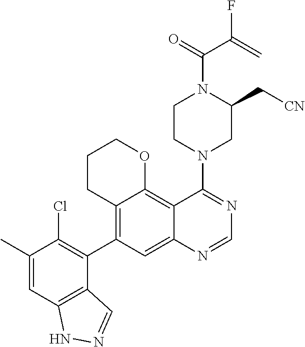 [SMILES: C=C(F)C(=O)N1CCN(c2ncnc3cc(-c4c(Cl)c(C)cc5[nH]ncc45)c4c(c23)OCCC4)C[C@@H]1CC#N]

To a stirred solution of 2-((2S)-4-(5-(5-chloro-6-methyl-1H-indazol-4-yl)-3,4-dihydro-2H-pyrano[2,3-f]quinazolin-10-yl)piperazin-2-yl)acetonitrile (5 mg, 0.0105 mmol) in THF (3 mL) was added 2-fluoroacrylic acid (1.9 mg, 0.021 mmol), followed by T₃P (6.68 mg, 0.021 mmol) and Et₃N (2.1 mg, 0.021 mmol). After the addition, the reaction was stirred for 20 min. The reaction mixture was diluted with EA (5 mL), washed with brine, dried over Na₂SO₄, concentrated and purified by Prep-TLC (CH₂Cl₂/MeOH=10:1) to give the target compound (1 mg). ¹H NMR (400 MHz, DMSO-d6) δ 13.34 (s, 1H), 8.61 (s, 1H), 7.65 (s, 1H), 7.58-7.52 (m, 1H), 7.14 (s, 1H), 5.48-5.22 (m, 2H), 4.40 (s, 2H), 3.97 (s, 2H), 3.00 (s, 6H), 2.52 (s, 3H), 2.43-2.18 (m, 2H), 2.05-1.96 (m, 3H) ppm. MS: M/e 546 (M+1)⁺.

### Compound A24: (S)-2-(1-acryloyl-4-(5-(2,6-dimethylphenyl)-3,4-dihydro-2H-pyrano[2,3-f]quinazolin-10-yl)piperazin-2-yl)acetonitrile

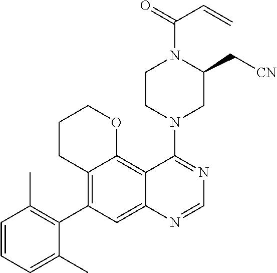 [SMILES: C=CC(=O)N1CCN(c2ncnc3cc(-c4c(C)cccc4C)c4c(c23)OCCC4)C[C@@H]1CC#N]

The compound A24 was synthesized starting from the corresponding starting materials according the similar procedures described as those of Compound A10 to give the target compound (2.2 mg). ¹H NMR (400 MHz, DMSO-d6) δ 8.47 (s, 1H), 7.27-7.13 (m, 3H), 6.96 (s, 1H), 6.80-6.67 (m, 1H), 6.20 (d, J=16.8 Hz, 1H), 5.78 (d, J=10.4 Hz, 1H), 5.32 (s, 1H), 4.97 (1H), 4.52-4.02 (m, 3H), 3.92-3.47 (m, 1H), 3.29-2.82 (m, 5H), 2.25-2.16 (m, 2H), 2.04-1.91 (m, 8H) ppm. MS: M/e 468 (M+1)⁺.

### Compound A25: 2-((2S)-1-acryloyl-4-(5-(5,6-dimethyl-1H-indazol-4-yl)-3,4-dihydro-2H-pyrano[2,3-f]quinazolin-10-yl)piperazin-2-yl)acetonitrile

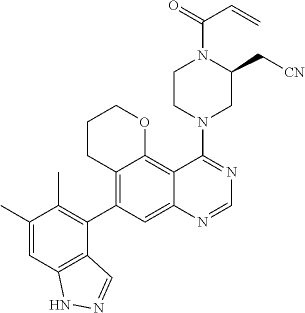 [SMILES: C=CC(=O)N1CCN(c2ncnc3cc(-c4c(C)c(C)cc5[nH]ncc45)c4c(c23)OCCC4)C[C@@H]1CC#N]

The compound A25 was synthesized starting from the corresponding starting materials according the similar procedures described as those of Compound A10 to give the target compound (2 mg). ¹H NMR (400 MHz, DMSO-d6) δ 12.95 (s, 1H), 8.49 (d, J=5.2 Hz, 1H), 7.39 (d, J=4.8 Hz, 2H), 7.07 (s, 1H), 7.0-6.78 (m, 1H), 6.20 (d, J=16.8 Hz, 1H), 5.79 (d, J=10.4 Hz, 1H), 4.97 (1H), 4.55-4.21 (m, 3H), 4.12-3.91 (m, 1H), 3.22-2.78 (m, 6H), 2.43 (s, 3H), 2.33-2.24 (s, 2H), 2.02-1.91 (s, 5H) ppm. MS: M/e 508 (M+1)⁺.

### Compound A26: 2-((2S)-1-acryloyl-4-(5-(6-chloro-5-methyl-1H-indazol-4-yl)-3,4-dihydro-2H-pyrano[2,3-f]quinazolin-10-yl)piperazin-2-yl)acetonitrile

### Step A: tert-butyl (2S)-4-(5-(6-chloro-5-methyl-1-(tetrahydro-2H-pyran-2-yl)-1H-indazol-4-yl)-3,4-dihydro-2H-pyrano[2,3-f]quinazolin-10-yl)-2-(cyanomethyl)piperazine-1-carboxylate

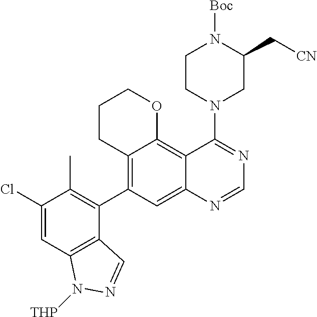

To a stirred solution of tert-butyl (S)-2-(cyanomethyl)-4-(5-(((trifluoromethyl)sulfonyl)oxy)-3,4-dihydro-2H-pyrano[2,3-f]quinazolin-10-yl)piperazine-1-carboxylate (80 mg, 0.144 mmol), 6-chloro-5-methyl-1-(tetrahydro-2H-pyran-2-yl)-4-(4,4,5,5-tetramethyl-1,3,2-dioxaborolan-2-yl)-1H-indazole (64.8 mg, 0.172 mmol) in sat. aq. Na₂CO₃/dioxane (0.5 mL/5 mL) was added PddppfCl₂ (10.5 mg, 0.0144 mmol) under N₂. After the addition, the reaction was stirred for 3 hour at 100° C. The reaction mixture was diluted with EA (20 mL), washed with H₂O, brine, dried over Na₂SO₄, concentrated and purified by Prep-TLC (petroleum ether/EA=1:1) to give the target compound (10 mg, 10.5%). MS: M/e 658 (M+1)⁺.

### Step B: 2-((2S)-1-acryloyl-4-(5-(6-chloro-5-methyl-1H-indazol-4-yl)-3,4-dihydro-2H-pyrano[2,3-f]quinazolin-10-yl)piperazin-2-yl)acetonitrile

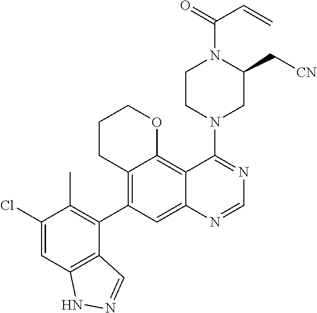 [SMILES: C=CC(=O)N1CCN(c2ncnc3cc(-c4c(C)c(Cl)cc5[nH]ncc45)c4c(c23)OCCC4)C[C@@H]1CC#N]

To a stirred solution of the product of A (10 mg, 0.015 mmol) in CH₂Cl₂ (5 mL) was added TFA (0.5 mL). After the addition, the reaction was stirred overnight. The reaction mixture was concentrated to give the residue, which was dissolved in EA (10 mL) and washed with aq.sat. Na₂CO₃, brine, dried over Na₂SO₄, concentrated to give the intermediate, which was dissolved in CH₃CN (3 mL), sat. aq.NaHCO₃ (0.5 mL) was added, then a solution of acryloyl chloride (1.36 mg, 0.015 mmol) in CH₃CN (0.5 mL) was added. The reaction mixture was stirred at rt for 1 hour. EA (10 mL) was added and the mixture was washed with brine, dried over Na₂SO₄, concentrated and purified by Prep-TLC (CH₂Cl₂/MeOH=10:1) to give the target compound (2.1 mg). ¹H NMR (400 MHz, DMSO-d6) δ 13.25 (s, 1H), 8.50 (d, J=5.6 Hz, 1H), 7.74 (s, 1H), 7.52 (s, 1H), 7.12 (s, 1H), 7.02-6.76 (m, 1H), 6.20 (d, J=16.4 Hz, 1H), 5.79 (d, J=10.4 Hz, 1H), 4.97 (1H), 4.53-4.23 (m, 4H), 4.18-3.89 (m, 2H), 3.24-2.79 (m, 4H), 2.36-2.21 (m, 2H), 2.15 (d, J=12.4 Hz, 3H), 2.03-1.90 (m, 2H) ppm. MS: M/e 528 (M+1)⁺.

### Compound A27: (S)-2-(1-acryloyl-4-(5-(3-chloro-4-hydroxyphenyl)-3,4-dihydro-2H-pyrano[2,3-f]quinazolin-10-yl)piperazin-2-yl)acetonitrile

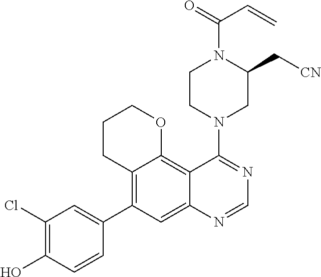 [SMILES: C=CC(=O)N1CCN(c2ncnc3cc(-c4ccc(O)c(Cl)c4)c4c(c23)OCCC4)C[C@@H]1CC#N]

The compound A27 was synthesized starting from the corresponding starting materials according the similar procedures described as those of Compound A21 to give the target compound (4 mg). ¹H NMR (400 MHz, DMSO-d6) δ 10.47 (s, 1H), 8.46 (s, 1H), 7.40 (s, 1H), 7.22 (d, J=8.4 Hz, 1H), 7.15-7.05 (m, 2H), 6.97-6.78 (m, 1H), 6.19 (d, J=17.2 Hz, 1H), 5.78 (d, J=10.4 Hz, 1H), 4.96 (1H), 4.48-4.02 (m, 4H), 3.86 (d, J=12.0 Hz, 1H), 3.26 (d, J=13.2 Hz, 1H), 3.19-2.85 (m, 4H), 2.74-2.56 (m, 2H), 1.98-1.91 (m, 2H) ppm. MS: M/e 490 (M+1)⁺.

### Compound A28: (S)-2-(1-acryloyl-4-(5-(3-fluoro-4-hydroxyphenyl)-3,4-dihydro-2H-pyrano[2,3-f]quinazolin-10-yl)piperazin-2-yl)acetonitrile

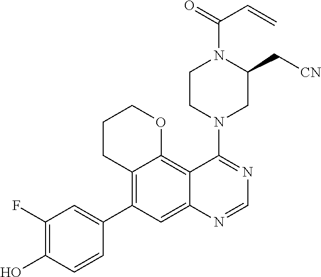 [SMILES: C=CC(=O)N1CCN(c2ncnc3cc(-c4ccc(O)c(F)c4)c4c(c23)OCCC4)C[C@@H]1CC#N]

The compound A28 was synthesized starting from the corresponding starting materials according the similar procedures described as those of Compound A21 to give the target compound (4 mg). ¹H NMR (400 MHz, DMSO-d6) δ 10.11 (s, 1H), 8.46 (s, 1H), 7.24 (d, J=12.0 Hz, 1H), 7.13 (s, 1H), 7.06 (s, 2H), 7.01-6.74 (m, 1H), 6.19 (d, J=16.8 Hz, 1H), 5.78 (d, J=10.4 Hz, 1H), 4.96 (1H), 4.52-3.98 (m, 4H), 3.86 (d, J=12.0 Hz, 1H), 3.26 (d, J=13.2 Hz, 1H), 3.17-2.86 (m, 4H), 2.77-2.59 (m, 2H), 2.01-1.89 (m, 2H) ppm. MS: M/e 474 (M+1)⁺.

### Compound A29: 2-((2S)-1-acryloyl-4-(5-(5,6-dichloro-1H-indazol-4-yl)-3,4-dihydro-2H-pyrano[2,3-f]quinazolin-10-yl)piperazin-2-yl)acetonitrile

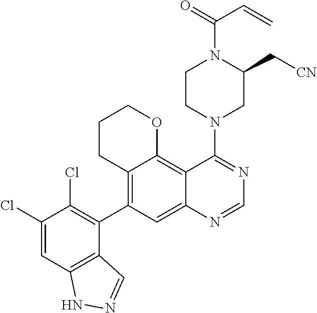 [SMILES: C=CC(=O)N1CCN(c2ncnc3cc(-c4c(Cl)c(Cl)cc5[nH]ncc45)c4c(c23)OCCC4)C[C@@H]1CC#N]

The compound A29 was synthesized starting from the corresponding starting materials according the similar procedures described as those of Compound A26 to give the target compound (10 mg). ¹H NMR (400 MHz, DMSO-d6) δ 13.58 (s, 1H), 8.55 (s, 1H), 7.99 (s, 1H), 7.67 (s, 1H), 7.18 (d, J=3.2 Hz, 1H), 7.02-6.76 (m, 1H), 6.20 (d, J=16.8 Hz, 1H), 5.80 (d, J=10.0 Hz, 1H), 5.00 (1H), 4.55-4.27 (m, 4H), 4.15-3.85 (m, 2H), 3.25-2.81 (m, 4H), 2.45-2.17 (m, 2H), 2.01-1.89 (m, 2H) ppm. MS: M/e 548 (M+1)⁺.

### Compound A30: 2-((2S)-4-(5-(2-fluoro-6-methylphenyl)-3,4-dihydro-2H-pyrano[2,3-f]quinazolin-10-yl)-1-(2-fluoroacryloyl)piperazin-2-yl)acetonitrile

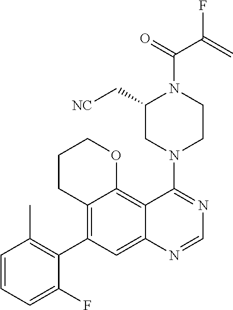 [SMILES: C=C(F)C(=O)N1CCN(c2ncnc3cc(-c4c(C)cccc4F)c4c(c23)OCCC4)C[C@@H]1CC#N]

The compound A30 was synthesized starting from the corresponding starting materials according the similar procedures described as those of Compound A10 to get the product (15 mg,). ¹H NMR (400 MHz, DMSO-d₆) δ 8.49 (s, 1H), 7.45-7.33 (m, 1H), 7.23 (d, J=5.9 Hz, 1H), 7.16 (t, J=8.8 Hz, 1H), 7.13-7.07 (m, 1H), 5.46-5.19 (m, 2H), 4.93 (s, 1H), 4.39 (s, 1H), 4.33 (s, 2H), 3.96-3.86 (m, 1H), 3.80-3.40 (m, 1H), 3.31-3.23 (m, 1H), 3.22-2.75 (m, 4H), 2.43-2.23 (m, 2H), 2.04 (d, J=5.9 Hz, 3H), 1.98 (s, 2H). MS: M/e 490 (M+1)⁺

### Compound A31: (S)-2-(1-acryloyl-4-(5-(2-fluoro-3-methylphenyl)-3,4-dihydro-2H-pyrano[2,3-f]quinazolin-10-yl)piperazin-2-yl)acetonitrile

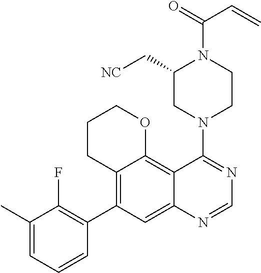 [SMILES: C=CC(=O)N1CCN(c2ncnc3cc(-c4cccc(C)c4F)c4c(c23)OCCC4)C[C@@H]1CC#N]

The compound A31 was synthesized starting from the corresponding starting materials according the similar procedures described as those of Compound A10 to get the product (15 mg). ¹H NMR (400 MHz, DMSO-d₆) δ 8.47 (s, 1H), 7.37 (d, J=7.4 Hz, 1H), 7.27-7.16 (m, 2H), 7.15 (s, 1H), 6.84 (s, 1H), 6.23-6.15 (m, 1H), 5.84-5.73 (m, 1H), 5.14-4.75 (m, 1H), 4.57-4.07 (m, 4H), 4.06-3.38 (m, 2H), 3.33 (s, 1H), 3.30-3.22 (m, 1H), 3.09 (s, 2H), 2.93 (s, 2H), 2.31 (s, 3H), 1.97 (s, 2H). MS: M/e 472 (M+1)⁺

### Compound A32: (S)-2-(1-acryloyl-4-(5-(8-chloronaphthalen-1-yl)-3,4-dihydro-2H-pyrano[2,3-f]quinazolin-10-yl)piperazin-2-yl)acetonitrile

### Step A: tert-butyl (S)-4-(5-(8-chloronaphthalen-1-yl)-3,4-dihydro-2H-pyrano[2,3-f]quinazolin-10-yl)-2-(cyanomethyl)piperazine-1-carboxylate

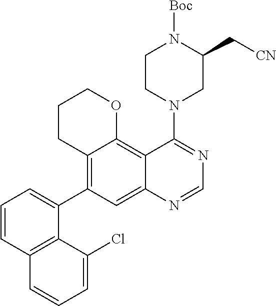 [SMILES: CC(C)(C)OC(=O)N1CCN(c2ncnc3cc(-c4cccc5cccc(Cl)c45)c4c(c23)OCCC4)C[C@@H]1CC#N]

To a stirred solution of tert-butyl (S)-2-(cyanomethyl)-4-(5-(((trifluoromethyl)sulfonyl)oxy)-3,4-dihydro-2H-pyrano[2,3-f]quinazolin-10-yl)piperazine-1-carboxylate (80 mg, 0.144 mmol), 2-(8-chloronaphthalen-1-yl)-4,4,5,5-tetramethyl-1,3,2-dioxaborolane (50 mg, 0.172 mmol) in sat. aq.Na₂CO₃/dioxane (0.5 mL/5 mL) was added PddppfCl₂ (10.5 mg, 0.0144 mmol) under N₂. After the addition, the reaction was stirred for 3 hours at 100° C. The reaction mixture was diluted with EA (20 mL), washed with H₂O, brine, dried over Na₂SO₄, concentrated and purified by Prep-TLC (petroleum ether/EA=1:1) to give the target compound (30 mg, 36.5%). MS: M/e 570 (M+1)⁺.

### Step B: (S)-2-(1-acryloyl-4-(5-(8-chloronaphthalen-1-yl)-3,4-dihydro-2H-pyrano[2,3-f]quinazolin-10-yl)piperazin-2-yl)acetonitrile

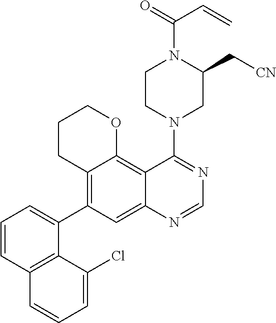 [SMILES: C=CC(=O)N1CCN(c2ncnc3cc(-c4cccc5cccc(Cl)c45)c4c(c23)OCCC4)C[C@@H]1CC#N]

To a stirred solution of the product of A (30 mg, 0.053 mmol) in CH₂Cl₂ (5 mL) was added HCl/dioxane (4.0 M, 3 mL). After the addition, the reaction was stirred for 3 hours. The reaction mixture was concentrated to give the residue, which was treated with CH₃CN (3 mL), sat. aq.NaHCO₃ (0.5 mL) was added, then a solution of acryloyl chloride (4.76 mg, 0.053 mmol) in CH₃CN (0.5 mL) was added. The reaction mixture was stirred at RT for 1 hour. EA (10 mL) was added and the mixture was washed with brine, dried over Na₂SO₄, concentrated and purified by Prep-TLC (CH₂Cl₂/MeOH=10:1) to give the target compound (8 mg). ¹H NMR (400 MHz, DMSO-d6) δ 8.53 (s, 1H), 8.11 (d, J=8.0 Hz, 2H), 7.72-7.57 (m, 2H), 7.57-7.49 (m, 1H), 7.6-7.361 (m, 1H), 7.16 (s, 1H), 7.01-6.76 (m, 1H), 6.20 (d, J=16.8 Hz, 1H), 5.79 (d, J=10.4 Hz, 1H), 4.99 (1H), 4.51-3.83 (m, 5H), 3.27-2.81 (m, 4H), 2.31-2.12 (m, 2H), 2.03-1.86 (m, 2H) ppm. MS: M/e 524 (M+1)⁺.

### Compound A33: 2-((2S)-1-acryloyl-4-(5-(2-fluoro-6-methylphenyl)-3,4-dihydro-2H-pyrano[2,3-f]quinazolin-10-yl)piperazin-2-yl)acetonitrile

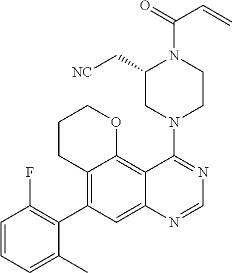 [SMILES: C=CC(=O)N1CCN(c2ncnc3cc(-c4c(C)cccc4F)c4c(c23)OCCC4)C[C@@H]1CC#N]

the compound A33 was synthesized starting from the corresponding starting materials according the similar procedures described as those of Compound A10 to get the product (12 mg). ¹H NMR (400 MHz, DMSO-d₆) δ 8.52 (s, 1H), 7.43-7.36 (m, 1H), 7.23 (d, J=6.4 Hz, 1H), 7.17 (t, J=8.7 Hz, 1H), 7.09 (s, 1H), 6.82 (s, 1H), 6.20 (d, J=16.8 Hz, 1H), 5.79 (d, J=10.2 Hz, 1H), 4.38 (s, 3H), 3.90 (s, 1H), 2.94 (s, 2H), 2.46-2.19 (m, 3H), 2.04 (d, J=5.1 Hz, 3H), 1.98 (s, 2H), 1.23 (s, 2H), 1.18 (d, J=5.9 Hz, 2H) ppm. MS: M/e 472 (M+1)⁺

### Compound A34: (S)-2-(1-acryloyl-4-(5-(2,6-difluorophenyl)-3,4-dihydro-2H-pyrano[2,3-f]quinazolin-10-yl)piperazin-2-yl)acetonitrile

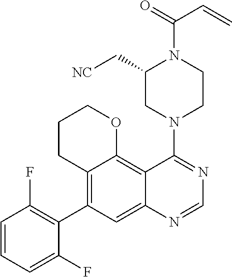 [SMILES: C=CC(=O)N1CCN(c2ncnc3cc(-c4c(F)cccc4F)c4c(c23)OCCC4)C[C@@H]1CC#N]

The compound A34 was synthesized starting from the corresponding starting materials according the similar procedures described as those of Compound A10 to get the product (17 mg). ¹H NMR (400 MHz, DMSO-d₆)) δ 8.48 (s, 1H), 7.71-7.43 (m, 1H), 7.37-7.16 (m, 3H), 6.84 (s, 1H), 6.19 (d, J=16.2 Hz, 1H), 5.78 (d, J=10.0 Hz, 1H), 5.10-4.80 (m, 1H), 4.43-4.27 (m, 3H), 3.88 (d, J=12.2 Hz, 1H), 3.20-2.83 (m, 5H), 2.00 (d, J=7.0 Hz, 3H), 1.46 (d, J=7.0 Hz, 1H), 0.87-0.83 (m, 1H) ppm. MS: M/e 476 (M+1)⁺

### Compound B1: 2-((2S)-1-acryloyl-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)piperazin-2-yl)acetonitrile

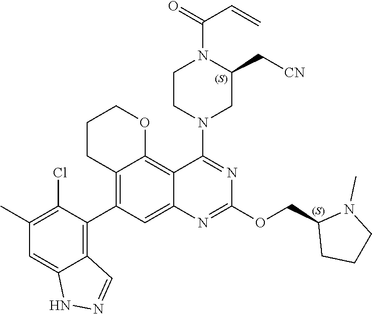

To a mixture of the 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)piperazin-2-yl)acetonitrile (76 mg) and saturated NaHCO₃ aq. (0.5 mL) in CH₃CN (6 mL) was added a solution of acryloyl chloride (11.6 mg, 0.13 mmol) in CH₃CN (1 mL) dropwise slowly. The resulting mixture was stirred at RT for 30 mins. The reaction mixture was diluted with EtOAc (10 mL) and washed with water (15 mL). extracted with EtOAc (15 mL). The organic layers were dried with MgSO₄, filtered and evaporated to afford crude product. The crude product was purified by prep-TLC (DCM:MeOH=20:1) to give the target compound (20 mg, 24% yield). 1H NMR (400 MHz, DMSO-d6) δ 13.42 (s, 1H), 7.68 (s, 1H), 7.59 (s, 1H), 7.06-6.86 (m, 2H), 6.27 (d, J=16 Hz, 1H), 5.86 (d, J=12 Hz, 1H), 5.03-4.92 (m, 1H), 4.85-4.02 (m, 8H), 3.52 (s, 1H), 3.27-2.52 (m, 8H), 2.41-1.92 (m, 9H), 1.29 (s, 3H) ppm. MS: M/e 641 (M+1)⁺.

### Compound B2: 1-(4-(5-(2,6-difluorophenyl)-8-(3-(dimethylamino)azetidin-1-yl)-3,4-dihydro-2H-pyrano[2,3-f]quinazolin-10-yl)piperazin-1-yl)prop-2-en-1-one

The compound B2 was synthesized starting from the corresponding starting materials according the similar procedures described as those of Compound B3 to get the product (2 mg). ¹H NMR (400 MHz, DMSO-d6) δ 7.61-7.50 (m, 1H), 7.25 (t, J=7.6 Hz, 2H), 6.91-6.79 (m, 2H), 6.16 (d, J=16.4 Hz, 1H), 5.73 (d, J=10.8 Hz, 1H), 4.29 (s, 2H), 4.13 (s, 2H), 3.94 (s, 2H), 3.72 (d, J=13.2 Hz, 4H), 3.51 (s, 4H), 3.01 (s, 1H), 2.43-2.10 (m, 8H), 1.98-1.88 (m, 2H) ppm. MS: M/e 535 (M+1)⁺.

### Compound B3: 2-((2S)-1-acryloyl-4-(5-(5-chloro-6-methyl-1H-indazol-4-yl)-8-(((3S,4S)-4-methoxy-1-methylpyrrolidin-3-yl)oxy)-3,4-dihydro-2H-pyrano[2,3-f]quinazolin-10-yl)piperazin-2-yl)acetonitrile

### Step A: 4-bromo-2-fluoro-6-hydroxybenzonitrile

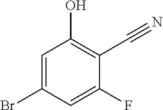 [SMILES: N#Cc1c(O)cc(Br)cc1F]

Sodium hydride (25 g, 0.6 mol, 60% in mineral oil) was added to DMF (500 ml) batchwise at room temperature. Then the mixture of 4-bromo-2,6-difluorobenzonitrile (54 g, 0.25 mol) and 2-(methylsulfonyl)ethan-1-ol (46.3 g, 0.37 mol) in DMF (200 ml) was added dropwise at 0-5° C. The mixture was stirred for 1 hours while gradually warming to ambient temperature. The reaction mixture was quenched with water. The solid was filtered to give the product. Yield 95%. 1H NMR (400 MHz, DMSO-d₆) δ 12.19 (s, 1H), 7.96 (s, 1H), 7.27 (dd, J=9.0, 1.2 Hz, 1H), 7.03 (s, 1H) ppm. LC/MS [M+H]⁺ 216.0

### Step B: 2-(allyloxy)-4-bromo-6-fluorobenzonitrile

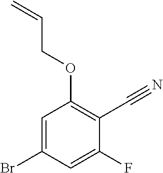

4-bromo-2-fluoro-6-hydroxybenzonitrile (48 g, 0.22 mol) was dissolved in MeCN (300 ml) followed by addition of K₂CO₃ (46 g, 0.33 mmol) and 3-bromoprop-1-ene (32 g, 0.27 mol). The mixture was stirred for 4 hours at 60° C. The reaction mixture was cooled to rt and filtered. The filtered was concentrated and diluted with EA, washed with water, dried over sodium sulfate and concentrated to afford 2-(allyloxy)-4-bromo-6-fluorobenzonitrile (39 g). The product was used without further purification. Yield 70%. 1H-NMR (300 MHz, CDCl3) δ 7.03 (dd, 1H, J=8.1, 1.5 Hz), 6.94 (s, 1H), 6.10-5.98 (m, 1H), 5.53 (dd, 1H, J=17.4, 1.2 Hz), 5.42 (dd, 1H, J=10.5, 1.2 Hz), 4.69 (d, 2H, J=4.8, 1.5 Hz) ppm. LC/MS [M+H]⁺ 256

### Step C: 3-allyl-4-bromo-6-fluoro-2-hydroxybenzonitrile

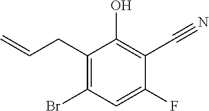

2-(allyloxy)-4-bromo-6-fluorobenzonitrile (36 g, 0.14 mol) was stirred in 1,2-dicholorbenzene (50 mL) at 175° C. for approximately 36 hours. The resulting mixture was cooled, filtration, and the filtrated cake was washed with PE to afford 3-allyl-4-bromo-6-fluoro-2-hydroxybenzonitrile as solid (20 g). Yield 56%. 1H NMR (300 MHz, CDCl3) δ 7.10 (d, 1H, J=8.1 Hz), 5.98-5.85 (m, 1H), 5.22-5.13 (m, 2H), 3.63 (d, 2H, J=6 Hz).

### Step D: 4-bromo-6-fluoro-2-hydroxy-3-(3-hydroxypropyl)benzonitrile

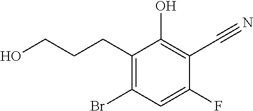 [SMILES: N#Cc1c(F)cc(Br)c(CCCO)c1O]

3-allyl-4-bromo-6-fluoro-2-hydroxybenzonitrile (20 g, 78 mmol) was dissolved in THF (100 mL) and cooled to 0° C. Borane-dimethyl sulfide complex (93 mL, 93 mmol, 1M in THF) was added dropwise over 10 minutes. The reaction mixture was stirred for 1 hour, then a mixture of 30% peroxide (18 ml) and aq.sat. NaHCO₃ (18 mL) was added dropwise while maintaining the reaction temperature below 10° C. The reaction was monitored by TLC until complete, then quenched with saturated aqueous sodium thiosulfate (˜500 mL). The reaction was acidified with conc. HCl (to pH ˜2), extracted with ethyl acetate, dried over sodium sulfate, concentrated, and purified on silica gel (5%˜90% ethyl acetate in PE) to afford 4-bromo-6-fluoro-2-hydroxy-3-(3-hydroxypropyl)benzonitrile. Yield 44%. ¹H-NMR (300 MHz, CDCl₃) δ 7.044 (d, 1H, J=8.1 Hz), 3.70 (t, 2H, J=5.7), 2.98 (t, 2H, J=6.3 Hz), 1.97 (m, 2H, J=5.7 Hz) ppm.

### Step E: 5-bromo-7-fluorochromane-8-carbonitrile

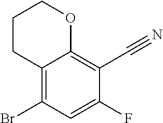 [SMILES: N#Cc1c(F)cc(Br)c2c1OCCC2]

4-Bromo-6-fluoro-2-hydroxy-3-(3-hydroxypropyl)benzonitrile (1.25 g, 4.56 mmol) and triphenylphosphine (1.2 g, 4.56 mol) were dissolved in THF (30 mL) and cooled to 0° C. DIAD (0.71 mL, 4.56 mmol) was added dropwise and the reaction was stirred for 30 minutes at 0° C. then concentrated and purified by silica gel (5-15% ethyl acetate in PE) to afford the product. Yield 73%. LC/MS [M+H]⁺ 256.

### Step F: 7-amino-5-bromochromane-8-carbonitrile

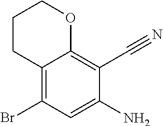 [SMILES: N#Cc1c(N)cc(Br)c2c1OCCC2]

To 5-bromo-7-fluorochromane-8-carbonitrile (2.00 g, 7.9 mmol) in 5 mL 1-Methyl-2-pyrrolidinone was added ammonium hydroxide (25˜28%, 5 mL). The colorless solution was heated at 120° C. for overnight in a sealed vial. Then the reaction was cooled to rt. The reaction was filtered, and the filtrate cake was dried to afford the product (1.5 g).

### Step G: 7-amino-5-bromochromane-8-carboxamide

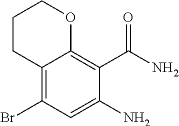 [SMILES: NC(=O)c1c(N)cc(Br)c2c1OCCC2]

A solution of 7-amino-5-bromochromane-8-carbonitrile (2 g, 7.90 mmol) in DMSO (25 mL) was treated with 30% aqueous hydrogen peroxide (17 mL, 31.6 mmol) and a 1. M aqueous potassium hydroxide solution (25 mL) then heated at 40° C. for 1 h. The reaction mixture was cooled, treated with water and cooled in an ice-bath. The resulting precipitate was collected by filtration, washed with water and dried in vacuo to obtain 7-amino-5-bromochromane-8-carboxamide (1.8 g, 88%).

### Step H: 5-bromo-8-chloro-2,3,4,9-tetrahydro-10H-pyrano[2,3-f]quinazolin-10-one

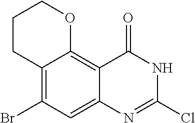 [SMILES: O=c1[nH]c(Cl)nc2cc(Br)c3c(c12)OCCC3]

A solution of 7-amino-5-bromochromane-8-carboxamide (5.1 g, 19 mmol) in dioxane (25 mL) was added thiophosgene (8.7 g, 75 mmol) dropwise. The solution was stirred at room temperature for 1 h then heated to 105° C. for 1 h. The reaction mixture was cooled. The resulting precipitate was collected by filtration and dried in vacuo to obtain 5-bromo-8-chloro-2,3,4,9-tetrahydro-10H-pyrano[2,3-f]quinazolin-10-one (4 g, 68%).

### Step I: 5-bromo-8-(((3S,4S)-4-methoxy-1-methylpyrrolidin-3-yl)oxy)-2,3,4,9-tetrahydro-10H-pyrano[2,3-f]quinazolin-10-one

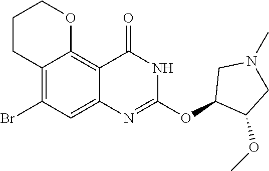 [SMILES: CO[C@H]1CN(C)C[C@@H]1Oc1nc2cc(Br)c3c(c2c(=O)[nH]1)OCCC3]

To a solution of the 5-bromo-8-chloro-2,3,4,9-tetrahydro-10H-pyrano[2,3-f]quinazolin-10-one (90 mg, 0.29 mmol) and (3S,4S)-4-methoxy-1-methylpyrrolidin-3-ol (112 mg, 0.86 mmol) in THF (3 mL) was added NaH (60% in mineral oil, 34.4 mg, 0.86 mmol). The resulting mixture was stirred at 65° C. for another 4 hours. The reaction mixture was quenched with AcOH (1 mL) and the solvent was removed under vacuum. The crude product was purified by prep-TLC (DCM:MeOH=20:1) to give the target compound (75 mg, 64% yield). MS: M/e 410.1 (M+1)⁺.

### Step J: tert-butyl (S)-4-(5-bromo-8-(((3S,4S)-4-methoxy-1-methylpyrrolidin-3-yl)oxy)-3,4-dihydro-2H-pyrano[2,3-f]quinazolin-10-yl)-2-(cyanomethyl)piperazine-1-carboxylate

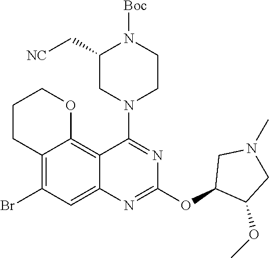 [SMILES: CO[C@H]1CN(C)C[C@@H]1Oc1nc(N2CCN(C(=O)OC(C)(C)C)[C@@H](CC#N)C2)c2c3c(c(Br)cc2n1)CCCO3]

To a solution of the 5-bromo-8-(((3S,4S)-4-methoxy-1-methylpyrrolidin-3-yl)oxy)-2,3,4,9-tetrahydro-10H-pyrano[2,3-f]quinazolin-10-one (75 mg, 0.18 mmol) and tert-butyl (S)-2-(cyanomethyl)piperazine-1-carboxylate (61 mg, 0.27 mmol) in CH₃CN (30 mL) were added PyBOP (121.7 mg, 0.23 mmol) and DBU (55 mg, 0.36 mmol). The resulting mixture was stirred at RT for another 2 hours. The reaction solvent was removed under vacuum. The crude product was dissolved with EA (100 mL) and washed with brine and water. The organic layer was concentrated to obtained crude product which further purified by prep-TLC (DCM:MeOH=20:1) to give the target compound (97 mg, 85.8% yield). MS: M/e 617 (M+1)⁺.

### Step K: tert-butyl (2S)-4-(5-(5-chloro-6-methyl-1-(tetrahydro-2H-pyran-2-yl)-1H-indazol-4-yl)-8-(((3S,4S)-4-methoxy-1-methylpyrrolidin-3-yl)oxy)-3,4-dihydro-2H-pyrano[2,3-f]quinazolin-10-yl)-2-(cyanomethyl)piperazine-1-carboxylate

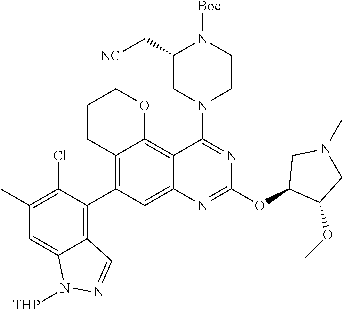

To a mixture of the tert-butyl (S)-4-(5-bromo-8-(((3S,4S)-4-methoxy-1-methylpyrrolidin-3-yl)oxy)-3,4-dihydro-2H-pyrano[2,3-f]quinazolin-10-yl)-2-(cyanomethyl)piperazine-1-carboxylate (97 mg, 0.16 mmol) and 5-chloro-6-methyl-1-(tetrahydro-2H-pyran-2-yl)-4-(4,4,5,5-tetramethyl-1,3,2-dioxaborolan-2-yl)-1H-indazole (79 mg, 0.21 mmol) in 1,4-dioxane (8 mL) and water (1 mL) were added Pd(dppf)Cl₂ (12 mg, 0.016 mmol) and K₃PO₄ (102 mg, 0.48 mmol). The resulting mixture was stirred at 100° C. for 2 hours. The reaction solvent was removed under reduce pressure to afford crude product. The crude product was purified by prep-TLC (DCM:MeOH=20:1) to give the target compound (86 mg, 69.3% yield). MS: M/e 787 (M+1)⁺.

### Step L: 2-((2S)-4-(5-(5-chloro-6-methyl-1H-indazol-4-yl)-8-(((3S,4S)-4-methoxy-1-methylpyrrolidin-3-yl)oxy)-3,4-dihydro-2H-pyrano[2,3-f]quinazolin-10-yl)piperazin-2-yl)acetonitrile

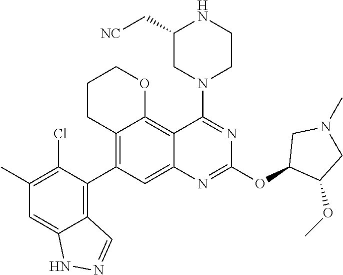

tert-butyl (2S)-4-(5-(5-chloro-6-methyl-1-(tetrahydro-2H-pyran-2-yl)-1H-indazol-4-yl)-8-(((3S,4S)-4-methoxy-1-methylpyrrolidin-3-yl)oxy)-3,4-dihydro-2H-pyrano[2,3-f]quinazolin-10-yl)-2-(cyanomethyl)piperazine-1-carboxylate (86 mg, 0.11 mmol) was added to a solution of TFA (0.5 mL) in DCM (5 mL) and EA (15 mL). The resulting mixture was stirred at RT for 4 hour. The reaction solvent was removed under reduce pressure to afford the target compound (86 mg, crude) which was used to next step directly without further purification. MS: M/e 603 (M+1)⁺.

### Step M: 2-((2S)-1-acryloyl-4-(5-(5-chloro-6-methyl-1H-indazol-4-yl)-8-(((3S,4S)-4-methoxy-1-methylpyrrolidin-3-yl)oxy)-3,4-dihydro-2H-pyrano[2,3-f]quinazolin-10-yl)piperazin-2-yl)acetonitrile

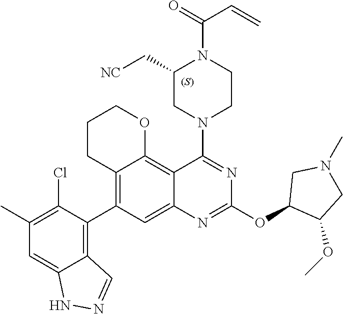

To a mixture of 2-((2S)-4-(5-(5-chloro-6-methyl-1H-indazol-4-yl)-8-(((3S,4S)-4-methoxy-1-methylpyrrolidin-3-yl)oxy)-3,4-dihydro-2H-pyrano[2,3-f]quinazolin-10-yl)piperazin-2-yl)acetonitrile (46 mg, 0.08 mmol) and saturated NaHCO₃ aq. (0.5 mL) in CH₃CN (6 mL) was added a solution of acryloyl chloride (7 mg, 0.08 mmol) in CH₃CN (1 mL) dropwise. The resulting mixture was stirred at RT for 1 hour. The reaction mixture was diluted with EtOAc (30 mL) and washed with water (30 mL×2). The organic layers were dried with MgSO₄, filtered and evaporated to afford crude product. The crude product was purified by prep-TLC (DCM:MeOH=20:1) to give the target compound (7.06 mg, 14% yield). 1H NMR (400 MHz, CD3OD) δ 7.63-7.42 (m, 2H), 7.00-6.86 (m, 2H), 6.30 (d, J=16.4 Hz, 1H), 5.85 (s, 1H), 5.37 (s, 1H), 5.19 (s, 1H), 4.44-4.07 (m, 6H), 3.41 (s, 6H), 3.24 (s, 1H), 2.99 (s, 2H), 2.81-2.65 (m, 3H), 2.57 (s, 4H), 2.38 (s, 3H), 2.28 (s, 1H), 2.01 (s, 2H) ppm. MS: M/e 657 (M+1)⁺.

### Compound B4: 2-((2S)-1-acryloyl-4-(5-(5-chloro-6-methyl-1H-indazol-4-yl)-8-(3-(dimethylamino)azetidin-1-yl)-3,4-dihydro-2H-pyrano[2,3-f]quinazolin-10-yl)piperazin-2-yl)acetonitrile

### Step A: 5-bromo-8-(3-(dimethylamino)azetidin-1-yl)-2,3,4,9-tetrahydro-10H-pyrano[2,3-f]quinazolin-10-one

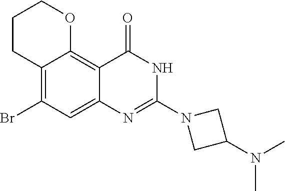 [SMILES: CN(C)C1CN(c2nc3cc(Br)c4c(c3c(=O)[nH]2)OCCC4)C1]

To a solution of the 5-bromo-8-chloro-2,3,4,9-tetrahydro-10H-pyrano[2,3-f]quinazolin-10-one (71 mg, 0.23 mmol) and N,N-dimethylazetidin-3-amine (80 mg, 0.46 mmol) in iPrOH (15 mL) was added DIPEA (296.7 mg, 2.3 mmol). The resulting mixture was stirred at 90° C. for another 3 hours. The reaction solvent was removed under vacuum. The crude product was purified by prep-TLC (DCM:MeOH=20:1) to give the target compound (68 mg, 70.8% yield). MS: M/e 379 (M+1)⁺.

### Step B: tert-butyl (S)-4-(5-bromo-8-(3-(dimethylamino)azetidin-1-yl)-3,4-dihydro-2H-pyrano[2,3-f]quinazolin-10-yl)-2-(cyanomethyl)piperazine-1-carboxylate

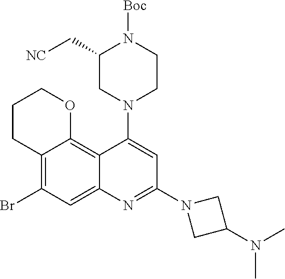 [SMILES: CN(C)C1CN(c2cc(N3CCN(C(=O)OC(C)(C)C)[C@@H](CC#N)C3)c3c4c(c(Br)cc3n2)CCCO4)C1]

To a solution of 5-bromo-8-(3-(dimethylamino)azetidin-1-yl)-2,3,4,9-tetrahydro-10H-pyrano[2,3-f]quinazolin-10-one (68 mg, 0.18 mmol) and tert-butyl (S)-2-(cyanomethyl)piperazine-1-carboxylate (61 mg, 0.27 mmol) in CH₃CN (10 mL) were added PyBOP (119.6 mg, 0.23 mmol) and DBU (55 mg, 0.36 mmol). The resulting mixture was stirred at RT for another 2 hours. The reaction solvent was removed under vacuum. The crude product was dissolved with EA (100 mL) and washed with brine and water. The organic layer was concentrated to obtained crude product which further purified by prep-TLC (DCM:MeOH=20:1) to give the target compound (186 mg, crude). MS: M/e 586 (M+1)⁺.

### Step C: tert-butyl (2S)-4-(5-(5-chloro-6-methyl-1-(tetrahydro-2H-pyran-2-yl)-1H-indazol-4-yl)-8-(3-(dimethylamino)azetidin-1-yl)-3,4-dihydro-2H-pyrano[2,3-f]quinazolin-10-yl)-2-(cyanomethyl)piperazine-1-carboxylate

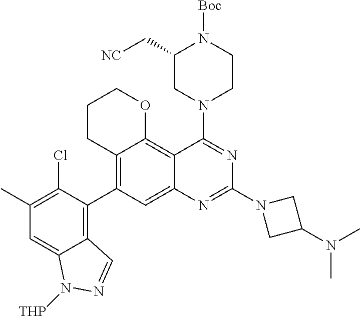

To a mixture of tert-butyl (S)-4-(5-bromo-8-(3-(dimethylamino)azetidin-1-yl)-3,4-dihydro-2H-pyrano[2,3-f]quinazolin-10-yl)-2-(cyanomethyl)piperazine-1-carboxylate (186 mg crude, 0.32 mmol) and 5-chloro-6-methyl-1-(tetrahydro-2H-pyran-2-yl)-4-(4,4,5,5-tetramethyl-1,3,2-dioxaborolan-2-yl)-1H-indazole (180 mg, 0.47 mmol) in 1,4-dioxane (8 mL) and water (1 mL) were added Pd(dppf)Cl₂ (17.5 mg, 0.02 mmol) and K₃PO₄ (202 mg, 0.95 mmol). The resulting mixture was stirred at 100° C. for 2 hours. The reaction solvent was removed under reduce pressure to afford crude product. The crude product was purified by prep-TLC (DCM:MeOH=20:1) to give the target compound (112 mg, crude). MS: M/e 756 (M+1)⁺.

### Step D: 2-((2S)-4-(5-(5-chloro-6-methyl-1H-indazol-4-yl)-8-(3-(dimethylamino)azetidin-1-yl)-3,4-dihydro-2H-pyrano[2,3-f]quinazolin-10-yl)piperazin-2-yl)acetonitrile

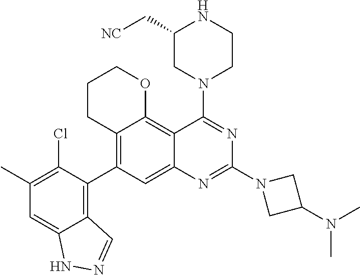

tert-butyl (2S)-4-(5-(5-chloro-6-methyl-1-(tetrahydro-2H-pyran-2-yl)-1H-indazol-4-yl)-8-(3-(dimethylamino)azetidin-1-yl)-3,4-dihydro-2H-pyrano[2,3-f]quinazolin-10-yl)-2-(cyanomethyl)piperazine-1-carboxylate (112 mg crude, 0.15 mmol) was added to a solution of TFA (0.5 mL) and DCM (5 mL). The resulting mixture was stirred at RT for 4 hour. The reaction solvent was removed under reduce pressure to afford the target compound (80 mg, 94% yield) which was used to next step directly without further purification. MS: M/e 572 (M+1)⁺.

### Step E: 2-((2S)-1-acryloyl-4-(5-(5-chloro-6-methyl-1H-indazol-4-yl)-8-(3-(dimethylamino)azetidin-1-yl)-3,4-dihydro-2H-pyrano[2,3-f]quinazolin-10-yl)piperazin-2-yl)acetonitrile

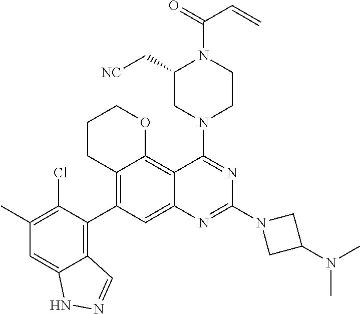

To a mixture of 2-((2S)-4-(5-(5-chloro-6-methyl-1H-indazol-4-yl)-8-(3-(dimethylamino)azetidin-1-yl)-3,4-dihydro-2H-pyrano[2,3-f]quinazolin-10-yl)piperazin-2-yl)acetonitrile (80 mg, 0.14 mmol) and saturated NaHCO₃ aq. (0.5 mL) in CH₃CN (6 mL) was added a solution of acryloyl chloride (12 mg, 0.14 mmol) in CH₃CN (1 mL) dropwise. The resulting mixture was stirred at RT for 1 hour. The reaction mixture was diluted with EtOAc (30 mL) and washed with water (30 mL×2). The organic layers were dried with MgSO₄, filtered and evaporated to afford crude product. The crude product was purified by prep-TLC (DCM:MeOH=20:1) to give the target compound (7.9 mg, 10% yield). 1H NMR (400 MHz, CD3OD) δ 7.45 (d, J=9.8 Hz, 2H), 6.84 (s, 1H), 6.72 (s, 1H), 6.20 (d, J=16.6 Hz, 1H), 5.76 (s, 1H), 4.42 (m, 2H), 4.37-4.13 (m, 5H), 3.97 (s, 4H), 3.39 (m, 2H), 2.82 (m, 2H), 2.43 (m, 5H), 2.19 (s, 7H), 1.89 (s, 2H) ppm. MS: M/e 626 (M+1)⁺.

### Compound B5: 2-((2S)-1-acryloyl-4-(5-(5-chloro-6-methyl-1H-indazol-4-yl)-8-(2-(dimethylamino)ethoxy)-3,4-dihydro-2H-pyrano[2,3-f]quinazolin-10-yl)piperazin-2-yl)acetonitrile

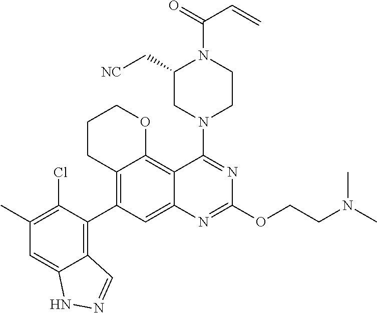

The compound B5 was synthesized starting from the corresponding starting materials according the similar procedures described as those of Compound B4 to give the target compound (7.46 mg). 1H NMR (400 MHz, CD3OD) δ 7.46 (s, 1H), 7.42 (s, 1H), 6.93 (m, 2H), 6.20 (d, J=16.6 Hz, 1H), 5.76 (s, 1H), 5.26-4.97 (m, 1H), 4.50-4.03 (m, 6H), 3.34 (3H), 2.87 (s, 4H), 2.47-2.39 (s, 11H), 2.20-2.11 (m, 1H), 1.91 (s, 2H) ppm, MS: M/e 615 (M+1)⁺.

### Compound B6: 1-(4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)piperazin-1-yl)prop-2-en-1-one

### Step A: tert-butyl (S)-4-(5-bromo-8-((1-methylpyrrolidin-2-yl)methoxy)-3,4-dihydro-2H-pyrano[2,3-f]quinazolin-10-yl)piperazine-1-carboxylate

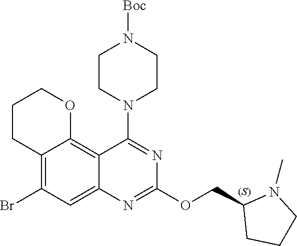

To a solution of (S)-5-bromo-8-((1-methylpyrrolidin-2-yl)methoxy)-2,3,4,9-tetrahydro-10H-pyrano[2,3-f]quinazolin-10-one (85 mg, 0.21 mmol) and tert-butyl piperazine-1-carboxylate (55.8 mg, 0.3 mmol) in CH₃CN (15 mL) were added PyBOP (196.0 mg, 0.38 mmol) and DBU (64 mg, 0.42 mmol). The resulting mixture was stirred at RT for another 3 hours. The reaction solvent was removed under vacuum. The crude product was dissolved with EA (100 mL) and washed with brine and water. The organic layer was concentrated to obtained crude product which further purified by prep-TLC (DCM:MeOH=20:1) to give the target compound (65 mg, crude). MS: M/e 562 (M+1)⁺.

### Step B: tert-butyl 4-(5-(5-chloro-6-methyl-1-(tetrahydro-2H-pyran-2-yl)-1H-indazol-4-yl)-8-(((S)-1-methylpyrrolidin-2-yl)methoxy)-3,4-dihydro-2H-pyrano[2,3-f]quinazolin-10-yl)piperazine-1-carboxylate

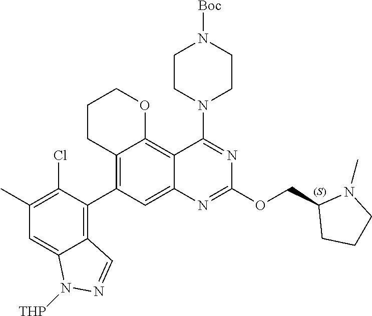

To a mixture of tert-butyl (S)-4-(5-bromo-8-((1-methylpyrrolidin-2-yl)methoxy)-3,4-dihydro-2H-pyrano[2,3-f]quinazolin-10-yl)piperazine-1-carboxylate (65 mg, crude, 0.12 mmol) and 5-chloro-6-methyl-1-(tetrahydro-2H-pyran-2-yl)-4-(4,4,5,5-tetramethyl-1,3,2-dioxaborolan-2-yl)-1H-indazole (64 mg, 0.17 mmol) in 1,4-dioxane (5 mL) and water (0.5 mL) were added Pd(dppf)Cl₂ (6.6 mg, 0.01 mmol) and K₃PO₄ (76.3 mg, 0.36 mmol). The resulting mixture was stirred at 95° C. for 2 hours. The reaction solvent was removed under reduce pressure to afford crude product. The crude product was purified by prep-TLC (DCM:MeOH=20:1) to give the target compound (43 mg, crude). MS: M/e 732 (M+1)⁺.

### Step C: 5-(5-chloro-6-methyl-1H-indazol-4-yl)-8-(((S)-1-methylpyrrolidin-2-yl)methoxy)-10-(piperazin-1-yl)-3,4-dihydro-2H-pyrano[2,3-f]quinazoline

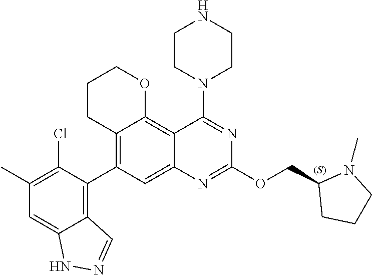

tert-butyl 4-(5-(5-chloro-6-methyl-1-(tetrahydro-2H-pyran-2-yl)-1H-indazol-4-yl)-8-(((S)-1-methylpyrrolidin-2-yl)methoxy)-3,4-dihydro-2H-pyrano[2,3-f]quinazolin-10-yl)piperazine-1-carboxylate (43 mg, 0.09 mmol) was added to a solution of TFA (0.5 mL) in DCM (5 mL) and EA (15 mL). The resulting mixture was stirred at RT for 4 hour. The reaction solvent was removed under reduce pressure to afford the target compound (30 mg, 100% yield) which was used to next step directly without further purification. MS: M/e 548 (M+1)⁺.

### Step D: 1-(4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)piperazin-1-yl)prop-2-en-1-one

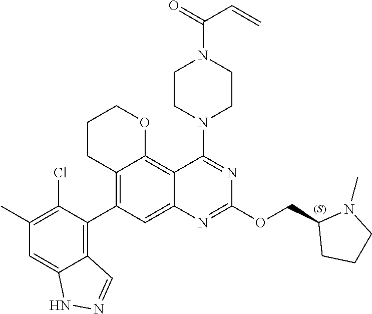

To a mixture of 5-(5-chloro-6-methyl-1H-indazol-4-yl)-8-(((S)-1-methylpyrrolidin-2-yl)methoxy)-10-(piperazin-1-yl)-3,4-dihydro-2H-pyrano[2,3-f]quinazoline (30 mg, 0.05 mmol) and saturated NaHCO₃ aq. (0.5 mL) in CH₃CN (6 mL) was added a solution of acryloyl chloride (12 mg, 0.064 mmol) in CH₃CN (1 mL) dropwise. The resulting mixture was stirred at RT for 1 hour. The reaction mixture was diluted with EtOAc (30 mL) and washed with water (30 mL×2). The organic layers were dried with MgSO₄, filtered and evaporated to afford crude product. The crude product was purified by prep-TLC (DCM:MeOH=10:1) to give the target compound (3.36 mg, 10% yield). 1H NMR (400 MHz, CD₃OD) δ 7.55 (s, 1H), 7.52 (s, 1H), 6.99 (s, 1H), 6.84 (dd, J=16.9, 10.8 Hz, 1H), 6.26 (d, J=16.8 Hz, 1H), 5.80 (d, J=10.9 Hz, 1H), 4.44 (s, 2H), 4.36 (s, 2H), 3.87 (s, 4H), 3.75 (s, 4H), 2.85 (s, 4H), 2.57 (s, 4H), 2.46 (m, 2H), 2.36-2.05 (m, 2H), 1.98 (s, 2H), 1.85-1.76 (m, 3H) ppm. MS: M/e 602 (M+1)⁺.

### Compound B7: 1-(4-(8-(3-(dimethylamino)azetidin-1-yl)-5-(2-fluoro-6-hydroxyphenyl)-3,4-dihydro-2H-pyrano[2,3-f]quinazolin-10-yl)piperazin-1-yl)prop-2-en-1-one

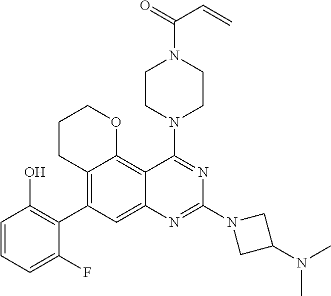 [SMILES: C=CC(=O)N1CCN(c2nc(N3CC(N(C)C)C3)nc3cc(-c4c(O)cccc4F)c4c(c23)OCCC4)CC1]

The compound B7 was synthesized starting from the corresponding starting materials according the similar procedures described as those of Compound B6 to give the target compound (2 mg). ¹H NMR (400 MHz, DMSO-d6) δ 7.26 (s, 1H), 7.13-6.61 (m, 5H), 6.17 (d, J=16.8 Hz, 1H), 5.75 (d, J=10.0 Hz, 1H), 4.61-4.01 (m, 6H), 3.89-3.62 (m, 6H), 3.09-2.58 (m, 5H), 2.41-2.15 (m, 4H), 2.05-1.87 (m, 3H) ppm. MS: M/e 533 (M+1)⁺.

### Compound B8: 2-((2S)-4-(8-(3-(dimethylamino)azetidin-1-yl)-5-(2-fluoro-6-hydroxyphenyl)-3,4-dihydro-2H-pyrano[2,3-f]quinazolin-10-yl)-1-(2-fluoroacryloyl)piperazin-2-yl)acetonitrile

### Step A: tert-butyl (S)-4-(5-bromo-8-(3-(dimethylamino)azetidin-1-yl)-3,4-dihydro-2H-pyrano[2,3-f]quinazolin-10-yl)-2-(cyanomethyl)piperazine-1-carboxylate

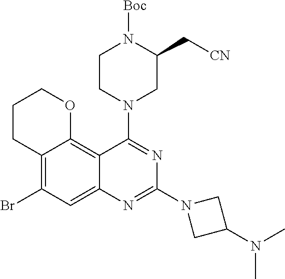 [SMILES: CN(C)C1CN(c2nc(N3CCN(C(=O)OC(C)(C)C)[C@@H](CC#N)C3)c3c4c(c(Br)cc3n2)CCCO4)C1]

To a stirred mixture of 5-bromo-8-(3-(dimethylamino)azetidin-1-yl)-2,3,4,9-tetrahydro-10H-pyrano[2,3-f]quinazolin-10-one (53 mg, 0.14 mmol) and tert-butyl (S)-2-(cyanomethyl)piperazine-1-carboxylate (47 mg, 0.21 mmol) in CH₃CN (20 mL) was added pyBOP (95 mg, 0.182 mmol), followed by DBU (43 mg, 0.28 mmol), then stirred for 2 hours at 30° C., then over a weekend at room temperature. The mixture was diluted with EA (20 mL), washed with brine, dried over Na₂SO₄, concentrated and purified by Prep-TLC (CH₂Cl₂/MeOH=10:1) to give the target compound (crude), which was directly used to the next step without further purification. MS: M/e 586 (M+1)⁺.

### Step B: tert-butyl (2S)-4-(5-(2-(benzyloxy)-6-fluorophenyl)-8-(3-(dimethylamino)azetidin-1-yl)-3,4-dihydro-2H-pyrano[2,3-f]quinazolin-10-yl)-2-(cyanomethyl)piperazine-1-carboxylate

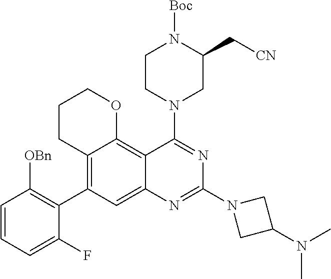 [SMILES: CN(C)C1CN(c2nc(N3CCN(C(=O)OC(C)(C)C)[C@@H](CC#N)C3)c3c4c(c(-c5c(F)cccc5OCc5ccccc5)cc3n2)CCCO4)C1]

To a stirred solution of the product of step A (0.14 mmol) and (2-(benzyloxy)-6-fluorophenyl)boronic acid (41 mg, 0.168 mmol) in dioxane (5 mL) was added aq.K₃PO₄ (89 mg in 0.5 mL of H₂O), then PddppfCl₂ (10.2 mg, 0.014 mmol) was added. After the addition, the reaction was stirred at 100° C. for 2 hours under N₂. The reaction mixture was diluted with EA (10 mL), washed with brine, dried over Na₂SO₄, concentrated to give the residue, which was purified by Prep-TLC (CH₂Cl₂/MeOH=10:1) to give the target compound (30 mg, 30.3%). MS: M/e 708 (M+1)⁺.

### Step D: 2-((2S)-4-(8-(3-(dimethylamino)azetidin-1-yl)-5-(2-fluoro-6-hydroxyphenyl)-3,4-dihydro-2H-pyrano[2,3-f]quinazolin-10-yl)-1-(2-fluoroacryloyl)piperazin-2-yl)acetonitrile

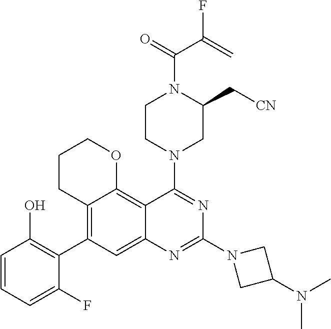 [SMILES: C=C(F)C(=O)N1CCN(c2nc(N3CC(N(C)C)C3)nc3cc(-c4c(O)cccc4F)c4c(c23)OCCC4)C[C@@H]1CC#N]

Pd/C (20 mg) was added to a stirred solution of the product of step C (30 mg, 0.04 mmol) in THF/EtOH (3 mL/3 mL). Then the mixture was stirred overnight under H₂ (1 atm). The reaction mixture was filtered and the filtrate was concentrated to give the residue, which was dissolved in CH₂Cl₂ (3 mL), HCl/dioxane (4.0 M, 2 mL) was added and the mixture was stirred for 2 hours. The reaction mixture was concentrated to give the intermediate, which was dissolved in THF (5 mL), Et₃N (17 mg, 0.017 mmol) was added, followed by 2-fluoroacrylic acid (7.6 mg, 0.084 mmol) and T₃P (53 mg, 0.084 mmol). Then the mixture was stirred for an hour. The reaction mixture was diluted with EA (10 mL), washed with brine, dried over Na₂SO₄, concentrated, purified by Prep-TLC (CH₂Cl₂/MeOH=5:1) to give the target compound (5 mg). ¹H NMR (400 MHz, DMSO-d6) δ 7.28-7.17 (m, 1H), 6.85-6.68 (m, 3H), 5.45-5.20 (m, 2H), 4.38-3.75 (m, 8H), 3.15-2.87 (m, 4H), 2.40-2.10 (m, 9H), 2.00-1.85 (m, 5H) ppm. MS: M/e 590 (M+1)⁺.

### Compound B9: (S)-2-(4-(5-(2,3-difluorophenyl)-8-(3-(dimethylamino)azetidin-1-yl)-3,4-dihydro-2H-pyrano[2,3-f]quinazolin-10-yl)-1-(2-fluoroacryloyl)piperazin-2-yl)acetonitrile

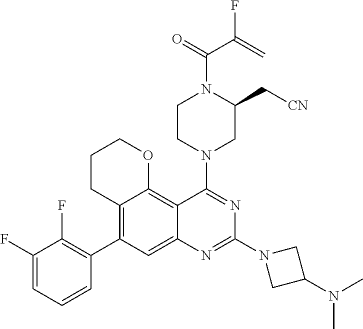 [SMILES: C=C(F)C(=O)N1CCN(c2nc(N3CC(N(C)C)C3)nc3cc(-c4cccc(F)c4F)c4c(c23)OCCC4)C[C@@H]1CC#N]

The compound B9 was synthesized starting from the corresponding starting materials according the similar procedures described as those of Compound B8 to give the product (10 mg). ¹H NMR (400 MHz, DMSO-d6) δ 7.52-7.50 (m, 1H), 7.32 (s, 1H), 7.18 (s, 1H), 6.82 (s, 1H), 5.42-5.23 (m, 2H), 4.32 (s, 2H), 4.15 (br.s, 1H), 4.07 (s, 2H), 3.83 (s, 2H), 3.28-3.15 (m, 2H), 3.01 (s, 6H), 2.33 (s, 2H), 2.14 (s, 4H), 1.92 (s, 2H), 1.73 (s, 3H) ppm. MS: M/e 592 (M+1)⁺

### Compound B10: 1-(4-(8-(3-(dimethylamino)azetidin-1-yl)-5-(naphthalen-1-yl)-3,4-dihydro-2H-pyrano[2,3-f]quinazolin-10-yl)piperazin-1-yl)prop-2-en-1-one

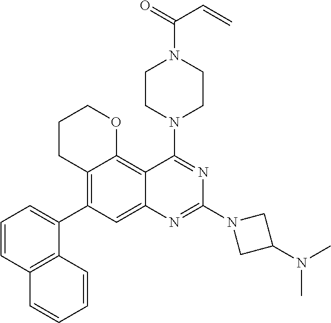 [SMILES: C=CC(=O)N1CCN(c2nc(N3CC(N(C)C)C3)nc3cc(-c4cccc5ccccc45)c4c(c23)OCCC4)CC1]

The compound B10 was synthesized starting from the corresponding starting materials according the similar procedures described as those of Compound B3 to give the target compound (5 mg). ¹H NMR (400 MHz, DMSO-d6) δ 8.01 (t, J=8.0 Hz, 2H), 7.64-7.53 (m, 2H), 7.52-7.43 (m, 2H), 7.40 (d, J=6.8 Hz, 1H), 6.98-6.81 (m, 2H), 6.16 (d, J=10.4 Hz, 1H), 5.75 (d, J=10.4 Hz, 1H), 4.37-4.03 (m, 7H), 3.83-3.54 (m, 9H), 2.75-2.66 (m, 1H), 2.44-1.95 (m, 6H), 1.83 (s, 2H) ppm. MS: M/e 549 (M+1)⁺.

### Compound B11: 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

### Step A: (S)-5-bromo-8-((1-methylpyrrolidin-2-yl)methoxy)-2,3,4,9-tetrahydro-10H-pyran[2,3-f]quinazolin-10-one

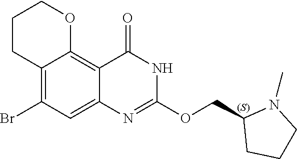

To a solution of the 5-bromo-8-chloro-2,3,4,9-tetrahydro-10H-pyrano[2,3-f]quinazolin-10-one (360 mg, 1.15 mmol) and (S)-(1-methylpyrrolidin-2-yl)methanol (395 mg, 3.44 mmol) in THF (20 mL) was added NaH (60% in mineral oil, 165.6 mg, 4.14 mmol). The resulting mixture was stirred at 65° C. for another 4 hours. The reaction mixture was quenched with AcOH (3 mL) and the solvent was removed under vacuum. The crude product was purified by prep-TLC (DCM:MeOH=20:1) to give the target compound (400 mg, 88% yield). MS: M/e 394 (M+1)⁺.

### Step B: tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate

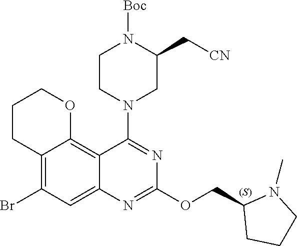

To a solution of the (S)-5-bromo-8-((1-methylpyrrolidin-2-yl)methoxy)-2,3,4,9-tetrahydro-10H-pyrano[2,3-f]quinazolin-10-one (400 mg, 1.02 mmol) and tert-butyl (S)-2-(cyanomethyl)piperazine-1-carboxylate (343 mg, 1.53 mmol) in CH₃CN (30 mL) were added PyBOP (688 mg, 1.33 mmol) and DBU (309 mg, 2.04 mmol). The resulting mixture was stirred at RT for another 3 hours. The reaction solvent was removed under vacuum. The crude product was dissolved with EA (100 mL) and washed with brine and water. The organic layer was concentrated to obtained crude product which further purified by prep-TLC (DCM:MeOH=20:1) to give the target compound (388 mg, 63.5% yield). MS: M/e 601 (M+1)⁺.

### Step C: tert-butyl (2S)-4-(5-(5-chloro-6-methyl-1-(tetrahydro-2H-pyran-2-yl)-1H-indazol-4-yl)-8-(((S)-1-methylpyrrolidin-2-yl)methoxy)-3,4-dihydro-2H-pyrano[2,3-f]quinazolin-10-yl)-2-(cyanomethyl)piperazine-1-carboxylate

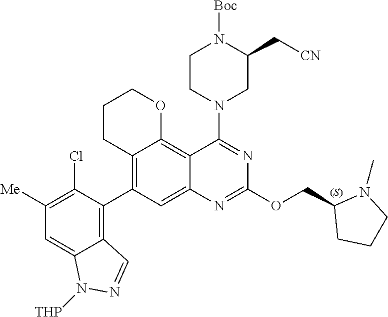

To a mixture of the tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (388 mg, 0.65 mmol) and 5-chloro-6-methyl-1-(tetrahydro-2H-pyran-2-yl)-4-(4,4,5,5-tetramethyl-1,3,2-dioxaborolan-2-yl)-1H-indazole (365 mg, 0.97 mmol) in 1,4-dioxane (15 mL) and water (2 mL) were added Pd(dppf)Cl₂ (36.6 mg, 0.05 mmol) and K₃PO₄ (413.4 mg, 1.95 mmol). The resulting mixture was stirred at 100° C. for 2 hours. The reaction solvent was removed under reduce pressure to afford crude product. The crude product was purified by prep-TLC (DCM:MeOH=20:1) to give the target compound (392 mg, 78.7% yield). MS: M/e 771 (M+1)⁺.

### Step D: 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)piperazin-2-yl)acetonitrile

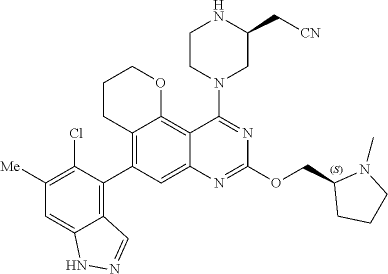

tert-butyl (2S)-4-(5-(5-chloro-6-methyl-1-(tetrahydro-2H-pyran-2-yl)-1H-indazol-4-yl)-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (392 mg, 0.5 mmol) was added to a solution of HCl/1,4-dioxane (4M, 5 mL) and EA (15 mL). The resulting mixture was stirred at RT for 4 hour. The reaction solvent was removed under reduce pressure to afford the target compound (306 mg, crude) which was used to next step directly without further purification. MS: M/e 587 (M+1)⁺.

### Step E: 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

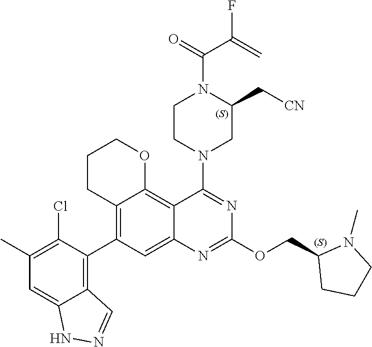

To a solution of the 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)piperazin-2-yl)acetonitrile (76 mg, 0.13 mmol) and 2-fluoroacryloyl chloride (35.1 mg, 0.39 mmol) in THF (10 mL) were added T₃P (50% in EA)(248 mg, 0.39 mmol) and Et₃N (40 mg, 0.39 mmol). The resulting mixture was stirred at RT for 2 hours. The reaction mixture was diluted with EtOAc (20 mL) and washed with NaHCO₃ aq (15 mL). The washings were extracted with EtOAc (15 mL). The organic layers were dried with MgSO₄, filtered and evaporated to afford crude product. The crude product was purified by prep-TLC (DCM:MeOH=20:1) to give the target compound (25.2 mg, 30% yield). 1H NMR (400 MHz, CD3OD) δ 7.46 (s, 1H), 7.42 (s, 1H), 6.92 (d, J=3.5 Hz, 1H), 5.30-5.18 (m, 2H), 4.41-4.06 (m, 8H), 3.45-3.29 (m, 1H), 3.15-2.81 (m, 5H), 2.69 (s, 1H), 2.47 (s, 3H), 2.45 (s, 3H), 2.43-1.58 (m, 9H) ppm. MS: M/e 659 (M+1)⁺.

### Compound B12 and Compound B13: 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer1) and 2-((2S)-4-(5-(5-chloro-6-methyl-1H-indazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 2)

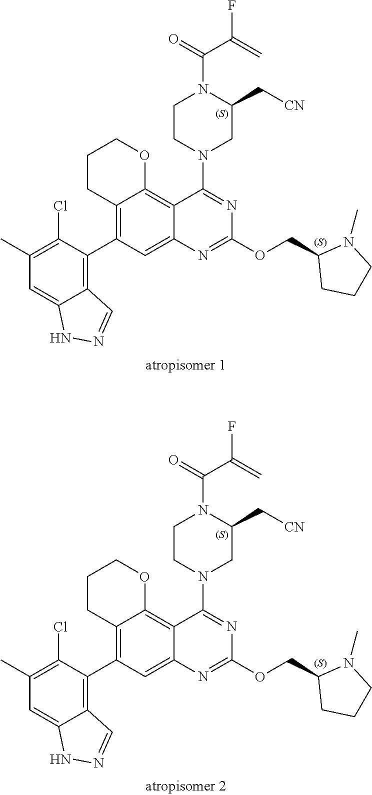

The Compound B11 (64.8 mg) was separated by chiral Prep-HPLC to afford the Compound B12 (atropisomer 1, 34.3 mg) and Compound B13 (atropisomer 2, 28.0 mg)

Prep-HPLC condition

| Column | CHIRALPAK OD-H |
|---|---|
| Column size | 2 cm × 25 cm, 5 um |
| Injection | 3.5 mL |
| Mobile phase | Hex(2mMNH3-MeOH):EtOH = 90:10 |
| Flow rate | 20 mL/min |
| Wavelength | UV 220 nm |
| Temperature | 25° C. |
| Sample solution | 6.5 mg/ml in EtOH |
| Prep-HPLC equipment | Prep-HPLC-Gilson |

Compound B12 (atropisomer 1): 1H NMR (400 MHz, CD3OD) δ 7.56 (s, 1H), 7.52 (s, 1H), 7.02 (s, 1H), 5.37-5.25 (m, 2H), 4.48-4.39 (m, 5H), 4.35 (s, 1H), 4.11 (d, J=11.6 Hz, 2H), 3.44 (d, J=13.9 Hz, 1H), 3.24-2.95 (m, 5H), 2.87-2.75 (m, 1H), 2.53 (s, 3H), 2.51 (s, 3H), 2.38-1.62 (m, 9H) ppm. MS: M/e 659 (M+1)⁺.

Compound B13 (atropisomer 2): 1H NMR (400 MHz, CD3OD) δ 7.55 (s, 1H), 7.52 (s, 1H), 7.01 (s, 1H), 5.33 (dd, J=33.1, 12.5 Hz, 2H), 4.51-4.08 (m, 8H), 3.49 (d, J=11.4 Hz, 1H), 3.18-2.81 (m, 6H), 2.57 (s, 3H), 2.55 (s, 3H), 2.53-2.38 (m, 2H), 2.31-2.07 (m, 2H), 2.00-1.66 (m, 5H) ppm. MS: M/e 659 (M+1)⁺.

### Compound B14: 1-(4-(5-(2,3-difluorophenyl)-8-(((2S,4R)-4-fluoro-1-methylpyrrolidin-2-yl)methoxy)-3,4-dihydro-2H-pyrano[2,3-f]quinazolin-10-yl)piperazin-1-yl)prop-2-en-1-one

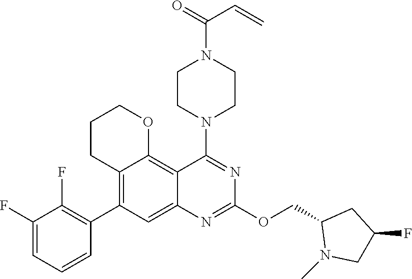 [SMILES: C=CC(=O)N1CCN(c2nc(OC[C@@H]3C[C@@H](F)CN3C)nc3cc(-c4cccc(F)c4F)c4c(c23)OCCC4)CC1]

The compound B14 was synthesized starting from the corresponding starting materials according the similar procedures described as those of Compound B15 to get the product (10 mg). ¹H NMR (400 MHz, CD₃OD) δ 7.36-7.24 (m, 2H), 7.11 (t, J=8.0 Hz, 1H), 7.01 (s, 1H), 6.83-6.79 (m, 1H), 6.26 (d, J=16.0 Hz, 1H), 5.79 (d, J=8.0 Hz, 1H), 5.21-5.18 (m, 1H), 4.48 (d, J=4.0 Hz, 2H), 4.45 (s, 2H), 3.84 (s, 4H), 3.65 (s, 4H), 3.56-3.48 (m, 1H), 3.19 (s, 1H), 2.78-2.62 (m, 3H), 2.59 (s, 3H), 2.67-2.59 (m, 1H), 2.02 (s, 3H) ppm. MS: M/e 568 (M+1)⁺

### Compound B15: 1-(4-(5-(5-chloro-6-methyl-1H-indazol-4-yl)-8-(((2S,4R)-4-fluoro-1-methylpyrrolidin-2-yl)methoxy)-3,4-dihydro-2H-pyrano[2,3-f]quinazolin-10-yl)piperazin-1-yl)prop-2-en-1-one

### Step A: ((2S,4R)-4-fluoro-1-methylpyrrolidin-2-yl)methanol

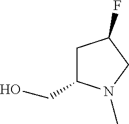

To a disperse of LiAlH₄ (616 mg, 16.2 mmol) in THF solution (20 mL) at 0° C., 1-(tert-butyl) 2-methyl (2S,4R)-4-fluoropyrrolidine-1,2-dicarboxylate (2 g, 8.1 mmol) in THF (5 mL) was added dropwise. The resulting mixture was heated at 70° C. for 2 hrs. Then the solution was cooled down, quenched with 0.7 mL of water, 0.7 mL of 15% NaOH solution and 2.1 mL of water subsequently. After slurried for 10 mins, the solid was filtered and the filtrate was concentrated to get the product (710 mg, 65%). ¹H NMR (400 MHz, DMSO-d6) δ 5.19-5.05 (m, 1H), 4.47 (br.s, 1H), 3.44-3.29 (m, 3H), 2.58-2.26 (m, 2H), 2.22 (s, 3H), 2.04-1.93 (m, 1H), 1.81-1.68 (m, 1H) ppm. MS: M/e 134 (M+1)⁺

### Step B: 5-bromo-8-(((2S,4R)-4-fluoro-1-methylpyrrolidin-2-yl)methoxy)-3,4-dihydro-2H-pyrano[2,3-f]quinazolin-10-ol

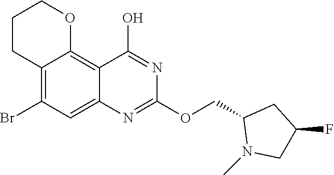 [SMILES: CN1C[C@H](F)C[C@H]1COc1nc(O)c2c3c(c(Br)cc2n1)CCCO3]

NaH (60%, 101 mg, 2.5 mmol) was added to a solution of ((2S,4R)-4-fluoro-1-methylpyrrolidin-2-yl)methanol (290 mg, 2.17 mmol) in THF (15 mL). After stirred for 30 mins, 5-bromo-8-chloro-3,4-dihydro-2H-pyrano[2,3-f]quinazolin-10-ol (280 mg, 0.72 mmol) was added and the mixture was heated at 65° C. for 5 hrs. The solution was quenched with NH₄Cl solution and extracted with EA. The organic layer was dried, concentrated and purified by CombiFlash (DCM:NH₃·MeOH (7M)=85:15) to get the product (230 mg, 77%). MS: M/e 412 (M+1)⁺

### Step C: tert-butyl 4-(5-bromo-8-(((2S,4R)-4-fluoro-1-methylpyrrolidin-2-yl)methoxy)-3,4-dihydro-2H-pyrano[2,3-f]quinazolin-10-yl)piperazine-1-carboxylate

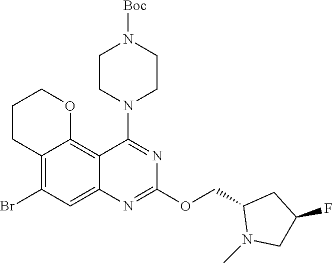 [SMILES: CN1C[C@H](F)C[C@H]1COc1nc(N2CCN(C(=O)OC(C)(C)C)CC2)c2c3c(c(Br)cc2n1)CCCO3]

A solution of 5-bromo-8-(((2S,4R)-4-fluoro-1-methylpyrrolidin-2-yl)methoxy)-3,4-dihydro-2H-pyrano[2,3-f]quinazolin-10-ol (260 mg, 0.63 mmol) and tert-butyl piperazine-1-carboxylate (177 mg, 0.95 mmol) in CH₃CN (30 mL) was stirred for a minute, then added with PyBOP (426 mg, 0.82 mmol), followed with DBU (192 mg, 1.26 mmol). The resulting mixture was stirred at r.t overnight and filtered. The filtrate was diluted with DCM (10 mL), washed with brine, dried over Na₂SO₄, concentrated and purified by CombiFlash (DCM/NH₃·MeOH (7M)=90:10) to give the product (235 mg, 64%). MS: M/e 580 (M+1)⁺.

### Step D: tert-butyl 4-(5-(5-chloro-6-methyl-1-(tetrahydro-2H-pyran-2-yl)-1H-indazol-4-yl)-8-(((2S,4R)-4-fluoro-1-methylpyrrolidin-2-yl)methoxy)-3,4-dihydro-2H-pyrano[2,3-f]quinazolin-10-yl)piperazine-1-carboxylate

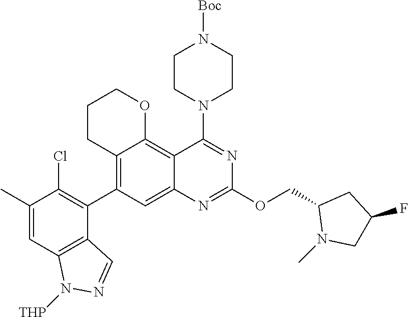

A solution of tert-butyl 4-(5-bromo-8-(((2S,4R)-4-fluoro-1-methylpyrrolidin-2-yl)methoxy)-3,4-dihydro-2H-pyrano[2,3-f]quinazolin-10-yl)piperazine-1-carboxylate (60 mg, 0.10 mmol), 5-chloro-6-methyl-1-(tetrahydro-2H-pyran-2-yl)-4-(4,4,5,5-tetramethyl-1,3,2-dioxaborolan-2-yl)-1H-indazole (47 mg, 0.12 mmol), Pd(dppf)₂Cl₂ (15 mg, 0.02 mmol) and K₃PO₄ (64 mg, 0.30 mmol) in dioxane/water (5 mL/0.5 mL) was heated at 95° C. for 2 hours under N₂ balloon. The reaction mixture was concentrated, diluted with EA (10 mL), washed with brine, dried over Na₂SO₄. The crude product was purified by Prep-TLC (DCM/MeOH=10:1) to give the product (13 mg, 17%) MS: M/e 750 (M+1)⁺.

### Step E: 5-(5-chloro-6-methyl-1H-indazol-4-yl)-8-(((2S,4R)-4-fluoro-1-methylpyrrolidin-2-yl)methoxy)-10-(piperazin-1-yl)-3,4-dihydro-2H-pyrano[2,3-f]quinazoline

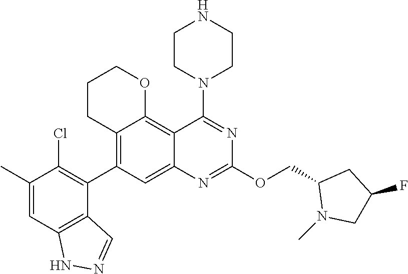 [SMILES: Cc1cc2[nH]ncc2c(-c2cc3nc(OC[C@@H]4C[C@@H](F)CN4C)nc(N4CCNCC4)c3c3c2CCCO3)c1Cl]

HCl/dioxane solution (4 M, 1 mL) was added to a solution of tert-butyl 4-(5-(5-chloro-6-methyl-1-(tetrahydro-2H-pyran-2-yl)-1H-indazol-4-yl)-8-(((2S,4R)-4-fluoro-1-methylpyrrolidin-2-yl)methoxy)-3,4-dihydro-2H-pyrano[2,3-f]quinazolin-10-yl)piperazine-1-carboxylate (13 mg, 0.02 mmol) in EA (2 mL). After stirred at r.t for 2 hrs, the solution was evaporated to get the crude product, which was used in the next step directly (9 mg, 100%). MS: M/e 566 (M+1)⁺

### Step F: 1-(4-(5-(5-chloro-6-methyl-1H-indazol-4-yl)-8-(((2S,4R)-4-fluoro-1-methylpyrrolidin-2-yl)methoxy)-3,4-dihydro-2H-pyrano[2,3-f]quinazolin-10-yl)piperazin-1-yl)prop-2-en-1-one

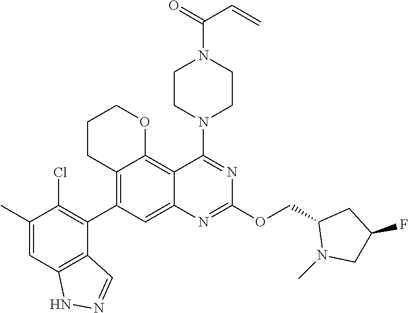 [SMILES: C=CC(=O)N1CCN(c2nc(OC[C@@H]3C[C@@H](F)CN3C)nc3cc(-c4c(Cl)c(C)cc5[nH]ncc45)c4c(c23)OCCC4)CC1]

NaHCO₃ solution (saturated, 0.3 mL) was added to a solution of 5-(5-chloro-6-methyl-1H-indazol-4-yl)-8-(((2S,4R)-4-fluoro-1-methylpyrrolidin-2-yl)methoxy)-10-(piperazin-1-yl)-3,4-dihydro-2H-pyrano[2,3-f]quinazoline (9 mg, crude) in CH₃CN (2 mL), followed with acryloyl chloride (1.8 mg, 0.02 mmol). The reaction mixture was stirred at r.t for 1 hr. The solution was evaporated, added with water (4 mL) and extracted with EA (8 mL). The organic layer was dried, concentrated and purified by prep—TLC (DCM:MeOH (7M NH3)=10:1) to get the product (2 mg, 20%). ¹H NMR (400 MHz, CD₃OD) δ 7.56 (s, 1H), 7.52 (s, 1H), 7.00 (s, 1H), 6.87-6.80 (m, 1H), 6.27 (d, J=16.0 Hz, 1H), 5.80 (d, J=8.0 Hz, 1H), 5.34-5.20 (m, 1H), 4.54-4.50 (m, 2H), 4.37 (s, 2H), 3.87 (s, 4H), 3.77 (s, 4H), 3.69-3.48 (m, 2H), 3.00-2.90 (m, 2H), 2.75 (s, 3H), 2.57 (s, 3H), 2.55-2.17 (m, 3H), 1.99 (s, 2H) ppm. MS: M/e 620 (M+1)⁺

### Compound B16: 1-(4-(8-(3-(dimethylamino)azetidin-1-yl)-5-(2-fluorophenyl)-3,4-dihydro-2H-pyrano[2,3-f]quinazolin-10-yl)piperazin-1-yl)prop-2-en-1-one

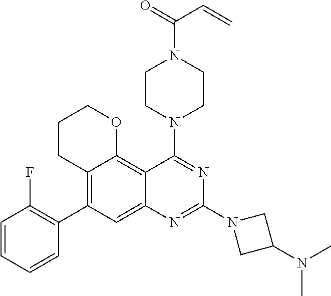 [SMILES: C=CC(=O)N1CCN(c2nc(N3CC(N(C)C)C3)nc3cc(-c4ccccc4F)c4c(c23)OCCC4)CC1]

The compound B16 was synthesized starting from the corresponding starting materials according the similar procedures described as those of Compound B3 to obtain the title compound (21 mg). ¹H NMR (400 MHz, CD₃OD) δ 7.50-7.38 (m, 1H), 7.33-7.23 (m, 2H), 7.20 (t, J=9.2 Hz, 1H), 6.92 (s, 1H), 6.82 (dd, J=16.8, 11.2 Hz, 1H), 6.25 (d, J=16.4 Hz, 1H), 5.78 (d, J=10.4 Hz, 1H), 4.33 (s, 2H), 4.21 (t, J=8.0 Hz, 2H), 4.02-3.91 (m, 2H), 3.89-3.76 (m, 4H), 3.70-3.54 (m, 4H), 3.27-3.18 (m, 1H), 2.66-2.36 (m, 2H), 2.24 (s, 6H), 2.05-1.92 (m, 2H) ppm. MS: M/e 517 (M+1)⁺.

### Compound B17: 1-(4-(5-(3-chlorophenyl)-8-(3-(dimethylamino)azetidin-1-yl)-3,4-dihydro-2H-pyrano[2,3-f]quinazolin-10-yl)piperazin-1-yl)prop-2-en-1-one

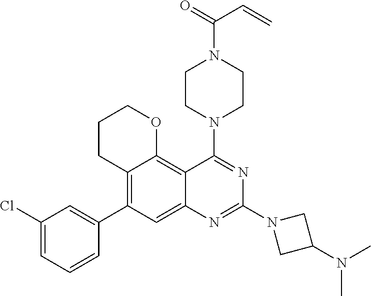 [SMILES: C=CC(=O)N1CCN(c2nc(N3CC(N(C)C)C3)nc3cc(-c4cccc(Cl)c4)c4c(c23)OCCC4)CC1]

The compound B17 was synthesized starting from the corresponding starting materials according the similar procedures described as those of Compound B3 to obtain the title compound (22 mg). ¹H NMR (400 MHz, CD₃OD) δ 7.49-7.38 (m, 2H), 7.37 (s, 1H), 7.28 (d, J=7.2 Hz, 1H), 6.90 (s, 1H), 6.82 (dd, J=16.4, 10.4 Hz, 1H), 6.25 (d, J=17.2 Hz, 1H), 5.78 (d, J=10.8 Hz, 1H), 4.42-4.29 (m, 2H), 4.21 (t, J=8.0 Hz, 2H), 4.02-3.92 (m, 2H), 3.90-3.76 (m, 4H), 3.71-3.54 (m, 4H), 3.28-3.18 (m, 1H), 2.59 (t, J=6.4 Hz, 2H), 2.24 (s, 6H), 2.04-1.90 (m, 2H) ppm. MS: M/e 533 (M+1)⁺.

### Compound B18: 1-(4-(8-(3-(dimethylamino)azetidin-1-yl)-5-(o-tolyl)-3,4-dihydro-2H-pyrano[2,3-f]quinazolin-10-yl)piperazin-1-yl)prop-2-en-1-one

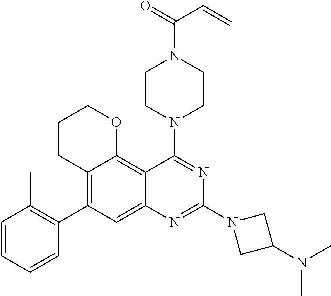 [SMILES: C=CC(=O)N1CCN(c2nc(N3CC(N(C)C)C3)nc3cc(-c4ccccc4C)c4c(c23)OCCC4)CC1]

The compound B18 was synthesized starting from the corresponding starting materials according the similar procedures described as those of Compound B3 to obtain the title compound (24 mg,). ¹H NMR (400 MHz, CD₃OD) δ 7.34-7.19 (m, 3H), 7.07 (d, J=7.2 Hz, 1H), 6.88-6.76 (m, 2H), 6.25 (d, J=16.8 Hz, 1H), 5.78 (d, J=10.8 Hz, 1H), 4.35-4.26 (m, 2H), 4.21 (t, J=8.0 Hz, 2H), 4.02-3.90 (m, 2H), 3.92-3.77 (m, 4H), 3.72-3.54 (m, 4H), 3.27-3.16 (m, 1H), 2.47-2.35 (m, 1H), 2.34-2.16 (m, 7H), 2.09 (s, 3H), 2.02-1.89 (m, 2H) ppm, MS: M/e 513 (M+1)⁺.

### Compound B19: 2-(10-(4-acryloylpiperazin-1-yl)-8-(3-(dimethylamino)azetidin-1-yl)-3,4-dihydro-2H-pyrano[2,3-f]quinazolin-5-yl)benzonitrile

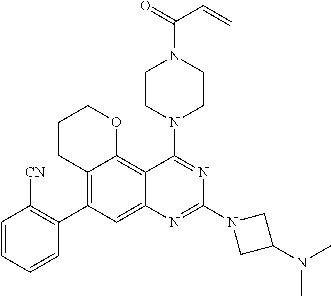 [SMILES: C=CC(=O)N1CCN(c2nc(N3CC(N(C)C)C3)nc3cc(-c4ccccc4C#N)c4c(c23)OCCC4)CC1]

The compound B19 was synthesized starting from the corresponding starting materials according the similar procedures described as those of Compound B3 to give the target compound (10 mg). ¹H NMR (400 MHz, CD₃OD) δ 7.89 (d, J=7.6 Hz, 1H), 7.79 (t, J=7.6 Hz, 1H), 7.63 (t, J=7.6 Hz, 1H), 7.49 (d, J=7.6 Hz, 1H), 6.93 (s, 1H), 6.82 (dd, J=16.4, 11.2 Hz, 1H), 6.27 (d, J=16.0 Hz, 1H), 5.80 (d, J=10.8 Hz, 1H), 4.45-4.27 (m, 4H), 4.12-4.05 (m, 2H), 3.90-3.75 (m, 8H), 3.39 (s, 1H), 2.55-2.47 (m, 2H), 2.32 (s, 6H), 2.07-1.98 (m, 2H) ppm. MS: M/e 524 (M+1)⁺.

### Compound B20: 2-((S)-4-(4-(8-chloronaphthalen-1-yl)-7-(((2S,4R)-4-methoxy-1-methylpyrrolidin-2-yl)methoxy)furo[2,3-f]quinazolin-9-yl)-1-(2-fluoroacryloyl)piperazin-2-yl)acetonitrile

### Step A: ((2S,4R)-4-methoxy-1-methylpyrrolidin-2-yl)methanol

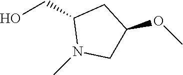

To a solution of tert-butyl (2S,4R)-4-methoxy-1-methylpyrrolidine-2-carboxylate (3.7 g, 14.3 mmol) in THF (50 mL) was added LiAlH₄ (1.1 g, 28.6 mmol). The reaction mixture was stirred at rt for 1 h and 70° C. for 4 h. H₂O was added to the mixture and the mixture was extracted with ethyl acetate. The combined organic extracts were dried over sodium sulfate, filtered and evaporated. The crude product was purified by column chromatography to give the title product (1.5 g, 72%). MS: m/e: 146 (M+1)⁺.

### Step B: 4-bromo-7-(((2S,4R)-4-methoxy-1-methylpyrrolidin-2-yl)methoxy)furo[2,3-f]quinazolin-9(8H)-one

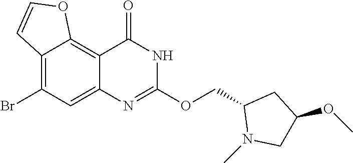 [SMILES: CO[C@@H]1C[C@@H](COc2nc3cc(Br)c4ccoc4c3c(=O)[nH]2)N(C)C1]

To a solution of ((2S,4R)-4-methoxy-1-methylpyrrolidin-2-yl)methanol (556 mg, 4 mmol) in THF (10 ml) was added NaH (200 mg, 5 mmol). The resulting mixture was stirred at rt for 0.5 h. 4-bromo-7-chlorofuro[2,3-f]quinazolin-9(8H)-one (2 mmol, 600 mg) was added and the reaction was stirred at 70° C. for 4 h. H₂O was added and the mixture was extracted with ethyl acetate. The combined organic extracts were dried over sodium sulfate, filtered and evaporated. The crude product was purified by column chromatography to give the title product (400 mg, 49.1%). MS: m/e: 408 (M+1)⁺.

### Step C: tert-butyl (S)-4-(4-bromo-7-(((2S,4R)-4-methoxy-1-methylpyrrolidin-2-yl)methoxy)furo[2,3-f]quinazolin-9-yl)-2-(cyanomethyl)piperazine-1-carboxylate

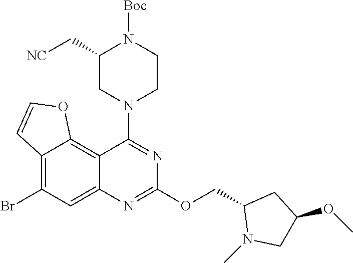 [SMILES: CO[C@@H]1C[C@@H](COc2nc(N3CCN(C(=O)OC(C)(C)C)[C@@H](CC#N)C3)c3c(cc(Br)c4ccoc43)n2)N(C)C1]

To a solution of 4-bromo-7-(((2S,4R)-4-methoxy-1-methylpyrrolidin-2-yl)methoxy)furo[2,3-f]quinazolin-9(8H)-one (400 mg, 1 mmol) in MeCN (10 ml) was added tert-butyl (S)-2-(cyanomethyl)piperazine-1-carboxylate (337 mg, 1.5 mmol), PyBOP (603 mg, 1.5 mol) and DBU (300 mg, 2 mmol). The resulting mixture was stirred at rt overnight. H₂O was added and the mixture was extracted with ethyl acetate. The combined organic extracts were dried over sodium sulfate, filtered and evaporated. The crude product was purified by column chromatography to give the title product (350 mg, 57%). MS: m/e: 615 (M+1)⁺.

### Step D: tert-butyl (S)-4-(4-(8-chloronaphthalen-1-yl)-7-(((2S,4R)-4-methoxy-1-methylpyrrolidin-2-yl)methoxy)furo[2,3-f]quinazolin-9-yl)-2-(cyanomethyl)piperazine-1-carboxylate

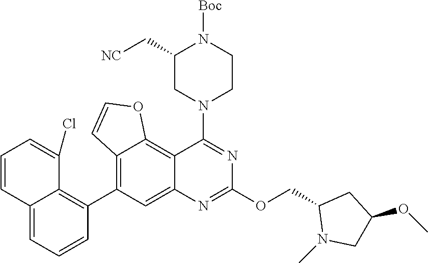 [SMILES: CO[C@@H]1C[C@@H](COc2nc(N3CCN(C(=O)OC(C)(C)C)[C@@H](CC#N)C3)c3c(cc(-c4cccc5cccc(Cl)c45)c4ccoc43)n2)N(C)C1]

To a solution of tert-butyl (S)-4-(4-bromo-7-(((2S,4R)-4-methoxy-1-methylpyrrolidin-2-yl)methoxy)furo[2,3-f]quinazolin-9-yl)-2-(cyanomethyl)piperazine-1-carboxylate (50 mg, 0.08 mmol) in Dioxane (5 mL) was added 2-(8-chloronaphthalen-1-yl)-4,4,5,5-tetramethyl-1,3,2-dioxaborolane (43 mg, 0.15 mmol), pd(dppf)₂Cl₂ (7.31 mg, 0.01 mmol) and a solution of aqueous K₃PO₄ (0.5 ml). The reaction mixture was stirred at 95° C. under N₂ for overnight. H₂O was added and the mixture was extracted with ethyl acetate. The combined organic extracts were dried over sodium sulfate, filtered and evaporated. The crude product was purified by column chromatography to give the title product (15 mg, 26.5%). MS: m/e: 697 (M+1)⁺.

### Step E: 2-((S)-4-(4-(8-chloronaphthalen-1-yl)-7-(((2S,4R)-4-methoxy-1-methylpyrrolidin-2-yl)methoxy)furo[2,3-f]quinazolin-9-yl)piperazin-2-yl)acetonitrile

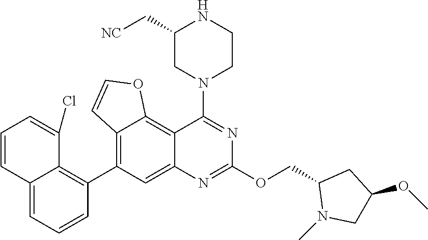 [SMILES: CO[C@@H]1C[C@@H](COc2nc(N3CCN[C@@H](CC#N)C3)c3c(cc(-c4cccc5cccc(Cl)c45)c4ccoc43)n2)N(C)C1]

To a solution of HCl in Dioxane (4 M, 5 ml) was added tert-butyl (S)-4-(4-(8-chloronaphthalen-1-yl)-7-(((2S,4R)-4-methoxy-1-methylpyrrolidin-2-yl)methoxy)furo[2,3-f]quinazolin-9-yl)-2-(cyanomethyl)piperazine-1-carboxylate (15 mg, 0.021 mmol). The resulting mixture was stirred at rt for 4 hours. The mixture was concentrated in vacuo. The crude product was used directly in next step. MS: M/e 597 (M+1)⁺.

### Step F: 2-((S)-4-(4-(8-chloronaphthalen-1-yl)-7-(((2S,4R)-4-methoxy-1-methylpyrrolidin-2-yl)methoxy)furo[2,3-f]quinazolin-9-yl)-1-(2-fluoroacryloyl)piperazin-2-yl)acetonitrile

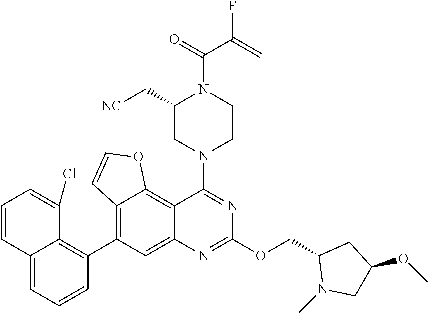 [SMILES: C=C(F)C(=O)N1CCN(c2nc(OC[C@@H]3C[C@@H](OC)CN3C)nc3cc(-c4cccc5cccc(Cl)c45)c4ccoc4c23)C[C@@H]1CC#N]

To a mixture of the product of step E (10 mg, 0.016 mmol) in THF (5 mL) was added 2-fluoroacrylic acid (4.5 mg 0.05 mmol), T₃P (31.8 mg, 0.1 mmol) and Et₃N (20 mg, 0.2 mmol). The reaction was stirred at rt for overnight. H₂O was added and the mixture was extracted with ethyl acetate. The combined organic extracts were dried over sodium sulfate, filtered and evaporated. The residue was purified by prep-HPLC to give the target compound (2.7 mg, 25.2%). ¹H NMR (400 MHz, CD₃OD) δ 8.35 (d, J=7.9 Hz, 1H), 8.26 (d, J=10.4 Hz, 2H), 7.91 (s, 1H), 7.77 (s, 4H), 6.64 (d, J=7.4 Hz, 1H), 5.67-5.51 (m, 2H), 4.81 (s, 2H), 4.64 (d, J=13.4 Hz, 1H), 4.49 (s, 2H), 4.28 (s, 1H), 3.99 (s, 1H), 3.92 (s, 1H), 3.74 (s, 3H), 3.38 (s, 2H), 2.89 (s, 3H), 2.82 (s, 1H), 2.42 (s, 2H), 2.28 (s, 2H), 1.52 (s, 2H) ppm. MS: M/e 669 (M+1)⁺.

### Compound B21: 1-(4-(5-(2-chlorophenyl)-8-(3-(dimethylamino)azetidin-1-yl)-3,4-dihydro-2H-pyrano[2,3-f]quinazolin-10-yl)piperazin-1-yl)prop-2-en-1-one

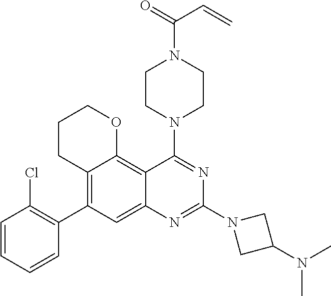 [SMILES: C=CC(=O)N1CCN(c2nc(N3CC(N(C)C)C3)nc3cc(-c4ccccc4Cl)c4c(c23)OCCC4)CC1]

The compound B21 was synthesized starting from the corresponding starting materials according the similar procedures described as those of Compound B3 to obtain the title compound (19 mg). ¹H NMR (400 MHz, CD₃OD) δ 7.56-7.45 (m, 1H), 7.44-7.36 (m, 2H), 7.32-7.21 (m, 1H), 6.90-6.76 (m, 2H), 6.25 (dd, J=16.8, 1.6 Hz, 1H), 5.78 (dd, J=10.8, 2.0 Hz, 1H), 4.30 (t, J=5.2 Hz, 2H), 4.21 (t, J=8.0 Hz, 2H), 4.02-3.93 (m, 2H), 3.88-3.76 (m, 4H), 3.69-3.55 (m, 4H), 3.27-3.09 (m, 1H), 2.53-2.41 (m, 1H), 2.41-2.31 (m, 1H), 2.24 (s, 6H), 2.04-1.92 (m, 2H) ppm. MS: M/e 533 (M+1)⁺.

### Compound B22: 1-(4-(8-(3-(dimethylamino)azetidin-1-yl)-5-(2-(trifluoromethyl)phenyl)-3,4-dihydro-2H-pyrano[2,3-f]quinazolin-10-yl)piperazin-1-yl)prop-2-en-1-one

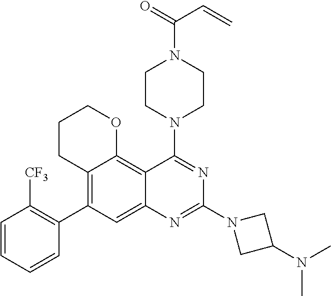 [SMILES: C=CC(=O)N1CCN(c2nc(N3CC(N(C)C)C3)nc3cc(-c4ccccc4C(F)(F)F)c4c(c23)OCCC4)CC1]

The compound B22 was synthesized starting from the corresponding starting materials according the similar procedures described as those of Compound B3 to obtain the title compound (25 mg). ¹H NMR (400 MHz, CD₃OD) δ 7.82 (d, J=7.6 Hz, 1H), 7.68 (t, J=7.6 Hz, 1H), 7.59 (t, J=7.6 Hz, 1H), 7.32 (d, J=7.6 Hz, 1H), 6.89-6.77 (m, 2H), 6.25 (dd, J=16.8, 1.6 Hz, 1H), 5.78 (dd, J=10.4, 2.0 Hz, 1H), 4.38-4.16 (m, 4H), 4.03-3.94 (m, 2H), 3.91-3.75 (m, 4H), 3.69-3.55 (m, 4H), 3.27-3.18 (m, 1H), 2.49-2.35 (m, 1H), 2.33-2.13 (m, 7H), 2.05-1.87 (m, 2H) ppm. MS: M/e 567 (M+1)⁺.

### Compound B23: 1-(4-(5-(8-chloronaphthalen-1-yl)-8-((2-(dimethylamino)ethyl)(methyl)amino)-3,4-dihydro-2H-pyrano[2,3-f]quinazolin-10-yl)piperazin-1-yl)prop-2-en-1-one

### Step A: 5-bromo-8-((2-(dimethylamino)ethyl)(methyl)amino)-2,3,4,9-tetrahydro-10H-pyrano[2,3-f]quinazolin-10-one

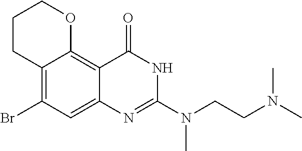 [SMILES: CN(C)CCN(C)c1nc2cc(Br)c3c(c2c(=O)[nH]1)OCCC3]

A solution of 5-bromo-8-chloro-2,3,4,9-tetrahydro-10H-pyrano[2,3-f]quinazolin-10-one (100 mg, 0.32 mmol), N1,N1,N2-trimethylethane-1,2-diamine (64.6 mg, 0.63 mmol) and DIEA (204.1 mg, 1.58 mmol) in THF (4 ml) was stirred at 70° C. overnight. After completed, the solution was concentrated under reduced pressure. The residue was slurried in H₂O and then filtered to afford product (80 mg, 66%). MS: M/e 381,383 (M+1)⁺.

### Step B: tert-butyl 4-(5-bromo-8-((2-(dimethylamino)ethyl)(methyl)amino)-3,4-dihydro-2H-pyrano[2,3-f]quinazolin-10-yl)piperazine-1-carboxylate

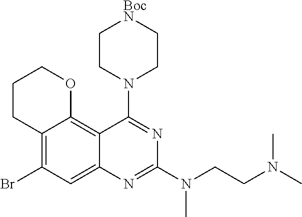 [SMILES: CN(C)CCN(C)c1nc(N2CCN(C(=O)OC(C)(C)C)CC2)c2c3c(c(Br)cc2n1)CCCO3]

To a stirred solution of 5-bromo-8-((2-(dimethylamino)ethyl)(methyl)amino)-2,3,4,9-tetrahydro-10H-pyrano[2,3-f]quinazolin-10-one (80 mg, 0.21 mmol), tert-butyl piperazine-1-carboxylate (58.6 mg, 0.32 mmol) in MeCN (10 ml), was added PyBOP (142 mg, 0.27 mmol) and DBU (64 mg, 0.42 mmol). The mixture was stirred at rt overnight. After completed, the solution was concentrated under reduced pressure. The residue was purified by prep-TLC with DCM:MeOH (10:1) to afford crude product (190 mg), which was used directly for the next step without further purification. MS: M/e 549,551 (M+1)⁺.

### Step C: tert-butyl 4-(5-(8-chloronaphthalen-1-yl)-8-((2-(dimethylamino)ethyl)(methyl)amino)-3,4-dihydro-2H-pyrano[2,3-f]quinazolin-10-yl)piperazine-1-carboxylate

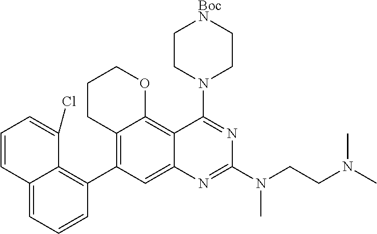 [SMILES: CN(C)CCN(C)c1nc(N2CCN(C(=O)OC(C)(C)C)CC2)c2c3c(c(-c4cccc5cccc(Cl)c45)cc2n1)CCCO3]

A solution of tert-butyl 4-(5-bromo-8-((2-(dimethylamino)ethyl)(methyl)amino)-3,4-dihydro-2H-pyrano[2,3-f]quinazolin-10-yl)piperazine-1-carboxylate (190 mg, crude), 2-(8-chloronaphthalen-1-yl)-4,4,5,5-tetramethyl-1,3,2-dioxaborolane (100 mg, 0.35 mmol), Pd(dppf)Cl₂ (25 mg, 0.034 mmol) and K₃PO₄ (220 mg, 1.04 mmol) in dioxane (10 ml) and H₂O (1.5 ml) was stirred at 100° C. for 2 h under N₂. After completed, the mixture was poured into H₂O (10 ml), extracted with EA (10 ml×2). The organic layer was washed with brine (10 ml), dried and concentrated under reduced pressure. The residue was purified by prep-TLC with DCM:MeOH (20:1) to afford product (74 mg). MS: M/e 631 (M+1)⁺.

### Step D: N′-(5-(8-chloronaphthalen-1-yl)-10-(piperazin-1-yl)-3,4-dihydro-2H-pyrano[2,3-f]quinazolin-8-yl)-N¹,N²,N²-trimethylethane-1,2-diamine

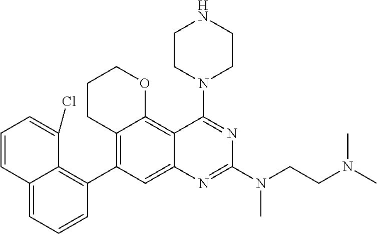 [SMILES: CN(C)CCN(C)c1nc(N2CCNCC2)c2c3c(c(-c4cccc5cccc(Cl)c45)cc2n1)CCCO3]

A solution of tert-butyl 4-(5-(8-chloronaphthalen-1-yl)-8-((2-(dimethylamino)ethyl)(methyl)amino)-3,4-dihydro-2H-pyrano[2,3-f]quinazolin-10-yl)piperazine-1-carboxylate (74 mg, 0.12 mmol) and TFA (0.5 ml) in DCM (6 ml) was stirred at rt for 1 h. After completed, the solution was concentrated under reduced pressure to afford product (60 mg, 96%), which was used directly for the next step without further purification. MS: M/e 531 (M+1)⁺.

### Step E: 1-(4-(5-(8-chloronaphthalen-1-yl)-8-((2-(dimethylamino)ethyl)(methyl)amino)-3,4-dihydro-2H-pyrano[2,3-f]quinazolin-10-yl)piperazin-1-yl)prop-2-en-1-one

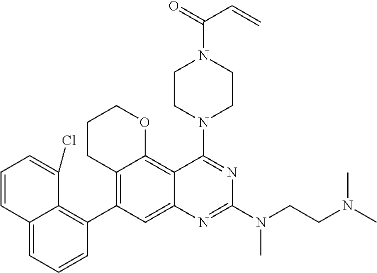 [SMILES: C=CC(=O)N1CCN(c2nc(N(C)CCN(C)C)nc3cc(-c4cccc5cccc(Cl)c45)c4c(c23)OCCC4)CC1]

To a stirred solution of N′-(5-(8-chloronaphthalen-1-yl)-10-(piperazin-1-yl)-3,4-dihydro-2H-pyrano[2,3-f]quinazolin-8-yl)-N¹,N²,N²-trimethylethane-1,2-diamine (60 mg, 0.11 mmol) in MeCN (2 ml), was added aq. NaHCO₃ (sat., 0.1 mL) and then acryloyl chloride (12.3 mg, 0.14 mmol). The mixture was stirred at rt for 2 h. After completed, the solution was concentrated under reduced pressure. The residue was purified by prep-TLC with DCM:MeOH (15:1) to afford product (9.78 mg). 1H NMR (400 MHz, DMSO-d₆) δ 8.05-8.15 (m, 2H), 7.60-7.70 (m, 2H), 7.47-7.59 (m, 1H), 7.36-7.446 (m, 1H), 6.78-7.00 (m, 2H), 6.10-6.25 (m, 1H), 5.65-5.82 (s, 1H), 4.24 (s, 2H), 3.70-4.00 (m, 7H), 3.44-3.57 (m, 4H), 3.12-3.21 (m, 4H), 2.65-2.86 (m, 6H), 1.95-2.25 (s, 2H), 1.85 (s, 2H) ppm, MS: M/e 585 (M+1)⁺.

### Compound B24: 1-(4-(5-(8-chloronaphthalen-1-yl)-8-(4-methylpiperazin-1-yl)-3,4-dihydro-2H-pyrano[2,3-f]quinazolin-10-yl)piperazin-1-yl)prop-2-en-1-one

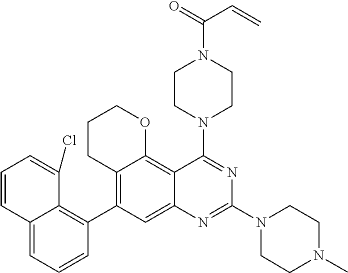 [SMILES: C=CC(=O)N1CCN(c2nc(N3CCN(C)CC3)nc3cc(-c4cccc5cccc(Cl)c45)c4c(c23)OCCC4)CC1]

The compound B24 was synthesized starting from the corresponding starting materials according the similar procedures described as those of Compound B23 to afford product (6.77 mg). 1H NMR (400 MHz, CD3OD) δ 8.01 (d, J=7.7 Hz, 1H), 7.95 (d, J=7.6 Hz, 1H), 7.59 (t, J=8.1 Hz, 1H), 7.55 (d, J=6.7 Hz, 1H), 7.46 (t, J=7.8 Hz, 1H), 7.36 (d, J=7.0 Hz, 1H), 6.95 (s, 1H), 6.89-6.78 (m, 1H), 6.26 (d, J=16.6 Hz, 1H), 5.79 (d, J=10.3 Hz, 1H), 4.28 (s, 2H), 3.87 (d, J=14.7 Hz, 8H), 3.63 (s, 4H), 2.62 (s, 4H), 2.41 (s, 3H), 2.15-2.32 (m, 2H), 1.93 (s, 2H) ppm, MS: M/e 583 (M+1)⁺.

### Compound B25: 1-(4-(5-(5-chloro-6-methyl-1H-indazol-4-yl)-8-(3-(dimethylamino)pyrrolidin-1-yl)-3,4-dihydro-2H-pyrano[2,3-f]quinazolin-10-yl)piperazin-1-yl)prop-2-en-1-one

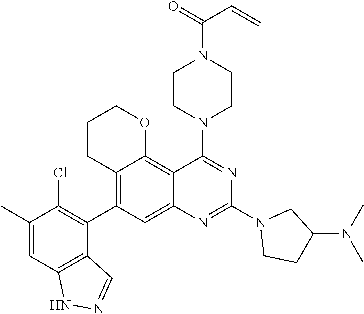

The compound B25 was synthesized starting from the corresponding starting materials according the similar procedures described as those of Compound B23 to afford product (4.1 mg). 1H NMR (400 MHz, CD3OD) δ 7.55 (s, 2H), 6.98 (s, 1H), 6.83 (dd, J=16.8, 10.8 Hz, 1H), 6.26 (d, J=16.6 Hz, 1H), 5.80 (d, J=11.0 Hz, 1H), 4.35 (s, 2H), 3.80-4.08 (m, 7H), 3.65-3.80 (m, 4H), 3.55-3.64 (m, 1H), 3.39-3.48 (m, 1H), 3.03 (s, 1H), 2.78 (s, 1H), 2.57 (s, 3H), 2.40 (s, 6H), 2.18-2.26 (m, 1H), 1.89-2.02 (m, 3H) ppm, MS: M/e 601 (M+1)⁺.

### Compound B26: 1-(4-(5-(8-chloronaphthalen-1-yl)-8-(3-((dimethylamino)methyl)azetidin-1-yl)-3,4-dihydro-2H-pyrano[2,3-f]quinazolin-10-yl)piperazin-1-yl)prop-2-en-1-one

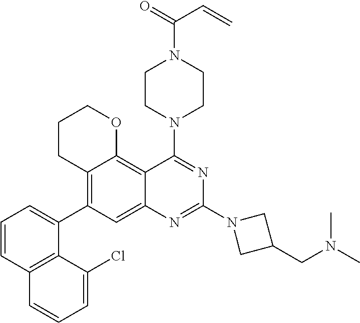 [SMILES: C=CC(=O)N1CCN(c2nc(N3CC(CN(C)C)C3)nc3cc(-c4cccc5cccc(Cl)c45)c4c(c23)OCCC4)CC1]

The compound B26 was synthesized starting from the corresponding starting materials according the similar procedures described as those of Compound B23 to afford product (5.27 mg). 1H NMR (400 MHz, CD3OD) δ 8.04 (d, J=7.7 Hz, 1H), 7.97 (d, J=7.4 Hz, 1H), 7.61 (t, J=7.4 Hz, 1H), 7.56 (d, J=7.1 Hz, 1H), 7.47 (d, J=7.2 Hz, 1H), 7.34 (d, J=6.1 Hz, 1H), 6.94 (s, 1H), 6.89-6.78 (m, 1H), 6.27 (d, J=16.3 Hz, 1H), 5.80 (d, J=10.6 Hz, 1H), 4.41-4.25 (m, 4H), 3.95 (s, 2H), 3.85 (s, 4H), 3.76 (s, 4H), 3.05 (d, J=21.5 Hz, 3H), 2.55 (s, 6H), 2.27 (s, 2H), 1.94 (s, 2H) ppm. MS: M/e 597 (M+1)⁺.

### Compound B27: 1-(4-(5-(3,4-dihydroquinolin-1(2H)-yl)-8-(((2S,4R)-4-fluoro-1-methylpyrrolidin-2-yl)methoxy)-3,4-dihydro-2H-pyrano[2,3-f]quinazolin-10-yl)piperazin-1-yl)prop-2-en-1-one

### Step A: tert-butyl 4-(5-(3,4-dihydroquinolin-1(2H)-yl)-8-(((2S,4R)-4-fluoro-1-methylpyrrolidin-2-yl)methoxy)-3,4-dihydro-2H-pyrano[2,3-f]quinazolin-10-yl)piperazine-1-carboxylate

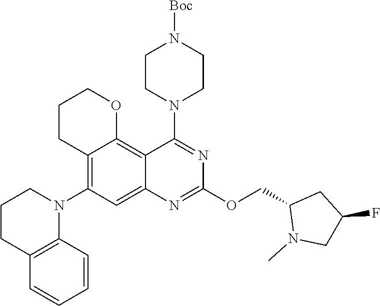 [SMILES: CN1C[C@H](F)C[C@H]1COc1nc(N2CCN(C(=O)OC(C)(C)C)CC2)c2c3c(c(N4CCCc5ccccc54)cc2n1)CCCO3]

A solution of tert-butyl 4-(5-bromo-8-(((2S,4R)-4-fluoro-1-methylpyrrolidin-2-yl)methoxy)-3,4-dihydro-2H-pyrano[2,3-f]quinazolin-10-yl)piperazine-1-carboxylate (63 mg, 0.11 mmol), 1,2,3,4-tetrahydroquinoline (30 mg, 0.22 mmol), Pd₂(dba)₃ (10 mg, 0.01 mmol), BINAP (9 mg, 0.01 mmol) and t-BuONa (21 mg, 0.22 mmol) in toluene (5 mL) was heated at 100° C. under N₂ balloon for 4 hrs. The solution was evaporated, slurried with EA (5 mL) and filtered. The filtrate was concentrated and purified by prep-TLC (DCM:MeOH (7 M NH₃)=15:1) to get the product (30 mg, 44%). MS: M/e 633 (M+1)⁺

### Step B: 5-(3,4-dihydroquinolin-1(2H)-yl)-8-(((2S,4R)-4-fluoro-1-methylpyrrolidin-2-yl)methoxy)-10-(piperazin-1-yl)-3,4-dihydro-2H-pyrano[2,3-f]quinazoline

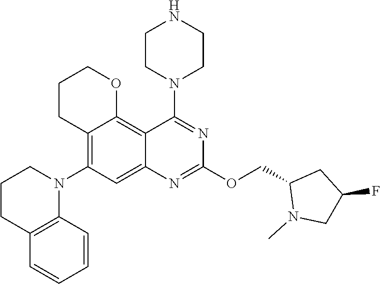 [SMILES: CN1C[C@H](F)C[C@H]1COc1nc(N2CCNCC2)c2c3c(c(N4CCCc5ccccc54)cc2n1)CCCO3]

HCl/dioxane solution (4 M, 2 mL) was added to a solution of tert-butyl 4-(5-(3,4-dihydroquinolin-1(2H)-yl)-8-(((2S,4R)-4-fluoro-1-methylpyrrolidin-2-yl)methoxy)-3,4-dihydro-2H-pyrano[2,3-f]quinazolin-10-yl)piperazine-1-carboxylate (30 mg, 0.05 mmol) in EA (2 mL). After stirred at r.t for 4 hrs, the solution was evaporated to get the crude product, which was used in the next step directly (27 mg, 100%). MS: M/e 533 (M+1)⁺

### Step C: 1-(4-(5-(3,4-dihydroquinolin-1(2H)-yl)-8-(((2S,4R)-4-fluoro-1-methylpyrrolidin-2-yl)methoxy)-3,4-dihydro-2H-pyrano[2,3-f]quinazolin-10-yl)piperazin-1-yl)prop-2-en-1-one

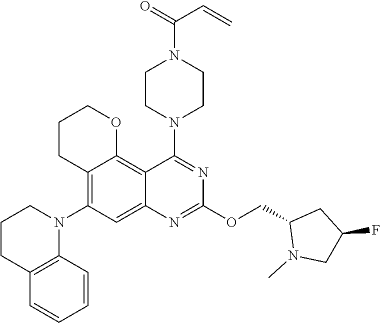 [SMILES: C=CC(=O)N1CCN(c2nc(OC[C@@H]3C[C@@H](F)CN3C)nc3cc(N4CCCc5ccccc54)c4c(c23)OCCC4)CC1]

NaHCO₃ solution (saturated, 0.3 mL) was added to a solution of 5-(3,4-dihydroquinolin-1(2H)-yl)-8-(((2S,4R)-4-fluoro-1-methylpyrrolidin-2-yl)methoxy)-10-(piperazin-1-yl)-3,4-dihydro-2H-pyrano[2,3-f]quinazoline (25 mg, crude) in CH₃CN (3 mL), followed with acryloyl chloride (6 mg, 0.06 mmol). The reaction mixture was stirred at r.t for 30 mins. The solution was evaporated, added with water (4 mL) and extracted with EA (8 mL). The organic layer was dried, concentrated and purified by preparative CombiFlash (DCM:MeOH (7M NH3)=12:1) to get the product (8 mg, 30%). ¹H NMR (400 MHz, CD₃OD) δ 7.02 (d, J=8.0 Hz, 1H), 6.93 (s, 1H), 6.86-6.79 (m, 2H), 6.65 (t, J=8.0 Hz, 1H), 6.24 (s, 1H), 6.22 (s, 1H), 5.78 (t, J=8.0 Hz, 1H), 5.26-5.12 (m, 1H), 4.44 (d, J=4.0 Hz, 2H), 4.33 (s, 2H), 3.83 (s, 4H), 3.55 (s, 4H), 3.49-3.14 (m, 3H), 3.20-3.14 (m, 1H), 2.90-2.58 (m, 5H), 2.54 (s, 3H), 2.30-2.20 (m, 1H), 2.01-1.91 (m, 5H) ppm. MS: M/e 587 (M+1)⁺.

### Compound B28: 1-(4-(4-(5-chloro-6-methyl-1H-indazol-4-yl)-7-(((S)-1-methylpyrrolidin-2-yl)methoxy)-2,3-dihydrofuro[2,3-f]quinazolin-9-yl)piperazin-1-yl)prop-2-en-1-one

### Step A: 2-amino-4-bromo-6-fluorobenzonitrile

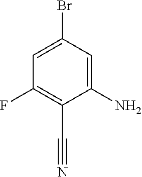 [SMILES: N#Cc1c(N)cc(Br)cc1F]

To a solution of compound 4-bromo-2,6-difluorobenzonitrile (700 g, 3.21 mol) in i-PrOH (1.40 L) was added NH₃—H₂O (2.80 L, 30% purity). The mixture was stirred at 80° C. for 30 hrs. TLC (Petroleum ether/Ethyl acetate=3/1, R_{f}=0.36) showed the reaction was complete. The mixture was poured into H₂O (7.00 L) and the solid was filtered and concentrated in volume to give target compound (600 g, 2.79 mol).

### Step B: 2-(allyloxy)-6-amino-4-bromobenzonitrile

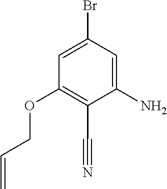

To a solution of compound prop-2-en-1-ol (81.0 g, 1.39 mol) in THF (450 mL) was added NaH (55.8 g, 1.39 mol, 60% purity) at 0° C. and heated to 50° C. Then compound 2-amino-4-bromo-6-fluorobenzonitrile (150 g, 695 mmol) in THF (750 mL) was added to the mixture at 50° C. The mixture was stirred at 60° C. for 2 hrs under N₂. The reaction mixture was poured into HCl (1 L, 0.5M) and extracted with DCM (0.5 L×2). The combined organic layers were washed by brine (0.5 L), dried over Na₂SO₄ and concentrated under pressure to give a residue. The residue was triturated with DCM (500 mL) at 40° C. for 30 min to give target compound (150 g). ¹H NMR: (400 MHz, DMSO) δ 6.58 (s, 1H), 6.57 (s, 1H), 6.43 (s, 2H), 6.04-5.97 (m, 1H), 5.43-5.39 (m, 1H), 5.30-5.26 (m, 1H), 4.64-4.62 (m, 2H).

### Step C: 3-allyl-6-amino-4-bromo-2-hydroxybenzonitrile

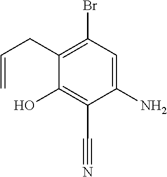

To a solution of the product of step B (150 g, 592 mmol) in DCM (750 mL) was added Et₂AlCl (1M, 1.18 L) at 20° C. The reaction mixture was stirred at 50° C. for 1 h. The reaction was cooled to 20° C. and quenched with HCl (1.25 L, 0.5M). Then the reaction was extracted with DCM (0.75 L×2). The combined organic layers were washed with brine, dried over Na₂SO₄, filtered, and concentrated under reduced pressure to give target compound (100 g). ¹H NMR: (400 MHz, DMSO) δ 10.0 (s, 1H), 6.58 (s, 1H), 5.95 (s, 2H), 5.85-5.74 (m, 1H), 4.97-4.93 (m, 1H), 4.89-4.83 (m, 1H), 3.37-3.32 (m, 2H).

### Step D: tert-butyl(4-allyl-5-bromo-2-cyano-3-hydroxyphenyl)(tert-butoxycarbonyl)carbamate

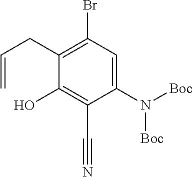 [SMILES: C=CCc1c(Br)cc(N(C(=O)OC(C)(C)C)C(=O)OC(C)(C)C)c(C#N)c1O]

To a solution of the product of step C (100 g, 395 mmol) in DCM (500 mL) was added TEA (80.0 g, 790 mmol), (Boc)₂O (258 g, 1.18 mol) and DMAP (9.65 g, 79.0 mmol). The mixture was stirred at 20° C. for 10 hrs. The mixture was poured into H₂O (0.75 L) and extracted with DCM (0.5 L×2). The combined organic layers were washed with brine (0.5 L), dried over Na₂SO₄ and concentrated under pressure to give a residue. The crude product was purified by column chromatography (SiO₂, Petroleum ether/Ethyl acetate=80/1 to 1/1) to give the target compound (125 g). ¹H NMR: (400 MHz, DMSO) δ 8.05 (s, 1H), 5.86-5.78 (m, 1H), 5.09-5.05 (m, 1H), 4.88-4.82 (m, 1H), 3.55-3.50 (m, 2H), 1.38 (s, 18H).

### Step E: tert-butyl (5-bromo-2-cyano-3-hydroxy-4-(2-oxoethyl)phenyl)(tert-butoxycarbonyl)carbamate

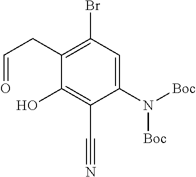 [SMILES: CC(C)(C)OC(=O)N(C(=O)OC(C)(C)C)c1cc(Br)c(CC=O)c(O)c1C#N]

To a solution of the product of step D (100 g, 220 mmol) in THF (400 mL) and H₂O (400 mL) was added OsO₄ (560 mg, 22.0 mol) and NaIO₄ (140 g, 660 mmol) at 0° C. The mixture was stirred at 20° C. for 2 hrs. The mixture was poured into H₂O (0.2 L) and extracted with EtOAc (0.2 L×2). The combined organic layers were dried over Na₂SO₄ and concentrated under pressure to give target compound (100 g), which was used into next step directly.

### Step F: tert-butyl (5-bromo-2-cyano-3-hydroxy-4-(2-hydroxyethyl)phenyl)(tert-butoxycarbonyl)carbamate

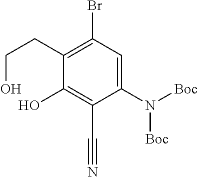 [SMILES: CC(C)(C)OC(=O)N(C(=O)OC(C)(C)C)c1cc(Br)c(CCO)c(O)c1C#N]

To a solution of the product of step E (crude product, 100 g) in THF (1.00 L) was added NaBH₄ (16.6 g, 440 mmol) at 0° C. The mixture was stirred at 20° C. for 2 hrs. The mixture was poured into H₂O (0.2 L) and extracted with EtOAc (0.2 L×2). The combined organic layers were dried over Na₂SO₄ and concentrated under pressure to give a residue. The residue was purified by column chromatography (SiO₂, Petroleum ether/Ethyl acetate=100/1 to 0/1) to give the title compound (28 g). ¹H NMR: (400 MHz, DMSO) δ 8.00 (s, 1H), 4.98-4.94 (m, 1H), 3.52-3.46 (m, 2H), 2.92-2.88 (m, 2H), 1.39 (s, 18H).

### Step G: tert-butyl (5-bromo-2-cyano-3-hydroxy-4-(2-hydroxyethyl)phenyl)carbamate

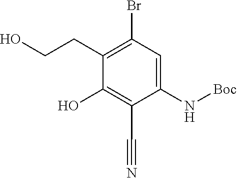 [SMILES: CC(C)(C)OC(=O)Nc1cc(Br)c(CCO)c(O)c1C#N]

To a solution of product of step F (140 g, 306 mmol) in MeOH (1.40 L) was added K₂CO₃ (127 g, 918 mmol). The mixture was stirred at 70° C. for 3 hrs. The mixture was concentrated to remove MeOH. The residue was diluted with EtOAc (1.00 L) and washed with brine (500 mL×2). The combined organic layer was dried over Na₂SO₄ and concentrated to give to give target compound (100 g). ¹H NMR: (400 MHz, DMSO) δ 8.10 (s, 1H), 6.34 (s, 1H), 6.17 (s, 1H), 3.46-3.42 (m, 2H), 2.78-2.74 (m, 2H), 2.92-2.88 (m, 1H), 1.43 (s, 9H).

### Step H: tert-butyl (4-bromo-7-cyano-2,3-dihydrobenzofuran-6-yl)carbamate

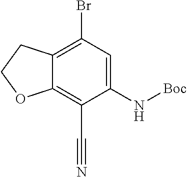 [SMILES: CC(C)(C)OC(=O)Nc1cc(Br)c2c(c1C#N)OCC2]

To a solution of the product of step G (100 g, 280 mmol) in THF (500 mL) was added PPh₃ (80.8 g, 308 mmol) and DIAD (62.3 g, 308 mmol) at 0° C. The mixture was stirred at 0° C. for 2 hrs. The mixture was extracted with H₂O (500 mL) and EtOAc (500 mL×3). The combined organic layers were dried over Na₂SO₄ and concentrated under pressure to give a residue. The residue was purified by column chromatography (SiO₂, Petroleum ether/Ethyl acetate=100/1 to 50/1) to give the target compound (80.0 g, 236 mmol).

### Step I: 6-amino-4-bromo-2,3-dihydrobenzofuran-7-carboxamide

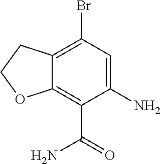 [SMILES: NC(=O)c1c(N)cc(Br)c2c1OCC2]

To a solution of TMSI (14.1 g, 70.8 mmol) in DCM (20 mL) was added tert-butyl (4-bromo-7-cyano-2,3-dihydrobenzofuran-6-yl)carbamate (20.0 g, 58.8 mmol) in DCM (100 mL) at 0° C. The mixture was stirred at 0° C. for 1 h. The mixture was poured into H₂O (100 mL) and extracted with DCM (50.0 mL×2). The combined organic layers were dried over Na₂SO₄ and concentrated under pressure to give a residue. The residue was purified by column chromatography (SiO₂, Petroleum ether/Ethyl acetate=100/1 to 0/1) to give the target compound (4.00 g, 16.8 mmol) ¹H NMR: (400 MHz, DMSO) δ 6.42 (s, 1H), 6.16 (s, 2H), 4.69-4.64 (m, 2H), 3.08-3.02 (m, 2H). LC/MS: M+H=239. 6-amino-4-bromo-2,3-dihydrobenzofuran-7-carbonitrile (238 mg, 1 mmol) in DMSO (4 mL) was treated with 30% aqueous hydrogen peroxide (0.4 mL, 6 mmol) and added 1 M aqueous potassium hydroxide solution (1.5 mL) dropwise. The mixture was stirred 4 hours. The reaction mixture was cooled, treated with water and cooled in an ice-bath. The resulting precipitate was collected by filtration, washed with water and dried in vacuo to obtain the product (200 mg, 78%). LC/MS [M+H]⁺ 257

### Step J: 4-bromo-7-chloro-3,8-dihydrofuro[2,3-f]quinazolin-9(2H)-one

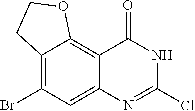 [SMILES: O=c1[nH]c(Cl)nc2cc(Br)c3c(c12)OCC3]

To a solution of 6-amino-4-bromo-2,3-dihydrobenzofuran-7-carboxamide (57 mg, 0.23 mmol) in 1,4-dioxane (4 mL) was added CSCl₂ (76.8 mg, 0.67 mmol). The resulting mixture was stirred at RT for 1 hour, then stirred at 100° C. for another 2 hours. The reaction solvent was removed under vacuum. The crude product (80 mg as a HCl salt) was used to next step directly without further purification MS: M/e 300.9 (M+1)⁺.

### Step K: (S)-4-bromo-7-((1-methylpyrrolidin-2-yl)methoxy)-3,8-dihydrofuro[2,3-f]quinazolin-9(2H)-one

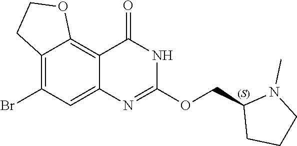

To a solution of the 4-bromo-7-chloro-3,8-dihydrofuro[2,3-f]quinazolin-9(2H)-one (66.7 mg, 0.22 mmol) and (S)-(1-methylpyrrolidin-2-yl)methanol (75.9 mg, 0.66 mmol) in THF (4 mL) was added NaH (60% in mineral oil) (30 mg, 0.73 mmol). The resulting mixture was stirred at 65° C. for another 6 hours. The reaction mixture was quenched with AcOH (0.5 mL) and the solvent was removed under vacuum. The crude product was purified by prep-TLC (DCM:MeOH=20:1) to give the target compound (20 mg, crude). MS: M/e 380.1 (M+1)⁺.

### step L: tert-butyl (S)-4-(4-bromo-7-((1-methylpyrrolidin-2-yl)methoxy)-2,3-dihydrofuro[2,3-f]quinazolin-9-yl)piperazine-1-carboxylate

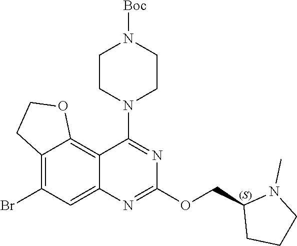

To a solution of (S)-4-bromo-7-((1-methylpyrrolidin-2-yl)methoxy)-3,8-dihydrofuro[2,3-f]quinazolin-9(2H)-one (50 mg, 0.13 mmol) and tert-butyl piperazine-1-carboxylate (36.6 mg, 0.2 mmol) in CH₃CN (10 mL) were added PyBOP (89 mg, 0.17 mmol) and DBU (40 mg, 0.26 mmol). The resulting mixture was stirred at RT for another 3 hours. The reaction solvent was removed under vacuum. The crude product was dissolved with EA (100 mL) and washed with brine and water. The organic layer was concentrated to obtained crude product which further purified by prep-TLC (DCM:MeOH=20:1) to give the target compound (50 mg, crude). MS: M/e 548 (M+1)⁺.

### Step M: tert-butyl 4-(4-(5-chloro-6-methyl-1-(tetrahydro-2H-pyran-2-yl)-1H-indazol-4-yl)-7-(((S)-1-methylpyrrolidin-2-yl)methoxy)-2,3-dihydrofuro[2,3-f]quinazolin-9-yl)piperazine-1-carboxylate

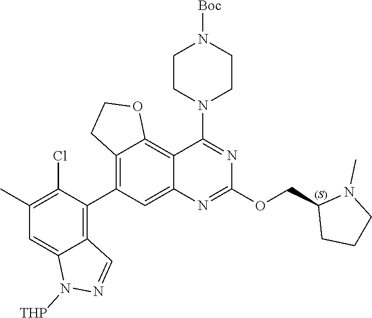

To a mixture of tert-butyl (S)-4-(4-bromo-7-((1-methylpyrrolidin-2-yl)methoxy)-2,3-dihydrofuro[2,3-f]quinazolin-9-yl)piperazine-1-carboxylate (130 mg, crude, 0.12 mmol) and 5-chloro-6-methyl-1-(tetrahydro-2H-pyran-2-yl)-4-(4,4,5,5-tetramethyl-1,3,2-dioxaborolan-2-yl)-1H-indazole (50 mg, 0.09 mmol) in 1,4-dioxane (5 mL) and water (0.5 mL) were added Pd(dppf)Cl₂ (5 mg, 0.007 mmol) and K₃PO₄ (58.1 mg, 0.27 mmol). The resulting mixture was stirred at 95° C. for 2 hours. The reaction solvent was removed under reduce pressure to afford crude product. The crude product was purified by prep-TLC (DCM:MeOH=20:1) to give the target compound (30 mg, crude). MS: M/e 718 (M+1)⁺.

### Step N: 4-(5-chloro-6-methyl-1H-indazol-4-yl)-7-(((S)-1-methylpyrrolidin-2-yl)methoxy)-9-(piperazin-1-yl)-2,3-dihydrofuro[2,3-f]quinazoline

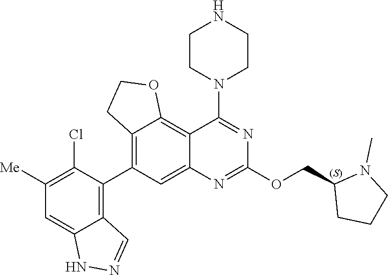

tert-butyl 4-(4-(5-chloro-6-methyl-1-(tetrahydro-2H-pyran-2-yl)-1H-indazol-4-yl)-7-(((S)-1-methylpyrrolidin-2-yl)methoxy)-2,3-dihydrofuro[2,3-f]quinazolin-9-yl)piperazine-1-carboxylate (30 mg, crude) was added to a solution of 4 M HCl in 1,4-dioxane(4 mL) and EA (2 mL). The resulting mixture was stirred at RT for 4 hour. The reaction solvent was removed under reduce pressure to afford the target compound (18.5 mg, crude) which was used to next step directly without further purification. MS: M/e 534 (M+1)⁺.

### Step O: 1-(4-(4-(5-chloro-6-methyl-1H-indazol-4-yl)-7-(((S)-1-methylpyrrolidin-2-yl)methoxy)-2,3-dihydrofuro[2,3-f]quinazolin-9-yl)piperazin-1-yl)prop-2-en-1-one

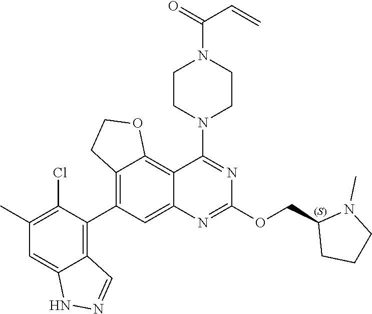

To a mixture of 4-(5-chloro-6-methyl-1H-indazol-4-yl)-7-(((S)-1-methylpyrrolidin-2-yl)methoxy)-9-(piperazin-1-yl)-2,3-dihydrofuro[2,3-f]quinazoline (18.5 mg, 0.034 mmol) and saturated NaHCO₃ aq. (0.5 mL) in CH₃CN (6 mL) was added a solution of acryloyl chloride (3 mg, 0.034 mmol) in CH₃CN (1 mL) dropwise. The resulting mixture was stirred at RT for 1 hour. The reaction mixture was diluted with EtOAc (30 mL) and washed with water (30 mL×2). The organic layers were dried with MgSO₄, filtered and evaporated to afford crude product. The crude product was purified by prep-TLC (DCM:MeOH=10:1) to give the target compound (2.92 mg, 15% yield). 1H NMR (400 MHz, CD₃OD) δ 7.60 (s, 1H), 7.56 (s, 1H), 7.09 (s, 1H), 6.84 (dd, J=16.6, 10.7 Hz, 1H), 6.26 (d, J=16.7 Hz, 1H), 5.80 (d, J=10.5 Hz, 1H), 4.84-4.61 (m, 2H), 4.52 (s, 2H), 3.82 (s, 4H), 3.74 (s, 4H), 3.43-3.22 (m, 2H), 2.83-2.67 (m, 3H), 2.54 (s, 3H), 2.51 (s, 3H), 2.15 (1H), 1.89-1.73 (m, 3H) ppm. MS: M/e 588 (M+1)⁺.

### Compound B29: 2-((S)-4-(5-(2-amino-7-fluorobenzo[d]thiazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

### Step A: tert-butyl (S)-4-(5-(2-((tert-butoxycarbonyl)amino)-7-fluorobenzo[d]thiazol-4-yl)-8-(((S)-1-methylpyrrolidin-2-yl)methoxy)-3,4-dihydro-2H-pyrano[2,3-f]quinazolin-10-yl)-2-(cyanomethyl)piperazine-1-carboxylate

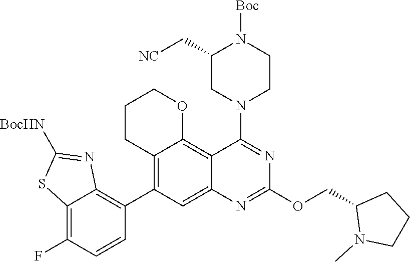 [SMILES: CN1CCC[C@H]1COc1nc(N2CCN(C(=O)OC(C)(C)C)[C@@H](CC#N)C2)c2c3c(c(-c4ccc(F)c5sc(NC(=O)OC(C)(C)C)nc45)cc2n1)CCCO3]

To a solution of tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (30 mg, 0.05 mmol) in Dioxane (5 mL) was added (2-((tert-butoxycarbonyl)amino)-7-fluorobenzo[d]thiazol-4-yl)boronic acid (31 mg, 0.1 mmol), pd(dppf)₂Cl₂ (7.31 mg, 0.01 mmol) and a solution of aqueous K₃PO₄ (0.5 ml). The reaction mixture was stirred at 95° C. under N₂ for overnight. H₂O was added and the mixture was extracted with ethyl acetate. The combined organic extracts were dried over sodium sulfate, filtered and evaporated. The crude product was purified by column chromatography to give the title product (10 mg, 25.3%). MS: m/e: 789 (M+1)⁺.

### Step B: 2-((S)-4-(5-(2-amino-7-fluorobenzo[d]thiazol-4-yl)-8-(((S)-1-methylpyrrolidin-2-yl)methoxy)-3,4-dihydro-2H-pyrano[2,3-f]quinazolin-10-yl)piperazin-2-yl)acetonitrile

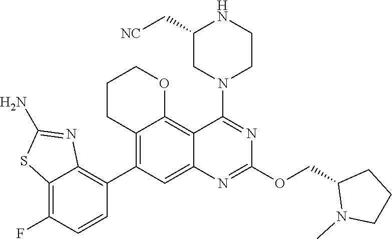 [SMILES: CN1CCC[C@H]1COc1nc(N2CCN[C@@H](CC#N)C2)c2c3c(c(-c4ccc(F)c5sc(N)nc45)cc2n1)CCCO3]

To a solution of HCl in Dioxane (4 M, 5 ml) was added tert-butyl (S)-4-(5-(2-((tert-butoxycarbonyl)amino)-7-fluorobenzo[d]thiazol-4-yl)-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (10 mg, 0.012 mmol). The resulting mixture was stirred at rt for 4 hours. The mixture was concentrated in vacuo. The crude product was used directly in next step. MS: M/e 589 (M+1)⁺.

### Step C: 2-((S)-4-(5-(2-amino-7-fluorobenzo[d]thiazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

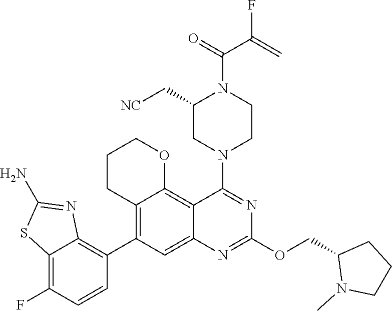 [SMILES: C=C(F)C(=O)N1CCN(c2nc(OC[C@@H]3CCCN3C)nc3cc(-c4ccc(F)c5sc(N)nc45)c4c(c23)OCCC4)C[C@@H]1CC#N]

To a mixture of the product of step B (8 mg, crude) in THF (5 mL) was added 2-fluoroacrylic acid (1.8 mg 0.02 mmol), HATU (7.6 mg, 0.02 mmol) and DIEA (12.9 mg, 0.1 mmol). The reaction was stirred at rt for overnight. H₂O was added and the mixture was extracted with ethyl acetate. The combined organic extracts were dried over sodium sulfate, filtered and evaporated. The residue was purified by prep-HPLC to give the target compound (4 mg, 47.7% for two steps). ¹H NMR (400 MHz, CD₃OD) δ 7.14 (s, 1H), 7.06 (s, 1H), 6.96 (t, J=8.7 Hz, 1H), 5.42-5.30 (m, 2H), 4.78 (d, J=11.7 Hz, 1H), 4.57 (s, 2H), 4.40 (s, 2H), 4.36 (s, 1H), 4.10 (d, J=12.7 Hz, 1H), 3.74 (s, 1H), 3.63 (s, 1H), 3.47 (d, J=9.8 Hz, 1H), 3.13 (s, 1H), 3.02 (s, 1H), 2.99 (s, 3H), 2.36 (d, J=7.5 Hz, 1H), 2.24-2.09 (m, 2H), 2.02 (s, 5H), 1.30 (s, 4H) ppm. MS: M/e 661 (M+1)⁺.

### Compound B30: 2-((2S)-4-(4-(5-chloro-6-methyl-1H-indazol-4-yl)-7-(((S)-1-methylpyrrolidin-2-yl)methoxy)-2,3-dihydrofuro[2,3-f]quinazolin-9-yl)-1-(2-fluoroacryloyl)piperazin-2-yl)acetonitrile

### Step A: tert-butyl (S)-4-(4-bromo-7-(((S)-1-methylpyrrolidin-2-yl)methoxy)-2,3-dihydrofuro[2,3-f]quinazolin-9-yl)-2-(cyanomethyl)piperazine-1-carboxylate

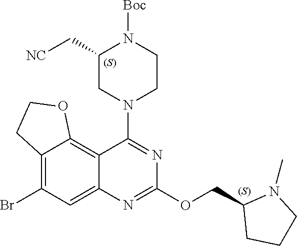

To a solution of the (S)-4-bromo-7-((1-methylpyrrolidin-2-yl)methoxy)-3,8-dihydrofuro[2,3-f]quinazolin-9(2H)-one (20 mg, 0.05 mmol) and tert-butyl (S)-2-(cyanomethyl)piperazine-1-carboxylate (17.8 mg, 0.08 mmol) in CH₃CN (8 mL) were added PyBOP (33.8 mg, 0.065 mmol) and DBU (15.2 mg, 0.1 mmol). The resulting mixture was stirred at RT for another 2 hours. The reaction solvent was removed under vacuum. The crude product was dissolved with EA (20 mL) and washed with brine and water. The organic layer was concentrated to obtained crude product which further purified by prep-TLC (DCM:MeOH=20:1) to give the target compound (20 mg, crude). MS: M/e 587 (M+1)⁺.

### Step B: tert-butyl (2S)-4-(4-(5-chloro-6-methyl-1-(tetrahydro-2H-pyran-2-yl)-1H-indazol-4-yl)-7-(((S)-1-methylpyrrolidin-2-yl)methoxy)-2,3-dihydrofuro[2,3-f]quinazolin-9-yl)-2-(cyanomethyl)piperazine-1-carboxylate

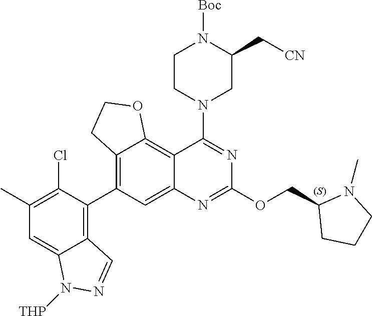

To a mixture of the tert-butyl (S)-4-(4-bromo-7-(((S)-1-methylpyrrolidin-2-yl)methoxy)-2,3-dihydrofuro[2,3-f]quinazolin-9-yl)-2-(cyanomethyl)piperazine-1-carboxylate (20 mg, 0.034 mmol) and 5-chloro-6-methyl-1-(tetrahydro-2H-pyran-2-yl)-4-(4,4,5,5-tetramethyl-1,3,2-dioxaborolan-2-yl)-1H-indazole (19 mg, 0.051 mmol) in 1,4-dioxane (5 mL) and water (0.5 mL) were added Pd(dppf)Cl₂ (2 mg, 0.003 mmol) and K₃PO₄ (22 mg, 0.1 mmol). The resulting mixture was stirred at 100° C. for 2 hours. The reaction solvent was removed under reduce pressure to afford crude product. The crude product was purified by prep-TLC (DCM:MeOH=20:1) to give the target compound (24 mg, crude). MS: M/e 757 (M+1)⁺.

### Step C: 2-((2S)-4-(4-(5-chloro-6-methyl-1H-indazol-4-yl)-7-(((S)-1-methylpyrrolidin-2-yl)methoxy)-2,3-dihydrofuro[2,3-f]quinazolin-9-yl)piperazin-2-yl)acetonitrile

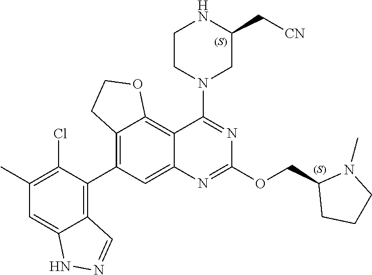

tert-butyl (2S)-4-(4-(5-chloro-6-methyl-1-(tetrahydro-2H-pyran-2-yl)-1H-indazol-4-yl)-7-(((S)-1-methylpyrrolidin-2-yl)methoxy)-2,3-dihydrofuro[2,3-f]quinazolin-9-yl)-2-(cyanomethyl)piperazine-1-carboxylate (24 mg, 0.03 mmol) was added to a solution of 4 N HCl in 1,4-dioxane(5 mL) and EA (2 mL). The resulting mixture was stirred at RT for 2 hours. The reaction solvent was removed under reduce pressure to afford the target compound (18 mg, crude) which was used to next step directly without further purification. MS: M/e 573 (M+1)⁺.

### Step D: 2-((2S)-4-(4-(5-chloro-6-methyl-1H-indazol-4-yl)-7-(((S)-1-methylpyrrolidin-2-yl)methoxy)-2,3-dihydrofuro[2,3-f]quinazolin-9-yl)-1-(2-fluoroacryloyl)piperazin-2-yl)acetonitrile

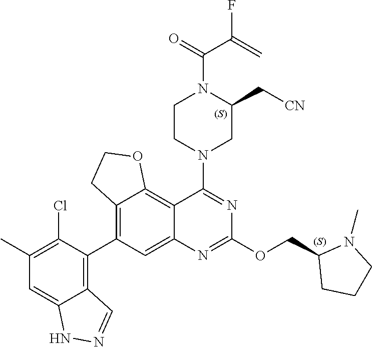

To a solution of 2-((2S)-4-(4-(5-chloro-6-methyl-1H-indazol-4-yl)-7-(((S)-1-methylpyrrolidin-2-yl)methoxy)-2,3-dihydrofuro[2,3-f]quinazolin-9-yl)piperazin-2-yl)acetonitrile (18 mg, 0.03 mmol) and 2-fluoroacryloyl chloride (5.4 mg, 0.06 mmol) in THF (5 mL) were added T₃P (50% in EA)(57 mg, 0.09 mmol) and Et₃N (30.3 mg, 0.3 mmol). The resulting mixture was stirred at RT for 2 hours. The reaction mixture was diluted with EtOAc (20 mL) and washed with NaHCO₃ aq.sat. NaHCO₃ (15 mL). The washings were extracted with EtOAc (15 mL). The organic layers were dried with MgSO₄, filtered and evaporated to afford crude product. The crude product was purified byprep-TLC (DCM:MeOH=20:1) to give the target compound (1.11 mg, 5% yield). 1H NMR (400 MHz, CD₃OD) δ 7.57 (s, 2H), 7.13 (s, 1H), 5.38 (d, J=18.4 Hz, 1H), 5.30 (d, J=8.0 Hz, 1H), 4.50 (s, 2H), 4.44-4.29 (m, 2H), 4.19 (d, J=10.8 Hz, 2H), 3.25-3.16 (m, 5H), 3.07-2.79 (m, 7H), 2.61 (s, 3H), 2.58 (s, 3H), 2.17 (s, 1H), 1.96-1.71 (m, 3H) ppm. MS: M/e 645 (M+1)⁺.

### Compound B31: 2-((S)-1-(2-fluoroacryloyl)-4-(5-(2-fluorophenyl)-8-(((S)-1-methylpyrrolidin-2-yl)methoxy)-3,4-dihydro-2H-pyrano[2,3-f]quinazolin-10-yl)piperazin-2-yl)acetonitrile

### Step A: tert-butyl (S)-2-(cyanomethyl)-4-(5-(2-fluorophenyl)-8-(((S)-1-methylpyrrolidin-2-yl)methoxy)-3,4-dihydro-2H-pyrano[2,3-f]quinazolin-10-yl)piperazine-1-carboxylate

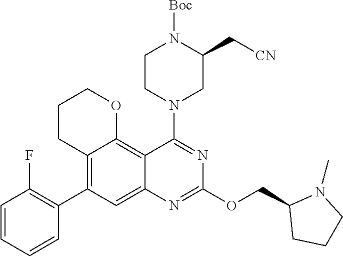 [SMILES: CN1CCC[C@H]1COc1nc(N2CCN(C(=O)OC(C)(C)C)[C@@H](CC#N)C2)c2c3c(c(-c4ccccc4F)cc2n1)CCCO3]

To a stirred solution of tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (100 mg, 0.167 mmol) and (2-fluorophenyl)boronic acid (28 mg, 0.2 mmol) in dioxane/aq. sat.K₃PO₄ (5 mL/0.5 mL) was added PddppfCl₂ (12.2 mg, 0.0167 mmol). After the addition, the reaction was stirred at 100° C. for 2 hours under N₂. The reaction mixture was diluted with EA (10 mL), washed with brine, dried over Na₂SO₄, concentrated to give the residue, which was purified by Prep-TLC (CH₂Cl₂/MeOH=10:1) to give the target compound (90 mg, 87.4%). MS: M/e 617 (M+1)⁺.

### Step B: 2-((S)-1-(2-fluoroacryloyl)-4-(5-(2-fluorophenyl)-8-(((S)-1-methylpyrrolidin-2-yl)methoxy)-3,4-dihydro-2H-pyrano[2,3-f]quinazolin-10-yl)piperazin-2-yl)acetonitrile

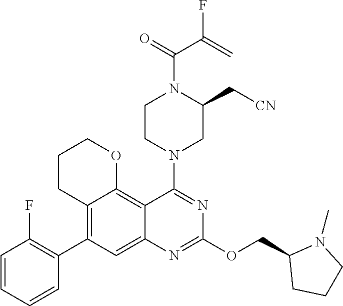 [SMILES: C=C(F)C(=O)N1CCN(c2nc(OC[C@@H]3CCCN3C)nc3cc(-c4ccccc4F)c4c(c23)OCCC4)C[C@@H]1CC#N]

To a stirred solution of the product A (90 mg, 0.146 mmol) in EA (3 mL) was added HCl/dioxane (4 M, 3 mL). After stirred for an hour, the reaction mixture was concentrated, basified with aq. sat, Na₂CO₃ and extracted with EA (20 mL). The organic layer was washed with brine, dried over Na₂SO₄, concentrated to give the intermediate, which was dissolved in THF (5 mL), 2-fluoroacrylic acid (26.4 mg, 0.292 mmol) was added, then T₃P (W/W=50%, 93 mg, 0.292 mmol) followed by Et₃N (44 mg, 0.438 mmol). After the addition, the reaction was stirred for 2 hours. The reaction mixture was diluted with EA (15 mL), washed with brine, dried over Na₂SO₄, concentrated and purified by Prep-TLC (CH₂Cl₂/MeOH=10:1) to give the target compound (8 mg). ¹H NMR (400 MHz, CD₃OD) δ 7.54-7.43 (m, 1H), 7.35-7.27 (m, 2H), 7.22 (t, J=9.2 Hz, 1H), 7.04 (s, 1H), 5.45-5.25 (m, 2H), 4.75-4.63 (m, 1H), 4.58-4.32 (m, 4H), 4.08 (d, J=13.2 Hz, 1H), 3.57-3.42 (m, 4H), 3.25-3.19 (m, 2H), 3.10-2.71 (m, 7H), 2.56 (s, 2H), 2.29 (s, 1H), 2.13-1.90 (m, 5H) ppm. MS: M/e 589 (M+1)⁺.

### Compound B32: 2-((S)-4-(5-(2-cyclopropylphenyl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

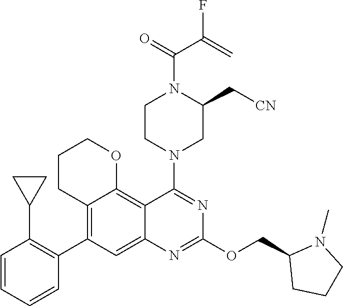 [SMILES: C=C(F)C(=O)N1CCN(c2nc(OC[C@@H]3CCCN3C)nc3cc(-c4ccccc4C4CC4)c4c(c23)OCCC4)C[C@@H]1CC#N]

The Compound B32 was synthesized starting from the corresponding starting materials according the similar procedures described as those of compound B31 to give the target compound (15 mg). ¹H NMR (400 MHz, CD₃OD) δ 7.31 (t, J=7.6 Hz, 1H), 7.25-7.27 (m, 1H), 7.07 (dd, J=18.4, 7.6 Hz, 1H), 7.00 (s, 1H), 6.92 (t, J=8.4 Hz, 1H), 5.43-5.26 (m, 2H), 4.66-4.27 (m, 5H), 4.18-4.05 (m, 1H), 3.67-3.41 (m, 4H), 3.26-3.11 (m, 4H), 3.09-2.87 (m, 2H), 2.75 (s, 3H), 2.60-2.39 (m, 2H), 2.29-1.80 (m, 6H), 1.63-1.47 (m, 1H), 0.90-0.60 (m, 4H) ppm. MS: M/e 611 (M+1)⁺.

### Compound B33: 2-((S)-1-(2-fluoroacryloyl)-4-(8-(((S)-1-methylpyrrolidin-2-yl)methoxy)-5-(2-(trifluoromethyl)phenyl)-3,4-dihydro-2H-pyrano[2,3-f]quinazolin-10-yl)piperazin-2-yl)acetonitrile

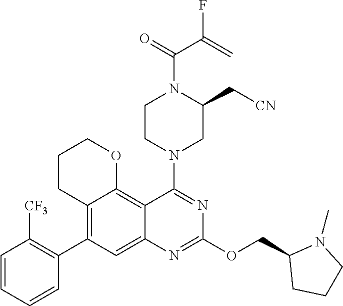 [SMILES: C=C(F)C(=O)N1CCN(c2nc(OC[C@@H]3CCCN3C)nc3cc(-c4ccccc4C(F)(F)F)c4c(c23)OCCC4)C[C@@H]1CC#N]

The Compound B33 was synthesized starting from the corresponding starting materials according the similar procedures described as those of compound B31 to give the target compound (15 mg). ¹H NMR (400 MHz, CD₃OD) δ 7.86-7.81 (m, 1H), 7.74-7.66 (m, 1H), 7.65-7.58 (m, 1H), 7.33 (dd, J=17.6, 7.6 Hz, 1H), 6.97 (s, 1H), 5.45-5.23 (m, 2H), 4.54-4.23 (m, 6H), 4.13-3.97 (m, 1H), 3.51-3.37 (m, 2H), 3.25-2.89 (m, 6H), 2.6 (s, 3H), 2.56-2.41 (m, 2H), 2.35-2.09 (m, 2H), 2.01 (s, 2H), 1.94-1.70 (m, 3H) ppm. MS: M/e 639 (M+1)⁺.

### Compound B34: 1-(4-(4-(5-chloro-6-methyl-1H-indazol-4-yl)-7-(((S)-1-methylpyrrolidin-2-yl)methoxy)furo[2,3-f]quinazolin-9-yl)piperazin-1-yl)prop-2-en-1-one

### Step A: 4-bromo-3-(2,3-dihydroxypropyl)-6-fluoro-2-hydroxybenzonitrile

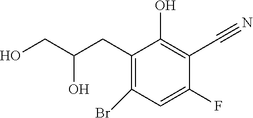 [SMILES: N#Cc1c(F)cc(Br)c(CC(O)CO)c1O]

A round bottom flask was charged with 3-allyl-4-bromo-6-fluoro-2-hydroxybenzonitrile (12.1 g, 47 mmol, 1.0 eq) and dissolved with THF/H₂O (1:1, 80 mL). NMO (13.9 g, 118 mmol, 2.5 equiv) was added to the reaction mixture. K₂OsO₄ 2H₂O (1.7 g, 10 mol %) was added, and the reaction mixture was stirred overnight at rt. The solvent was removed under reduced pressure. The aqueous layer was adjusted PH to 2 with the iN HCl. The mixture was extracted with EtOAc. The combined organic layers were washed with brine, dried over anhydrous Na₂SO₄, filtered, and concentrated in vacuo to give the crude product 12 g. 1H NMR (400 MHz, DMSO-d₆) δ 7.38 (d, J=8.6 Hz, 1H), 4.03 (q, J=6.5 Hz, 1H), 3.77 (s, 1H), 3.01 (d, J=14.2 Hz, 1H), 2.93-2.79 (m, 1H), 1.99 (s, 2H), 1.18 (t, J=6.9 Hz, 2H) ppm.

### Step B: 4-bromo-6-fluoro-2-hydroxy-2,3-dihydrobenzofuran-7-carbonitrile

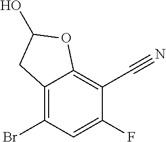 [SMILES: N#Cc1c(F)cc(Br)c2c1OC(O)C2]

4-bromo-3-(2,3-dihydroxypropyl)-6-fluoro-2-hydroxybenzonitrile (12 g, 41 mmol) was dissolved in EA (50 ml) and sodium periodate (13 g, 62 mmol) in water (50 ml). The reaction was stirred for 4 hours at room temperature. The mixture is concentrated and extracted the aqueous residue extracted three times with ethyl acetate. The combined organic phases were washed with sat. NaCl solution, dried (Na₂SO₄) and concentrated in vacuum to give the product. 1H NMR (400 MHz, DMSO-d₆) δ 8.21 (s, 1H), 7.33 (d, J=9.6 Hz, 1H), 6.36 (s, 1H), 3.41 (d, J=11.4 Hz, 1H), 2.83 (d, J=16.6 Hz, 1H) ppm. LC/MS MS: M/e 258 [M+H]⁺

### Step C: 4-bromo-6-fluorobenzofuran-7-carbonitrile

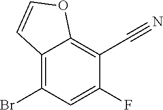 [SMILES: N#Cc1c(F)cc(Br)c2ccoc12]

4-bromo-6-fluoro-2-hydroxy-2,3-dihydrobenzofuran-7-carbonitrile (6 g) was dissolved in DCM (100 ml) followed by addition of sulfuric acid (con., 4.5 ml) dropwise. The mixture was stirred at rt for 2 hours. The reaction was quenched by sat. NaHCO₃, extracted with DCM, dried (Na₂SO₄) and concentrated in vacuum to give the product (5.1 g). 1H NMR (400 MHz, DMSO-d₆) δ 8.38 (s, 1H), 7.92 (d, J=9.7 Hz, 1H), 7.15 (s, 1H) ppm. LC/MS MS: M/e 240 [M+H]⁺

### Step D: 6-amino-4-bromobenzofuran-7-carbonitrile

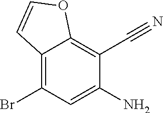 [SMILES: N#Cc1c(N)cc(Br)c2ccoc12]

To 4-bromo-6-fluorobenzofuran-7-carbonitrile (5.1 g) in 40 mL 1-Methyl-2-pyrrolidinone was added ammonium hydroxide (25˜28%, 30 mL). The solution was heated at 120° C. for overnight in a sealed vial. Then the reaction was cooled to rt. The mixture was diluted with water and extracted three times with ethyl acetate. The combined organic phases were washed with sat. NaCl solution, dried (Na₂SO₄) and concentrated in vacuum to give the product. (5 g). 1H NMR (400 MHz, DMSO-d₆) δ 7.92 (d, J=1.5 Hz, 1H), 6.98 (s, 1H), 6.88-6.76 (m, 1H), 6.57 (s, 2H). LC/MS [M+H]⁺ 237.

### Step E: 6-amino-4-bromobenzofuran-7-carboxamide

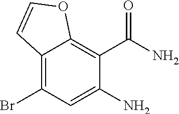 [SMILES: NC(=O)c1c(N)cc(Br)c2ccoc12]

A solution of 6-amino-4-bromobenzofuran-7-carbonitrile (5 g, 21 mmol) in DMSO (40 mL) was treated with 30% aqueous hydrogen peroxide (14 mL, 126 mmol) and added 1 M aqueous potassium hydroxide (4.7 g) solution (15 mL) dropwise. The mixture was stirred 4 hours. The reaction mixture was cooled, treated with water and cooled in an ice-bath. The resulting precipitate was collected by filtration, washed with water and dried in vacuo to obtain the product (4.4 g, 83%). 1H NMR (400 MHz, DMSO-d₆) δ 7.85 (s, 1H), 7.64 (s, 1H), 7.47 (s, 1H), 7.10 (s, 2H), 6.96 (s, 1H), 6.76 (s, 1H) ppm. LC/MS [M+H]⁺ 255.0

### Step F: 4-bromo-7-chlorofuro[2,3-f]quinazolin-9(8H)-one

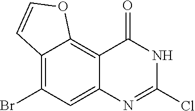 [SMILES: O=c1[nH]c(Cl)nc2cc(Br)c3ccoc3c12]

A solution of 6-amino-4-bromobenzofuran-7-carboxamide (4.4 g, 17 mmol) in dioxane (150 mL) was added thiophosgene (5.9 g, 52 mmol) dropwise. The solution was stirred at room temperature for 1 h then heated to 100° C. for 3 h. The reaction mixture was cooled. The resulting precipitate was collected by filtration and dried in vacuo to obtain the product (3.7 g, 72%). 1H NMR (400 MHz, DMSO-d₆) δ 13.56 (s, 1H), 8.37 (s, 1H), 7.78 (s, 1H), 7.10 (d, J=18.2 Hz, 1H). LC/MS [M+H]⁺ 299.0

### Step G: tert-butyl (S)-4-(4-bromo-7-((1-methylpyrrolidin-2-yl)methoxy)furo[2,3-f]quinazolin-9-yl)piperazine-1-carboxylate

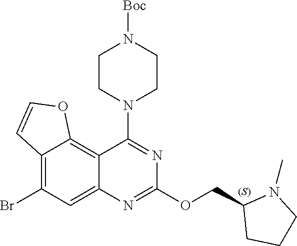

To a solution of (S)-4-bromo-7-((1-methylpyrrolidin-2-yl)methoxy)furo[2,3-f]quinazolin-9(8H)-one (85 mg, 0.21 mmol) and tert-butyl piperazine-1-carboxylate (55.8 mg, 0.3 mmol) in CH₃CN (15 mL) were added PyBOP (196.0 mg, 0.38 mmol) and DBU (64 mg, 0.42 mmol). The resulting mixture was stirred at RT for another 3 hours. The reaction solvent was removed under vacuum. The crude product was dissolved with EA (100 mL) and washed with brine and water. The organic layer was concentrated to obtained crude product which further purified by prep-TLC (DCM:MeOH=20:1) to give the target compound (130 mg, crude). MS: M/e 546 (M+1)⁺.

### Step H: tert-butyl 4-(4-(5-chloro-6-methyl-1-(tetrahydro-2H-pyran-2-yl)-1H-indazol-4-yl)-7-(((S)-1-methylpyrrolidin-2-yl)methoxy)furo[2,3-f]quinazolin-9-yl)piperazine-1-carboxylate

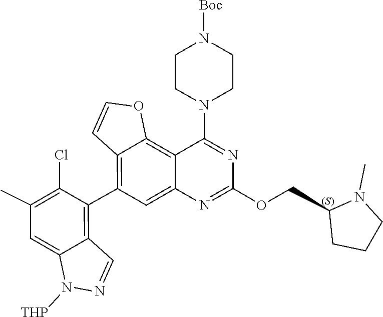

To a mixture of tert-butyl (S)-4-(4-bromo-7-((1-methylpyrrolidin-2-yl)methoxy)furo[2,3-f]quinazolin-9-yl)piperazine-1-carboxylate (130 mg, crude, 0.12 mmol) and 5-chloro-6-methyl-1-(tetrahydro-2H-pyran-2-yl)-4-(4,4,5,5-tetramethyl-1,3,2-dioxaborolan-2-yl)-1H-indazole (128 mg, 0.34 mmol) in 1,4-dioxane (10 mL) and water (1 mL) were added Pd(dppf)Cl₂ (13.2 mg, 0.02 mmol) and K₃PO₄ (152.6 mg, 0.72 mmol). The resulting mixture was stirred at 95° C. for 2 hours. The reaction solvent was removed under reduce pressure to afford crude product. The crude product was purified by prep-TLC (DCM:MeOH=20:1) to give the target compound (200 mg, crude). MS: M/e 716.3 (M+1)⁺.

### Step I: 4-(5-chloro-6-methyl-1H-indazol-4-yl)-7-(((S)-1-methylpyrrolidin-2-yl)methoxy)-9-(piperazin-1-yl)furo[2,3-f]quinazoline

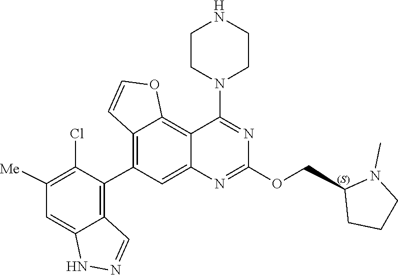

tert-butyl 4-(4-(5-chloro-6-methyl-1-(tetrahydro-2H-pyran-2-yl)-1H-indazol-4-yl)-7-(((S)-1-methylpyrrolidin-2-yl)methoxy)furo[2,3-f]quinazolin-9-yl)piperazine-1-carboxylate (200 mg, crude) was added to a solution of 4 M HCl in 1,4-dioxane(6 mL) and EA (10 mL). The resulting mixture was stirred at RT for 4 hour. The reaction solvent was removed under reduce pressure to afford the target compound (115 mg, 77.4% yield) which was used to next step directly without further purification. MS: M/e 532 (M+1)⁺.

### Step J: 1-(4-(4-(5-chloro-6-methyl-1H-indazol-4-yl)-7-(((S)-1-methylpyrrolidin-2-yl)methoxy)furo[2,3-f]quinazolin-9-yl)piperazin-1-yl)prop-2-en-1-one

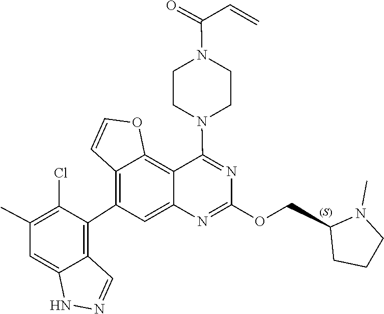

To a mixture of 4-(5-chloro-6-methyl-1H-indazol-4-yl)-7-(((S)-1-methylpyrrolidin-2-yl)methoxy)-9-(piperazin-1-yl)furo[2,3-f]quinazoline (115 mg, 0.216 mmol) and saturated NaHCO₃ aq. (0.5 mL) in CH₃CN (6 mL) was added a solution of acryloyl chloride (15 mg, 0.216 mmol) in CH₃CN (1 mL) dropwise. The resulting mixture was stirred at RT for 1 hour. The reaction mixture was diluted with EtOAc (30 mL) and washed with water (30 mL×2). The organic layers were dried with MgSO₄, filtered and evaporated to afford crude product. The crude product was purified by prep-TLC (DCM:MeOH=10:1) to give the target compound (37.68 mg, 30% yield). 1H NMR (400 MHz, CD₃OD) δ 7.99 (s, 1H), 7.63 (s, 1H), 7.59 (s, 1H), 7.54 (s, 1H), 6.87 (dd, J=16.7, 10.8 Hz, 1H), 6.57 (s, 1H), 6.29 (d, J=16.9 Hz, 1H), 5.81 (d, J=10.6 Hz, 1H), 4.55 (d, J=5.4 Hz, 2H), 4.00 (s, 4H), 3.83 (s, 4H), 3.21 (s, 1H), 3.01 (s, 1H), 2.54 (s, 3H), 2.52 (s, 3H), 2.46-2.42 (m, 1H), 2.19 (s, 1H), 1.92-1.85 (m, 3H) ppm. MS: M/e 586 (M+1)⁺.

### Compound B35: 2-((2S)-4-(4-(5-chloro-6-methyl-1H-indazol-4-yl)-7-(((S)-1-methylpyrrolidin-2-yl)methoxy)furo[2,3-f]quinazolin-9-yl)-1-(2-fluoroacryloyl)piperazin-2-yl)acetonitrile

### Step A: tert-butyl (2S)-4-(4-(5-chloro-6-methyl-1-(tetrahydro-2H-pyran-2-yl)-1H-indazol-4-yl)-7-(((S)-1-methylpyrrolidin-2-yl)methoxy)furo[2,3-f]quinazolin-9-yl)-2-(cyanomethyl)piperazine-1-carboxylate

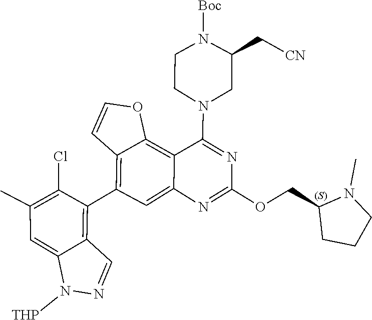

To a mixture of the tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (122 mg, 0.21 mmol) and 5-chloro-6-methyl-1-(tetrahydro-2H-pyran-2-yl)-4-(4,4,5,5-tetramethyl-1,3,2-dioxaborolan-2-yl)-1H-indazole (117.7 mg, 0.31 mmol) in 1,4-dioxane (8 mL) and water (1 mL) were added Pd(dppf)Cl₂ (12.2 mg, 0.017 mmol) and K₃PO₄ (133.4 mg, 0.63 mmol). The resulting mixture was stirred at 100° C. for 2 hours. The reaction solvent was removed under reduce pressure to afford crude product. The crude product was purified by prep-TLC (DCM:MeOH=20:1) to give the target compound (124 mg, 79.0% yield). MS: M/e 755 (M+1)⁺.

### Step B: 2-((2S)-4-(4-(5-chloro-6-methyl-1H-indazol-4-yl)-7-(((S)-1-methylpyrrolidin-2-yl)methoxy)furo[2,3-f]quinazolin-9-yl)piperazin-2-yl)acetonitrile

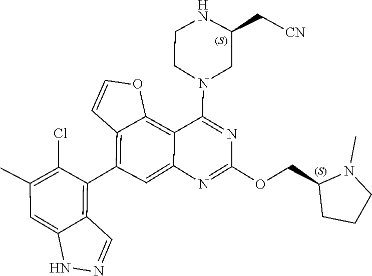

tert-butyl (2S)-4-(4-(5-chloro-6-methyl-1-(tetrahydro-2H-pyran-2-yl)-1H-indazol-4-yl)-7-(((S)-1-methylpyrrolidin-2-yl)methoxy)furo[2,3-f]quinazolin-9-yl)-2-(cyanomethyl)piperazine-1-carboxylate (124 mg, 16 mmol) was added to a solution of 4 N HCl in 1,4-dioxane (5 mL) and EA (5 mL). The resulting mixture was stirred at RT for 3 hours. The reaction solvent was removed under reduce pressure to afford the target compound (40 mg, crude) which was used to next step directly without further purification. MS: M/e 571 (M+1)⁺.

### Step C: 2-((2S)-4-(4-(5-chloro-6-methyl-1H-indazol-4-yl)-7-(((S)-1-methylpyrrolidin-2-yl)methoxy)furo[2,3-f]quinazolin-9-yl)-1-(2-fluoroacryloyl)piperazin-2-yl)acetonitrile

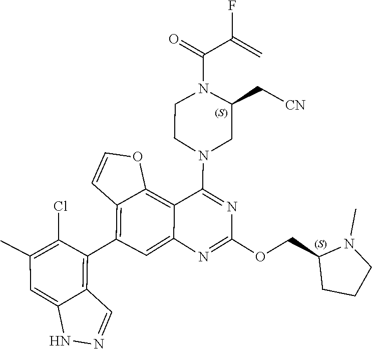

To a solution of 2-((2S)-4-(4-(5-chloro-6-methyl-1H-indazol-4-yl)-7-(((S)-1-methylpyrrolidin-2-yl)methoxy)furo[2,3-f]quinazolin-9-yl)piperazin-2-yl)acetonitrile (40 mg, 0.07 mmol) and 2-fluoroacryloyl chloride (12.6 mg, 0.14 mmol) in THF (8 mL) were added T₃P (50% in EA) (133.5 mg, 0.21 mmol) and Et₃N (35 mg, 0.35 mmol). The resulting mixture was stirred at RT for 2 hours. The reaction mixture was diluted with EtOAc (20 mL) and washed with NaHCO₃ sat. aq. (15 mL). The washings were extracted with EtOAc (15 mL). The organic layers were dried with MgSO₄, filtered and evaporated to afford crude product. The crude product was purified by prep-TLC (DCM:MeOH=20:1) to give the target compound (18.03 mg, 40% yield). 1H NMR (400 MHz, CD₃OD) δ 8.05 (s, 1H), 7.66-7.59 (m, 2H), 7.53 (d, J=4.6 Hz, 1H), 6.59 (s, 1H), 5.47-5.27 (m, 2H), 5.24-4.97 (m, 1H), 4.56 (d, J=4.7 Hz, 2H), 4.38-4.25 (m, 3H), 3.94 (s, 1H), 3.52 (d, J=13.2 Hz, 1H), 3.24-3.02 (m, 4H), 2.58 (s, 3H), 2.56 (s, 3H), 2.51-2.49 (m, 2H), 2.19-1.90 (m, 4H) ppm. MS: M/e 643 (M+1)⁺.

### Compound B36: 2-((2S)-4-(5-(5,6-dichloro-1H-indazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

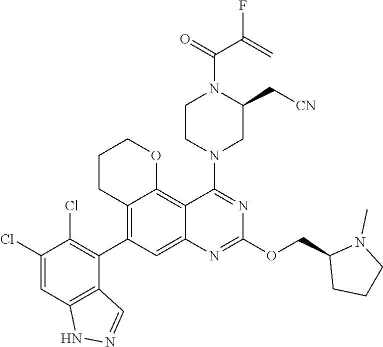 [SMILES: C=C(F)C(=O)N1CCN(c2nc(OC[C@@H]3CCCN3C)nc3cc(-c4c(Cl)c(Cl)cc5[nH]ncc45)c4c(c23)OCCC4)C[C@@H]1CC#N]

The Compound B36 was synthesized starting from the corresponding starting materials according the similar procedures described as those of compound B11 to give the target compound (3 mg). ¹H NMR (400 MHz, CD₃OD) δ 7.87 (s, 1H), 7.58 (s, 1H), 7.06 (s, 1H), 5.44-5.26 (m, 2H), 4.82 (s, 1H), 4.65-4.31 (m, 5H), 4.25-4.06 (m, 2H), 3.85 (s, 1H), 3.72-3.43 (m, 3H), 3.26-3.16 (m, 2H), 3.05 (s, 3H), 3.03-2.93 (m, 1H), 2.60-1.95 (m, 9H) ppm. MS: M/e 679 (M+1)⁺.

### Compound B37: 2-((S)-1-(2-fluoroacryloyl)-4-(5-(8-fluoronaphthalen-1-yl)-8-(((S)-1-methylpyrrolidin-2-yl)methoxy)-3,4-dihydro-2H-pyrano[2,3-f]quinazolin-10-yl)piperazin-2-yl)acetonitrile

### Step A: tert-butyl (S)-2-(cyanomethyl)-4-(8-(((S)-1-methylpyrrolidin-2-yl)methoxy)-5-(4,4,5,5-tetramethyl-1,3,2-dioxaborolan-2-yl)-3,4-dihydro-2H-pyrano[2,3-f]quinazolin-10-yl)piperazine-1-carboxylate

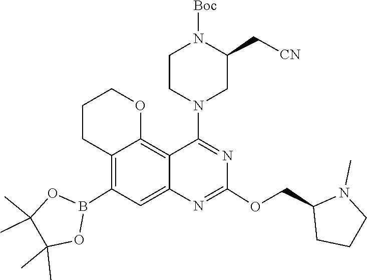 [SMILES: CN1CCC[C@H]1COc1nc(N2CCN(C(=O)OC(C)(C)C)[C@@H](CC#N)C2)c2c3c(c(B4OC(C)(C)C(C)(C)O4)cc2n1)CCCO3]

To a stirred solution of tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (100 mg, 0.167 mmol) and 4,4,4′,4′,5,5,5′,5′-octamethyl-2,2′-bi(1,3,2-dioxaborolane) (51 mg, 0.2 mmol) in dioxane (5 mL/0.5 mL) was added (33 mg, 0.334 mmol) and PddppfCl₂ (12.2 mg, 0.0167 mmol). After the addition, the reaction was stirred at 100° C. for 2 hours under N₂. The reaction mixture was diluted with EA (10 mL), washed with brine, dried over Na₂SO₄, concentrated to give the residue, which was purified by Prep-TLC (CH₂Cl₂/MeOH=10:1) to give the target compound (50 mg, 46.2%). MS: M/e 649 (M+1)⁺.

### Step B: tert-butyl (S)-2-(cyanomethyl)-4-(5-(8-fluoronaphthalen-1-yl)-8-(((S)-1-methylpyrrolidin-2-yl)methoxy)-3,4-dihydro-2H-pyrano[2,3-f]quinazolin-10-yl)piperazine-1-carboxylate

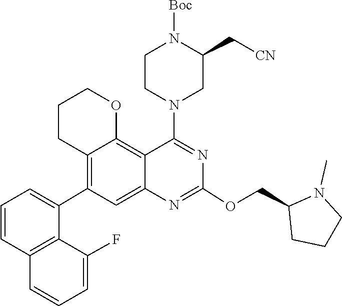 [SMILES: CN1CCC[C@H]1COc1nc(N2CCN(C(=O)OC(C)(C)C)[C@@H](CC#N)C2)c2c3c(c(-c4cccc5cccc(F)c45)cc2n1)CCCO3]

To a stirred solution of the product of step A (50 mg, 0.077 mmol) and 1-bromo-8-fluoronaphthalene (17.4 mg, 0.077 mmol) in dioxane/sat.aq. K₃PO₄ (5 mL/0.5 mL) was added PddppfCl₂ (5.6 mg, 0.0077 mmol). After the addition, the reaction was stirred at 100° C. for 2 hours under N₂. The reaction mixture was diluted with EA (10 mL), washed with brine, dried over Na₂SO₄, concentrated to give the residue, which was purified by Prep-TLC (CH₂Cl₂/MeOH=10:1) to give the target compound (20 mg, 39%). MS: M/e 667 (M+1)⁺.

### Step C: 2-((S)-1-(2-fluoroacryloyl)-4-(5-(8-fluoronaphthalen-1-yl)-8-(((S)-1-methylpyrrolidin-2-yl)methoxy)-3,4-dihydro-2H-pyrano[2,3-f]quinazolin-10-yl)piperazin-2-yl)acetonitrile

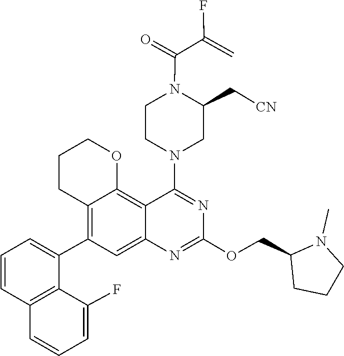 [SMILES: C=C(F)C(=O)N1CCN(c2nc(OC[C@@H]3CCCN3C)nc3cc(-c4cccc5cccc(F)c45)c4c(c23)OCCC4)C[C@@H]1CC#N]

To a stirred solution of the product B (20 mg, 0.03 mmol) in EA (3 mL) was added HCl/dioxane (4 M, 3 mL). After stirred for an hour, the reaction mixture was concentrated, basified with aq.sat. Na₂CO₃ and extracted with EA (20 mL). The organic layer was washed with brine, dried over Na₂SO₄, concentrated to give the intermediate, which was dissolved in THF (5 mL), 2-fluoroacrylic acid (5.4 mg, 0.06 mmol) was added, then T₃P (W/W=50%, 20 mg, 0.06 mmol) followed by Et₃N (9 mg, 0.09 mmol). After the addition, the reaction was stirred for 2 hours. The reaction mixture was diluted with EA (5 mL), washed with brine, dried over Na₂SO₄, concentrated and purified by Prep-TLC (CH₂Cl₂/MeOH=10:1) to give the target compound (4 mg). ¹H NMR (400 MHz, CD₃OD) δ 8.01 (d, J=8.8 Hz, 1H), 7.82 (d, J=8.4 Hz, 1H), 7.67-7.56 (m, 1H), 7.55-7.42 (m, 1H), 7.31 (dd, J=17.2, 7.2 Hz, 1H), 7.19-7.01 (m, 2H), 5.44-5.24 (m, 2H), 4.82 (s, 4H), 4.63-4.27 (m, 4H), 4.11 (d, J=12.4 Hz, 1H), 3.84-3.42 (m, 5H), 3.26-3.20 (m, 4H), 3.01 (s, 3H), 2.48-1.87 (m, 6H) ppm. MS: M/e 639 (M+1)⁺.

### Compound B38: 2-((S)-4-(4-(2,6-difluorophenyl)-7-(((S)-1-methylpyrrolidin-2-yl)methoxy)furo[2,3-f]quinazolin-9-yl)-1-(2-fluoroacryloyl)piperazin-2-yl)acetonitrile

### Step A: (S)-4-bromo-7-((1-methylpyrrolidin-2-yl)methoxy)furo[2,3-f]quinazolin-9(8H)-one

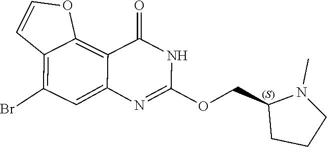

To a solution of the 4-bromo-7-chlorofuro[2,3-f]quinazolin-9(8H)-one (468 mg, 1.57 mmol) and (S)-(1-methylpyrrolidin-2-yl)methanol (542.8 mg, 4.71 mmol) in THF (10 mL) was added NaH (60% in mineral oil)(208 mg, 4.71 mmol). The resulting mixture was stirred at 65° C. for another 4 hours. The reaction mixture was quenched with AcOH (3 mL) and the solvent was removed under vacuum. The crude product was purified by prep-TLC (DCM:MeOH=20:1) to give the target compound (390 mg, 71% yield). MS: M/e 378 (M+1)⁺.

### Step B: tert-butyl (S)-4-(4-bromo-7-(((S)-1-methylpyrrolidin-2-yl)methoxy)furo[2,3-f]quinazolin-9-yl)-2-(cyanomethyl)piperazine-1-carboxylate

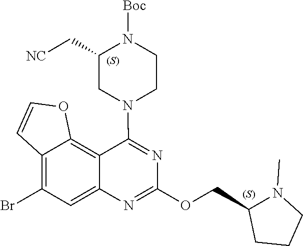

To a solution of the (S)-4-bromo-7-((1-methylpyrrolidin-2-yl)methoxy)furo[2,3-f]quinazolin-9(8H)-one (550 mg, 1.46 mmol) and tert-butyl (S)-2-(cyanomethyl)piperazine-1-carboxylate (492 mg, 2.18 mmol) in CH₃CN (50 mL) were added PyBOP (987.0 mg, 1.90 mmol) and DBU (443.8 mg, 2.92 mmol). The resulting mixture was stirred at RT for another 3 hours. The reaction solvent was removed under vacuum. The crude product was dissolved with EA (200 mL) and washed with brine and water. The organic layer was concentrated to obtained crude product which further purified by prep-TLC (DCM:MeOH=20:1) to give the target compound (440 mg, 52% yield). MS: M/e 585 (M+1)⁺.

### Step C: tert-butyl (S)-2-(cyanomethyl)-4-(4-(2,6-difluorophenyl)-7-(((S)-1-methylpyrrolidin-2-yl)methoxy)furo[2,3-f]quinazolin-9-yl)piperazine-1-carboxylate

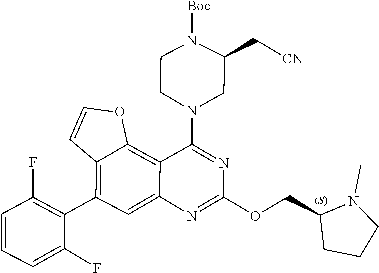

To a mixture of the tert-butyl (S)-4-(4-bromo-7-(((S)-1-methylpyrrolidin-2-yl)methoxy)furo[2,3-f]quinazolin-9-yl)-2-(cyanomethyl)piperazine-1-carboxylate (90 mg, 0.15 mmol) and (2,6-difluorophenyl)boronic acid (36.5 mg, 0.23 mmol) in 1,4-dioxane (10 mL) and water (1 mL) were added Pd(dppf)Cl₂ (8.4 mg, 0.01 mmol) and K₃PO₄ (98 mg, 0.46 mmol). The resulting mixture was stirred at 100° C. for 2 hours. The reaction solvent was removed under reduce pressure to afford crude product. The crude product was purified by prep-TLC (DCM:MeOH=20:1) to give the target compound (66 mg, 69.3% yield). MS: M/e 619.3 (M+1)⁺.

### Step D: 2-((S)-4-(4-(2,6-difluorophenyl)-7-(((S)-1-methylpyrrolidin-2-yl)methoxy)furo[2,3-f]quinazolin-9-yl)piperazin-2-yl)acetonitrile

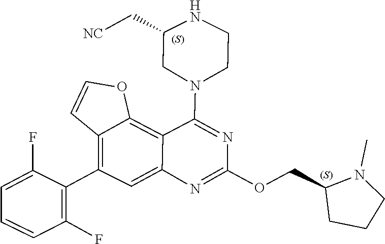

tert-butyl (S)-2-(cyanomethyl)-4-(4-(2,6-difluorophenyl)-7-(((S)-1-methylpyrrolidin-2-yl)methoxy)furo[2,3-f]quinazolin-9-yl)piperazine-1-carboxylate (66 mg, 0.11 mmol) was added to a solution of 4 N HCl in 1,4-dioxane(4 mL). The resulting mixture was stirred at RT for 1 hour. The reaction solvent was removed under reduce pressure to afford the target compound (30 mg, 54% yield) which was used to next step directly without further purification. MS: M/e 519 (M+1)⁺.

### Step E: 2-((S)-4-(4-(2,6-difluorophenyl)-7-(((S)-1-methylpyrrolidin-2-yl)methoxy)furo[2,3-f]quinazolin-9-yl)-1-(2-fluoroacryloyl)piperazin-2-yl)acetonitrile

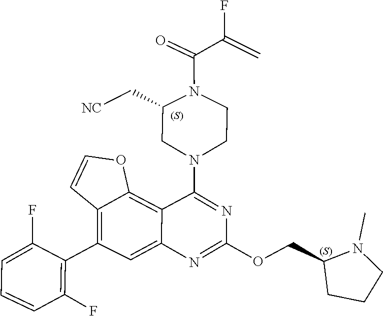

To a solution of the product of the step D (30 mg, 0.06 mmol) and 2-fluoroacryloyl chloride (10.4 mg, 0.11 mmol) in THF (5 mL) were added T₃P (50% in EA)(114.48 mg, 0.18 mmol) and Et₃N (30.3 mg, 0.30 mmol). The resulting mixture was stirred at RT for 1 hour. The reaction mixture was diluted with EtOAc (20 mL) and washed with NaHCO₃ aq (15 mL). The washings were extracted with EtOAc (15 mL). The organic layers were dried with MgSO₄, filtered and evaporated to afford crude product. The crude product was purified by prep-TLC (DCM:MeOH=20:1) to give the target compound (3.41 mg, 10% yield). 1H NMR (400 MHz, CD₃OD) δ 8.11 (s, 1H), 7.60 (d, J=18.4 Hz, 2H), 7.22 (t, J=8.8 Hz, 2H), 6.80 (s, 1H), 5.48-5.27 (m, 2H), 5.23-5.04 (m, 1H), 4.55 (d, J=5.7 Hz, 2H), 4.40 (d, J=13.3 Hz, 1H), 4.25 (d, J=12.2 Hz, 1H), 3.52 (d, J=13.2 Hz, 2H), 3.18 (4H), 2.98 (s, 1H), 2.58 (s, 3H), 2.50 (2H), 2.20 (s, 1H), 1.91 (s, 3H) ppm. MS: M/e 591 (M+1)⁺.

### Compound B39: 2-((2S)-4-(4-(2-fluoro-6-hydroxyphenyl)-7-(((S)-1-methylpyrrolidin-2-yl)methoxy)furo[2,3-f]quinazolin-9-yl)-1-(2-fluoroacryloyl)piperazin-2-yl)acetonitrile

### Step A: tert-butyl (2S)-4-(4-(2-(benzyloxy)-6-fluorophenyl)-7-(((S)-1-methylpyrrolidin-2-yl)methoxy)furo[2,3-f]quinazolin-9-yl)-2-(cyanomethyl)piperazine-1-carboxylate

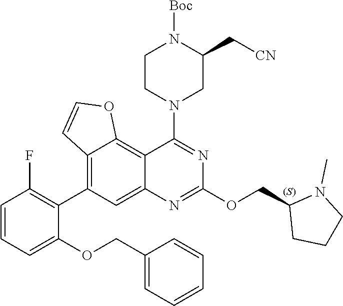

To a mixture of the tert-butyl (S)-4-(4-bromo-7-(((S)-1-methylpyrrolidin-2-yl)methoxy)furo[2,3-f]quinazolin-9-yl)-2-(cyanomethyl)piperazine-1-carboxylate (90 mg, 0.15 mmol) and (2-(benzyloxy)-6-fluorophenyl)boronic acid (56.5 mg, 0.23 mmol) in 1,4-dioxane (10 mL) and water (1 mL) were added Pd(dppf)Cl₂ (8.4 mg, 0.01 mmol) and K₃PO₄ (98 mg, 0.46 mmol). The resulting mixture was stirred at 100° C. for 2 hours. The reaction solvent was removed under reduce pressure to afford crude product. The crude product was purified by prep-TLC (DCM:MeOH=20:1) to give the target compound (48 mg, 44% yield). MS: M/e 707 (M+1)⁺.

### Step B: 2-((2S)-4-(4-(2-fluoro-6-hydroxyphenyl)-7-(((S)-1-methylpyrrolidin-2-yl)methoxy)furo[2,3-f]quinazolin-9-yl)piperazin-2-yl)acetonitrile

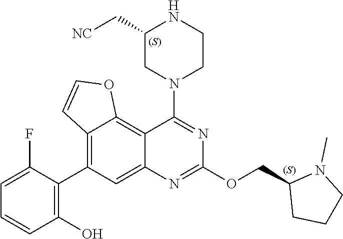

To a solution of the product of Step A (48 mg, 0.67 mmol) in DCM (5 mL) was added BBr₃ aq (1 M, 0.34 mL, 0.34 mmol) at 0° C. under an ice-bath. The resulting mixture was stirred at RT for 1 hour. The reaction was quenched with NaHCO₃ aq and extracted with DCM (20 mL×2). The organic layer was dried over Na₂SO₄ and concentrated under reduce pressure to to afford the target compound (50 mg, crude) which was used to next step directly without further purification. MS: M/e 517 (M+1)⁺.

### Step C: 2-((2S)-4-(4-(2-fluoro-6-hydroxyphenyl)-7-(((S)-1-methylpyrrolidin-2-yl)methoxy)furo[2,3-f]quinazolin-9-yl)-1-(2-fluoroacryloyl)piperazin-2-yl)acetonitrile

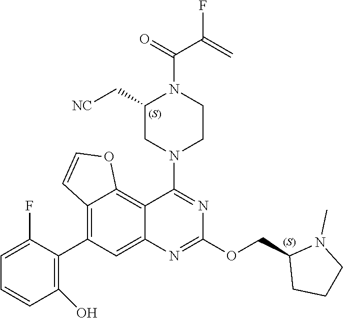

To a solution of the product of Step B (50 mg crude, 0.06 mmol,) and 2-fluoroacryloyl chloride (17.4 mg, 0.19 mmol) in THF (5 mL) were added T₃P (50% in EA)(183 mg, 0.29 mmol) and Et₃N (48 mg, 0.48 mmol). The resulting mixture was stirred at RT for 1 hour. The reaction mixture was diluted with EtOAc (20 mL) and washed with NaHCO₃ aq (15 mL). The water phase was extracted with EtOAc (15 mL). The combined organic layers were dried with MgSO₄, filtered and evaporated to afford crude product. The crude product was purified by prep-TLC (DCM:MeOH=20:1) to give the target compound (0.2 mg, 0.3% yield). 1H NMR (400 MHz, CD3OD) δ 8.06 (s, 1H), 7.61 (s, 1H), 7.32 (s, 1H), 6.78 (s, 3H), 5.33 (s, 2H), 4.70 (s, 2H), 4.45 (s, 1H), 4.29 (s, 2H), 3.95 (s, 2H), 3.73 (s, 1H), 3.50 (s, 2H), 3.12 (s, 5H), 2.88-2.73 (m, 1H), 2.44 (s, 1H), 2.12 (s, 4H) ppm. MS: M/e 589 (M+1)⁺.

### Compound B40: 2-((S)-1-(2-fluoroacryloyl)-4-(7-(((S)-1-methylpyrrolidin-2-yl)methoxy)-4-(2-(trifluoromethyl)phenyl)furo[2,3-f]quinazolin-9-yl)piperazin-2-yl)acetonitrile

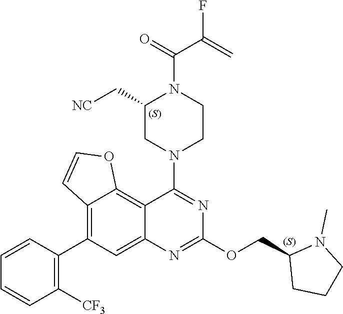

The Compound B40 was synthesized starting from the corresponding starting materials according the similar procedures described as those of compound B38 to give the target compound (4.87 mg) 1H NMR (400 MHz, CD3OD) δ 8.07 (s, 1H), 7.92 (s, 1H), 7.74 (2H), 7.48 (s, 2H), 6.62 (s, 1H), 5.52-5.27 (m, 2H), 5.23-5.01 (m, 1H), 4.65-4.23 (m, 5H), 4.15-3.75 (m, 1H), 3.53 (s, 1H), 3.15 (s, 3H), 2.91 (s, 1H), 2.60 (s, 3H), 2.46 (s, 1H), 2.18 (s, 1H), 1.88 (s, 4H) ppm. MS: M/e 623 (M+1)⁺.

### Compound B41: 2-((S)-4-(4-(8-chloronaphthalen-1-yl)-7-(((S)-1-methylpyrrolidin-2-yl)methoxy)furo[2,3-f]quinazolin-9-yl)-1-(2-fluoroacryloyl)piperazin-2-yl)acetonitrile

### Step A: tert-butyl (S)-4-(4-(8-chloronaphthalen-1-yl)-7-(((S)-1-methylpyrrolidin-2-yl)methoxy)furo[2,3-f]quinazolin-9-yl)-2-(cyanomethyl)piperazine-1-carboxylate

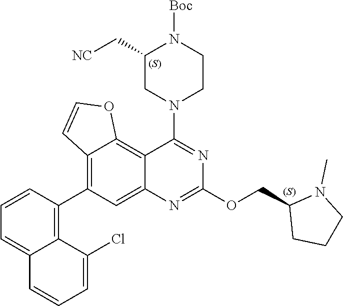

To a mixture of the Tert-butyl (S)-4-(4-bromo-7-(((S)-1-methylpyrrolidin-2-yl)methoxy)furo[2,3-f]quinazolin-9-yl)-2-(cyanomethyl)piperazine-1-carboxylate (90 mg, 0.15 mmol) and 2-(8-chloronaphthalen-1-yl)-4,4,5,5-tetramethyl-1,3,2-dioxaborolane (66 mg, 0.23 mmol) in 1,4-dioxane (10 mL) and water (1 mL) were added Pd(dppf)Cl₂ (8.4 mg, 0.01 mmol) and K₃PO₄ (98 mg, 0.46 mmol). The resulting mixture was stirred at 100° C. for 2 hours. The reaction solvent was removed under reduce pressure to afford crude product. The crude product was purified byprep-TLC (DCM:MeOH=20:1) to give the target compound (60 mg, 80% yield). MS: M/e 667 (M+1)⁺.

### Step B: 2-((S)-4-(4-(8-chloronaphthalen-1-yl)-7-(((S)-1-methylpyrrolidin-2-yl)methoxy)furo[2,3-f]quinazolin-9-yl)piperazin-2-yl)acetonitrile

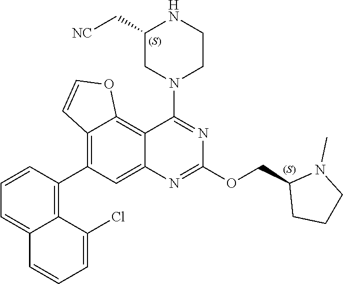

The product of Step A (80 mg, 0.12 mmol) was added to a solution of 4 N HCl in 1,4-dioxane(4 mL). The resulting mixture was stirred at RT for 1 hour. The reaction solvent was removed under reduce pressure to afford the target compound (35 mg, 51% yield) which was used to next step directly without further purification. MS: M/e 567 (M+1)⁺.

### Step C: 2-((S)-4-(4-(8-chloronaphthalen-1-yl)-7-(((S)-1-methylpyrrolidin-2-yl)methoxy)furo[2,3-f]quinazolin-9-yl)-1-(2-fluoroacryloyl)piperazin-2-yl)acetonitrile

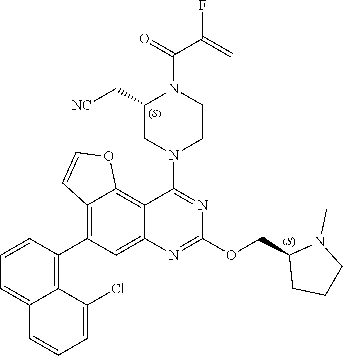

To a solution of the product of Step B (35 mg, 0.06 mmol) and 2-fluoroacryloyl chloride (11 mg, 0.12 mmol) in THF (5 mL) were added T₃P (50% in EA)(115 mg, 0.18 mmol) and Et₃N (30.3 mg, 0.30 mmol). The resulting mixture was stirred at RT for 1 hour. The reaction mixture was diluted with EtOAc (20 mL) and washed with NaHCO₃ aq (15 mL). The washings were extracted with EtOAc (15 mL). The organic layers were dried with MgSO₄, filtered and evaporated to afford crude product. The crude product was purified by prep-TLC (DCM:MeOH=20:1) to give the target compound (10.35 mg, 26% yield). 1H NMR (400 MHz, CD3OD) δ 8.11 (s, 1H), 8.00 (s, 2H), 7.66 (s, 1H), 7.53 (s, 4H), 6.39 (s, 1H), 5.48-5.25 (m, 2H), 4.53 (s, 2H), 4.45-4.17 (m, 3H), 3.53 (s, 1H), 3.22-3.15 (m, 5H), 2.89 (s, 1H), 2.58 (s, 3H), 2.43 (s, 1H), 2.17 (s, 1H), 1.87 (s, 4H) ppm. MS: M/e 639 (M+1)⁺.

### Compound B42: 2-((2S)-4-(5-(6-chloro-5-fluoro-1H-indazol-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

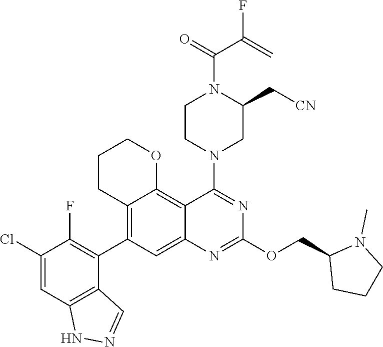 [SMILES: C=C(F)C(=O)N1CCN(c2nc(OC[C@@H]3CCCN3C)nc3cc(-c4c(F)c(Cl)cc5[nH]ncc45)c4c(c23)OCCC4)C[C@@H]1CC#N]

The Compound B42 was synthesized starting from the corresponding starting materials according the similar procedures described as those of compound B11 to give the target compound. ¹H NMR (400 MHz, CD₃OD) δ 7.84-7.80 (m, 1H), 7.77-7.72 (m, 1H), 7.18 (d, J=7.2 Hz, 1H), 5.47-5.27 (m, 2H), 4.72-4.35 (m, 5H), 4.27-4.12 (m, 2H), 3.92 (s, 1H), 3.71 (s, 2H), 3.60-3.47 (m, 2H), 3.13-2.95 (m, 5H), 2.95-2.55 (m, 2H), 2.49-2.35 (m, 2H), 2.31-2.95 (m, 6H) ppm. MS: M/e 663 (M+1)⁺.

### Compound B43: 2-((S)-1-(2-fluoroacryloyl)-4-(5-(8-methylnaphthalen-1-yl)-8-(((S)-1-methylpyrrolidin-2-yl)methoxy)-3,4-dihydro-2H-pyrano[2,3-f]quinazolin-10-yl)piperazin-2-yl)acetonitrile

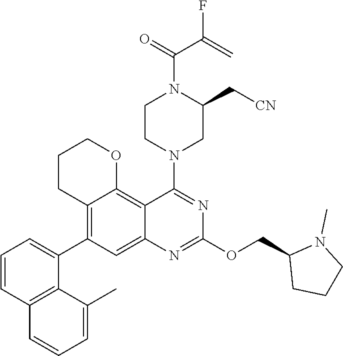 [SMILES: C=C(F)C(=O)N1CCN(c2nc(OC[C@@H]3CCCN3C)nc3cc(-c4cccc5cccc(C)c45)c4c(c23)OCCC4)C[C@@H]1CC#N]

The Compound B43 was synthesized starting from the corresponding starting materials according the similar procedures described as those of compound B31 to give the target compound (15 mg). ¹H NMR (400 MHz, CD₃OD) δ 7.96 (d, J=8.0 Hz, 1H), 7.85 (d, J=7.6 Hz, 1H), 7.58-7.48 (m, 1H), 7.47-7.35 (m, 1H), 7.34-7.19 (m, 2H), 7.14 (d, J=7.2 Hz, 1H), 5.48-5.27 (m, 2H), 4.83 (d, J=12.4 Hz, 1H), 4.67-4.08 (m, 6H), 3.87-3.45 (m, 5H), 3.4-3.14 (m, 2H), 3.04 (s, 5H), 2.49-2.13 (m, 4H), 2.09 (d, J=11.2 Hz, 5H), 1.99 (s, 2H) ppm. MS: M/e 635 (M+1)⁺.

### Compound B44: (S)-2-fluoro-1-(4-(4-(2-fluorophenyl)-7-((1-methylpyrrolidin-2-yl)methoxy)furo[2,3-f]quinazolin-9-yl)piperazin-1-yl)prop-2-en-1-one

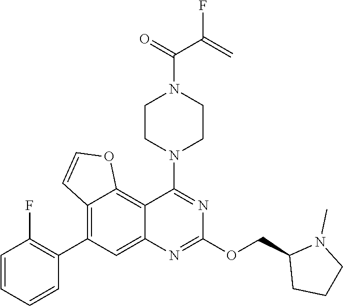 [SMILES: C=C(F)C(=O)N1CCN(c2nc(OC[C@@H]3CCCN3C)nc3cc(-c4ccccc4F)c4ccoc4c23)CC1]

The Compound B44 was synthesized starting from the corresponding starting materials according the similar procedures described as those of compound B41 to give the target compound (20 mg). ¹H NMR (400 MHz, CD₃OD) δ 8.15-8.02 (m, 1H), 7.75-7.50 (m, 3H), 7.49-7.29 (m, 2H), 7.00-6.85 (m, 1H), 5.48-5.21 (m, 2H), 4.82-4.58 (m, 2H), 3.91 (d, J=61.2 Hz, 8H), 3.61 (2H), 3.15-2.90 (m, 4H), 2.49-2.28 (m, 1H), 2.24-1.93 (m, 3H) ppm. MS: M/e 534 (M+1)⁺.

### Compound B45: (S)-1-(4-(4-(2-fluorophenyl)-7-((1-methylpyrrolidin-2-yl)methoxy)furo[2,3-f]quinazolin-9-yl)piperazin-1-yl)prop-2-en-1-one

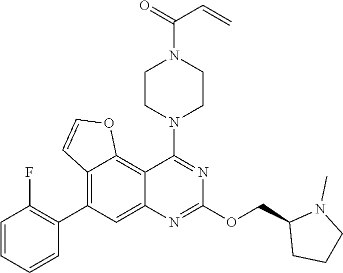 [SMILES: C=CC(=O)N1CCN(c2nc(OC[C@@H]3CCCN3C)nc3cc(-c4ccccc4F)c4ccoc4c23)CC1]

The Compound B45 was synthesized starting from the corresponding starting materials according the similar procedures described as those of compound B41 to give the target compound (4 mg) ¹H NMR (400 MHz, CD₃OD) ¹H NMR (400 MHz, CD3OD) δ 8.04 (s, 1H), 7.65-7.57 (m, 2H), 7.57-7.48 (m, 1H), 7.42-7.28 (m, 2H), 6.93-6.80 (m, 2H), 6.28 (d, J=16.8 Hz, 1H), 5.81 (d, J=10.4 Hz, 1H), 4.77 (d, J=12.3 Hz, 1H), 4.67-4.58 (m, 1H), 3.98 (s, 4H), 3.80 (s, 4H), 3.58 (2H), 3.10-2.97 (m, 1H), 2.94 (s, 3H), 2.34 (d, J=7.0 Hz, 1H), 2.05 (dd, J=19.2, 11.8 Hz, 3H) ppm. MS: M/e 516 (M+1)⁺.

### Compound B46: 1-(4-(4-(2-fluoro-6-hydroxyphenyl)-7-(((S)-1-methylpyrrolidin-2-yl)methoxy)furo[2,3-f]quinazolin-9-yl)piperazin-1-yl)prop-2-en-1-one

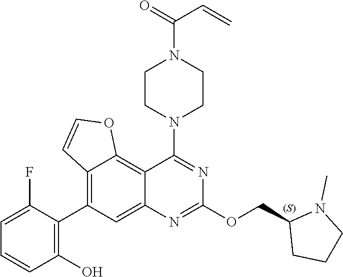

The Compound B46 was synthesized starting from the corresponding starting materials according the similar procedures described as those of compound B39 (18.78 mg). 1H NMR (400 MHz, CD3OD) δ 7.95 (s, 1H), 7.53 (s, 1H), 7.29 (s, 1H), 6.96-6.65 (m, 4H), 6.26 (d, J=17.2 Hz, 1H), 5.81 (s, 1H), 4.54 (s, 2H), 3.97 (s, 4H), 3.78 (s, 4H), 3.25-3.19 (m, 1H), 3.06 (s, 1H), 2.66 (s, 3H), 2.58 (s, 1H), 2.19 (s, 1H), 1.91 (s, 3H) ppm. MS: M/e 532 (M+1)⁺.

### Compound B47: 2-((S)-4-(5-(2′,3′-dihydrospiro[cyclopropane-1,1′-inden]-7′-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

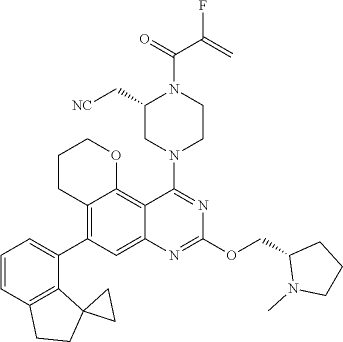 [SMILES: C=C(F)C(=O)N1CCN(c2nc(OC[C@@H]3CCCN3C)nc3cc(-c4cccc5c4C4(CC5)CC4)c4c(c23)OCCC4)C[C@@H]1CC#N]

The Compound B47 was synthesized starting from the corresponding starting materials according the similar procedures described as those of compound B31 to get the product (16 mg). ¹H NMR (400 MHz, DMSO-d₆) δ 7.25 (d, J=7.4 Hz, 1H), 7.16 (td, J=7.5, 3.4 Hz, 1H), 6.79 (d, J=3.7 Hz, 1H), 6.76-6.70 (m, 1H), 5.52-5.25 (m, 2H), 4.80-4.63 (m, 2H), 4.62-4.47 (m, 2H), 4.43-4.16 (m, 5H), 3.65-3.49 (m, 3H), 3.20-3.06 (m, 2H), 3.02-2.87 (m, 6H), 2.30-2.23 (m, 3H), 2.16 (d, J=9.3 Hz, 1H), 2.08-2.02 (m, 1H), 2.00-1.79 (m, 5H), 1.77-1.72 (m, 1H), 0.67 (s, 1H), 0.50 (s, 2H), 0.37 (s, 1H) ppm. MS: M/e 636 (M+1)⁺

### Compound B48: 2-((2S)-1-acryloyl-4-(5-(8-chloronaphthalen-1-yl)-8-((2-(dimethylamino)cyclopentyl)oxy)-3,4-dihydro-2H-pyrano[2,3-f]quinazolin-10-yl)piperazin-2-yl)acetonitrile

### Step A: tert-butyl (S)-4-(5-bromo-8-chloro-3,4-dihydro-2H-pyrano[2,3-f]quinazolin-10-yl)-2-(cyanomethyl)piperazine-1-carboxylate

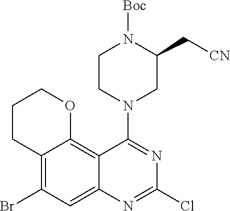 [SMILES: CC(C)(C)OC(=O)N1CCN(c2nc(Cl)nc3cc(Br)c4c(c23)OCCC4)C[C@@H]1CC#N]

To a stirred solution of 5-bromo-8-chloro-2,3,4,9-tetrahydro-10H-pyrano[2,3-f]quinazolin-10-one (3.14 g, 10 mmol) and tert-butyl (S)-2-(cyanomethyl)piperazine-1-carboxylate (3.37 g, 15 mmol) in CH₃CN (100 mL) was added PyBOP (5.7 g, 13 mmol) followed by DBU (3.1 g, 20 mmol). The mixture became clear immediately. After stirred for 2 hours, the reaction mixture was diluted with EA (100 mL), washed with brine, dried over Na₂SO₄, concentrated and purified by column chromatography (petroleum ether/EA=5:1˜1:1) to give the target compound (2.8 g, 53.5%). MS: M/e 522/524 (M+1)⁺.

### Step B: tert-butyl (S)-4-(8-chloro-5-(8-chloronaphthalen-1-yl)-3,4-dihydro-2H-pyrano[2,3-f]quinazolin-10-yl)-2-(cyanomethyl)piperazine-1-carboxylate

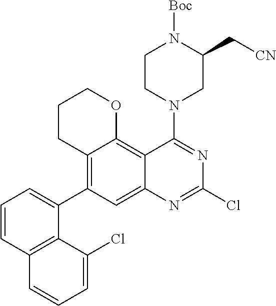 [SMILES: CC(C)(C)OC(=O)N1CCN(c2nc(Cl)nc3cc(-c4cccc5cccc(Cl)c45)c4c(c23)OCCC4)C[C@@H]1CC#N]

A mixture of the product of step A (1.045 g, 2 mmol), 2-(8-chloronaphthalen-1-yl)-4,4,5,5-tetramethyl-1,3,2-dioxaborolane (635 mg, 2.2 mmol) and PddppfCl₂ (146 mg, 0.2 mmol) in aq.K₃PO₄/dioxane (0.85 g in 3 mL H₂O, 20 mL) was stirred for 4 hours under N₂ at 100° C. The reaction mixture was diluted with EA (20 mL), washed with brine, dried over Na₂SO₄, concentrated to give the target compound (900 mg, 74.5%). MS: M/e 604 (M+1)⁺.

### Step C: tert-butyl (2S)-4-(5-(8-chloronaphthalen-1-yl)-8-((2-(dimethylamino)cyclopentyl)oxy)-3,4-dihydro-2H-pyrano[2,3-f]quinazolin-10-yl)-2-(cyanomethyl)piperazine-1-carboxylate

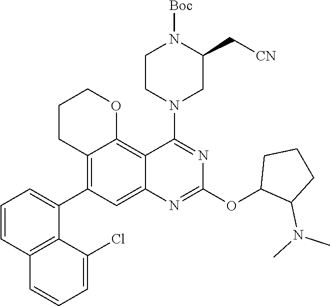 [SMILES: CN(C)C1CCCC1Oc1nc(N2CCN(C(=O)OC(C)(C)C)[C@@H](CC#N)C2)c2c3c(c(-c4cccc5cccc(Cl)c45)cc2n1)CCCO3]

To a stirred solution of 2-(dimethylamino)cyclopentan-1-ol (64 mg, 0.51 mmol) in THF (15 mL) was added NaH (60%, 23 mg, 0.58 mmol). After stirred for 30 min, the product of step B (100 mg, 0.165 mmol) was added and the mixture was stirred overnight at 70° C. The reaction mixture was quenched with aq.NH₄Cl, extracted with EA (10 mL×2). The combined organic layers were washed with brine, dried over Na₂SO₄, concentrated and purified by Prep-TLC (CH₂Cl₂/MeOH=10:1) to give the target compound (55 mg, 47.8%). MS: M/e 697 (M+1)⁺.

### Step D: 2-((2S)-1-acryloyl-4-(5-(8-chloronaphthalen-1-yl)-8-((2-(dimethylamino)cyclopentyl)oxy)-3,4-dihydro-2H-pyrano[2,3-f]quinazolin-10-yl)piperazin-2-yl)acetonitrile

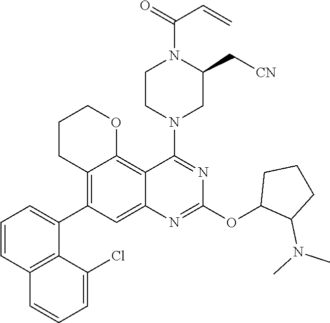 [SMILES: C=CC(=O)N1CCN(c2nc(OC3CCCC3N(C)C)nc3cc(-c4cccc5cccc(Cl)c45)c4c(c23)OCCC4)C[C@@H]1CC#N]

To a stirred solution of step C (55 mg, 0.079 mmol) in CH₂Cl₂ (3 mL) was added HCl/dioxane (4 M, 2 mL). After stirred for an hour, the reaction mixture was concentrated to give the residue, which was dissolved in CH₃CN (5 mL) and aq.NaHCO₃ (0.5 mL) was added, then a solution of acryloyl chloride (7.14 mg, 0.079 mmol) in CH₃CN (0.5 mL) was added. After the addition, the reaction mixture was stirred for 20 min. The reaction mixture was extracted with EA (10 mL), washed with brine, dried over Na₂SO₄, concentrated and purified by Prep-TLC (EA) to give the target compound (16 mg). ¹H NMR (400 MHz, CD₃OD) δ 8.04 (d, J=8.0, 1H), 7.98 (d, J=8.0, 1H), 7.68-7.29 (m, 4H), 7.13-7.02 (m, 1H), 6.83 (s, 1H), 6.30 (d, J=16.4 Hz, 1H), 5.85 (s, 1H), 5.60 (s, 1H), 5.19 (s, 1H), 4.48-4.06 (m, 5H), 3.86-3.41 (m, 3H), 3.25-3.07 (m, 2H), 2.92 (s, 7H), 2.45-2.22 (m, 4H), 1.99-1.89 (m, 6H) ppm. MS: M/e 651 (M+1)⁺.

### Compound B49: 1-((S)-4-(4-(8-chloronaphthalen-1-yl)-7-(((S)-1-methylpyrrolidin-2-yl)methoxy)furo[2,3-f]quinazolin-9-yl)-3-methylpiperazin-1-yl)prop-2-en-1-one

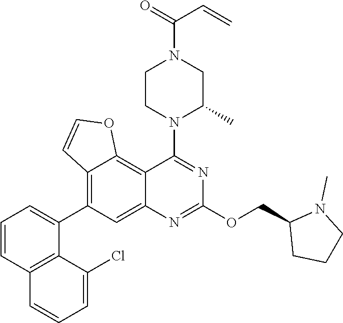 [SMILES: C=CC(=O)N1CCN(c2nc(OC[C@@H]3CCCN3C)nc3cc(-c4cccc5cccc(Cl)c45)c4ccoc4c23)[C@@H](C)C1]

The Compound B49 was synthesized starting from the corresponding starting materials according the similar procedures described as those of compound B34 to give the target compound (8 mg). ¹H NMR (400 MHz, CD₃OD) δ 8.11 (d, J=8.0 Hz, 1H), 8.02 (d, J=8.0 Hz, 1H), 7.95 (s, 1H), 7.70-7.63 (m, 1H), 7.59-7.47 (m, 4H), 6.99-6.77 (m, 1H), 6.39 (s, 1H), 6.31 (d, J=16.4 Hz, 1H), 5.83 (d, J=10.4 Hz, 1H), 4.83-4.48 (m, 3H), 4.32-4.03 (m, 2H), 3.94-3.62 (m, 4H), 3.50-3.88 (m, 1H), 3.26-3.16 (m, 1H), 3.06 (s, 3H), 2.79 (s, 1H), 2.48-2.34 (m, 1H), 2.26-2.01 (m, 3H), 1.40 (s, 3H) ppm. MS: M/e 596 (M+1)⁺.

### Compound B50: 2-((2S)-4-(5-(8-chloronaphthalen-1-yl)-8-((2-(dimethylamino)cyclopentyl)oxy)-3,4-dihydro-2H-pyrano[2,3-f]quinazolin-10-yl)-1-(2-fluoroacryloyl)piperazin-2-yl)acetonitrile

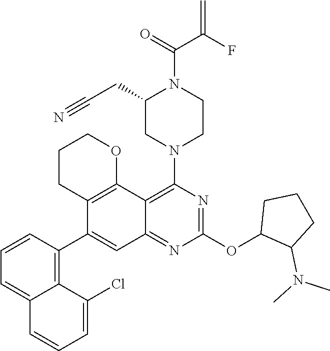 [SMILES: C=C(F)C(=O)N1CCN(c2nc(OC3CCCC3N(C)C)nc3cc(-c4cccc5cccc(Cl)c45)c4c(c23)OCCC4)C[C@@H]1CC#N]

To a stirred solution of tert-butyl (2S)-4-(5-(8-chloronaphthalen-1-yl)-8-((2-(dimethylamino)cyclopentyl)oxy)-3,4-dihydro-2H-pyrano[2,3-f]quinazolin-10-yl)-2-(cyanomethyl)piperazine-1-carboxylate (35 mg, 0.05 mmol) in CH₂Cl₂ (3 mL) was added HCl/dioxane (4 M, 2 mL). After stirred for an hour, the reaction mixture was concentrated to give the residue, which was basified to pH=10˜12 with aq.Na₂CO₃ and extracted with EA (10 mL). The organic layer was dried over Na₂SO₄, concentrated to give the intermediate, which was dissolved in THF (5 mL), 2-fluoroacrylic acid (9 mg, 0.1 mmol) was added, followed by T₃P (63.6 mg, 0.1 mmol) and Et₃N. After the addition, the reaction mixture was stirred overnight. The reaction mixture was diluted with H₂O (10 mL), extracted with EA (10 mL). The organic layer was washed with brine, dried over Na₂SO₄, concentrated and purified by Prep-TLC (CH₂Cl₂/MeOH=10:1) to give the target compound (6 mg). ¹H NMR (400 MHz, CD₃OD) δ 8.04 (d, J=8.0 Hz, 1H), 7.97 (d, J=8.0 Hz, 1H), 7.70-7.52 (m, 2H), 7.52-7.30 (m, 2H), 7.08 (s, 1H), 5.57 (s, 1H), 5.43-5.25 (m, 2H), 4.49-4.08 (m, 5H), 3.81 (s, 1H), 3.56-3.42 (m, 1H), 3.24-3.17 (m, 1H), 3.16-2.97 (m, 4H), 2.92 (s, 6H), 2.48-2.20 (m, 4H), 2.09-1.81 (m, 6H) ppm. MS: M/e 669 (M+1)⁺.

### Compound B51: (S)-1-(7-(4-(8-chloronaphthalen-1-yl)-7-((1-methylpyrrolidin-2-yl)methoxy)furo[2,3-f]quinazolin-9-yl)-2,7-diazaspiro[3.5]nonan-2-yl)prop-2-en-1-one

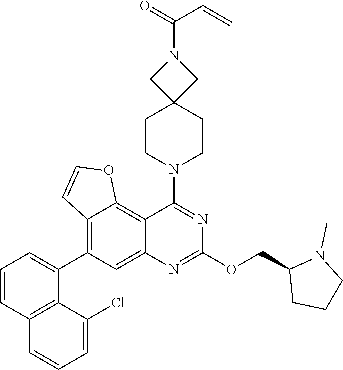 [SMILES: C=CC(=O)N1CC2(CCN(c3nc(OC[C@@H]4CCCN4C)nc4cc(-c5cccc6cccc(Cl)c56)c5ccoc5c34)CC2)C1]

The Compound B51 was synthesized starting from the corresponding starting materials according the similar procedures described as those of compound B34 to give the target compound (5 mg). ¹H NMR (400 MHz, CD₃OD) δ 8.11 (d, J=8.4 Hz, 1H), 8.02 (d, J=7.6 Hz, 1H), 7.92 (s, 1H), 7.69-7.62 (m, 1H), 7.57-7.45 (m, 4H), 6.50-6.34 (m, 2H), 6.28 (d, J=17.2 Hz, 1H), 5.77 (d, J=10.4 Hz, 1H), 4.69-4.61 (m, 1H), 4.17 (s, 2H), 3.99-3.81 (m, 3H), 3.81-3.63 (m, 4H), 3.27-3.16 (m, 2H), 3.06 (s, 3H), 2.79 (s, 2H), 2.46-2.34 (m, 1H), 2.26-2.00 (m, 6H) ppm. MS: M/e 622 (M+1)⁺.

### Compound B52: 1-((2R,5S)-4-(4-(8-chloronaphthalen-1-yl)-7-(((S)-1-methylpyrrolidin-2-yl)methoxy)furo[2,3-f]quinazolin-9-yl)-2,5-dimethylpiperazin-1-yl)prop-2-en-1-one

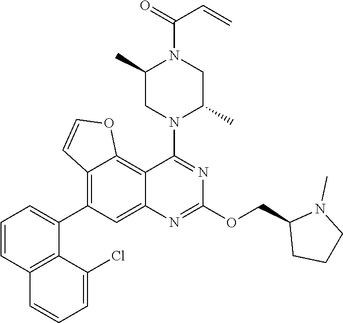 [SMILES: C=CC(=O)N1C[C@H](C)N(c2nc(OC[C@@H]3CCCN3C)nc3cc(-c4cccc5cccc(Cl)c45)c4ccoc4c23)C[C@H]1C]

The Compound B52 was synthesized starting from the corresponding starting materials according the similar procedures described as those of compound B34 to give the target compound (5 mg). ¹H NMR (400 MHz, CD₃OD) δ 8.17-7.91 (m, 3H), 7.72-7.60 (m, 1H), 7.60-7.46 (m, 4H), 6.94-6.77 (m, 1H), 6.44-6.25 (m, 2H), 5.86-5.76 (m, 1H), 4.72-4.61 (m, 1H), 4.61-4.29 (m, 1H), 4.23-3.79 (m, 5H), 3.71 (s, 1H), 3.10 (s, 3H), 2.75 (s, 1H), 2.49-2.36 (m, 1H), 2.29-2.21 (m, 3H), 1.29 (s, 8H) ppm. MS: M/e 610 (M+1)⁺.

### Compound B53: 2-((S)-4-(5-(8-chloronaphthalen-1-yl)-8-(((2S,4R)-4-fluoro-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

### Step A: 5-bromo-8-(((2S,4R)-4-fluoro-1-methylpyrrolidin-2-yl)methoxy)-2,3,4,9-tetrahydro-10H-pyrano[2,3-f]quinazolin-10-one

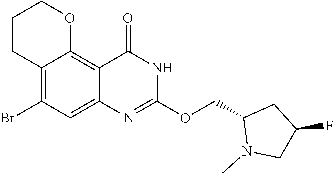 [SMILES: CN1C[C@H](F)C[C@H]1COc1nc2cc(Br)c3c(c2c(=O)[nH]1)OCCC3]

To a stirred mixture of ((2S,4R)-4-fluoro-1-methylpyrrolidin-2-yl)methanol (127 mg, 0.95 mmol) and NaH (60%, 84 mg, 2.1 mmol) in THF (3 ml), was added 5-bromo-8-chloro-2,3,4,9-tetrahydro-10H-pyrano[2,3-f]quinazolin-10-one (100 mg, 0.32 mmol) at rt. The mixture was stirred at 60° C. for 24 h. After completed, the solution was quenched with water (10 ml) and then extracted with EA (10 ml×2). The organic layer was washed with brine (10 ml), dried and concentrated under reduced pressure. The residue was purified by prep-TLC with DCM:MeOH (12:1) to afford product (40 mg, 31%). MS: M/e 412,414 (M+1)⁺.

### Step B: tert-butyl (S)-4-(5-bromo-8-(((2S,4R)-4-fluoro-1-methylpyrrolidin-2-yl)methoxy)-3,4-dihydro-2H-pyrano[2,3-f]quinazolin-10-yl)-2-(cyanomethyl)piperazine-1-carboxylate

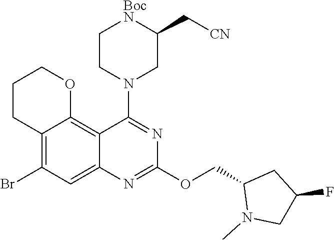 [SMILES: CN1C[C@H](F)C[C@H]1COc1nc(N2CCN(C(=O)OC(C)(C)C)[C@@H](CC#N)C2)c2c3c(c(Br)cc2n1)CCCO3]

To a stirred solution of 5-bromo-8-(((2S,4R)-4-fluoro-1-methylpyrrolidin-2-yl)methoxy)-2,3,4,9-tetrahydro-10H-pyrano[2,3-f]quinazolin-10-one (40 mg, 0.097 mmol), tert-butyl (S)-2-(cyanomethyl)piperazine-1-carboxylate (33 mg, 0.15 mmol) in MeCN (15 ml), was added PyBOP (66 mg, 0.13 mmol) and DBU (30 mg, 0.20 mmol). The mixture was stirred at rt overnight. After completed, the solution was concentrated under reduced pressure. The residue was purified by prep-TLC with DCM:MeOH (20:1) to afford crude product (60 mg), which was used directly for the next step without further purification. MS: M/e 619,621 (M+1)⁺.

### Step C: tert-butyl (S)-4-(5-(8-chloronaphthalen-1-yl)-8-(((2S,4R)-4-fluoro-1-methylpyrrolidin-2-yl)methoxy)-3,4-dihydro-2H-pyrano[2,3-f]quinazolin-10-yl)-2-(cyanomethyl)piperazine-1-carboxylate

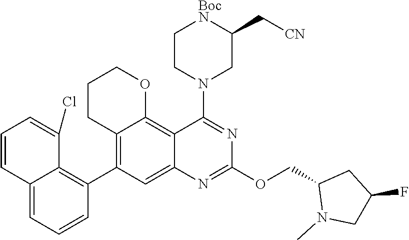 [SMILES: CN1C[C@H](F)C[C@H]1COc1nc(N2CCN(C(=O)OC(C)(C)C)[C@@H](CC#N)C2)c2c3c(c(-c4cccc5cccc(Cl)c45)cc2n1)CCCO3]

A solution of tert-butyl (S)-4-(5-bromo-8-(((2S,4R)-4-fluoro-1-methylpyrrolidin-2-yl)methoxy)-3,4-dihydro-2H-pyrano[2,3-f]quinazolin-10-yl)-2-(cyanomethyl)piperazine-1-carboxylate (60 mg), 2-(8-chloronaphthalen-1-yl)-4,4,5,5-tetramethyl-1,3,2-dioxaborolane (30.8 mg, 0.11 mmol), Pd(dppf)Cl₂ (7.1 mg, 0.0097 mmol) and K₃PO₄ (61.5 mg, 0.29 mmol) in dioxane (8 ml) and H₂O (1 ml) was stirred at 100° C. for 3 h under N₂. After completed, the mixture was poured into H₂O (10 ml), extracted with EA (10 ml×2). The organic layer was washed with brine (10 ml), dried and concentrated under reduced pressure. The residue was purified by prep-TLC with DCM:MeOH (15:1) to afford product (15 mg). MS: M/e 701 (M+1)⁺.

### Step D: 2-((S)-4-(5-(8-chloronaphthalen-1-yl)-8-(((2S,4R)-4-fluoro-1-methylpyrrolidin-2-yl)methoxy)-3,4-dihydro-2H-pyrano[2,3-f]quinazolin-10-yl)piperazin-2-yl)acetonitrile

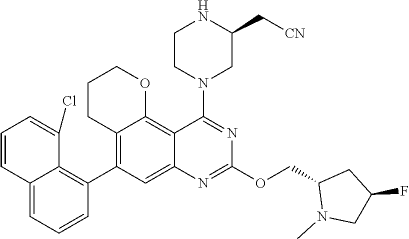 [SMILES: CN1C[C@H](F)C[C@H]1COc1nc(N2CCN[C@@H](CC#N)C2)c2c3c(c(-c4cccc5cccc(Cl)c45)cc2n1)CCCO3]

A solution of tert-butyl (S)-4-(5-(8-chloronaphthalen-1-yl)-8-(((2S,4R)-4-fluoro-1-methylpyrrolidin-2-yl)methoxy)-3,4-dihydro-2H-pyrano[2,3-f]quinazolin-10-yl)-2-(cyanomethyl)piperazine-1-carboxylate (15 mg, 0.021 mmol) and HCl/dioxane (4M, 1 ml) in DCM (2 ml) was stirred at rt for 1 h. After completed, the solution was concentrated under reduced pressure to afford product (12.9 mg, 100%), which was used directly for the next step without further purification. MS: M/e 601 (M+1)⁺.

### Step E: 2-((S)-4-(5-(8-chloronaphthalen-1-yl)-8-(((2S,4R)-4-fluoro-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

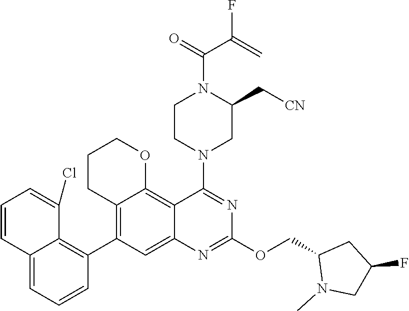 [SMILES: C=C(F)C(=O)N1CCN(c2nc(OC[C@@H]3C[C@@H](F)CN3C)nc3cc(-c4cccc5cccc(Cl)c45)c4c(c23)OCCC4)C[C@@H]1CC#N]

To a stirred solution of 2-((S)-4-(5-(8-chloronaphthalen-1-yl)-8-(((2S,4R)-4-fluoro-1-methylpyrrolidin-2-yl)methoxy)-3,4-dihydro-2H-pyrano[2,3-f]quinazolin-10-yl)piperazin-2-yl)acetonitrile (12.9 mg, 0.021 mmol), 2-fluoroacrylic acid (3.87 mg, 0.043 mmol) and TEA (4.34 mg, 0.043 mmol) in THF, was added T₃P (13.7 mg, 0.043 mmol) at rt and stirred for 24 h. After completed, the solution was concentrated under reduced pressure. The residue was purified by prep-TLC with DCM:MeOH (13:1) to afford product (3.09 mg). 1H NMR (400 MHz, CD3OD) δ 8.04 (d, J=8.6 Hz, 1H), 7.97 (d, J=8.6 Hz, 1H), 7.62 (s, 1H), 7.56 (s, 1H), 7.47 (s, 1H), 7.36 (d, J=17.0 Hz, 1H), 7.06 (s, 1H), 5.49 (d, J=16.8 Hz, 1H), 5.35-5.28 (m, 2H), 5.07 (d, J=14.4 Hz, 1H), 4.77-4.74 (m, 1H), 4.63 (s, 1H), 4.53-4.28 (m, 3H), 4.13 (s, 1H), 3.80-4.00 (m, 2H), 3.40-3.65 (m, 4H), 3.01 (s, 5H), 2.80 (s, 1H), 2.57 (d, J=16.6 Hz, 1H), 2.31 (s, 3H), 1.98 (s, 2H) ppm. MS: M/e 673 (M+1)⁺.

### Compound B54: 2-((S)-4-(5-(8-chloronaphthalen-1-yl)-8-(((2S,4S)-4-fluoro-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

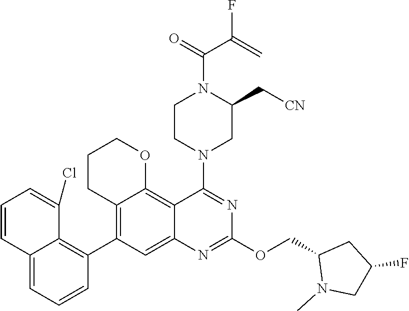 [SMILES: C=C(F)C(=O)N1CCN(c2nc(OC[C@@H]3C[C@H](F)CN3C)nc3cc(-c4cccc5cccc(Cl)c45)c4c(c23)OCCC4)C[C@@H]1CC#N]

The Compound B54 was synthesized starting from the corresponding starting materials according the similar procedures described as those of compound B53 to afford product (6.01 mg). 1H NMR (400 MHz, cd3od) δ 8.02 (d, J=7.7 Hz, 1H), 7.96 (d, J=7.1 Hz, 1H), 7.61 (d, J=6.9 Hz, 1H), 7.55 (s, 1H), 7.46 (s, 1H), 7.36 (d, 1H), 7.05 (s, 1H), 5.38 (d, J=19.7 Hz, 1H), 5.30 d, J=7.9 Hz, 1H), 5.25 (s, 0.5H), 5.11 (s, 0.5H), 4.51 (d, J=10.4 Hz, 2H), 4.41 (s, 2H), 4.29 (s, 1H), 4.10 (s, 1H), 3.33-3.55 (m, 3H), 3.08-3.27 (m, 3H), 2.97 (s, 2H), 2.48-2.68 (m, 5H), 2.22-2.39 (m, 2H), 1.98 (s, 2H), 1.29 (s, 2H) ppm. MS: M/e 673 (M+1)⁺.

### Compound B55: 2-((S)-4-(5-(8-chloronaphthalen-1-yl)-8-(((3R,4R)-4-methoxy-1-methylpyrrolidin-3-yl)oxy)-3,4-dihydro-2H-pyrano[2,3-f]quinazolin-10-yl)-1-(2-fluoroacryloyl)piperazin-2-yl)acetonitrile

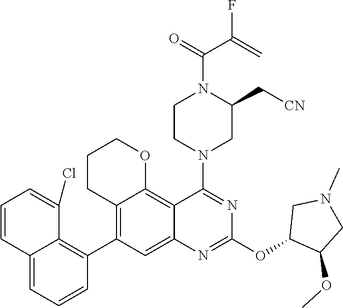 [SMILES: C=C(F)C(=O)N1CCN(c2nc(O[C@@H]3CN(C)C[C@H]3OC)nc3cc(-c4cccc5cccc(Cl)c45)c4c(c23)OCCC4)C[C@@H]1CC#N]

The Compound B55 was synthesized starting from the corresponding starting materials according the similar procedures described as those of compound B53 to afford product (2.40 mg). 1H NMR (400 MHz, CD3OD) δ 8.03 (d, J=8.2 Hz, 1H), 7.96 (d, J=8.0 Hz, 1H), 7.62 (d, J=8.0 Hz, 1H), 7.55 (s, 1H), 7.46 (s, 1H), 7.36 (d, J=27.0 Hz, 1H), 7.10-7.01 (m, 1H), 5.45 (s, 1H), 5.33 (d, J=18.8 Hz, 2H), 4.45-4.35 (m, 2H), 4.33-4.26 (m, 1H), 4.12 (s, 2H), 3.44 (s, 5H), 3.15 (s, 6H), 3.04-2.95 (m, 2H), 2.92-2.86 (m, 1H), 2.54 (s, 3H), 2.31 (s, 2H), 1.98 (s, 2H) ppm, MS: M/e 671 (M+1)⁺.

### Compound B56: (S)-1-(4-(4-(2,6-dimethylphenyl)-7-((1-methylpyrrolidin-2-yl)methoxy)furo[2,3-f]quinazolin-9-yl)piperazin-1-yl)prop-2-en-1-one

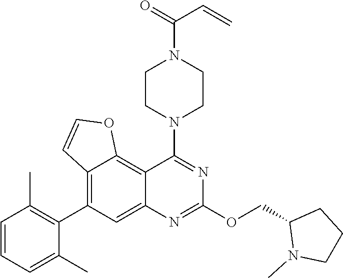 [SMILES: C=CC(=O)N1CCN(c2nc(OC[C@@H]3CCCN3C)nc3cc(-c4c(C)cccc4C)c4ccoc4c23)CC1]

The Compound B56 was synthesized starting from the corresponding starting materials according the similar procedures described as those of compound B38 give the target compound (0.5 mg). ¹H NMR (400 MHz, CD3OD) δ 8.02 (s, 1H), 7.34 (s, 1H), 7.27 (s, 1H), 7.21 (s, 1H), 6.86 (s, 1H), 6.53 (s, 1H), 6.29 (d, J=16.8 Hz, 1H), 5.82 (d, J=11.1 Hz, 1H), 5.34 (s, 1H), 4.69 (s, 2H), 4.00 (s, 4H), 3.85 (s, 4H), 3.10 (s, 3H), 2.42 (s, 1H), 2.19 (s, 2H), 1.99 (s, 6H), 1.60 (s, 2H), 0.90 (s, 2H) ppm. MS: M/e 526 (M+1)⁺.

### Compound B57: 1-(4-(4-(2-chloro-6-fluorophenyl)-7-(((S)-1-methylpyrrolidin-2-yl)methoxy)furo[2,3-f]quinazolin-9-yl)piperazin-1-yl)prop-2-en-1-one

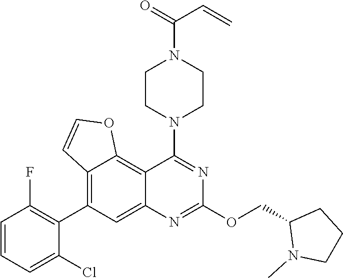 [SMILES: C=CC(=O)N1CCN(c2nc(OC[C@@H]3CCCN3C)nc3cc(-c4c(F)cccc4Cl)c4ccoc4c23)CC1]

The Compound B57 was synthesized starting from the corresponding starting materials according the similar procedures described as those of compound B38 to give the target compound (2 mg). ¹H NMR (400 MHz, CD3OD) δ 8.04 (s, 1H), 7.53 (s, 2H), 7.49 (s, 1H), 7.31 (s, 1H), 6.86 (s, 1H), 6.69 (s, 1H), 6.28 (d, J=17.3 Hz, 1H), 5.81 (d, J=10.4 Hz, 1H), 4.66 (s, 2H), 3.99 (s, 4H), 3.82 (s, 4H), 3.07 (s, 3H), 2.75 (s, 1H) 2.42 (s, 1H), 2.19 (s, 2H), 2.09 (s, 2H), 2.05-2.00 (m, 1H) ppm. MS: M/e 550 (M+1)⁺.

### Compound B58: (S)-1-(4-(4-(2-cyclopropylphenyl)-7-((1-methylpyrrolidin-2-yl)methoxy)furo[2,3-f]quinazolin-9-yl)piperazin-1-yl)prop-2-en-1-one

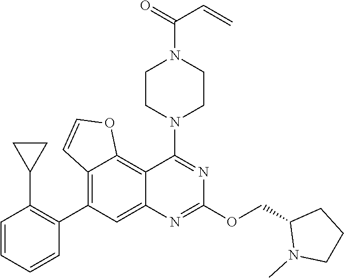 [SMILES: C=CC(=O)N1CCN(c2nc(OC[C@@H]3CCCN3C)nc3cc(-c4ccccc4C4CC4)c4ccoc4c23)CC1]

The Compound B58 was synthesized starting from the corresponding starting materials according the similar procedures described as those of compound B38 give the target compound (2.5 mg). ¹H NMR (400 MHz, CD3OD) δ 8.02 (s, 1H), 7.53 (s, 1H), 7.39 (s, 1H), 7.29 (s, 2H), 7.07 (s, 1H), 6.86 (s, 1H), 6.77 (s, 1H), 6.28 (d, J=16.0 Hz, 1H), 5.81 (d, J=10.1 Hz, 1H), 4.67 (s, 2H), 3.99 (s, 4H), 3.82 (s, 4H), 3.10 (s, 3H), 2.43 (s, 1H), 2.21 (s, 2H), 2.11 (s, 2H), 2.03 (s, 1H), 1.72 (s, 1H), 0.90 (s, 1H), 0.76 (s, 2H), 0.71 (s, 2H) ppm. MS: M/e 538 (M+1)⁺.

### Compound B59: (S)-1-(4-(4-(2,6-dichlorophenyl)-7-((1-methylpyrrolidin-2-yl)methoxy)furo[2,3-f]quinazolin-9-yl)piperazin-1-yl)prop-2-en-1-one

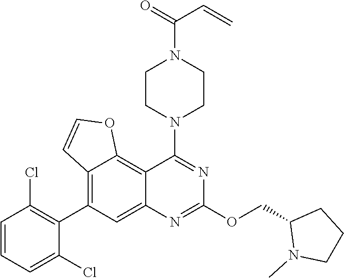 [SMILES: C=CC(=O)N1CCN(c2nc(OC[C@@H]3CCCN3C)nc3cc(-c4c(Cl)cccc4Cl)c4ccoc4c23)CC1]

The Compound B59 was synthesized starting from the corresponding starting materials according the similar procedures described as those of compound B38 to give the target compound (0.86 mg). ¹H NMR (400 MHz, DMSO-d₆) δ 8.13 (s, 1H), 7.69 (s, 2H), 7.59 (s, 1H), 7.54 (s, 1H), 6.94 (d, J=13.9 Hz, 1H), 6.71 (s, 1H), 6.38 (d, J=17.0 Hz, 1H), 5.91 (d, J=9.2 Hz, 1H), 4.93-4.87 (m, 1H), 4.73 (s, 1H), 4.09 (s, 4H), 3.92 (s, 4H), 3.81-3.44 (m, 2H), 3.10 (s, 3H), 2.48 (s, 1H), 2.23 (s, 2H), 2.14 (s, 2H) ppm. MS: M/e 566 (M+1)⁺.

### Compound B60: 2-((S)-4-(5-(8-chloronaphthalen-1-yl)-8-(((S)-1,2-dimethylpyrrolidin-2-yl)methoxy)-3,4-dihydro-2H-pyrano[2,3-f]quinazolin-10-yl)-1-(2-fluoroacryloyl)piperazin-2-yl)acetonitrile

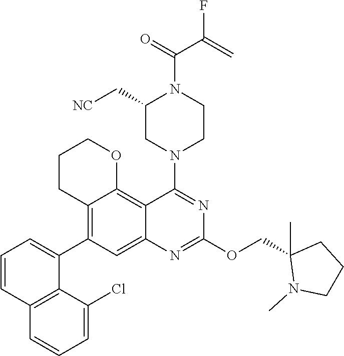 [SMILES: C=C(F)C(=O)N1CCN(c2nc(OC[C@]3(C)CCCN3C)nc3cc(-c4cccc5cccc(Cl)c45)c4c(c23)OCCC4)C[C@@H]1CC#N]

The Compound B60 was synthesized starting from the corresponding starting materials according the similar procedures described as those of compound B48 to get the product (22 mg). ¹H NMR (400 MHz, DMSO-d₆) δ 8.12 (d, J=7.8 Hz, 1H), 8.07 (d, J=7.3 Hz, 1H), 7.85-7.58 (m, 2H), 7.54 (s, 1H), 7.46-7.32 (m, 1H), 6.97 (s, 1H), 5.46-5.21 (m, 2H), 4.46-4.30 (m, 2H), 4.30-4.13 (m, 3H), 3.95-3.84 (m, 1H), 3.82 (d, J=10.3 Hz, 1H), 3.68 (d, J=10.4 Hz, 1H), 3.49 (t, J=10.2 Hz, 1H), 3.28-3.22 (m, 1H), 3.21-2.85 (m, 4H), 2.65 (d, J=11.9 Hz, 1H), 2.47-2.41 (m, 2H), 2.29 (s, 3H), 2.25-2.08 (m, 3H), 2.06-1.94 (m, 1H), 1.89 (s, 2H), 1.24 (s, 3H) ppm. MS: M/e 669 (M+1)⁺

### Compound B61: 2-((S)-4-(5-(8-chloronaphthalen-1-yl)-8-(((1R,2R)-2-(dimethylamino)cyclohexyl)oxy)-3,4-dihydro-2H-pyrano[2,3-f]quinazolin-10-yl)-1-(2-fluoroacryloyl)piperazin-2-yl)acetonitrile

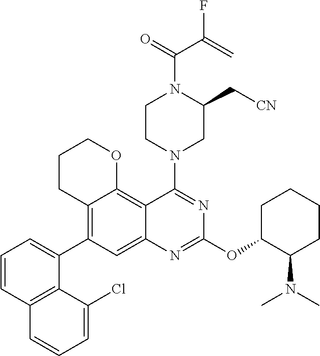 [SMILES: C=C(F)C(=O)N1CCN(c2nc(O[C@@H]3CCCC[C@H]3N(C)C)nc3cc(-c4cccc5cccc(Cl)c45)c4c(c23)OCCC4)C[C@@H]1CC#N]

The Compound B61 was synthesized starting from the corresponding starting materials according the similar procedures described as those of compound B48 to give the target compound (4 mg). ¹H NMR (400 MHz, CD₃OD) δ 8.06-7.93 (m, 2H), 7.68-7.31 (m, 4H), 7.07 (s, 1H), 5.43-5.25 (m, 2H), 4.50-4.22 (m, 4H), 4.15-4.05 (m, 1H), 3.68-3.42 (m, 3H), 3.27-2.96 (m, 4H), 2.88 (s, 6H), 2.53-2.11 (m, 5H), 1.98 (s, 3H), 1.77-1.41 (m, 5H) ppm. MS: M/e 683 (M+1)⁺.

### Compound B62: 1-((2R,5S)-4-(4-(5-chloro-6-methyl-1H-indazol-4-yl)-7-(((S)-1-methylpyrrolidin-2-yl)methoxy)furo[2,3-f]quinazolin-9-yl)-2,5-dimethylpiperazin-1-yl)prop-2-en-1-one

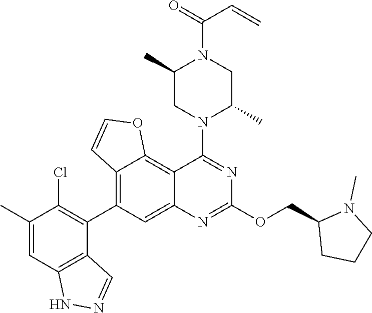 [SMILES: C=CC(=O)N1C[C@H](C)N(c2nc(OC[C@@H]3CCCN3C)nc3cc(-c4c(Cl)c(C)cc5[nH]ncc45)c4ccoc4c23)C[C@H]1C]

The Compound B62 was synthesized starting from the corresponding starting materials according the similar procedures described as those of compound B34 to give the target compound (8 mg). ¹H NMR (400 MHz, CD₃OD) δ 8.03 (s, 1H), 7.65 (s, 1H), 7.62 (s, 1H), 7.54 (d, J=17.6 Hz, 1H), 6.95-6.76 (m, 1H), 6.60 (s, 1H), 6.35-6.25 (m, 1H), 5.87-5.77 (m, 1H), 4.71-4.49 (m, 2H), 4.39-4.15 (m, 1H), 4.13-3.76 (m, 4H), 3.66 (s, 1H), 3.23-3.17 (m, 2H), 3.05 (s, 3H), 2.62 (s, 3H), 2.48-2.01 (m, 5H), 1.30 (s, 6H) ppm. MS: M/e 614 (M+1)⁺.

### Compound B63: 1-((S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-3-methylpiperazin-1-yl)prop-2-en-1-one

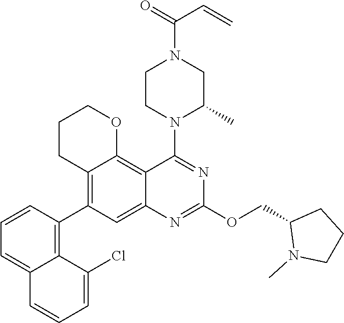 [SMILES: C=CC(=O)N1CCN(c2nc(OC[C@@H]3CCCN3C)nc3cc(-c4cccc5cccc(Cl)c45)c4c(c23)OCCC4)[C@@H](C)C1]

The Compound B63 was synthesized starting from the corresponding starting materials according the similar procedures described as those of compound B23 to give the target compound. ¹H NMR (400 MHz, CD₃OD) δ 8.06 (d, J=8.4 Hz, 1H), 7.99 (d, J=8.0 Hz, 1H), 7.70-7.60 (m, 1H), 7.60-7.54 (m, 1H), 7.53-7.42 (m, 1H), 7.41-7.32 (m, 1H), 7.02 (s, 1H), 6.95-6.76 (m, 1H), 6.31 (d, J=16.4 Hz, 1H), 5.83 (d, J=10.4 Hz, 1H), 4.79-4.61 (m, 2H), 4.43-4.30 (m, 2H), 4.16-3.93 (m, 2H), 3.80-3.66 (m, 2H), 3.08 (s, 3H), 2.50-2.29 (m, 3H), 2.20-1.95 (m, 5H), 1.53-1.10 (m, 9H). MS: M/e 612 (M+1)⁺.

### Compound B64: (S)-2-(4-(5-(8-chloronaphthalen-1-yl)-8-((1-methylazetidin-3-yl)oxy)-3,4-dihydro-2H-pyrano[2,3-f]quinazolin-10-yl)-1-(2-fluoroacryloyl)piperazin-2-yl)acetonitrile

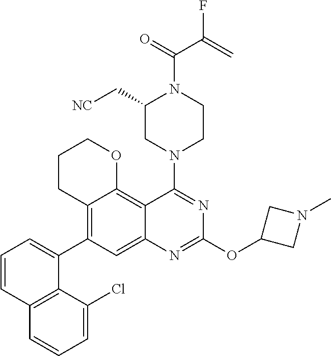 [SMILES: C=C(F)C(=O)N1CCN(c2nc(OC3CN(C)C3)nc3cc(-c4cccc5cccc(Cl)c45)c4c(c23)OCCC4)C[C@@H]1CC#N]

The Compound B64 was synthesized starting from the corresponding starting materials according the similar procedures described as those of compound B48 to get the product (18 mg). ¹H NMR (400 MHz, DMSO-d₆) δ 8.13-8.07 (m, 2H), 7.63 (s, 2H), 7.53 (s, 1H), 7.39 (1H), 6.93 (s, 1H), 5.49-5.18 (m, 2H), 5.08 (s, 1H), 4.34 (s, 1H), 4.22 (2H), 3.90 (s, 1H), 3.72 (s, 2H), 3.33 (s, 5H), 2.97 (s, 4H), 2.28 (s, 3H), 2.17 (s, 2H), 1.89 (s, 2H) ppm. MS: M/e 627 (M+1)

### Compound B65: 2-((S)-4-(4-(8-chloronaphthalen-1-yl)-7-(((2S,4R)-4-fluoro-1-methylpyrrolidin-2-yl)methoxy)furo[2,3-f]quinazolin-9-yl)-1-(2-fluoroacryloyl)piperazin-2-yl)acetonitrile

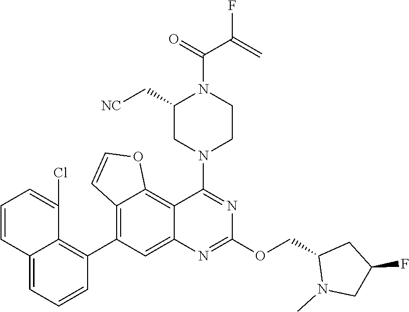 [SMILES: C=C(F)C(=O)N1CCN(c2nc(OC[C@@H]3C[C@@H](F)CN3C)nc3cc(-c4cccc5cccc(Cl)c45)c4ccoc4c23)C[C@@H]1CC#N]

The Compound B65 was synthesized starting from the corresponding starting materials according the similar procedures described as those of compound B34 to give the target compound (1.15 mg). ¹H NMR (400 MHz, cd₃od) δ 8.10 (d, J=8.4 Hz, 1H), 8.00 (s, 2H), 7.65 (s, 2H), 7.53 (s, 3H), 6.38 (s, 1H), 5.42 (s, 1H), 5.31 (s, 1H), 4.58 (s, 2H), 4.38 (d, J=14.1 Hz, 2H), 4.24 (s, 2H), 4.17 (s, 1H), 3.49 (s, 2H), 3.14 (s, 2H), 2.79 (s, 2H), 2.64 (s, 3H), 2.03 (s, 2H) ppm. MS: M/e 657 (M+1)⁺.

### Compound B66: 2-((S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

### Step A: tert-butyl (S)-4-(5-bromo-8-(((S)-1-methylpyrrolidin-2-yl)methoxy)-3,4-dihydro-2H-pyrano[2,3-f]quinazolin-10-yl)-2-(cyanomethyl)piperazine-1-carboxylate

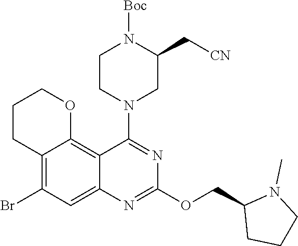 [SMILES: CN1CCC[C@H]1COc1nc(N2CCN(C(=O)OC(C)(C)C)[C@@H](CC#N)C2)c2c3c(c(Br)cc2n1)CCCO3]

To a stirred mixture of (S)-5-bromo-8-((1-methylpyrrolidin-2-yl)methoxy)-2,3,4,9-tetrahydro-10H-pyrano[2,3-f]quinazolin-10-one (2.0 g, 5.1 mmol) and tert-butyl piperazine-1-carboxylate (1.71 g, 7.6 mmol) in CH₃CN (200 mL) was added PyBOP (2.93 g, 6.63 mmol), followed by DBU (1.55 g, 10.2 mmol). After the addition, the reaction mixture was stirred overnight. The mixture was diluted with EtOAc (100 mL), washed with brine, dried over Na₂SO₄, concentrated and purified by combi-flash. to give the target compound (2 g, 65.4%). MS: M/e 601/603 (M+1)⁺.

### Step B: tert-butyl (S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-2-(cyanomethyl)piperazine-1-carboxylate

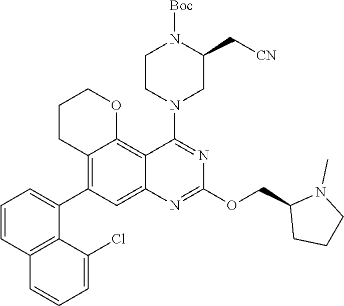 [SMILES: CN1CCC[C@H]1COc1nc(N2CCN(C(=O)OC(C)(C)C)[C@@H](CC#N)C2)c2c3c(c(-c4cccc5cccc(Cl)c45)cc2n1)CCCO3]

To a stirred solution of the product of step A (200 mg, 0.33 mmol) and 2-(8-chloronaphthalen-1-yl)-4,4,5,5-tetramethyl-1,3,2-dioxaborolane (115 mg, 0.39 mmol) in dioxane/sat. aq. K₃PO₄ (10 mL/1 mL) was added PddppfCl₂ (24 mg, 0.033 mmol). After the addition, the reaction was stirred at 100° C. for 2 hours under N₂. The reaction mixture was diluted with EtOAc (10 mL), washed with brine, dried over Na₂SO₄, concentrated to give the residue, which was purified by column chromatography (CH₂Cl₂/MeOH=30:1˜10:1) to give the target compound (150 mg, 72.1%). MS: M/e 683 (M+1)⁺.

### Step C: 2-((S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

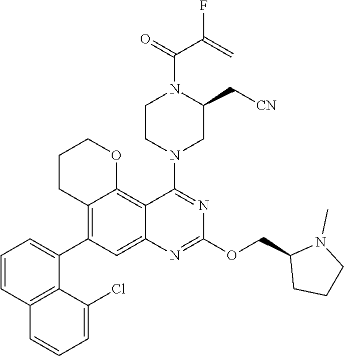 [SMILES: C=C(F)C(=O)N1CCN(c2nc(OC[C@@H]3CCCN3C)nc3cc(-c4cccc5cccc(Cl)c45)c4c(c23)OCCC4)C[C@@H]1CC#N]

To a stirred solution of the product of step B (150 mg, 0.24 mmol) in EtOAc (5 mL) was added HCl/dioxane (4 M, 5 mL). After stirred for an hour, the reaction mixture was concentrated, basified with aq.sat.Na₂CO₃ and extracted with EtOAc (20 mL). The organic layer was washed with brine, dried over Na₂SO₄, concentrated to give the intermediate, which was dissolved in THF (10 mL), 2-fluoroacrylic acid (43 mg, 0.48 mmol) was added, then T₃P (305 mg, 0.48 mmol) followed by Et₃N (72.7 mg, 0.72 mmol). After the addition, the reaction was stirred for 2 hours. The reaction mixture was diluted with EtOAc (15 mL), washed with brine, dried over Na₂SO₄, concentrated and purified by Prep-TLC (CH₂Cl₂/MeOH=10:1) to give the target compound (45 mg, 31%)¹H NMR (400 MHz, CD₃OD) δ 8.12-7.93 (m, 2H), 7.69-7.28 (m, 4H), 7.07 (s, 1H), 5.47-5.25 (m, 2H), 5.00 (s, 1H), 4.65-4.24 (m, 4H), 4.13 (s, 1H), 3.90-3.44 (m, 5H), 3.25-3.16 (m, 5H), 3.05 (s, 3H), 2.48-1.92 (m, 8H) ppm. MS: M/e 655 (M+1)⁺.

### Compound B67 and B68: 2-((S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 1) and 2-((S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 2)

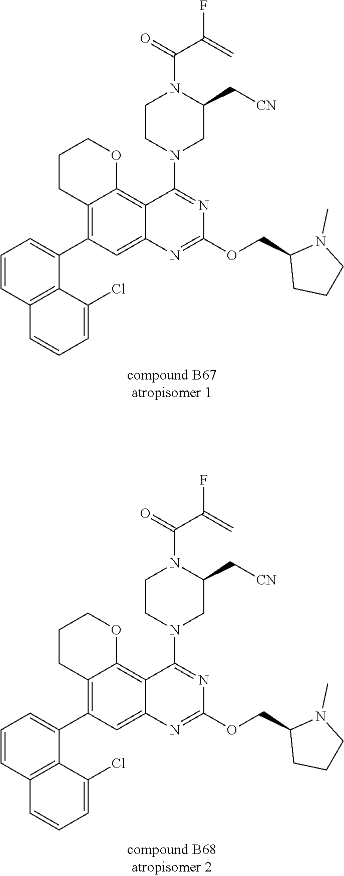 [SMILES: C=C(F)C(=O)N1CCN(c2nc(OC[C@@H]3CCCN3C)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc5cccc(Cl)c45)c4c(c23)OCCC4)C[C@@H]1CC#N]

The compound B66 was purified by preparative Chiral—HPLC(twice) to afford compound B67 (atropisomer 1) and B68 (atropisomer 2)

Prep-HPLC condition

| Column | CHIRAL ART Cellulose-SB |
|---|---|
| Column size | 2 cm × 25 cm, 5 um |
| Injection | 1.0 mL |
| Mobile phase | Hex:DCM = 3:1(0.5% 2M NH3-MeOH)]: |
| | EtOH = 50:50 |
| Flow rate | 20 mL/min |
| Wavelength | UV 220 nm |
| Temperature | 25° C. |
| Sample solution | 8.07 mg/ml in (EtOH:DCM = 1:1) |
| Prep-HPLC-equipment | Prep-HPLC-Gilson |

Prep-HPLC condition (Remove impurities)

| Column | CHIRALPAK IE |
|---|---|
| Column size | 2 cm × 25 cm, 5 um |
| Injection | 1.0 mL |
| Mobile phase | [Hex:DCM = 3:1(0.5% 2M NH3-MeOH)]: |
| | IPA = 80:20 |
| Flow rate | 20 mL/min |
| Wavelength | UV 220 nm |
| Temperature | 25° C. |
| Sample solution | 7 mg/ml in (EtOH:DCM = 1:1) |
| Prep-HPLC-equipment | Prep-HPLC-Gilson |

compound B67 (atropisomer 1) ¹H NMR (400 MHz, CD₃OD) δ 8.02 (d, J=8.0 Hz, 1H), 7.95 (d, J=8.0 Hz, 1H), 7.66-7.34 (m, 4H), 7.06 (s, 1H), 5.44-5.24 (m, 2H), 4.92 (s, 1H), 4.54-4.33 (m, 4H), 4.33-4.04 (m, 3H), 3.63 (s, 1H), 3.45 (d, J=13.2 Hz, 1H), 3.27-3.04 (m, 3H), 3.05-2.75 (m, 2H), 2.52 (s, 3H), 2.45-2.18 (m, 3H), 2.18-2.05 (m, 1H), 1.96 (s, 2H), 1.89-1.69 (m, 3H) ppm. MS: M/e 655 (M+1)⁺.

Compound B68 (atropisomer 2): ¹H NMR (400 MHz, CD₃OD) δ 8.02 (d, J=8.0 Hz, 1H), 7.96 (d, J=8.0 Hz, 1H), 7.65-7.29 (m, 4H), 7.04 (s, 1H), 5.44-5.23 (m, 2H), 5.00 (s, 1H), 4.55-4.34 (m, 4H), 4.34-4.00 (m, 3H), 3.63 (s, 1H), 3.43 (d, J=13.2 Hz, 1H), 3.25-3.06 (m, 3H), 3.05-2.79 (m, 2H), 2.52 (s, 3H), 2.46-2.05 (m, 4H), 1.97 (s, 2H), 1.90-1.68 (m, 3H) ppm. MS: M/e 655 (M+1)⁺.

### Compound B69: 2-((2S)-4-(5-(8-chloronaphthalen-1-yl)-8-((4-(dimethylamino)tetrahydrofuran-3-yl)oxy)-3,4-dihydro-2H-pyrano[2,3-f]quinazolin-10-yl)-1-(2-fluoroacryloyl)piperazin-2-yl)acetonitrile

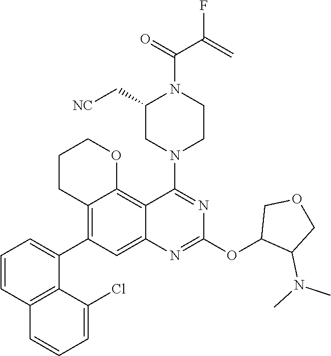 [SMILES: C=C(F)C(=O)N1CCN(c2nc(OC3COCC3N(C)C)nc3cc(-c4cccc5cccc(Cl)c45)c4c(c23)OCCC4)C[C@@H]1CC#N]

The Compound B69 was synthesized starting from the corresponding starting materials according the similar procedures described as those of compound B48 to give the target compound (8 mg). ¹H NMR (400 MHz, CD₃OD) δ 8.03 (d, J=7.6 Hz, 1H), 7.67-7.51 (m, 2H), 7.51-7.29 (m, 2H), 7.09-7.02 (m, 1H), 5.53 (d, J=18.0 Hz, 1H), 5.44-5.23 (m, 2H), 4.50-4.05 (m, 7H), 3.95-3.83 (m, 1H), 3.77 (s, 1H), 3.50-3.38 (m, 1H), 3.24-2.75 (m, 6H), 2.44 (s, 6H), 2.38-2.21 (m, 3H), 1.98 (s, 2H) ppm. MS: M/e 671 (M+1)⁺.

### Compound B70: 2-((S)-4-(5-(7-chloronaphthalen-1-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

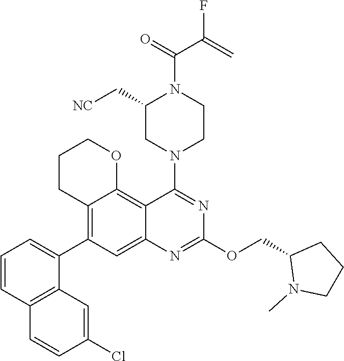 [SMILES: C=C(F)C(=O)N1CCN(c2nc(OC[C@@H]3CCCN3C)nc3cc(-c4cccc5ccc(Cl)cc45)c4c(c23)OCCC4)C[C@@H]1CC#N]

The Compound B70 was synthesized starting from the corresponding starting materials according the similar procedures described as those of compound B48 to get the product (31 mg). ¹H NMR (400 MHz, DMSO-d₆) δ 8.09-8.03 (m, 2H), 7.66 (s, 1H), 7.58 (d, J=8.6 Hz, 1H), 7.49 (d, J=7.3 Hz, 1H), 7.40 (s, 1H), 6.98 (s, 1H), 5.48-5.21 (m, 2H), 4.41-4.23 (m, 4H), 4.13 (s, 1H), 3.96 (2H), 3.22-2.90 (m, 7H), 2.55 (s, 1H), 2.35 (s, 4H), 2.20-2.07 (m, 2H), 1.89 (s, 3H), 1.67 (s, 3H) ppm. MS: M/e 655 (M+1)⁺

### Compound B71: 2-((S)-4-(5-(8-chloronaphthalen-1-yl)-8-(((R)-1-methylpyrrolidin-3-yl)oxy)-3,4-dihydro-2H-pyrano[2,3-f]quinazolin-10-yl)-1-(2-fluoroacryloyl)piperazin-2-yl)acetonitrile

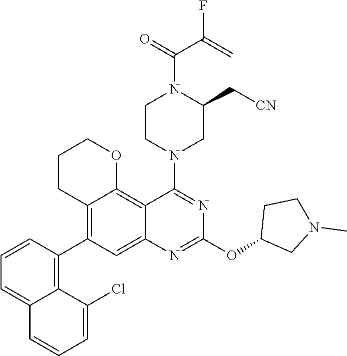 [SMILES: C=C(F)C(=O)N1CCN(c2nc(O[C@@H]3CCN(C)C3)nc3cc(-c4cccc5cccc(Cl)c45)c4c(c23)OCCC4)C[C@@H]1CC#N]

The Compound B71 was synthesized starting from the corresponding starting materials according the similar procedures described as those of compound B48 to get the product (3.2 mg). ¹H NMR (400 MHz, CD₃OD) δ 8.01 (dd, J=26.0, 8.0 Hz, 2H), 7.66-7.52 (m, 2H), 7.50-7.41 (m, 1H), 7.41-7.29 (m, 1H), 7.04 (d, J=6.0 Hz, 1H), 5.44-5.26 (m, 2H), 4.47-4.37 (m, 2H), 4.35-4.18 (m, 2H), 4.17-4.08 (m, 1H), 3.76-3.63 (m, 3H), 3.55-3.40 (m, 2H), 3.26-3.15 (m, 2H), 3.01 (s, 3H), 2.73-2.57 (m, 2H), 2.34-2.24 (m, 2H), 2.01-1.94 (m, 2H), 1.39-1.30 (m, 4H) ppm. MS: M/e 641 (M+1)⁺.

### Compound B72: 2-((S)-4-(5-(8-chloronaphthalen-1-yl)-8-(((S)-4-methylmorpholin-3-yl)methoxy)-3,4-dihydro-2H-pyrano[2,3-f]quinazolin-10-yl)-1-(2-fluoroacryloyl)piperazin-2-yl)acetonitrile

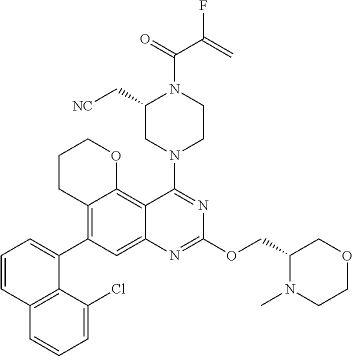 [SMILES: C=C(F)C(=O)N1CCN(c2nc(OC[C@@H]3COCCN3C)nc3cc(-c4cccc5cccc(Cl)c45)c4c(c23)OCCC4)C[C@@H]1CC#N]

The Compound B72 was synthesized starting from the corresponding starting materials according the similar procedures described as those of compound B48 to get the product (16 mg,). ¹H NMR (400 MHz, DMSO-d₆) δ 8.12 (d, J=7.8 Hz, 1H), 8.07 (d, J=7.8 Hz, 1H), 7.70-7.59 (m, 2H), 7.54 (s, 1H), 7.42-7.36 (m, 1H), 6.97 (d, J=1.6 Hz, 1H), 5.45-5.24 (m, 2H), 5.08-4.68 (m, 1H), 4.44-4.32 (m, 2H), 4.28-4.18 (m, 3H), 3.93-3.80 (m, 2H), 3.68 (d, J=10.4 Hz, 1H), 3.51-3.45 (m, 1H), 3.37 (s, 1H), 3.31-2.80 (m, 6H), 2.65 (d, J=11.8 Hz, 1H), 2.40 (s, 1H), 2.29 (s, 3H), 2.24-2.11 (m, 3H), 1.89 (s, 2H) ppm. MS: M/e 671 (M+1)⁺

### Compound B73: 2-((S)-4-(5-(8-chloronaphthalen-1-yl)-8-(((1R,3S,5R)-2-methyl-2-azabicyclo[3.1.0]hexan-3-yl)methoxy)-3,4-dihydro-2H-pyrano[2,3-f]quinazolin-10-yl)-1-(2-fluoroacryloyl)piperazin-2-yl)acetonitrile

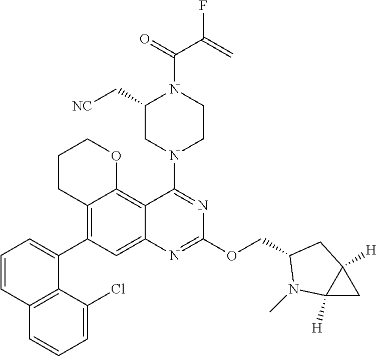 [SMILES: C=C(F)C(=O)N1CCN(c2nc(OC[C@@H]3C[C@H]4C[C@H]4N3C)nc3cc(-c4cccc5cccc(Cl)c45)c4c(c23)OCCC4)C[C@@H]1CC#N]

The Compound B73 was synthesized starting from the corresponding starting materials according the similar procedures described as those of compound B48 get the product (8 mg). ¹H NMR (400 MHz, DMSO-d₆) δ 8.15-8.05 (m, 2H), 7.65 (2H), 7.54 (s, 1H), 7.39 (d, J=19.5 Hz, 1H), 6.95 (s, 1H), 5.44-5.24 (m, 2H), 4.35 (s, 1H), 4.25 (s, 2H), 4.23-3.99 (m, 3H), 3.92-3.84 (m, 1H), 3.27-3.18 (m, 1H), 3.04-2.94 (m, 2H), 2.67 (s, 2H), 2.33 (s, 4H), 2.17 (s, 2H), 2.11-1.97 (m, 2H), 1.89 (s, 2H), 1.80 (s, 1H), 1.33 (s, 1H), 1.24 (s, 2H), 0.60 (s, 1H) ppm. MS: M/e 667 (M+1)⁺

### Compound B74: 2-((2S)-4-(5-(8-chloronaphthalen-1-yl)-8-(3-(dimethylamino)piperidin-1-yl)-3,4-dihydro-2H-pyrano[2,3-f]quinazolin-10-yl)-1-(2-fluoroacryloyl)piperazin-2-yl)acetonitrile

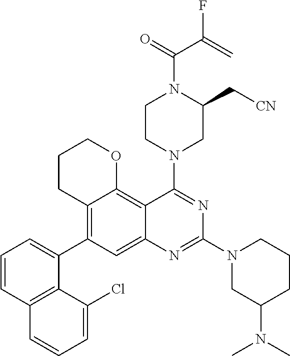 [SMILES: C=C(F)C(=O)N1CCN(c2nc(N3CCCC(N(C)C)C3)nc3cc(-c4cccc5cccc(Cl)c45)c4c(c23)OCCC4)C[C@@H]1CC#N]

The Compound B74 was synthesized starting from the corresponding starting materials according the similar procedures described as those of compound B48 to give the target compound (2.44 mg). ¹H NMR (400 MHz, DMSO-d₆) δ 8.17-8.12 (m, 2H), 7.74-7.69 (m, 2H), 7.63-7.58 (m, 1H), 7.43 (s, 1H), 5.49-5.44 (m, 2H), 4.34 (s, 5H), 4.15-3.80 (m, 2H), 3.76-3.51 (m, 5H), 2.89 (s, 6H), 2.34-2.06 (m, 5H), 1.99-1.70 (m, 4H), 1.55 (s, 4H) ppm. MS: M/e 668 (M+1)⁺.

### Compound B75: 2-((S)-4-(5-(8-chloronaphthalen-1-yl)-8-(((R)-1-methylpyrrolidin-3-yl)methoxy)-3,4-dihydro-2H-pyrano[2,3-f]quinazolin-10-yl)-1-(2-fluoroacryloyl)piperazin-2-yl)acetonitrile

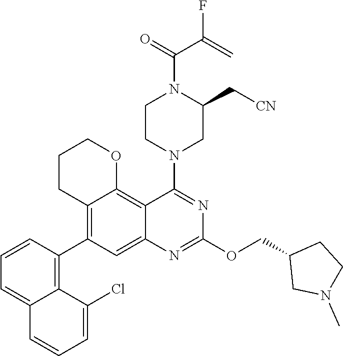 [SMILES: C=C(F)C(=O)N1CCN(c2nc(OC[C@@H]3CCN(C)C3)nc3cc(-c4cccc5cccc(Cl)c45)c4c(c23)OCCC4)C[C@@H]1CC#N]

The Compound B75 was synthesized starting from the corresponding starting materials according the similar procedures described as those of compound B48 to obtain the title compound (5.5 mg). ¹H NMR (400 MHz, CD₃OD) δ 8.03 (d, J=8.0 Hz, 1H), 7.96 (d, J=7.2 Hz, 1H), 7.65-7.51 (m, 2H), 7.50-7.41 (m, 1H), 7.40-7.29 (m, 1H), 7.03 (d, J=5.2 Hz, 1H), 5.45-5.23 (m, 2H), 5.16-4.92 (m, 1H), 4.52-4.35 (m, 4H), 4.34-4.14 (m, 2H), 4.14-4.05 (m, 1H), 3.72-3.56 (m, 1H), 3.52-3.41 (m, 1H), 3.27-2.94 (m, 7H), 2.95-2.75 (m, 2H), 2.69 (s, 3H), 2.32-2.23 (m, 2H), 2.02-1.81 (m, 3H) ppm. MS: M/e 655 (M+1)⁺.

### Compound B76: 1-(4-(4-(2-chloro-6-methylphenyl)-7-(((S)-1-methylpyrrolidin-2-yl)methoxy)furo[2,3-f]quinazolin-9-yl)piperazin-1-yl)prop-2-en-1-one

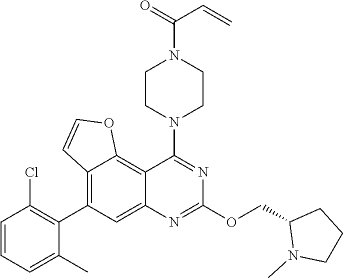 [SMILES: C=CC(=O)N1CCN(c2nc(OC[C@@H]3CCCN3C)nc3cc(-c4c(C)cccc4Cl)c4ccoc4c23)CC1]

The Compound B76 was synthesized starting from the corresponding starting materials according the similar procedures described as those of compound B38 give the target compound (1.24 mg). ¹H NMR (400 MHz, CD3OD) δ 8.00 (s, 1H), 7.58 (d, J=12.0 Hz, 1H), 7.41 (s, 1H), 7.35 (s, 2H), 6.86 (1H), 6.54 (s, 1H), 6.28 (d, J=16.8 Hz, 1H), 5.81 (d, J=10.5 Hz, 1H), 4.52 (s, 2H), 3.99 (s, 4H), 3.81 (s, 4H), 3.15 (s, 1H), 2.92 (s, 1H), 2.59 (s, 3H), 2.45 (s, 1H), 2.17 (s, 1H), 2.06 (s, 3H), 1.87 (s, 2H), 1.82-1.73 (m, 1H) ppm. MS: M/e 546 (M+1)⁺.

### Compound B77: 2-((S)-1-(2-fluoroacryloyl)-4-(4-(4-methyl-2-(trifluoromethyl)phenyl)-7-(((S)-1-methylpyrrolidin-2-yl)methoxy)furo[2,3-f]quinazolin-9-yl)piperazin-2-yl)acetonitrile

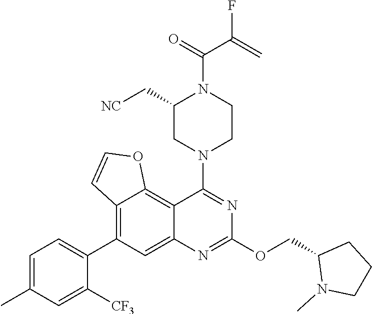 [SMILES: C=C(F)C(=O)N1CCN(c2nc(OC[C@@H]3CCCN3C)nc3cc(-c4ccc(C)cc4C(F)(F)F)c4ccoc4c23)C[C@@H]1CC#N]

The Compound B77 was synthesized starting from the corresponding starting materials according the similar procedures described as those of compound B38 to give the target compound (0.63 mg). ¹H NMR (400 MHz, CD₃OD) δ 8.06 (s, 1H), 7.72 (s, 1H), 7.58 (s, 1H), 7.47 (s, 1H), 7.39-7.31 (m, 1H), 6.62 (s, 1H), 5.34 (m, 2H), 4.62 (s, 3H), 4.40 (s, 2H), 3.71 (s, 2H), 3.50 (d, J=15.4 Hz, 2H), 3.13 (s, 2H), 2.93 (s, 3H), 2.19 (s, 3H), 2.02 (d, J=12.6 Hz, 5H), 1.60 (m, 2H) ppm. MS: M/e 637 (M+1)⁺.

### Compound B78: 2-((S)-4-(5-(2,3-dihydro-1H-inden-4-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

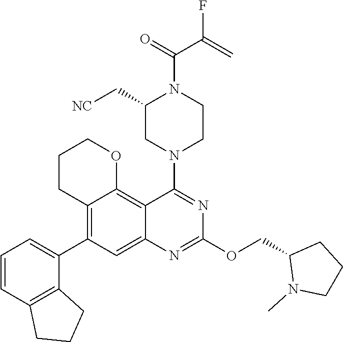 [SMILES: C=C(F)C(=O)N1CCN(c2nc(OC[C@@H]3CCCN3C)nc3cc(-c4cccc5c4CCC5)c4c(c23)OCCC4)C[C@@H]1CC#N]

The Compound B78 was synthesized starting from the corresponding starting materials according the similar procedures described as those of compound B31 to get the product (15 mg). ¹H NMR (400 MHz, DMSO-d₆) δ 7.28-7.23 (m, 2H), 6.99 (s, 1H), 6.87 (s, 1H), 5.45-5.24 (m, 2H), 4.68-4.15 (m, 6H), 3.89 (d, J=11.1 Hz, 1H), 3.31-3.24 (m, 2H), 3.20-2.81 (m, 9H), 2.73-2.55 (m, 3H), 2.34-2.28 (m, 3H), 2.13-1.62 (m, 9H) ppm. MS: M/e 611 (M+1)⁺

### Compound B79: 2-((2S)-4-(4-(5-chloro-6-methyl-1H-indazol-4-yl)-7-(3-(dimethylamino)azetidin-1-yl)furo[2,3-f]quinazolin-9-yl)-1-(2-fluoroacryloyl)piperazin-2-yl)acetonitrile

### Step A: 4-bromo-7-(3-(dimethylamino)azetidin-1-yl)furo[2,3-f]quinazolin-9(8H)-one

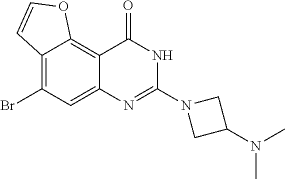 [SMILES: CN(C)C1CN(c2nc3cc(Br)c4ccoc4c3c(=O)[nH]2)C1]

A mixture of 4-bromo-7-chlorofuro[2,3-f]quinazolin-9(8H)-one (300 mg, 1.0 mmol), N,N-dimethylazetidin-3-amine dihydrochloride (350 mg, 2.0 mmol), and DIEA (758 mg, 5.8 mmol) in isopropyl alcohol (5 mL) was stirred at 80° C. for 3 hrs. The mixture was concentrated, treated with 10 mL of brine and extracted with EA (10 mL×3). The combined extracts was washed with brine (10 mL×3), dried, concentrated and purified by column chromatography (DCM/MeOH (7 M NH3)=50:1-20:1) to obtain the title compound (160 mg, yield: 44%). MS: M/e 363, 365 (M+1)⁺.

### Step B: tert-butyl (S)-4-(4-bromo-7-(3-(dimethylamino)azetidin-1-yl)furo[2,3-f]quinazolin-9-yl)-2-(cyanomethyl)piperazine-1-carboxylate

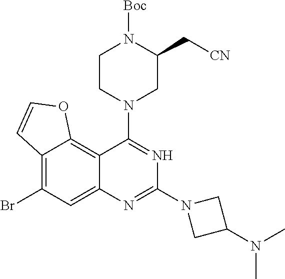 [SMILES: CN(C)C1CN(c2nc(N3CCN(C(=O)OC(C)(C)C)[C@@H](CC#N)C3)c3c(cc(Br)c4ccoc43)n2)C1]

To a mixture of 4-bromo-7-(3-(dimethylamino)azetidin-1-yl)furo[2,3-f]quinazolin-9(8H)-one (80 mg, 0.22 mmol), tert-butyl (S)-2-(cyanomethyl)piperazine-1-carboxylate (100 mg, 0.44 mmol) and PyBOP (145 mg, 0.33 mmol) in MeCN (4 mL) was added DBU (85 mg, 0.56 mmol) at rt and the mixture was stirred at rt for 2 hrs. The mixture was quenched with brine, extracted with EA (10 mL×2). The combined organics was concentrated and purified by prep-TLC to obtain an intermediate 1-(9-((1H-benzo[d][1,2,3]triazol-1-yl)oxy)-4-bromofuro[2,3-f]quinazolin-7-yl)-N,N-dimethylazetidin-3-amine (30 mg). A mixture of the intermediate above (65 mg, 0.13 mmol), tert-butyl (S)-2-(cyanomethyl)piperazine-1-carboxylate (50 mg, 0.22 mmol) and DIEA (100 mg, 0.77 mmol) in MeCN (3 mL) was stirred at rt for 16 hrs. 85 mg of DBU was added and the resulted mixture was at rt for 2 days. The mixture was treated with brine (10 mL), extracted with EA (10 mL×3). The combined extracts was washed with brine (10 mL×3), dried, concentrated and purified by prep-TLC (DCM/MeOH (7 M NH3)=15:1) to obtain the title compound (52 mg, yield: 70%). MS: M/e 570, 572 (M+1)⁺.

### Step C: tert-butyl (2S)-4-(4-(5-chloro-6-methyl-1-(tetrahydro-2H-pyran-2-yl)-1H-indazol-4-yl)-7-(3-(dimethylamino)azetidin-1-yl)furo[2,3-f]quinazolin-9-yl)-2-(cyanomethyl)piperazine-1-carboxylate

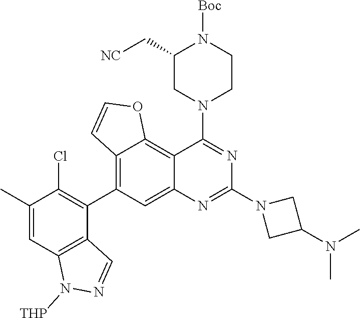

A mixture of 5-chloro-6-methyl-1-(tetrahydro-2H-pyran-2-yl)-4-(4,4,5,5-tetramethyl-1,3,2-dioxaborolan-2-yl)-1H-indazole (38 mg, 0.1 mmol), tert-butyl (S)-4-(4-bromo-7-(3-(dimethylamino)azetidin-1-yl)furo[2,3-f]quinazolin-9-yl)-2-(cyanomethyl)piperazine-1-carboxylate (52 mg, 0.09 mmol), Pd(dppf)Cl₂ (7 mg, 0.01 mmol), K₃PO₄ (69 mg, 0.32 mmol) and H₂O (0.3 mL) in Dioxane (2 mL) was stirred at 90° C. under N₂ for 3 hrs. The mixture was cooled, treated with brine (5 mL), extracted with EA (10 mL×3). The combined extracts was washed with brine (15 mL×3), dried, concentrated and purified by prep-TLC (DCM/MeOH (7M NH3)=15:1) to obtain the title compound (35 mg, yield: 47%). MS: M/e 740 (M+1)⁺.

### Step D: 2-((2S)-4-(4-(5-chloro-6-methyl-1H-indazol-4-yl)-7-(3-(dimethylamino)azetidin-1-yl)furo[2,3-f]quinazolin-9-yl)piperazin-2-yl)acetonitrile

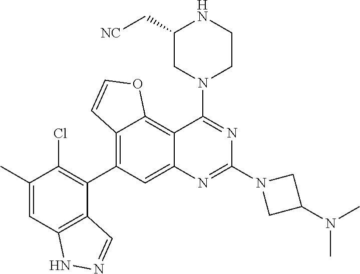

To a solution of tert-butyl (2S)-4-(4-(5-chloro-6-methyl-1-(tetrahydro-2H-pyran-2-yl)-1H-indazol-4-yl)-7-(3-(dimethylamino)azetidin-1-yl)furo[2,3-f]quinazolin-9-yl)-2-(cyanomethyl)piperazine-1-carboxylate (35 mg, 0.047 mmol) in CH₂Cl₂/MeOH (3:1, 3 mL) was added HCl/Dioxane (4N, 2 mL) at rt and stirred for 3 hrs. The mixture was concentrated to dryness, and diluted with (DCM/MeOH, 5:1, 10 mL), treated with Na2CO3 (solid, 50 mg), stirred for 10 min. The mixture was filtered and the filtrate was concentrated to dryness to obtain the title compound (29 mg, crude). MS: M/e 556 (M+1)⁺.

### Step E: 2-((2S)-4-(4-(5-chloro-6-methyl-1H-indazol-4-yl)-7-(3-(dimethylamino)azetidin-1-yl)furo[2,3-f]quinazolin-9-yl)-1-(2-fluoroacryloyl)piperazin-2-yl)acetonitrile

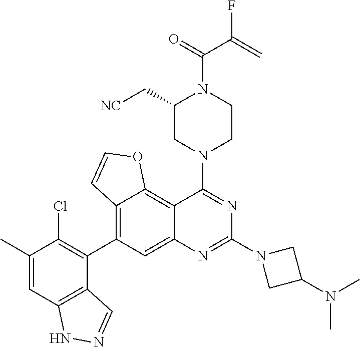

To a mixture of 2-((2S)-4-(4-(5-chloro-6-methyl-1H-indazol-4-yl)-7-(3-(dimethylamino)azetidin-1-yl)furo[2,3-f]quinazolin-9-yl)piperazin-2-yl)acetonitrile (29 mg, crude), 2-fluoroacrylic acid (14 mg, 0.157 mmol) and triethylamine (80 mg, 0.8 mmol) in THF (2 mL) was added a solution of T₃P (50 mg, 0.157 mmol) in THF (1 mL) at rt and the resulted mixture was stirred at rt for 3 hrs. The mixture was treated with NaHCO₃ to pH-8, and the mixture was extracted with EA (5 mL×3), the combined organic layers was washed with brine (10 mL×3), dried over Na₂SO₄, concentrated and purified by prep-TLC (CH₂Cl₂/MeOH (7 M NH₃)=15:1) and prep-HPLC to obtain the title compound (5 mg, yield: 15% for 2 steps). ¹H NMR (400 MHz, CD₃OD) δ 7.96 (s, 1H), 7.62 (s, 1H), 7.54 (s, 1H), 7.50 (s, 1H), 6.51 (s, 1H), 5.49-5.25 (m, 2H), 4.45-4.14 (m, 5H), 4.14-4.04 (m, 2H), 3.52-3.33 (m, 3H), 3.26-3.02 (m, 3H), 2.87-2.67 (m, 1H), 2.61 (s, 3H), 2.33 (s, 6H) ppm. MS: M/e 628 (M+1)⁺.

### Compound B80: (S)-2-(4-(4-(8-chloronaphthalen-1-yl)-7-(3-(cyanomethyl)-3-(dimethylamino)azetidin-1-yl)furo[2,3-f]quinazolin-9-yl)-1-(2-fluoroacryloyl)piperazin-2-yl)acetonitrile

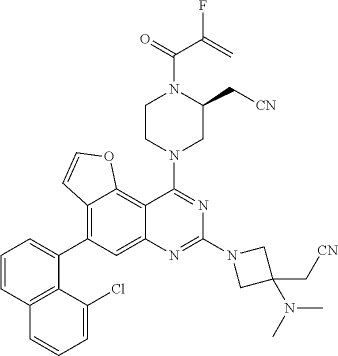 [SMILES: C=C(F)C(=O)N1CCN(c2nc(N3CC(CC#N)(N(C)C)C3)nc3cc(-c4cccc5cccc(Cl)c45)c4ccoc4c23)C[C@@H]1CC#N]

The Compound B80 was synthesized starting from the corresponding starting materials according the similar procedures described as those of compound B79 to give the target compound (3 mg). ¹H NMR (400 MHz, CD₃OD) δ 8.09 (d, J=7.6 Hz, 1H), 8.01 (d, J=7.6 Hz, 1H), 7.91 (s, 1H), 7.69-7.60 (m, 1H), 7.58-7.46 (m, 3H), 7.45-7.40 (m, 1H), 6.33 (s, 1H), 5.48-5.26 (m, 2H), 5.09 (s, 1H), 4.39 (d, J=14.0 Hz, 1H), 4.27-4.13 (m, 5H), 3.55-3.43 (m, 1H), 3.27-3.06 (m, 5H), 3.03 (s, 2H), 2.33 (s, 6H) ppm. MS: M/e 663 (M+1)⁺.

### Compound B81: 2-((S)-1-(2-fluoroacryloyl)-4-(8-(((S)-1-methylpyrrolidin-2-yl)methoxy)-5-(5,6,7,8-tetrahydronaphthalen-1-yl)-3,4-dihydro-2H-pyrano[2,3-f]quinazolin-10-yl)piperazin-2-yl)acetonitrile

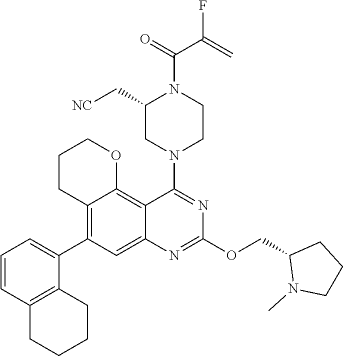 [SMILES: C=C(F)C(=O)N1CCN(c2nc(OC[C@@H]3CCCN3C)nc3cc(-c4cccc5c4CCCC5)c4c(c23)OCCC4)C[C@@H]1CC#N]

The Compound B81 was synthesized starting from the corresponding starting materials according the similar procedures described as those of compound B31 to get the product (21 mg). ¹H NMR (400 MHz, DMSO-d₆) δ 7.17 (s, 1H), 7.12 (s, 1H), 6.89 (s, 1H), 6.81 (s, 1H), 5.49-5.18 (m, 2H), 4.48-4.17 (m, 5H), 3.89 (s, 1H), 3.24 (s, 2H), 3.03 (4H), 2.81 (s, 3H), 2.63 (s, 4H), 2.32 (s, 2H), 2.22 (s, 4H), 1.93 (s, 2H), 1.82-1.69 (m. 8H) ppm. MS: M/e 625 (M+1)⁺

### Compound B82: 2-((S)-1-acryloyl-4-(5-(8-chloronaphthalen-1-yl)-8-(((S)-1-methylpyrrolidin-2-yl)methoxy)-3,4-dihydro-2H-pyrano[2,3-f]quinazolin-10-yl)piperazin-2-yl)acetonitrile

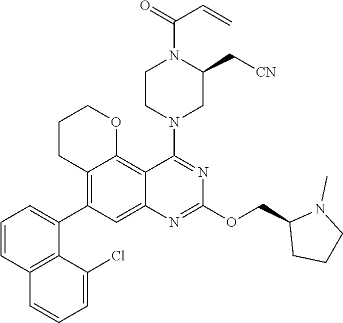 [SMILES: C=CC(=O)N1CCN(c2nc(OC[C@@H]3CCCN3C)nc3cc(-c4cccc5cccc(Cl)c45)c4c(c23)OCCC4)C[C@@H]1CC#N]

To a stirred solution of tert-butyl (S)-4-(5-(8-chloronaphthalen-1-yl)-8-(((S)-1-methylpyrrolidin-2-yl)methoxy)-3,4-dihydro-2H-pyrano[2,3-f]quinazolin-10-yl)-2-(cyanomethyl)piperazine-1-carboxylate (20 mg, 0.029 mmol) in CH₂Cl₂ (3 mL) was added HCl/dioxane (4 M, 2 mL). After stirred for an hour, the reaction mixture was concentrated to give the residue, which was dissolved in CH₃CN (5 mL) and aq.NaHCO₃ (0.5 mL) was added, then a solution of acryloyl chloride (2.65 mg, 0.029 mmol) in CH₃CN (0.5 mL) was added. After the addition, the reaction mixture was stirred for 20 min. The reaction mixture was extracted with EtOAc (10 mL), washed with brine, dried over Na₂SO₄, concentrated and purified by Prep-TLC (CH₂Cl₂/MeOH=10:1) to give the target compound (5 mg). ¹H NMR (400 MHz, CD₃OD) δ 8.04 (d, J=7.6 Hz, 1H), 7.98 (d, J=7.6 Hz, 1H), 7.67-7.53 (m, 2H), 7.52-7.29 (m, 2H), 7.07 (s, 1H), 6.82 (s, 1H), 6.30 (d, J=16.8 Hz, 1H), 5.84 (d, J=11.2 Hz, 1H), 4.84 (s, 1H), 4.65-4.05 (m, 6H), 3.89 (s, 1H), 3.75-3.42 (m, 2H), 3.28-3.18 (m, 3H), 3.07 (s, 3H), 3.02-2.79 (m, 3H), 2.47-1.89 (m, 8H) ppm. MS: M/e 637 (M+1)⁺.

### Compound B83: 1-(4-(7-(3-(dimethylamino)azetidin-1-yl)-4-(2-(trifluoromethyl)phenyl)furo[2,3-f]quinazolin-9-yl)piperazin-1-yl)prop-2-en-1-one

### Step A: 4-bromo-7-(3-(dimethylamino)azetidin-1-yl)furo[2,3-f]quinazolin-9(8H)-one

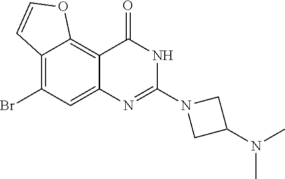 [SMILES: CN(C)C1CN(c2nc3cc(Br)c4ccoc4c3c(=O)[nH]2)C1]

To a stirred solution of 4-bromo-7-chlorofuro[2,3-f]quinazolin-9(8H)-one (150 mg, 0.5 mmol) in IPA (10 mL) was added N,N-dimethylazetidin-3-amine hydrochloride (173 mg, 1 mmol) and DIPEA (258 mg, 2 mmol). After the addition, the reaction was stirred at 90° C. for 3 hours. The reaction mixture was concentrated to give the residue, which was washed with H₂O (10 mL) and filtered. The cake was collected, dried to give the target compound (126 mg, 69.4%). MS: M/e 363/365 (M+1)⁺.

### Step B: tert-butyl 4-(4-bromo-7-(3-(dimethylamino)azetidin-1-yl)furo[2,3-f]quinazolin-9-yl)piperazine-1-carboxylate

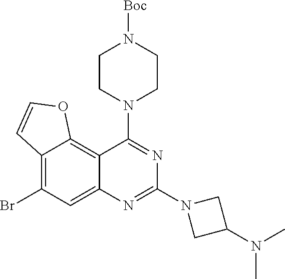 [SMILES: CN(C)C1CN(c2nc(N3CCN(C(=O)OC(C)(C)C)CC3)c3c(cc(Br)c4ccoc43)n2)C1]

To a stirred mixture of the product of step A (126 mg, 0.347 mmol) and tert-butyl piperazine-1-carboxylate (96.8 mg, 0.52 mmol) in CH₃CN (10 mL) was added PyBOP (200 mg, 0.45 mmol), followed by DBU (106.2 mg, 0.694 mmol). After the addition, the reaction was stirred overnight. The reaction mixture was quenched with H₂O, extracted with EtOAc (20 mL×2). The combined organic layers were washed with brine, dried over Na₂SO₄, concentrated and purified by Prep-TLC (CH₂Cl₂/MeOH=10:1) to give the target compound (100 mg, 54.3%). MS: M/e 531/533 (M+1)⁺.

### Step C: tert-butyl 4-(7-(3-(dimethylamino)azetidin-1-yl)-4-(2-(trifluoromethyl)phenyl)furo[2,3-f]quinazolin-9-yl)piperazine-1-carboxylate

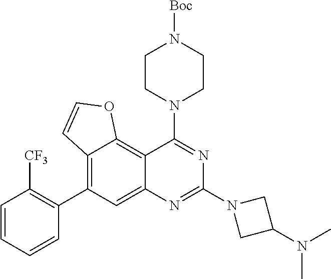 [SMILES: CN(C)C1CN(c2nc(N3CCN(C(=O)OC(C)(C)C)CC3)c3c(cc(-c4ccccc4C(F)(F)F)c4ccoc43)n2)C1]

A mixture of the product of step B (100 mg, 0.188 mmol) and (2-(trifluoromethyl)phenyl)boronic acid (43 mg, 0.226 mmol) in aq.K₃PO₄/dioxane (0.5 mL/5 mL) was added PddppfCl₂ (14 mg, 0.0188 mmol). After the addition, the reaction was stirred at 100° C. for 2 hours under N₂. The reaction mixture was diluted with EtOAc (10 mL), washed with brine, dried over Na₂SO₄, concentrated to give the residue, which was purified by Prep-TLC (CH₂Cl₂/MeOH=10:1) to give the target compound (50 mg, 44.7%). MS: M/e 597 (M+1)⁺.

### Step D: 1-(4-(7-(3-(dimethylamino)azetidin-1-yl)-4-(2-(trifluoromethyl)phenyl)furo[2,3-f]quinazolin-9-yl)piperazin-1-yl)prop-2-en-1-one

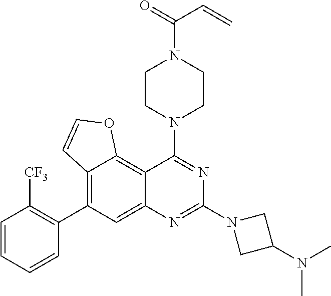 [SMILES: C=CC(=O)N1CCN(c2nc(N3CC(N(C)C)C3)nc3cc(-c4ccccc4C(F)(F)F)c4ccoc4c23)CC1]

To a stirred solution of step C (50 mg, 0.084 mmol) in CH₂Cl₂ (2 mL) was added HCl/dioxane (4 M, 2 mL). After stirred for an hour, the reaction mixture was concentrated to give the residue, which was dissolved in CH₃CN/aq.NaHCO₃ (5 mL/0.5 mL), then a solution of acryloyl chloride (7.6 mg, 0.084 mmol) in CH₃CN (0.5 mL) was added. After the addition, the reaction mixture was stirred for 20 min. The reaction mixture was extracted with EtOAc (10 mL), washed with brine, dried over Na₂SO₄, concentrated and purified by Prep-TLC (EtOAc) to give the target compound (8 mg). ¹H NMR (400 MHz, CD₃OD) δ 7.93-7.86 (m, 2H), 7.79-7.63 (m, 2H), 7.46 (d, J=6.4 Hz, 1H), 7.34 (s, 1H), 6.92-6.79 (m, 1H), 6.51 (s, 1H), 6.27 (d, J=16.8 Hz, 1H), 5.80 (d, J=10.4 Hz, 1H), 4.34-4.25 (m, 2H), 4.11-3.89 (m, 6H), 3.77-3.60 (m, 4H), 3.33 (s, 1H), 2.31 (s, 6H) ppm. MS: M/e 551 (M+1)⁺.

### Compound B84: 2-((2S)-4-(5-(8-chloronaphthalen-1-yl)-8-((1-methylpiperidin-3-yl)oxy)-3,4-dihydro-2H-pyrano[2,3-f]quinazolin-10-yl)-1-(2-fluoroacryloyl)piperazin-2-yl)acetonitrile

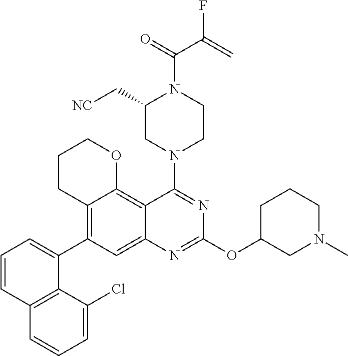 [SMILES: C=C(F)C(=O)N1CCN(c2nc(OC3CCCN(C)C3)nc3cc(-c4cccc5cccc(Cl)c45)c4c(c23)OCCC4)C[C@@H]1CC#N]

The Compound B84 was synthesized starting from the corresponding starting materials according the similar procedures described as those of compound B48 to get the product (12 mg). ¹H NMR (400 MHz, DMSO-d₆) δ 8.07 (2H), 7.62 (s, 2H), 7.53 (s, 1H), 7.39 (1H), 6.94 (s, 1H), 5.44-5.23 (m, 2H), 5.04 (s, 1H), 4.35 (s, 1H), 4.24 (s, 1H), 4.17 (s, 1H), 3.87 (s, 1H), 3.31-2.71 (m, 9H), 2.24-2.11 (m, 5H), 2.10-1.92 (m, 3H), 1.88 (s, 2H), 1.70 (s, 1H), 1.54 (s, 1H), 1.44-1.33 (m, 1H) ppm. MS: M/e 655 (M+1)⁺

### Compound B85: 2-((2S)-4-(5-(8-chloronaphthalen-1-yl)-8-((1-(dimethylamino)propan-2-yl)oxy)-3,4-dihydro-2H-pyrano[2,3-f]quinazolin-10-yl)-1-(2-fluoroacryloyl)piperazin-2-yl)acetonitrile

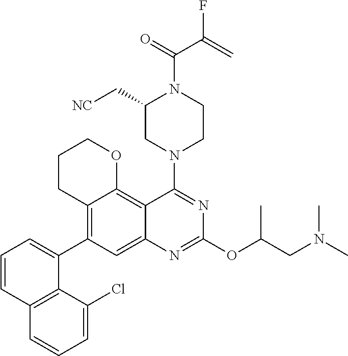 [SMILES: C=C(F)C(=O)N1CCN(c2nc(OC(C)CN(C)C)nc3cc(-c4cccc5cccc(Cl)c45)c4c(c23)OCCC4)C[C@@H]1CC#N]

The Compound B85 was synthesized starting from the corresponding starting materials according the similar procedures described as those of compound B48 to get the product (15 mg). ¹H NMR (400 MHz, DMSO-d₆) δ 8.08 (2H), 7.63 (s, 2H), 7.53 (s, 1H), 7.40 (1H), 6.93 (s, 1H), 5.45-5.23 (m, 3H), 4.35 (s, 1H), 4.26-4.14 (m, 2H), 3.88 (s, 1H), 3.29-2.73 (m, 8H), 2.33 (s, 1H), 2.25-1.99 (m, 8H), 1.89 (s, 2H), 1.27 (s, 3H) ppm. MS: M/e 643 (M+1)⁺

### Compound B86: (S)-2-(4-(5-(8-chloronaphthalen-1-yl)-8-(3-(dimethylamino)-3-(methoxymethyl)azetidin-1-yl)-3,4-dihydro-2H-pyrano[2,3-f]quinazolin-10-yl)-1-(2-fluoroacryloyl)piperazin-2-yl)acetonitrile

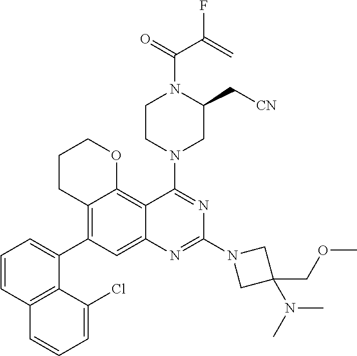 [SMILES: C=C(F)C(=O)N1CCN(c2nc(N3CC(COC)(N(C)C)C3)nc3cc(-c4cccc5cccc(Cl)c45)c4c(c23)OCCC4)C[C@@H]1CC#N]

### Step A: 1-(tert-butyl) 3-ethyl 3-(dimethylamino)azetidine-1,3-dicarboxylate

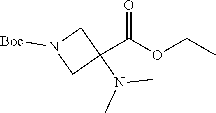 [SMILES: CCOC(=O)C1(N(C)C)CN(C(=O)OC(C)(C)C)C1]

To a stirred solution of 1-(tert-butyl) 3-ethyl 3-aminoazetidine-1,3-dicarboxylate (2 g, 8.2 mmol) in CH₂Cl₂ (50 mL) was added HCHO (30%, 4 mL). After stirred for an hour, NaBH(OAc)₃ (5.2 g, 24.6 mmol). After the addition, the reaction was stirred for 3 hours. The reaction was quenched with MeOH, concentrated to give the residue, which was diluted with EtOAc (50 mL) and washed with aq.NaHCO₃, brine, dried over Na₂SO₄, concentrated to give the target crude compound, then was purified by column chromatography (petroleum ether/EtOAc=5:1-3:1) to give the target compound (950 mg, 42.6%). MS: M/e 273 (M+1)⁺.

### Step B: tert-butyl 3-(dimethylamino)-3-(hydroxymethyl)azetidine-1-carboxylate

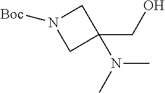 [SMILES: CN(C)C1(CO)CN(C(=O)OC(C)(C)C)C1]

To a stirred suspension of LAH (262 mg, 6.91 mmol) in THF (10 mL) was added dropwise a solution of the product of step A (940 mg, 3.45 mmol) in THF (5 mL) at 0° C. After the addition, the reaction was stirred for an hour. The reaction was quenched with H₂O (262 mg), then aq.NaOH (15%, 262 mg) followed by H₂O (786 mg). Then diluted with EtOAc (20 mL) and filtered. The filtrate was collected, concentrated to give the target compound (375 mg, 46.6%). MS: M/e 231 (M+1)⁺.

### Step C: tert-butyl 3-(dimethylamino)-3-(methoxymethyl)azetidine-1-carboxylate

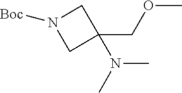 [SMILES: COCC1(N(C)C)CN(C(=O)OC(C)(C)C)C1]

To a stirred suspension of NaH (34.8 mg, 0.87 mmol) in THF (5 mL) was added a solution of the product of step B (100 mg, 0.435 mmol) in THF (3 mL) at 0° C. After stirred for half an hour, a solution of Mel (74 mg, 0.52 mmol) in THF (1 mL) was added dropwise and stirred for an hour. The reaction was quenched with aq.NH₄Cl, extracted with EtOAc (10 mL×2). The combined organic layers were washed with brine, dried over Na₂SO₄, concentrated to give crude target compound, which was directly used to the next step. MS: M/e 245 (M+1)⁺.

### Step D: 3-(methoxymethyl)-N,N-dimethylazetidin-3-amine hydrochloride

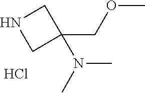

To a stirred solution of the product of C (crude, 0.435 mmol) in CH₂Cl₂ (5 mL) was added HCl/dioxane (4.0 M, 2 mL). After the addition, the reaction was stirred overnight. The reaction mixture was concentrated to give the target compound, which was directly used to the next step. MS: M/e 145 (M+1)⁺.

The Compound B86 was synthesized starting from the corresponding starting materials according the similar procedures described as those of compound B48 to give the target compound (5 mg). ¹H NMR (400 MHz, CD₃OD) δ 8.05 (d, J=8.0 Hz, 1H), 7.98 (d, J=8.0 Hz, 1H), 7.69-7.54 (m, 2H), 7.48 (t, J=8.0 Hz, 1H), 7.35 (d, J=6.0 Hz, 1H), 6.96 (s, 1H), 5.48-5.27 (m, 2H), 4.78-4.55 (m, 1H), 4.35 (s, 2H), 4.25-4.03 (m, 6H), 3.74 (s, 2H), 3.67-3.44 (m, 3H), 3.41 (s, 3H), 3.12-2.71 (m, 3H), 2.43 (s, 6H), 2.37-2.21 (m, 2H), 1.96 (s, 2H) ppm. MS: M/e 684 (M+1)⁺.

### Compound B87: 2-((S)-1-(2-fluoroacryloyl)-4-(7-(((S)-1-methylpyrrolidin-2-yl)methoxy)-4-(3-(trifluoromethyl)phenyl)furo[2,3-f]quinazolin-9-yl)piperazin-2-yl)acetonitrile

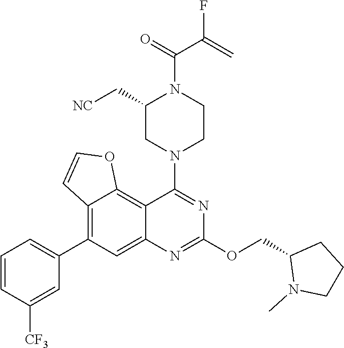 [SMILES: C=C(F)C(=O)N1CCN(c2nc(OC[C@@H]3CCCN3C)nc3cc(-c4cccc(C(F)(F)F)c4)c4ccoc4c23)C[C@@H]1CC#N]

The Compound B87 was synthesized starting from the corresponding starting materials according the similar procedures described as those of compound B38 to give the target compound (5 mg). ¹H NMR (400 MHz, CD₃OD) δ 8.17 (s, 1H), 7.99 (s, 2H), 7.83 (s, 2H), 7.70 (s, 1H), 7.11 (s, 1H), 5.40 (1H), 5.32 (s, 1H), 4.68 (s, 2H), 4.45 (d, J=14.8 Hz, 1H), 4.25 (d, J=13.5 Hz, 2H), 3.94 (s, 2H), 3.73 (s, 2H), 3.24-3.13 (m, 2H), 3.10 (s, 3H), 2.43 (s, 1H), 2.21 (s, 2H), 2.11 (s, 2H), 1.29 (m, 2H) ppm. MS: M/e 623 (M+1)⁺.

### Compound B88: (S)-2-(4-(5-(8-chloronaphthalen-1-yl)-8-(3-(dimethylamino)-3-(fluoromethyl)azetidin-1-yl)-3,4-dihydro-2H-pyrano[2,3-f]quinazolin-10-yl)-1-(2-fluoroacryloyl)piperazin-2-yl)acetonitrile

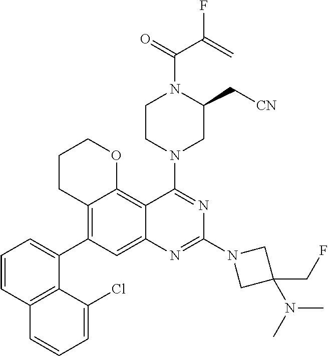 [SMILES: C=C(F)C(=O)N1CCN(c2nc(N3CC(CF)(N(C)C)C3)nc3cc(-c4cccc5cccc(Cl)c45)c4c(c23)OCCC4)C[C@@H]1CC#N]

### Step A: tert-butyl 3-(dimethylamino)-3-(fluoromethyl)azetidine-1-carboxylate

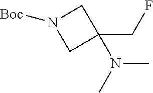 [SMILES: CN(C)C1(CF)CN(C(=O)OC(C)(C)C)C1]

To a stirred solution of tert-butyl 3-(dimethylamino)-3-(hydroxymethyl)azetidine-1-carboxylate (100 mg, 0.435 mmol) in CH₂Cl₂ (5 mL) was added Et₃N (87 mg, 0.87 mmol) followed by a solution of MsCl (74 mg, 0.65 mmol) in CH₂Cl₂ (2 mL) at 0° C. After the addition, the reaction mixture was stirred for an hour. The reaction mixture was washed with H₂O, extracted with CH₂Cl₂ (10 mL×2). The combined organic layers were washed with brine, dried over Na₂SO₄, concentrated to give the residue, which was dissolved in TBAF (1.0 M in dioxane, 5 mL) was stirred at 60° C. for an hour. The reaction mixture was concentrated, treated with EtOAc (20 mL), washed with H₂O, dried over Na₂SO₄, concentrated to give the target compound (crude, 100%), which was directly used to the next step. MS: M/e 233 (M+1)⁺.

### Step B: 3-(fluoromethyl)-N,N-dimethylazetidin-3-amine hydrochloride

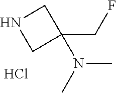

To a stirred solution of the product of A (crude, 0.435 mmol) in CH₂Cl₂ (5 mL) was added HCl/dioxane (4.0 M, 2 mL). After the addition, the reaction was stirred for 3 hours. The reaction mixture was concentrated to give the target compound, which was directly used to the next step. MS: M/e 133 (M+1)⁺.

The Compound B88 was synthesized starting from the corresponding starting materials according the similar procedures described as those of compound B48 to give the target compound (5 mg). ¹H NMR (400 MHz, CD₃OD) δ 8.06 (d, J=7.6 Hz, 1H), 7.98 (d, J=7.6 Hz, 1H), 7.67-7.54 (m, 2H), 7.52-7.44 (m, 1H), 7.35 (d, J=6.0 Hz, 1H), 6.96 (s, 1H), 5.46-5.25 (m, 2H), 4.92 (s, 1H), 4.80 (s, 1H), 4.72 (s, 1H), 4.36 (s, 2H), 4.25-4.07 (m, 5H), 3.79-3.45 (m, 4H), 3.27-3.18 (m, 1H), 2.99 (s, 2H), 2.41 (s, 6H), 2.36-2.22 (m, 2H), 1.97 (s, 2H) ppm. MS: M/e 672 (M+1)⁺.

### Compound B89: 2-((S)-1-(2-fluoroacryloyl)-4-(7-(((S)-1-methylpyrrolidin-2-yl)methoxy)-4-(4-(trifluoromethyl)phenyl)furo[2,3-f]quinazolin-9-yl)piperazin-2-yl)acetonitrile

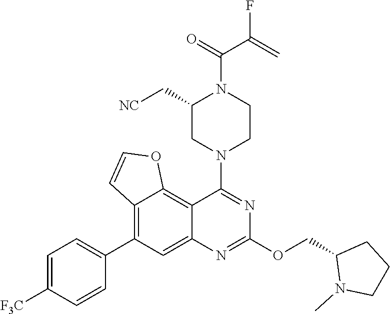 [SMILES: C=C(F)C(=O)N1CCN(c2nc(OC[C@@H]3CCCN3C)nc3cc(-c4ccc(C(F)(F)F)cc4)c4ccoc4c23)C[C@@H]1CC#N]

The Compound B89 was synthesized starting from the corresponding starting materials according the similar procedures described as those of compound B38 to give the target compound (1 mg). ¹H NMR (400 MHz, CD₃OD) δ 8.14 (d, J=7.7 Hz, 1H), 7.93 (s, 1H), 7.89 (s, 1H), 7.69 (s, 1H), 7.65 (s, 1H), 7.56 (s, 1H), 7.15 (s, 1H), 5.41 (s, 1H), 5.33 (d, J=16.9 Hz, 2H), 4.52 (d, J=8.4 Hz, 2H), 4.42-4.27 (m, 2H), 4.20 (t, J=14.5 Hz, 2H), 3.51 (d, J=14.2 Hz, 1H), 3.15 (d, J=17.1 Hz, 2H), 2.60 (s, 3H), 2.46 (s, 1H), 2.19 (s, 2H), 2.03 (s, 1H), 1.88 (s, 4H) ppm. MS: M/e 623 (M+1)⁺.

### Compound B90: (S)-2-(1-(5-(8-chloronaphthalen-1-yl)-10-(3-(cyanomethyl)-4-(2-fluoroacryloyl)piperazin-1-yl)-3,4-dihydro-2H-pyrano[2,3-f]quinazolin-8-yl)-3-(dimethylamino)azetidin-3-yl)acetonitrile

### Step A: tert-butyl 3-(cyanomethyl)-3-(dimethylamino)azetidine-1-carboxylate

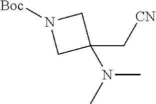 [SMILES: CN(C)C1(CC#N)CN(C(=O)OC(C)(C)C)C1]

A mixture of tert-butyl 3-(cyanomethylene)azetidine-1-carboxylate (300 mg, 1.55 mmol) in aq.NHMe₂ (40%)/MeOH (3 mL/2 mL) was stirred at 80° C. for 3 hours. Most MeOH was removed to give the aqueous layer, which was diluted with H₂O (10 mL) and extracted with EtOAc (30 mL). The organic layer was washed with brine, dried over Na₂SO₄, concentrated to give the target compound (320 mg, 86.4%). MS: M/e 240 (M+1)⁺.

### Step B: 2-(3-(dimethylamino)azetidin-3-yl)acetonitrile hydrochloride

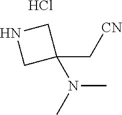

To a stirred solution of the product of step A (320 mg, 1.34 mmol) in CH₂Cl₂ (4 mL) was added HCl/dioxane (4.0 M, 4 mL). Then the mixture was stirred for 2 hours. The reaction mixture was concentrated to give the residue, which was washed with petroleum ether (20 mL) and filtered. The caked was collected, dried to give the target compound (300 mg, 100%). MS: M/e 140 (M+1)⁺.

### Step C: tert-butyl (S)-4-(5-(8-chloronaphthalen-1-yl)-8-(3-(cyanomethyl)-3-(dimethylamino)azetidin-1-yl)-3,4-dihydro-2H-pyrano[2,3-f]quinazolin-10-yl)-2-(cyanomethyl)piperazine-1-carboxylate

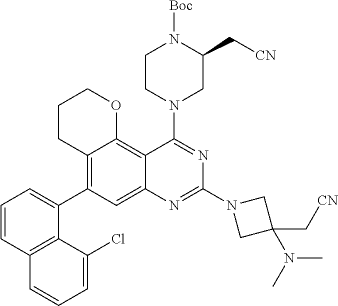 [SMILES: CN(C)C1(CC#N)CN(c2nc(N3CCN(C(=O)OC(C)(C)C)[C@@H](CC#N)C3)c3c4c(c(-c5cccc6cccc(Cl)c56)cc3n2)CCCO4)C1]

A mixture of tert-butyl (S)-4-(8-chloro-5-(8-chloronaphthalen-1-yl)-3,4-dihydro-2H-pyrano[2,3-f]quinazolin-10-yl)-2-(cyanomethyl)piperazine-1-carboxylate (80 mg, 0.133 mmol) and the product of step B (70.2 mg, 0.4 mmol) and DIPEA (103 mg, 0.8 mmol) in IPA (5 mL) was stirred at 80° C. overnight. The reaction mixture was concentrated to give the residue, which was diluted with H₂O (20 mL) and extracted with EtOAc (10 mL). The organic layer were washed with brine, dried over Na₂SO₄, concentrated and purified by Prep-TLC (CH₂Cl₂/MeOH=10:1) to give the target compound (60 mg, 63.8%). MS: M/e 707 (M+1)⁺.

### Step D: (S)-2-(1-(5-(8-chloronaphthalen-1-yl)-10-(3-(cyanomethyl)-4-(2-fluoroacryloyl)piperazin-1-yl)-3,4-dihydro-2H-pyrano[2,3-f]quinazolin-8-yl)-3-(dimethylamino)azetidin-3-yl)acetonitrile

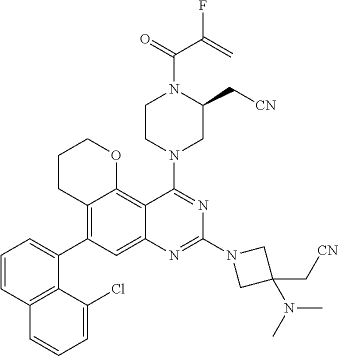 [SMILES: C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N]

To a stirred solution of step C (60 mg, 0.085 mmol) in EtOAc (3 mL) was added dioxane/HCl (4 M, 2 mL). After stirred for 3 hours, the reaction mixture was concentrated to give the residue, which was basified to pH=9˜10 with aq.Na₂CO₃ and extracted with EtOAc (15 mL). The organic layer was dried over Na₂SO₄ and concentrated to give the intermediate, which was dissolved in THF (5 mL), 2-fluoroacrylic acid (23 mg, 0.255 mmol) was added, followed by T₃P (W/W=1:1, 162 mg, 0.255 mmol) and Et₃N (51.5 mg, 0.51 mmol). After the addition, the reaction mixture was stirred for 2 hours. The reaction mixture was diluted with H₂O (10 mL), extracted with EtOAc (10 mL×2). The combined organic layers were washed with brine, dried over Na₂SO₄, concentrated and purified by Prep-TLC (CH₂Cl₂/MeOH=10:1) to give the target compound (6 mg). ¹H NMR (400 MHz, CD₃OD) δ 8.03 (d, J=8.0 Hz, 1H), 7.96 (d, J=8.0 Hz, 1H), 7.67-7.53 (m, 2H), 7.53-7.42 (m, 1H), 7.40-7.31 (m, 1H), 6.98 (s, 1H), 5.47-5.24 (m, 2H), 4.9 (s, 1H), 4.59-4.21 (m, 3H), 4.21-4.03 (m, 6H), 3.52-3.41 (m, 1H), 3.01 (s, 6H), 2.31 (s, 8H), 1.95 (s, 2H) ppm. MS: M/e 679 (M+1)⁺.

### Compound B91 and B92: (S)-2-(1-(5-(8-chloronaphthalen-1-yl)-10-(3-(cyanomethyl)-4-(2-fluoroacryloyl)piperazin-1-yl)-3,4-dihydro-2H-pyrano[2,3-f]quinazolin-8-yl)-3-(dimethylamino)azetidin-3-yl)acetonitrile (atropisomer 1) and (S)-2-(1-(5-(8-chloronaphthalen-1-yl)-10-(3-(cyanomethyl)-4-(2-fluoroacryloyl)piperazin-1-yl)-3,4-dihydro-2H-pyrano[2,3-f]quinazolin-8-yl)-3-(dimethylamino)azetidin-3-yl)acetonitrile (atropisomer 2)

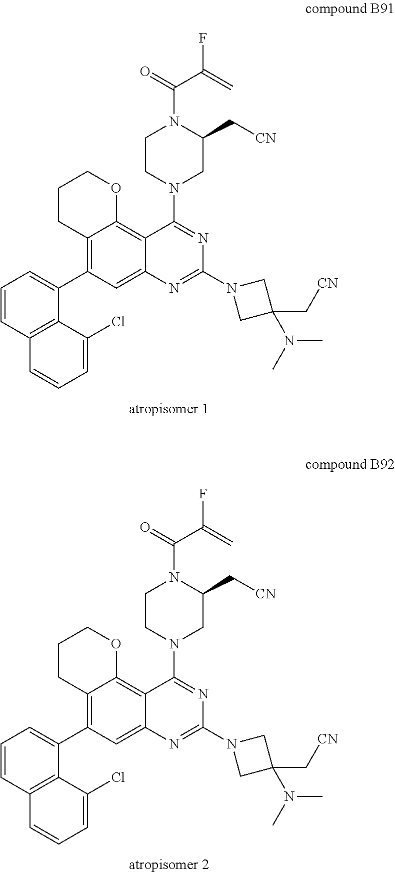 [SMILES: C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N]

The compound B90 was separated using preparative HPLC on a CHIRALART to afford the compound B91 (atropisomer 1) and B92 (atropisomer 2)

Prep-HPLC condition

| Column | CHIRAL Cellulose-SB |
|---|---|
| Column size | 2 cm × 25 cm, 5 um |
| Injection | 0.3 mL |
| Mobile phase | Hex(0.1% 2M NH₃-MEOH): EtOH = 50:50 |
| Flow rate | 20 mL/min |
| Wave length | UV 220 nm |
| Temperature | 25° C. |
| Sample solution | 40 mg/ml in ETOH:DCM = 2:1 |
| Prep-HPLC equipment | Prep-HPLC-Gilson |

Compound B91 (atropisomer 1) ¹H NMR (400 MHz, CD₃OD) δ 8.05 (d, J=8.0 Hz, 1H), 7.98 (d, J=8.0 Hz, 1H), 7.68-7.30 (m, 4H), 6.97 (s, 1H), 5.46-5.25 (m, 2H), 4.9 (s, 1H), 4.63 (s, 1H), 4.43-4.07 (m, 8H), 3.68-3.44 (m, 2H), 3.03 (s, 2H), 3.00-2.94 (m, 1H), 2.32 (s, 6H), 2.29-2.21 (m, 2H), 1.96 (s, 2H) ppm. MS: M/e 679 (M+1)⁺.

Compound B92 (atropisomer 2): ¹H NMR (400 MHz, CD₃OD) δ 8.04 (d, J=8.0 Hz, 1H), 7.97 (d, J=8.0 Hz, 1H), 7.66-7.30 (m, 4H), 6.97 (s, 1H), 5.45-5.28 (m, 2H), 4.9 (s, 1H), 4.60 (s, 1H), 4.39-4.05 (m, 8H), 3.60-3.39 (m, 2H), 3.02 (s, 2H), 3.01-2.91 (m, 2H), 2.31 (s, 6H), 2.30-2.17 (m, 2H), 1.96 (s, 2H) ppm. MS: M/e 679 (M+1)⁺.

### Compound B93: 2-((2S)-4-(5-(8-chloronaphthalen-1-yl)-8-(((4R)-4-methoxy-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

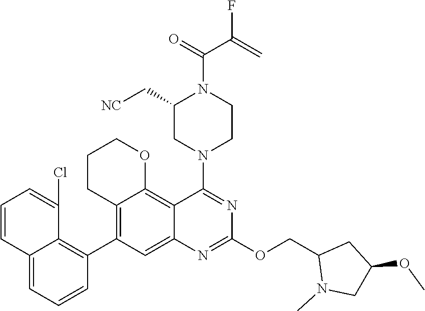 [SMILES: C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N]

The Compound B93 was synthesized starting from the corresponding starting materials according the similar procedures described as those of compound B48 to afford product (140 mg). ¹H NMR (400 MHz, CD₃OD) δ 8.03 (d, J=8.0 Hz, 1H), 7.96 (d, J=7.3 Hz, 1H), 7.61 (d, J=7.6 Hz, 1H), 7.55 (s, 1H), 7.46 (s, 1H), 7.36 (1H), 7.05 (d, J=5.3 Hz, 1H), 5.37 (1H), 5.30 (s, 1H), 4.49 (s, 2H), 4.41 (s, 2H), 4.29 (s, 1H), 4.10 (s, 1H), 4.01 (s, 1H), 3.48 (s, 3H), 3.25-3.15 (m, 2H), 3.13 (s, 1H), 3.00 (s, 1H), 2.81 (s, 2H), 2.62 (s, 4H), 2.57 (s, 1H), 2.31 (s, 2H), 2.19 (s, 2H), 2.06-1.95 (m, 4H) ppm. MS: M/e 685 (M+1)⁺.

The racemic mixture was chiral-separated twice to afford Compound B94: (isomer 1, 5.16 mg), Compound B95: (isomer 2, 17.48 mg), Compound B96: (isomer 3, 6.63 mg) and Compound B97: (isomer 4, 18.92 mg).

First Prep-HPLC condition

| Column | CHIRAL ART Cellulose-SB |
|---|---|
| Column size | 2 cm × 25 cm, 5 um |
| Injection | 1.0 mL |
| Mobile phase | [Hex:DCM = 3:1(0.5% 2M NH3-MeOH)]: |
| | MEOH = 50:50 |
| Flow rate | 20 mL/min |
| Wave length | UV 220 nm |
| Temperature | 25° C. |
| Sample solution | 20 mg/ml in MEOH:DCM = 2:1 |
| Prep-HPLC equipment | Prep-HPLC-Gilson |

Second Prep-HPLC condition

| Column | CHIRALPAK IE-3 |
|---|---|
| Column size | 2 cm × 25 cm, 5 um |
| Injection | 1.0 mL |
| Mobile phase | MTBE (0.5% 2M NH3-MeOH):IPA = 80:20 |
| Flow rate | 20 mL/min |
| Wave length | UV 220 nm |
| Temperature | 25° C. |
| Sample solution | 15 mg/ml in MEOH:DCM = 3:1 |
| Prep-HPLC equipment | Prep-HPLC-Gilson |

Compound B94: isomer 1: 1H NMR (400 MHz, CD3OD) δ 8.02 (d, J=7.9 Hz, 1H), 7.96 (d, J=8.1 Hz, 1H), 7.61 (t, J=8.0 Hz, 1H), 7.54 (d, J=8.0 Hz, 1H), 7.45 (t, J=8.0 Hz, 1H), 7.39 (d, J=8.1 Hz, 1H), 7.06 (s, 1H), 5.44-5.23 (m, 2H), 4.51-4.35 (m, 4H), 4.28 (s, 1H), 4.10 (d, J=12.5 Hz, 1H), 3.97 (s, 1H), 3.85-3.35 (m, 4H), 3.30-3.26 (m, 4H), 3.25-2.64 (m, 4H), 2.54 (s, 3H), 2.43 (s, 1H), 2.37-2.19 (m, 2H), 2.10 (s, 1H), 1.96 (s, 3H) ppm, MS: M/e 685 (M+1)⁺.

Compound B95: isomer B: 1H NMR (400 MHz, CD3OD) δ 8.02 (d, J=8.1 Hz, 1H), 7.96 (d, J=7.6 Hz, 1H), 7.61 (t, J=7.5 Hz, 1H), 7.54 (d, J=6.2 Hz, 1H), 7.46 (t, J=7.7 Hz, 1H), 7.38 (d, J=5.9 Hz, 1H), 7.05 (s, 1H), 5.47-5.19 (m, 2H), 4.53-4.34 (m, 4H), 4.27 (s, 1H), 4.11 (d, J=12.3 Hz, 1H), 3.90 (s, 1H), 3.83-3.38 (m, 2H), 3.28 (s, 5H), 3.25-3.05 (m, 3H), 3.03-2.92 (m, 1H), 2.80 (s, 1H), 2.50 (s, 3H), 2.46-2.36 (m, 2H), 2.35-2.19 (m, 2H), 1.97 (s, 2H), 1.82-1.71 (m, 1H) ppm, MS: M/e 685.6 (M+1)⁺.

Compound B96: isomer C: 1H NMR (400 MHz, CD3OD) δ 8.02 (d, J=8.2 Hz, 1H), 7.96 (d, J=7.8 Hz, 1H), 7.60 (t, J=7.8 Hz, 1H), 7.56 (d, J=7.2 Hz, 1H), 7.47 (t, J=7.6 Hz, 1H), 7.32 (d, J=7.5 Hz, 1H), 7.04 (s, 1H), 5.47-5.23 (m, 2H), 4.47-4.36 (m, 4H), 4.32 (s, 1H), 4.09 (d, J=12.7 Hz, 1H), 3.96 (s, 1H), 3.87-3.55 (m, 1H), 3.54-3.33 (m, 3H), 3.30-3.27 (m, 4H), 3.25-3.06 (m, 2H), 3.04-2.93 (m, 2H), 2.51 (s, 3H), 2.43-2.23 (m, 3H), 2.08 (s, 1H), 1.98 (s, 3H) ppm, MS: M/e 685 (M+1)⁺.

Compound B97: isomer 4: 1H NMR (400 MHz, CD3OD) δ 8.02 (d, J=8.3 Hz, 1H), 7.96 (d, J=8.1 Hz, 1H), 7.60 (t, J=7.1 Hz, 1H), 7.55 (d, J=7.2 Hz, 1H), 7.45 (t, J=7.8 Hz, 1H), 7.32 (d, J=6.7 Hz, 1H), 7.03 (s, 1H), 5.33 (dd, J=32.5, 12.5 Hz, 2H), 4.53-4.36 (m, 4H), 4.35-4.26 (s, 1H), 4.09 (d, J=12.7 Hz, 1H), 3.90 (s, 1H), 3.83-3.48 (m, 1H), 3.43 (d, J=14.3 Hz, 1H), 3.29-3.25 (m, 5H), 3.24-3.09 (m, 3H), 2.99 (d, J=10.7 Hz, 1H), 2.79 (s, 1H), 2.49 (s, 3H), 2.46-2.37 (m, 2H), 2.35-2.23 (m, 2H), 1.98 (s, 2H), 1.81-1.70 (m, 1H) ppm, MS: M/e 685 (M+1)⁺.

### Compound B98: 2-((S)-4-(5-(4-chloronaphthalen-1-yl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

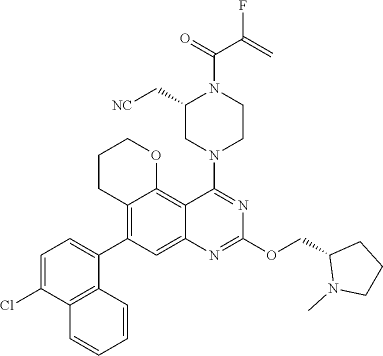 [SMILES: C=C(F)C(=O)N1CCN(c2nc(OC[C@@H]3CCCN3C)nc3cc(-c4ccc(Cl)c5ccccc45)c4c(c23)OCCC4)C[C@@H]1CC#N]

The Compound B98 was synthesized starting from the corresponding starting materials according the similar procedures described as those of compound B48 to get the product (33 mg). ¹H NMR (400 MHz, DMSO-d₆) δ 8.27 (d, J=9.0 Hz, 1H), 7.78 (s, 1H), 7.72 (s, 1H), 7.57 (s, 1H), 7.50 (s, 1H), 7.44-7.35 (m, 1H), 6.97 (s, 1H), 5.46-5.17 (m, 2H), 4.29 (s, 4H), 4.11 (s, 1H), 3.96 (s, 1H), 3.91 (s, 1H), 3.45-3.15 (m, 3H), 3.14-2.85 (m, 4H), 2.45-2.37 (m, 1H), 2.32 (s, 3H), 2.28-2.21 (m, 1H), 2.17-2.08 (m, 2H), 1.93-1.80 (m, 3H), 1.68-1.54 (m, 3H) ppm. MS: M/e 655 (M+1)⁺

### Compound B99:2-((S)-1-(2-fluoroacryloyl)-4-(5-(4-fluoronaphthalen-1-yl)-8-(((S)-1-methylpyrrolidin-2-yl)methoxy)-3,4-dihydro-2H-pyrano[2,3-f]quinazolin-10-yl)piperazin-2-yl)acetonitrile

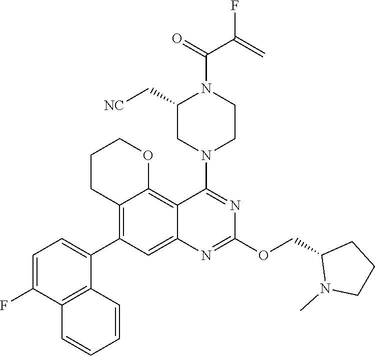 [SMILES: C=C(F)C(=O)N1CCN(c2nc(OC[C@@H]3CCCN3C)nc3cc(-c4ccc(F)c5ccccc45)c4c(c23)OCCC4)C[C@@H]1CC#N]

The Compound B99 was synthesized starting from the corresponding starting materials according the similar procedures described as those of compound B48 to get the product (45 mg). ¹H NMR (400 MHz, DMSO-d₆) δ 8.14 (d, J=8.2 Hz, 1H), 7.66 (t, J=7.6 Hz, 1H), 7.60-7.51 (m, 1H), 7.48-7.36 (m, 3H), 6.96 (d, J=6.2 Hz, 1H), 5.44-5.21 (m, 2H), 4.38-4.19 (m, 4H), 4.12-4.07 (m, 1H), 4.03-3.94 (m, 1H), 3.92-3.80 (m, 1H), 3.12-2.98 (m, 3H), 2.97-2.88 (m, 2H), 2.33-2.30 (m, 3H), 2.27-2.22 (m, 1H), 2.19-2.01 (m, 3H), 1.98-1.74 (m, 4H), 1.70-1.48 (m, 4H) ppm. MS: M/e 639 (M+1)⁺

### Compound B100: (S)-2-(4-(5-(8-chloronaphthalen-1-yl)-8-(3-(dimethylamino)azetidin-1-yl)-3,4-dihydro-2H-pyrano[2,3-f]quinazolin-10-yl)-1-(2-fluoroacryloyl)piperazin-2-yl)acetonitrile

### Step A: tert-butyl (S)-4-(5-(8-chloronaphthalen-1-yl)-8-(3-(dimethylamino)azetidin-1-yl)-3,4-dihydro-2H-pyrano[2,3-f]quinazolin-10-yl)-2-(cyanomethyl)piperazine-1-carboxylate

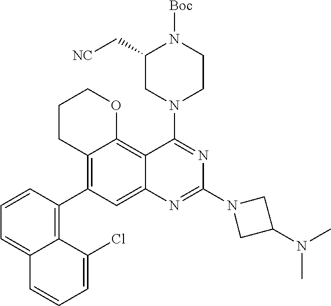 [SMILES: CN(C)C1CN(c2nc(N3CCN(C(=O)OC(C)(C)C)[C@@H](CC#N)C3)c3c4c(c(-c5cccc6cccc(Cl)c56)cc3n2)CCCO4)C1]

A mixture of tert-butyl (S)-4-(8-chloro-5-(8-chloronaphthalen-1-yl)-3,4-dihydro-2H-pyrano[2,3-f]quinazolin-10-yl)-2-(cyanomethyl)piperazine-1-carboxylate (100 mg, 0.165 mmol), N,N-dimethylazetidin-3-amine dihydrochloride (57 mg, 0.33 mmol), and DIEA (160 mg, 1.24 mmol) in isopropyl alcohol (3 mL) was stirred at 80° C. for 2 hrs. The mixture was concentrated, and diluted with 10 mL of CH₂Cl₂, washed with brine (5 mL×2), dried and concentrated to obtain the title compound (85 mg, crude). MS: M/e 668 (M+1)⁺.

### Step B: (S)-2-(4-(5-(8-chloronaphthalen-1-yl)-8-(3-(dimethylamino)azetidin-1-yl)-3,4-dihydro-2H-pyrano[2,3-f]quinazolin-10-yl)piperazin-2-yl)acetonitrile HCl salt

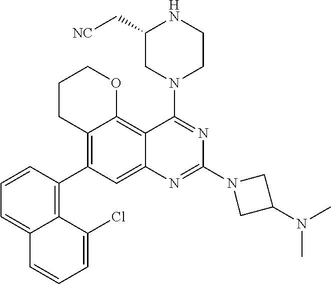 [SMILES: CN(C)C1CN(c2nc(N3CCN[C@@H](CC#N)C3)c3c4c(c(-c5cccc6cccc(Cl)c56)cc3n2)CCCO4)C1]

To a solution of tert-butyl (S)-4-(5-(8-chloronaphthalen-1-yl)-8-(3-(dimethylamino)azetidin-1-yl)-3,4-dihydro-2H-pyrano[2,3-f]quinazolin-10-yl)-2-(cyanomethyl)piperazine-1-carboxylate (85 mg, crude) in CH₂Cl₂/MeOH (3:1, 3 mL) was added HCl/Dioxane (4N, 1 mL) at rt and stirred for 2 hrs. The mixture was concentrated to dryness to obtain the title compound (70 mg, crude, HCl salt). MS: M/e 568 (M+1)⁺.

### Step C: (S)-2-(4-(5-(8-chloronaphthalen-1-yl)-8-(3-(dimethylamino)azetidin-1-yl)-3,4-dihydro-2H-pyrano[2,3-f]quinazolin-10-yl)-1-(2-fluoroacryloyl)piperazin-2-yl)acetonitrile

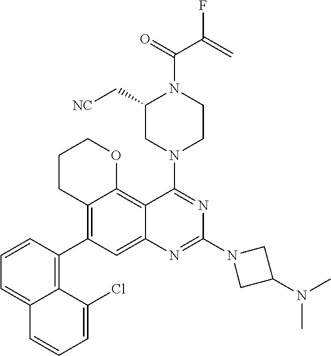 [SMILES: C=C(F)C(=O)N1CCN(c2nc(N3CC(N(C)C)C3)nc3cc(-c4cccc5cccc(Cl)c45)c4c(c23)OCCC4)C[C@@H]1CC#N]

To a mixture of the product of step B (70 mg, crude), 2-fluoroacrylic acid (22 mg, 0.24 mmol) and triethylamine (150 mg, 1.5 mmol) in THF (2 mL) was added T₃P (120 mg, 0.38 mmol) and the resulted mixture was stirred at rt for 16 hrs. The mixture was diluted with 20 mL of CH₂Cl₂, washed with NaHCO₃ (aq.) (5 mL×2), brine (5 mL×2), dried over Na₂SO₄, concentrated and purified by prep-TLC (CH₂Cl₂/MeOH (7M NH₃)=20:1) and prep-HPLC to obtain the title compound (7.5 mg, yield: 7% for 3 steps). ¹H NMR (400 MHz, CD₃OD) δ 8.05 (d, J=8.0 Hz, 1H), 7.98 (d, J=8.0 Hz, 1H), 7.66-7.53 (m, 2H), 7.51-7.43 (m, 1H), 7.38-7.29 (m, 1H), 6.96 (d, J=0.8 Hz, 1H), 5.54-5.20 (m, 2H), 5.08-4.90 (m, 1H), 4.73-4.53 (m, 1H), 4.42-4.28 (m, 4H), 4.25-4.02 (m, 4H), 3.79-3.37 (m, 4H), 3.11-2.87 (m, 2H), 2.41-2.30 (m, 6H), 2.31-2.21 (m, 2H), 2.03-1.89 (m, 2H) ppm. MS: M/e 640 (M+1)⁺.

### Compound B101 (atropisomer 1) and Compound B102 (atropisomer 2)

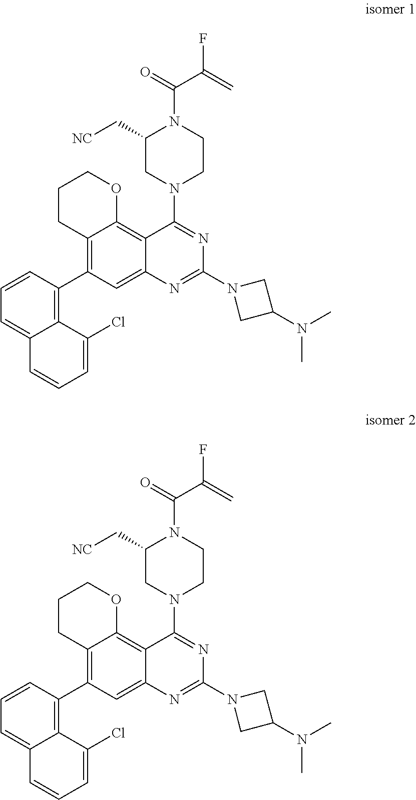 [SMILES: C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(N3CC(N(C)C)C3)nc3cc(-c4cccc5cccc(Cl)c45)c4c(c23)OCCC4)C[C@@H]1CC#N]

(Compound B100, 105 mg, 0.16 mmol) was purified by Prep-HPLC to afford Compound B101: (atropisomer 1, 38 mg) and Compound B102: (atropisomer 2, 35 mg).

Prep-HPLC condition

| Column | CHIRAL Cellulose-SB |
|---|---|
| Column size | 3 cm × 25 cm, 5 um |
| Injection | 4.0 mL |
| Mobile phase | Hex(0.5% 2M NH3-MEOH): ETOH = 50:50 |
| Flow rate | 45 mL/min |
| Wave length | UV 220 nm |
| Temperature | 35° C. |
| Sample solution | 3.3 mg/ml in ETOH:DCM = 3:1 |
| Prep-HPLC equipment | Prep-HPLC-YMC |

Compound 101 (atropisomer 1): ¹H NMR (400 MHz, CD₃OD) δ 8.01 (d, J=8.4 Hz, 1H), 7.95 (d, J=8.0 Hz, 1H), 7.65-7.57 (m, 1H), 7.57-7.50 (m, 1H), 7.48-7.40 (m, 1H), 7.40-7.32 (m, 1H), 7.01-6.94 (m, 1H), 5.36 (d, J=17.2 Hz, 1H), 5.28 (d, J=10.4 Hz, 1H), 4.34 (d, J=12.8 Hz, 2H), 4.29-4.18 (m, 3H), 4.06-3.93 (m, 3H), 3.39-3.32 (m, 2H), 3.27-3.19 (m, 2H), 3.19-2.91 (m, 4H), 2.30-2.17 (m, 8H), 1.99-1.88 (m, 2H) ppm. MS: M/e 640 (M+1)⁺.

Compound B102: (atropisomer 2): ¹H NMR (400 MHz, CD₃OD) δ 8.00 (d, J=8.0 Hz, 1H), 7.95 (d, J=8.0 Hz, 1H), 7.62-7.56 (m, 1H), 7.55 (d, J=7.2 Hz, 1H), 7.44 (t, J=7.6 Hz, 1H), 7.32 (d, J=7.2 Hz, 1H), 6.96 (s, 1H), 5.42-5.22 (m, 2H), 4.42-4.15 (m, 6H), 4.06-3.94 (m, 3H), 3.37-3.32 (m, 1H), 3.29-3.27 (m, 1H), 3.26-3.03 (m, 4H), 2.97 (dd, J=16.8, 6.4 Hz, 1H), 2.30-2.19 (m, 8H), 1.99-1.90 (m, 2H) ppm. MS: M/e 640 (M+1)⁺.

### Compound B103: 2-((S)-4-(5-(3-chloro-2-(trifluoromethyl)phenyl)-8-(((S)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

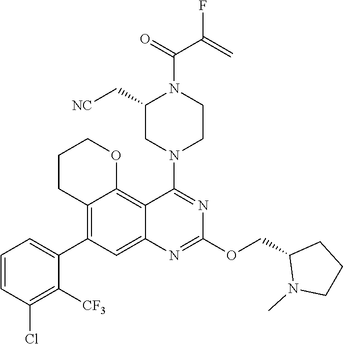 [SMILES: C=C(F)C(=O)N1CCN(c2nc(OC[C@@H]3CCCN3C)nc3cc(-c4cccc(Cl)c4C(F)(F)F)c4c(c23)OCCC4)C[C@@H]1CC#N]

The Compound B103 was synthesized starting from the corresponding starting materials according the similar procedures described as those of compound B66 to give the target compound (0.52 mg). ¹H NMR (400 MHz, CD₃OD) δ 8.50 (s, 1H), 7.72 (d, J=7.7 Hz, 1H), 7.65 (d, J=5.3 Hz, 1H), 7.22 (dd, J=17.1, 7.8 Hz, 1H), 5.40-5.28 (m, 2H), 4.69 (d, J=11.7 Hz, 1H), 4.61-4.50 (m, 2H), 4.45 (d, J=13.7 Hz, 2H), 4.34 (s, 1H), 4.06 (s, 1H), 3.48 (s, 4H), 3.00 (d, J=18.5 Hz, 3H), 2.89 (s, 3H), 2.50-2.35 (m, 2H), 2.31 (s, 1H), 2.04 (s, 5H), 1.30 (s, 2H) ppm. MS: M/e 673 (M+1)⁺.

### Compound B104 and B105: 2-((S)-4-(5-(8-chloronaphthalen-1-yl)-8-(((R)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 1) and 2-((S)-4-(5-(8-chloronaphthalen-1-yl)-8-(((R)-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 2)

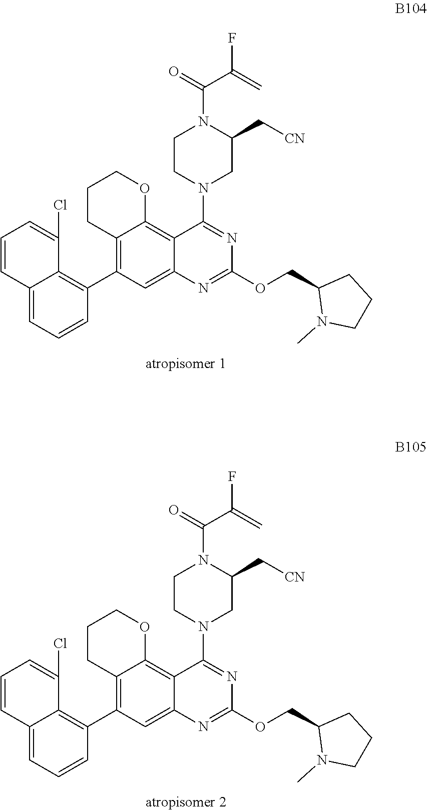 [SMILES: C=C(F)C(=O)N1CCN(c2nc(OC[C@H]3CCCN3C)nc3cc(-c4cccc5cccc(Cl)c45)c4c(c23)OCCC4)C[C@@H]1CC#N ; C=C(F)C(=O)N1CCN(c2nc(OC[C@H]3CCCN3C)nc3cc(-c4cccc5cccc(Cl)c45)c4c(c23)OCCC4)C[C@@H]1CC#N]

The target Compound was synthesized starting from the corresponding starting materials according the similar procedures described as those of compound B66 to get the product afford racemic product (75 mg). MS: M/e 655 (M+1)⁺. The racemic mixture was chiral-separated to afford Compound B104 (atropisomer 1, 17.81 mg) and Compound B105 (atropisomer 2 18.02 mg).

Prep-HPLC condition

| Column | CHIRAL Cellulose-SC |
|---|---|
| Column size | 2 cm × 25 cm, 5 um |
| Mobile phase A | MBTE |
| Mobile phase B | MeOH |
| Flow rate | 20 mL/min |
| Wave length | UV 220 nm |
| Temperature | 25° C. |
| Prep-HPLC equipment | Prep-HPLC-Gilson |

Compound B104 (atropisomer 1): 1H NMR (400 MHz, DMSO-d₆) δ 8.12 (d, J=8.4 Hz, 1H), 8.06 (d, J=8.1 Hz, 1H), 7.67 (t, J=7.7 Hz, 1H), 7.62 (d, J=7.4 Hz, 1H), 7.54 (t, J=7.5 Hz, 1H), 7.42 (d, J=7.0 Hz, 1H), 6.96 (s, 1H), 5.47-5.21 (m, 2H), 4.38-4.30 (m, 2H), 4.26 (s, 1H), 4.20-4.09 (m, 2H), 3.91 (d, J=13.0 Hz, 1H), 3.80-3.43 (m, 1H), 3.31-2.87 (m, 6H), 2.60-2.52 (m, 2H), 2.34 (s, 3H), 2.23-2.07 (m, 3H), 1.99-1.83 (m, 3H), 1.74-1.53 (m, 3H) ppm, MS: M/e 655 (M+1)⁺.

Compound B105 (atropisomer 2): 1H NMR (400 MHz, DMSO-d₆) δ 8.11 (d, J=8.2 Hz, 1H), 8.07 (d, J=8.1 Hz, 1H), 7.71-7.60 (m, 2H), 7.54 (t, J=7.9 Hz, 1H), 7.36 (d, J=6.8 Hz, 1H), 6.95 (s, 1H), 5.50-5.19 (m, 2H), 4.41-4.29 (m, 2H), 4.26 (s, 1H), 4.19-4.09 (m, 2H), 3.88 (d, J=12.9 Hz, 1H), 3.77-3.39 (m, 1H), 3.26-2.90 (m, 6H), 2.58-2.51 (m, 2H), 2.34 (s, 3H), 2.23-2.11 (m, 3H), 1.97-1.85 (m, 3H), 1.71-1.56 (m, 3H) ppm. MS: M/e 655 (M+1)⁺.

### Compound B106 and 107: 2-((S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 1) and 2-((S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile (atropisomer 2)

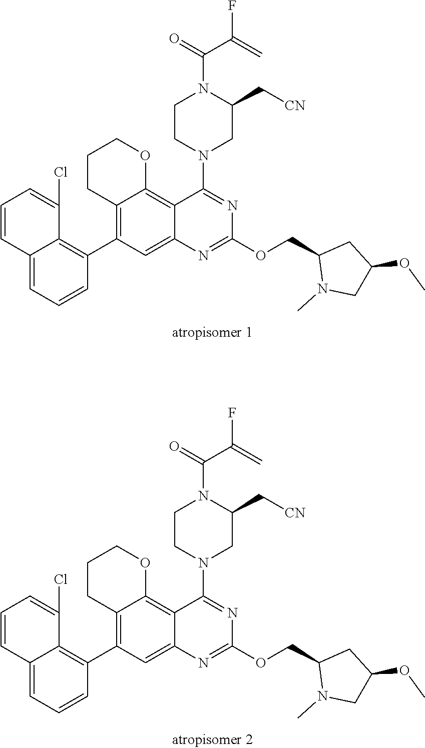 [SMILES: C=C(F)C(=O)N1CCN(c2nc(OC[C@H]3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OC[C@H]3C[C@@H](OC)CN3C)nc3cc(-c4cccc5cccc(Cl)c45)c4c(c23)OCCC4)C[C@@H]1CC#N]

### Step A: 1-(tert-butyl) 2-methyl (2R,4R)-4-methoxypyrrolidine-1,2-dicarboxylate

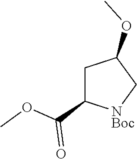 [SMILES: COC(=O)[C@H]1C[C@@H](OC)CN1C(=O)OC(C)(C)C]

To a stirred mixture of NaH (1 g, 25 mmol) and 1-(tert-butyl) 2-methyl (2R,4R)-4-hydroxypyrrolidine-1,2-dicarboxylate (2.5 g, 10 mmol) in DMF (25 ml) at rt for 0.5 h, was added Mel (4.35 g, 30.6 mmol). The mixture was stirred at rt overnight. After completed, the solution was quenched with water and then extracted with EA (20 ml×2). The organic layer was washed with brine (10 ml), dried and concentrated under reduced pressure to afford crude product (2.5 g), which was used directly for the next step without further purification. MS: M/e 260 (M+1)⁺.

### Step B: ((2R,4R)-4-methoxy-1-methylpyrrolidin-2-yl)methanol

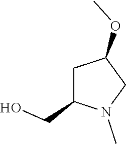

To a stirred mixture of 1-(tert-butyl) 2-methyl (2R,4R)-4-methoxypyrrolidine-1,2-dicarboxylate (2.5 g, 9.6 mmol) in THF (25 ml) at rt, was added LAH (0.73 g, 19.2 mmol). The mixture was stirred at 70° C. overnight. After completed, the solution was quenched with Na₂SO₄ 10H₂O, which was then filtered. The filtrate was dried and concentrated under reduced pressure to afford crude product (1.4 g), which was used directly for the next step without further purification. MS: M/e 146 (M+1)⁺.

### Step C: tert-butyl (S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)-2-(cyanomethyl)piperazine-1-carboxylate

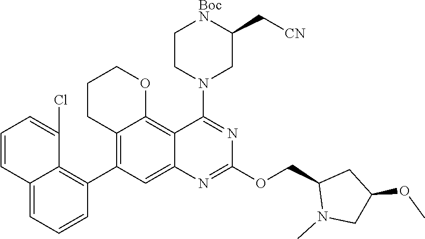 [SMILES: CO[C@@H]1C[C@H](COc2nc(N3CCN(C(=O)OC(C)(C)C)[C@@H](CC#N)C3)c3c4c(c(-c5cccc6cccc(Cl)c56)cc3n2)CCCO4)N(C)C1]

To a stirred mixture of ((2R,4R)-4-methoxy-1-methylpyrrolidin-2-yl)methanol (195 mg, 1.33 mmol) and NaH (60%, 53 mg, 1.33 mmol) in THF (20 ml) at rt for 3 h, was added tert-butyl (S)-4-(8-chloro-5-(8-chloronaphthalen-1-yl)-3,4-dihydro-2H-pyrano[2,3-f]quinazolin-10-yl)-2-(cyanomethyl)piperazine-1-carboxylate (400 mg, 0.66 mmol) at rt. The mixture was stirred at 70° C. overnight. After completed, the solution was quenched with water (15 ml) and then extracted with EA (15 ml×2). The organic layer was washed with brine (10 ml), dried and concentrated under reduced pressure. The residue was purified by flash column chromatography with 0-10% MeOH in DCM to afford product (330 mg, 70%). MS: M/e 713 (M+1)⁺.

### Step D: 2-((S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)piperazin-2-yl)acetonitrile. HCl salt

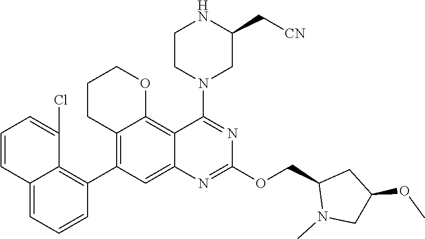 [SMILES: CO[C@@H]1C[C@H](COc2nc(N3CCN[C@@H](CC#N)C3)c3c4c(c(-c5cccc6cccc(Cl)c56)cc3n2)CCCO4)N(C)C1]

A solution of tert-butyl (S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)-2-(cyanomethyl)piperazine-1-carboxylate (330 mg, 0.46 mmol) and HCl/dioxane (4M, 10 ml) in DCM (10 ml) was stirred at rt for 1 h. After completed, the solution was concentrated under reduced pressure to afford product (280 mg, 100%), which was used directly for the next step without further purification. MS: M/e 613 (M+1)⁺.

### Step E: 2-((S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)-1-(2-fluoroacryloyl)piperazin-2-yl)acetonitrile

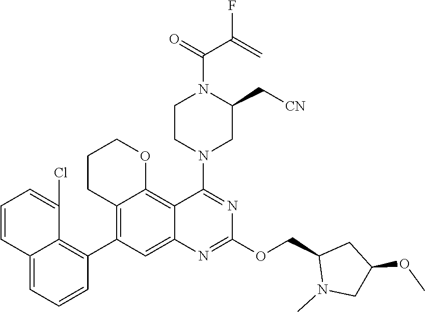 [SMILES: C=C(F)C(=O)N1CCN(c2nc(OC[C@H]3C[C@@H](OC)CN3C)nc3cc(-c4cccc5cccc(Cl)c45)c4c(c23)OCCC4)C[C@@H]1CC#N]

To a stirred solution of 2-((S)-4-(5-(8-chloronaphthalen-1-yl)-8-(((2R,4R)-4-methoxy-1-methylpyrrolidin-2-yl)methoxy)-3,4-dihydro-2H-pyrano[2,3-f]quinazolin-10-yl)piperazin-2-yl)acetonitrile (280 mg, 0.46 mmol), 2-fluoroacrylic acid (61.6 mg, 0.68 mmol) and DIEA (176 mg, 1.36 mmol) in DMF (10 ml), was added HATU (260 mg, 0.68 mmol) at rt and stirred for 2 h. After completed, the solution was poured into water (10 ml) and then extracted with EA (15 ml×2). The organic layer was washed with brine (10 ml), dried and concentrated under reduced pressure. The residue was purified by flash column chromatography with 0-10% MeOH in DCM to afford product (270 mg). MS: M/e 685 (M+1)⁺.

The racemic mixture was chiral-separated by twice to afford atropisomer 1 (39.06 mg) and atropisomer 2 (4.3 mg).

First Prep-HPLC Condition

| Column | CHIRAL Cellulose-SB |
|---|---|
| Column Size | 2 cm × 25 cm, 5 um |
| Mobile Phase A | METH (0.5% 2M NH₃-MEOH) |
| Mobile Phase B | MEOH |
| Flow Rate | 20 mL/min |
| Wave Length | UV 220 nm |
| Temperature | 25° C. |
| Prep-HPLC Equipment | Prep-HPLC-Gilson |

Second Prep-HPLC Condition

| Column | CHIRAL Cellulose-SB |
|---|---|
| Column Size | 2 cm × 25 cm, 5 um |
| Mobile Phase A | HEX (0.5% 2M NH₃-MEOH) |
| Mobile Phase B | ETOH |
| Flow Rate | 20 mL/min |
| Wave Length | UV 220 nm |
| Temperature | 25° C. |
| Prep-HPLC Equipment | Prep-HPLC-Gilson |

B106 (atropisomer 1): 1H NMR (400 MHz, DMSO-d) δ 8.12 (d, J=8.3 Hz, 1H), 8.06 (d, J=8.2 Hz, 1H), 7.67 (t, J=7.6 Hz, 1H), 7.62 (d, J=7.4 Hz, 1H), 7.53 (t, J=7.8 Hz, 1H), 7.42 (d, J=7.0 Hz, 1H), 6.97 (s, 1H), 5.51-5.16 (m, 2H), 4.92 (s, 1H), 4.42-4.15 (m, 6H), 3.91 (d, J=13.0 Hz, 1H), 3.82 (s, 1H), 3.40 (s, 1H), 3.27 (s, 1H), 3.16 (s, 3H), 3.12-2.91 (m, 4H), 2.70-2.52 (m, 2H), 2.40-2.21 (m, 5H), 2.20-2.10 (m, 2H), 1.88 (s, 2H) ppm. MS: M/e 685 (M+1)⁺.

B107 (atropisomer 2): 1H NMR (400 MHz, DMSO-d) δ 8.12 (d, J=7.7 Hz, 1H), 8.06 (d, J=8.1 Hz, 1H), 7.67 (t, J=7.6 Hz, 1H), 7.62 (d, J=7.4 Hz, 1H), 7.53 (t, J=7.6 Hz, 1H), 7.41 (d, J=7.2 Hz, 1H), 6.97 (s, 1H), 5.49-5.19 (m, 2H), 5.11-4.52 (m, 1H), 4.45-4.10 (m, 6H), 3.95-3.81 (m, 2H), 3.77-3.46 (m, 1H), 3.29-3.24 (m, 1H), 3.19 (s, 3H), 3.15-2.88 (m, 4H), 2.83-2.54 (m, 2H), 2.35 (s, 2H), 2.25-2.06 (m, 3H), 1.88 (s, 4H) ppm. MS: M/e 685 (M+1)⁺.

### Biochemical Functional Assay

The KRAS (aa 1-169) G12C, C51S, C80L, C118S with a His-tag was expressed, purified and loaded with GDP in house. All protein and substrate solutions were prepared in assay buffer containing 25 mM HEPES pH7.5, 10 mM MgCl2, and 0.01% Triton X-100. Purified GDP-loaded KRAS (aa 1-169) G12C, C51S, C80L, C118S protein was pre-incubated with a serially diluted compound at 24° C. for 3 hrs. Purified SOS1 (aa 564-1049) protein, GTPyS (Sigma) and GST-cRaf RBD (aa 1-149) were then added to each well and incubated at 24° C. for additional 3 hrs. This addition initiates the nucleotide exchange reaction and transition of inactive GDP loaded KRAS G12C to active GTPyS KRAS G12C which binds to GST-cRaf RBD. Following the incubation, Mab Anti-6HIS-T cryptate (Cisbio) and Mab Anti GST-XL665 (Cisbio) were added and further incubated at 24° C. for 3 hrs. The binding interaction between active GTPyS KRAS G12C and GST-cRaf RBD brings the Tb and XL665 into close proximity enabling an increased FRET signal (Ex337 nm, Em665 nm/620 nm). The inhibition percentage of nucleotide exchange reaction in presence of increasing concentrations of compounds was calculated based on the ratio of fluorescence at 665 nm to that at 620 nm detected on a BMG PHERAstar FSX instrument. The IC50 value of each compound was calculated from fitting the data to the four-parameter logistic model by Dotmatics.

TABLE 1
Compounds A series

| Compound No. | IC50 (nmol) | Compound No. | IC50 (nmol) |
|---|---|---|---|
| A1 | 5500 | A2 | 2120 |
| A3 | 559 | A4 | 89.6 |
| A5 | 1690 | A6 | 3540 |
| A7 | 43.1 | A8 | 82.1 |
| A9 | 218 | A10 | 598 |
| A11 | 799 | A12 | 1660 |
| A13 | 3310 | A14 | 79.4 |
| A15 | 92.6 | A16 | 89.8 |
| A17 | 31.7 | A18 | 37.8 |
| A19 | 691 | A20 | 14.9 |
| A21 | 5 | A22 | 256 |
| A23 | 346 | A24 | 262 |
| A25 | 15.5 | A26 | 9.9 |
| A27 | >10 uM | A28 | >10 uM |
| A29 | 9.3 | A30 | 1960 |
| A31 | 137 | A32 | 12 |
| A33 | 233 | A34 | 2110 |

TABLE 2
Compounds B series

| Compound No. | IC50 (nmol) | Compound No. | IC50 (nmol) |
|---|---|---|---|
| B1 | 3 | B2 | 2240 |
| B3 | 6.5 | B4 | 4.7 |
| B5 | 7.9 | B6 | 34 |
| B7 | 1090 | B8 | 255 |
| B9 | 456 | B10 | 235 |
| B11 | 7.81 | B12 | 4.6 |
| B13 | 325 | B14 | >10 uM |
| B15 | 107 | B16 | >10 uM |
| B17 | >10 uM | B18 | >10 uM |
| B19 | >10 uM | B20 | 16 |
| B21 | >10 uM | B22 | 568 |
| B23 | 202 | B24 | >10 uM |
| B25 | 138 | B26 | 239 |
| B27 | 321 | B28 | 113 |
| B29 | 4.51 | B30 | 9.6 |
| B31 | 984 | B32 | 226 |
| B33 | 51 | B34 | 65 |
| B35 | 8.4 | B36 | 47 |
| B37 | 8 | B38 | 913 |
| B39 | 1040 | B40 | 14 |
| B41 | 3.8 | B42 | 43 |
| B43 | 13 | B44 | 6350 |
| B45 | 2020 | B46 | 621 |
| B47 | 25.03 | B48 | 14 |
| B49 | 128 | B50 | 289 |
| B51 | 1010 | B52 | 654 |
| B53 | 28 | B54 | 40 |
| B55 | 134 | B56 | 632 |
| B57 | 1020 | B58 | 958 |
| B59 | 745 | B60 | 252 |
| B61 | 186 | B62 | 642 |
| B63 | 148 | B64 | 247 |
| B65 | 30 | B66 | 9.3 |
| B67 | 4.6 | B68 | 177 |
| B69 | 1220 | B70 | 35 |
| B71 | 304 | B72 | 194 |
| B73 | 50 | B74 | 837 |
| B75 | 44 | B76 | 279 |
| B77 | 535 | B78 | 12 |
| B79 | 59 | B80 | 222 |
| B81 | 48 | B82 | 6.2 |
| B83 | 1700 | B84 | 483 |
| B85 | 89 | B86 | 19 |
| B87 | 1520 | B88 | 92 |
| B89 | 691 | B90 | 37 |
| B91 | 15 | B92 | 381 |
| B93 | 11 | B94 | 4.63 |
| B95 | 11.5 | B96 | 140.4 |
| B97 | 507.9 | B98 | 461.9 |
| B99 | 152.3 | B100 | 18 |
| B101 | 6.53 | B102 | 354.4 |
| B103 | 6.95 | B104 | 8.16 |
| B105 | 289.8 | B106 | 9.54 |
| B107 | 3.26 | | |

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound of Formula (I): 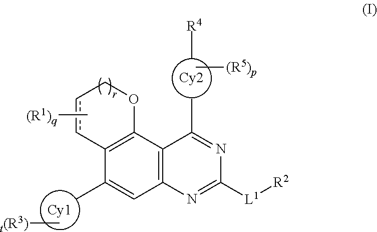
or a pharmaceutically acceptable salt thereof, or a stereoisomer thereof, wherein [symbol]
is a single bond or a double bond;
Cy1 is selected from —C₅₋₁₂cycloalkyl, 5- to 12-membered heterocyclyl, —C₆₋₁₂aryl or 5- to 12-membered heteroaryl;
Cy2 is selected from 4- to 12-membered cycloalkyl or 4- to 12-membered heterocyclyl;
L¹ is selected from a single bond, —CO—NR^{a}—, —NR^{a}—CO—, —O—, —NR^{a}—, —NR^{a}(CH₂)ₘ—, —S—, —(CH₂)ₘ—, —O—(CH₂)ₘ—, —O—CH(R^{a})—, —CH(R^{a})—, —CH(R^{a})(CH₂)ₘ—, —(CH₂)ₘ—O—, —CO—, —SO₂—, cycloalkylene, oxetandiyl, tetrahydrofurandiyl, tetrahydropyrandiyl, azetidindiyl, pyrrilidindiyl, piperidindiyl, or piperizindiyl;
R¹ is selected from —H, —C₁₋₈alkyl, —C₂₋₈alkenyl, —C₂₋₈alkynyl, —NR^{b}R^{c}, —C₃₋₁₂cycloalkyl, —C₃₋₁₂heterocyclyl, —C₆₋₁₂aryl, or —C₅₋₁₂heteroaryl, each of said —C₁₋₈alkyl, —C₂₋₈alkenyl, —C₂₋₈alkynyl, —C₃₋₁₂cycloalkyl, —C₃₋₁₂heterocyclyl, —C₆₋₁₂aryl, or —C₅₋₁₂heteroaryl is optionally substituted with at least one R⁶;
R² is selected from —H, —C₁₋₈alkyl, —NR^{b}R^{c}, —C₃₋₁₂cycloalkyl, 3- to 12-membered heterocyclyl, —C₆₋₁₂aryl, or 5- to 12-membered heteroaryl, wherein each of said —C₃₋₁₂cycloalkyl, 3- to 12-membered heterocyclyl, —C₆₋₁₂aryl, or 5- to 12-membered heteroaryl is optionally substituted with at least one R⁶;
R⁶, at each of its occurrences, is independently —C₁₋₈alkyl, halogen, hydroxy, oxo, —C₁₋₈alkoxy, —NR^{b}R^{c}, —C₃₋₈cycloalkyl, 3- to 8-membered heterocyclyl, —C₆₋₁₂aryl, or 5- to 12-membered heteroaryl, wherein each of said —C₁₋₈alkyl, —C₁₋₈alkoxy, —C₃₋₈cycloalkyl, 3- to 8-membered heterocyclyl, —C₆₋₁₂aryl, or 5- to 12-membered heteroaryl is optionally substituted with at least one halogen, hydroxy, amino, —C₁₋₈alkoxy, —NR^{b}R^{c}, —CN, cycloalkyl, heterocyclyl, aryl or heteroaryl;
R³ is selected from hydrogen, halogen, oxo, —C₁₋₈alkyl, —C₂₋₈alkenyl, —C₂₋₈alkynyl, —C₃₋₈cycloalkyl, 3- to 8-membered heterocyclyl, —C₆₋₁₂aryl, 5- to 8-membered heteroaryl, —OR^{a}, —NR^{a}R^{b} or —CN, wherein each of said —C₁₋₈alkyl, —C₂₋₈alkenyl, —C₂₋₈alkynyl, —C₃₋₈cycloalkyl, 3- to 8-membered heterocyclyl, —C₆₋₁₂aryl, or 5- to 12-membered heteroaryl is optionally substituted with at least one halogen, hydroxy, —C₁₋₈alkoxy, —C₃₋₈cycloalkyl, 3- to 8-membered heterocyclyl, —C₆₋₁₂aryl, or 5- to 12-membered heteroaryl; or
two R³ together with the atom(s) to which they are attached, form a 3- to 8-membered ring, said ring comprising 0, 1 or 2 additional heteroatom(s) independently selected from nitrogen, oxygen or optionally oxidized sulfur as ring member(s), said ring is optionally substituted with at least one halogen, hydroxy, amino, —C₁₋₈alkyl, —C₁₋₈alkoxy, —NR^{b}R^{c}, —CN, cycloalkyl, heterocyclyl, aryl or heteroaryl;
R⁵ is selected from —C₁₋₈alkyl, —C₂₋₈alkenyl, —C₂₋₈alkynyl, oxo, —NR^{b}R^{c}, —CO—NR^{b}R^{c}, cycloalkyl, heterocyclyl, aryl, or heteroaryl, —(CH₂)ₘ—CN, or hydrogen;
R⁴ is selected from 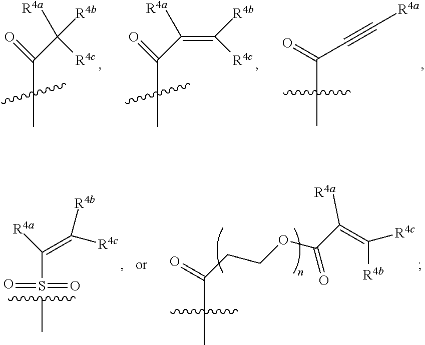
wherein each of R^{4a}, R^{4b} and R^{4c} is independently hydrogen, deuterium (D), cyano (CN), halogen, —OR^{a}, —C₁₋₈alkyl, —C₃₋₁₂cycloalkyl, —C₃₋₁₂heterocyclyl, —C₆₋₁₂aryl, —C₅₋₁₂heteroaryl, —NR^{b}R^{c}, or —CONR^{b}R^{c}, each of said —C₁₋₈alkyl, —C₃₋₁₂cycloalkyl, —C₃₋₁₂heterocyclyl, —C₆₋₁₂aryl or —C₅₋₁₂heteroaryl is optionally substituted with at least one R⁶;
wherein each of R^{a}, R^{b} and R^{c} is independently hydrogen, deuterium (D), cyano (CN), halogen, hydroxy, —C₁₋₈alkoxy, —C₁₋₈alkyl, —C₂₋₈alkenyl, —C₂₋₈alkynyl, —C₃₋₈cycloalkyl, 3- to 12-membered heterocyclyl, aryl, heteroaryl, —NR^{d}R^{e}, or —CO—NR^{d}R^{e}, each of said —C₁₋₈alkyl, —C₂₋₈alkenyl, —C₂₋₈alkynyl, cycloalkyl, heterocyclyl, aryl, or heteroaryl is optionally substituted with R^{f}; or
optionally (R^{a} and R^{b}), or (R^{a} and R^{c}) together with the atom(s) to which they are attached, form a 4- to 6 membered ring, said ring is optionally substituted with at least one R^{g};
each R^{f} is selected from halogen, hydroxy, oxo, —C₁₋₈alkoxy, —NR^{d}R^{e}, —CO—NR^{d}R^{e}, —NR^{d}—CO—R^{e}, cycloalkyl, heterocyclyl, aryl, or heteroaryl, wherein each of said —C₁₋₈alkoxy, cycloalkyl, heterocyclyl, aryl, or heteroaryl is optionally substituted with halogen, hydroxy or —C₁₋₄alkyl;
R^{a}, R^{e} and R^{g} are each independently hydrogen, deuterium (D), halogen, or —C₁₋₈alkyl; each of said —C₁₋₈alkyl is optionally substituted with at least one halogen, oxo, CF₃ or —COCH₃;
p, q and t are independently selected from 0, 1, 2, 3 or 4;
r is selected from 0, 1 or 2;
each m and n are independently 0, 1, 2, 3, 4, 5 or 6.

2. The compound according to claim 1, wherein Cy1 is selected from 6-, 7-, 8-, 9-, or 10-membered ring, wherein said ring is saturated or unsaturated ring comprising 0, 1 or 2 heteroatom(s) independently selected from nitrogen, oxygen or optionally oxidized sulfur as ring member(s).

3. The compound according to claim 1, wherein Cy1 is selected from naphthalene, benzene, benzopyrazole, pyridine, benzocyclopentane, benzothiazole, benzimidazole, indole, quinoline, isoquinoline, benzotetrahydrofuran, tetrahydroquinoline, tetrahydroisoquinoline, dihydroindole, 1,2-dihydronaphthalene, 1,4-dihydronaphthalene, or tetralin.

4. The compound according to claim 1, wherein R³ is selected from hydrogen, F, Cl, Br, I, oxo, methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole, —OH, —OCH₃, —OC₂H₅, —OC₃H₇, —OC₄H₉, —OC₅H₁₁, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉ or —CN, each of said methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole is optionally substituted with at least one F, Cl, Br, I, OH, —OCH₃, —OC₂H₅, —OC₃H₇, —OC₄H₉, —OC₅H₁₁, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, or benzopyrazole.

5. The compound according to claim 4, wherein R³ is selected from hydrogen, F, Cl, Br, I, oxo, —CH₃, —C₂H₅, —C₃H₇, —C₄H₉, —CF₃, cyclopropyl, cyclobutyl, cyclopentyl, —OH, —OCH₃, —OC₂H₅, —OC₃H₇, —OC₄H₉, —OC₅H₁₁, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉ or —CN.

6. The compound according to claim 1, wherein two R³ together with the atom(s) to which they are attached, form a 3-, 4-, 5-, 6-, 7- or 8-membered ring, said ring comprising 0, 1 or 2 additional heteroatom(s) independently selected from nitrogen or oxygen as ring member(s), said ring is optionally substituted with at least one F, Cl, Br, I, OH, NH₂, —CH₃, —C₂H₅, —C₃H₇, —C₄H₉, —C₅H₁₁, —OCH₃, —OC₂H₅, —OC₃H₇, —OC₄H₉, —OC₅H₁₁, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CN, cyclopropyl, cyclobutyl, cyclopentyl, cyclohexyl, cycloheptyl, cyclooctyl, azacyclopropyl, azacyclobutyl, tetrahydropyrrole, piperidine, piperazine, morpholine, propylene oxide, oxacyclobutane, oxacyclopentane, oxacyclohexane, phenyl, pyrrole, imidazole, pyrazole, thiazole, thiophene, oxazole, pyridine, indole, quinoline, isoquinoline, benzimidazole, benzothiazole, or benzopyrazole.

7. The compound according to claim 6, wherein two R together with the atom(s) to which they are attached, form a ring, said ring is selected from phenyl, pyrrole, imidazole, pyrazole, thiazole, thiophene, oxazole, pyridine, indole, quinoline, isoquinoline, benzimidazole, benzothiazole or benzopyrazole, each of said phenyl, pyrrole, imidazole, pyrazole, thiazole, thiophene, oxazole, pyridine, indole, quinoline, isoquinoline, benzimidazole, benzothiazole or benzopyrazole is optionally substituted with at least one F, Cl, Br, I, OH, NH₂, —CH₃, —C₂H₅, —C₃H₇, —C₄H₉, —C₅H₁₁, —OCH₃, —OC₂H₅, —OC₃H₇, —OC₄H₉, —OC₅H₁₁, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, or —CN.

8. The compound according to claim 1, wherein Cy1 is selected from

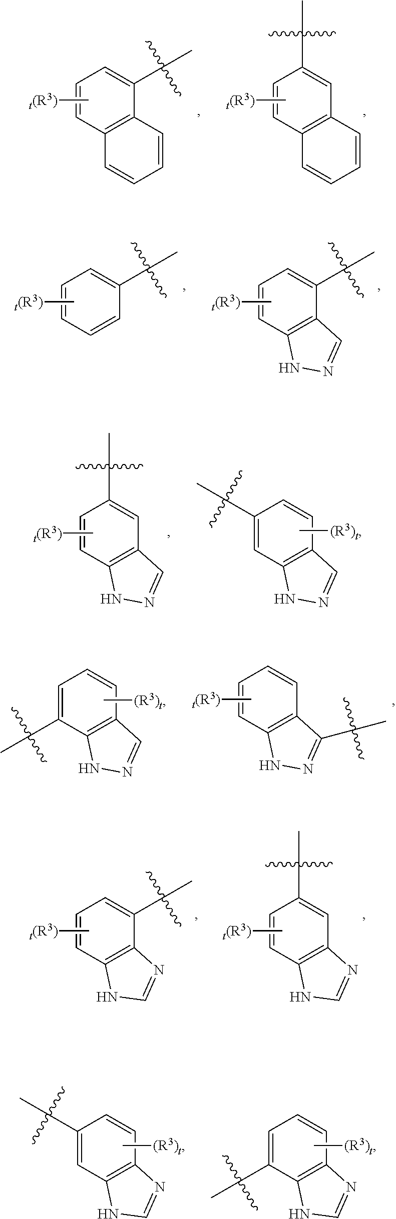

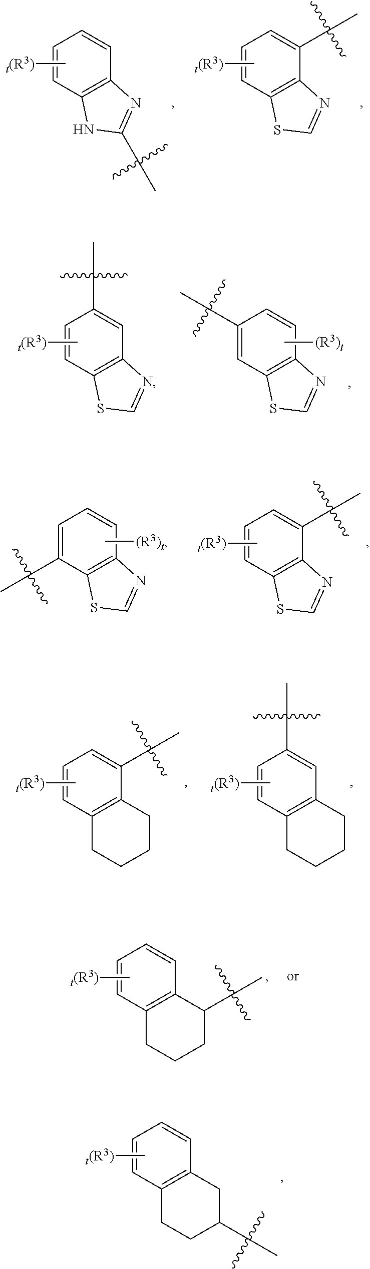

9. The compound according to claim 1, wherein 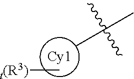
moiety is selected from

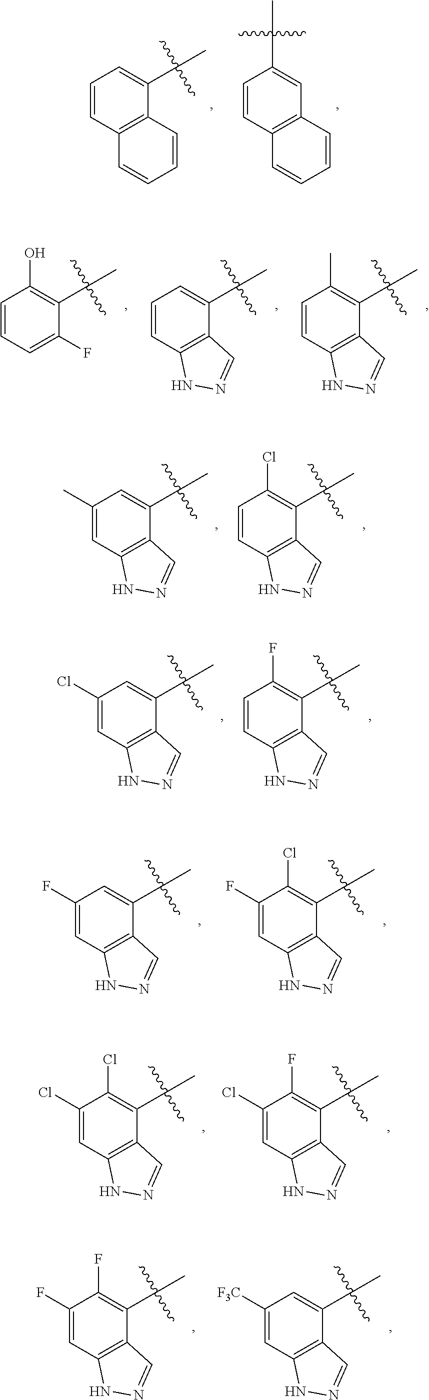

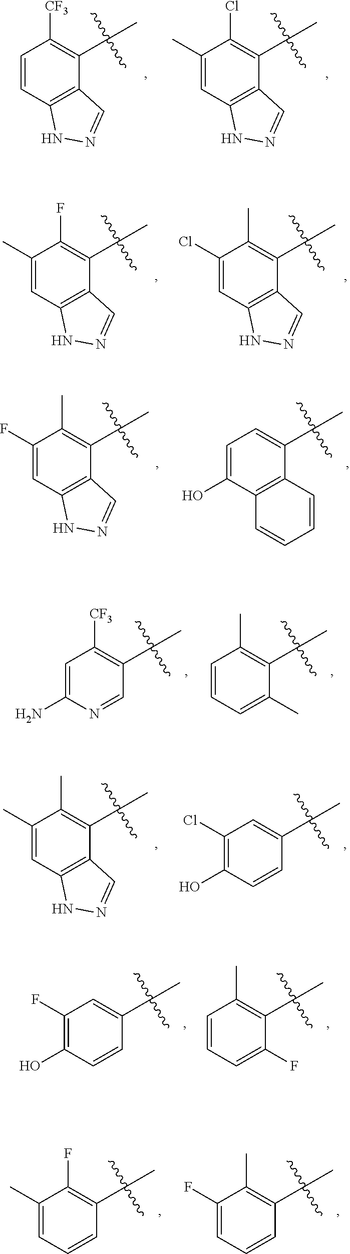

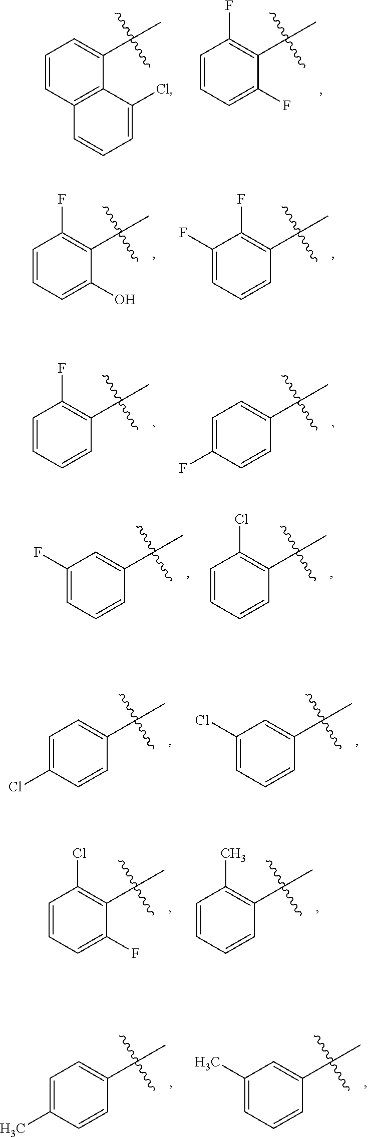

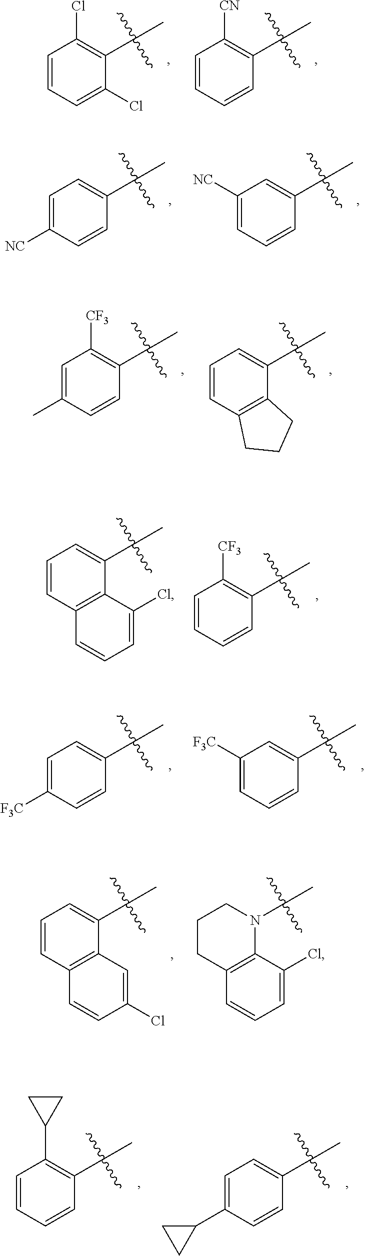

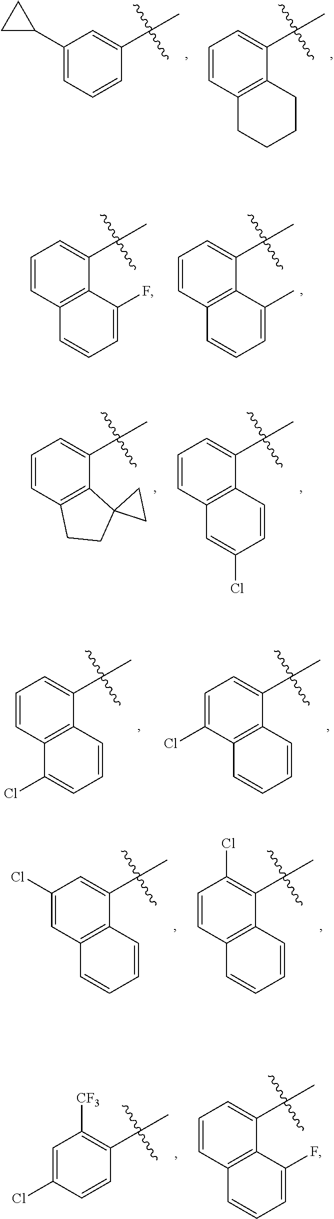

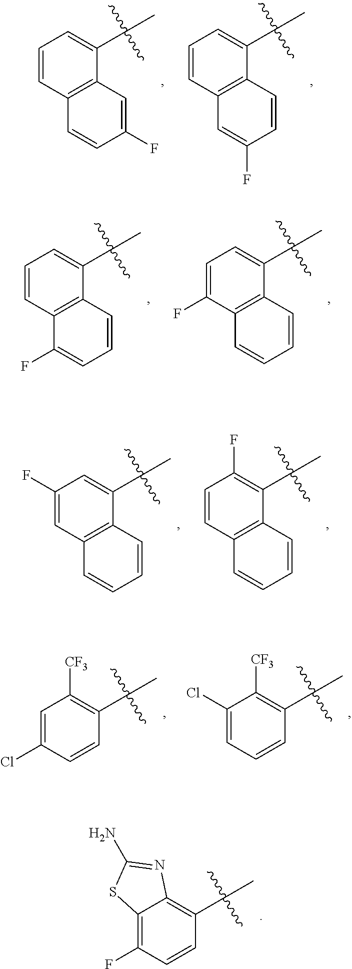

10. The compound according to claim 1, wherein Cy2 is selected from 6-, 7-, 8-, 9-, or 10-membered ring, said ring is saturated or unsaturated ring comprising 0, 1 or 2 additional heteroatom(s) independently selected from nitrogen, oxygen or optionally oxidized sulfur as ring member(s).

11. The compound according to claim 10, wherein Cy2 is selected from 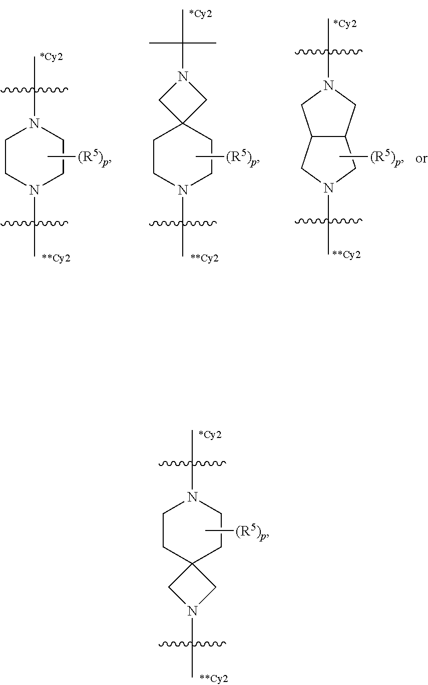
wherein *^{Cy2} refers to the position attached to R⁴ moiety, and **^{Cy2} refers to the position attached to the 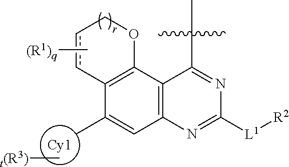
moiety.

12. The compound according to claim 11, wherein R⁵ is selected from —H, methyl, ethyl, propyl, butyl, amyl, hexyl, heptyl, octyl, cyclopropyl, cyclobutyl, cyclopentyl, cyclohexyl, cycloheptyl, cyclooctyl, phenyl, oxo, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CO—N(CH₃)₂, —CO—N(C₂H₅)₂, —CO—N(C₃H₇)₂, —CO—N(C₄H₉)₂, —CO—N(CH₃)C₂H₅, —CO—N(CH₃)C₃H₇, —CO—N(CH₃)C₄H₉, —CO—N(C₂H₅)C₃H₇, —CO—N(C₂H₅)C₄H₉, —CO—N(C₃H₇)C₄H₉, —CH₂—CN, —(CH₂)₂—CN, —(CH₂)₃—CN; preferably methyl or —CH₂—CN.

13. The compound according to claim 1, wherein R⁴ is selected from 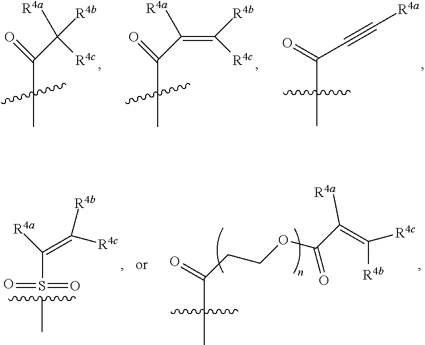
wherein each of R^{4a}, R^{4b} and R^{4c} is independently hydrogen, deuterium (D), cyano (CN), F, Cl, Br, I, hydroxy, —OCH₃, —OC₂H₅, —OC₃H₇, —OC₄Hg, —OC₅H₁₁, methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CO—NH₂, —CO—N(CH₃)₂, —CO—N(C₂H₅)₂, —CO—N(C₃H₇)₂, —CO—N(C₄H₉)₂, —CO—N(CH₃)C₂H₅, —CO—N(CH₃)C₃H₇, —CO—N(CH₃)C₄H₉, —CO—N(C₂H₅)C₃H₇, —CO—N(C₂H₅)C₄H₉, or —CO—N(C₃H₇)C₄H₉, each of said methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, benzopyrazole is optionally substituted with R^{f}; each R^{f} is selected from F, Cl, Br, I, oxo, hydroxy, —OCH₃, —OC₂H₅, —OC₃H₇, —OC₄H₉, —OC₅H₁₁, —NH₂, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉, —CO—NH₂, —CO—N(CH₃)₂, —CO—N(C₂H₅)₂, —CO—N(C₃H₇)₂, —CO—N(C₄H₉)₂, —CO—N(CH₃)C₂H₅, —CO—N(CH₃)C₃H₇, —CO—N(CH₃)C₄H₉, —CO—N(C₂H₅)C₃H₇, —CO—N(C₂H₅)C₄H₉, —CO—N(C₃H₇)C₄H₉, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, pyrrole, imidazole, pyrazole, thiazole, thiophene, oxazole, pyridine, indole, quinoline, isoquinoline, benzimidazole, benzothiazole, or benzopyrazole.

14. The compound according to claim 13, wherein R⁴ is selected from

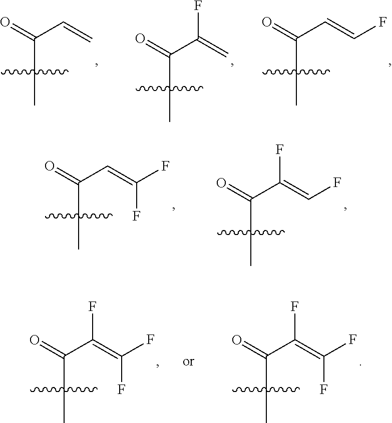

15. The compound according to claim 1, wherein the 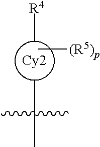
moiety is selected from

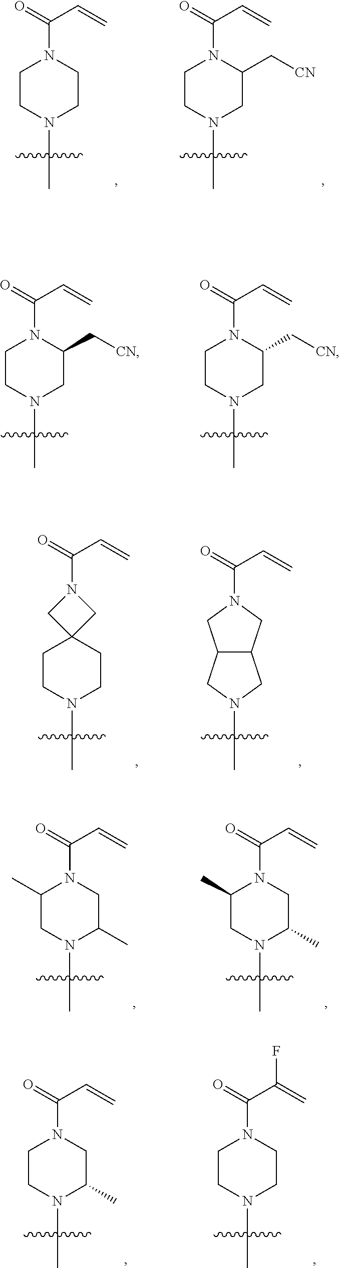

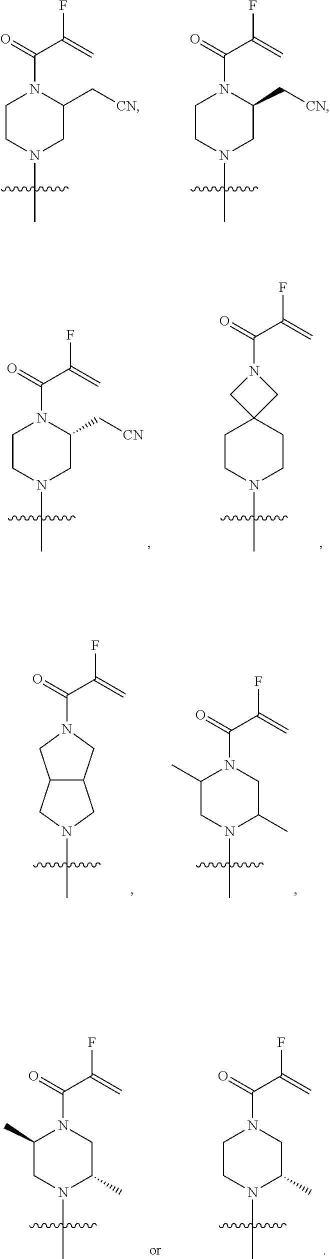

16. The compound according to claim 1, wherein R¹ is selected from —H, methyl, ethyl, propyl, butyl, amyl, hexyl, heptyl, octyl, cyclopropyl, cyclobutyl, cyclopentyl, cyclohexyl, cycloheptyl, cyclooctyl or phenyl, each of said methyl, ethyl, propyl, butyl, amyl, hexyl, heptyl, octyl, cyclopropyl, cyclobutyl, cyclopentyl, cyclohexyl, cycloheptyl, cyclooctyl or phenyl is optionally substituted with at least one R⁶.

17. The compound according to claim 16, wherein R¹ is selected from —H, methyl, ethyl, propyl, butyl, amyl, hexyl, heptyl, octyl, cyclopropyl, cyclobutyl, cyclopentyl, cyclohexyl, cycloheptyl, cyclooctyl or phenyl.

18. The compound according to claim 1, wherein R² is selected from —H, methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, or —N(C₃H₇)C₄H₉, each of said methyl, ethyl, propyl, butyl, amyl, hexyl, heptyl, octyl, cyclopropyl, cyclobutyl, cyclopentyl, cyclohexyl, cycloheptyl, cyclooctyl, phenyl, azacyclopropyl, azacyclobutyl, tetrahydropyrrole, piperidine, piperazine, morpholine, propylene oxide, oxacyclobutane, oxacyclopentane, oxacyclohexane, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉ is optionally substituted with at least one R⁶.

19. The compound according to claim 18, wherein R² is selected from H, —N(CH₃)₂, —N(C₂H₅)₂, —N(C₃H₇)₂, —N(C₄H₉)₂, —N(CH₃)C₂H₅, —N(CH₃)C₃H₇, —N(CH₃)C₄H₉, —N(C₂H₅)C₃H₇, —N(C₂H₅)C₄H₉, —N(C₃H₇)C₄H₉,

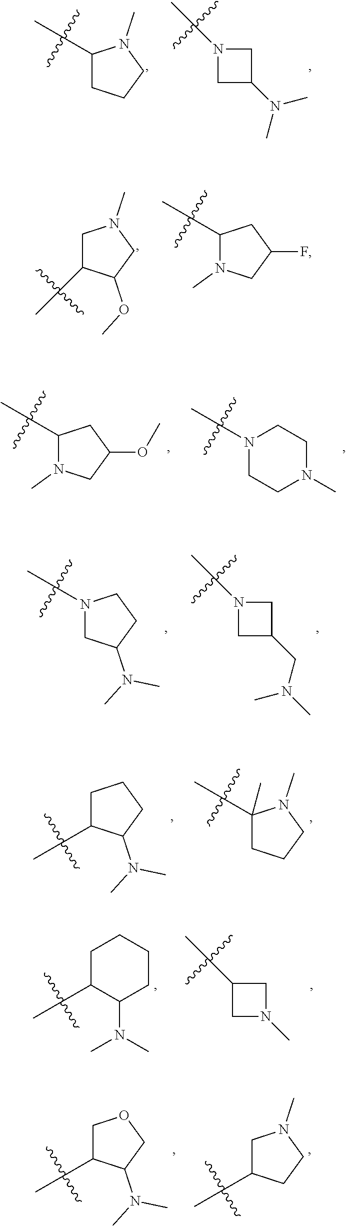

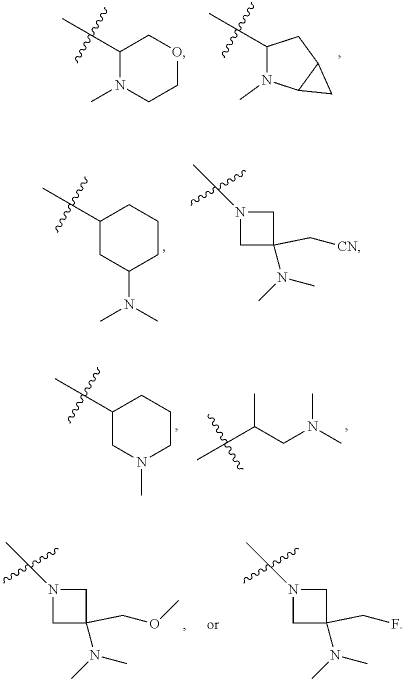

20. The compound according to claim 1, wherein the 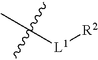
moiety is selected from —H,

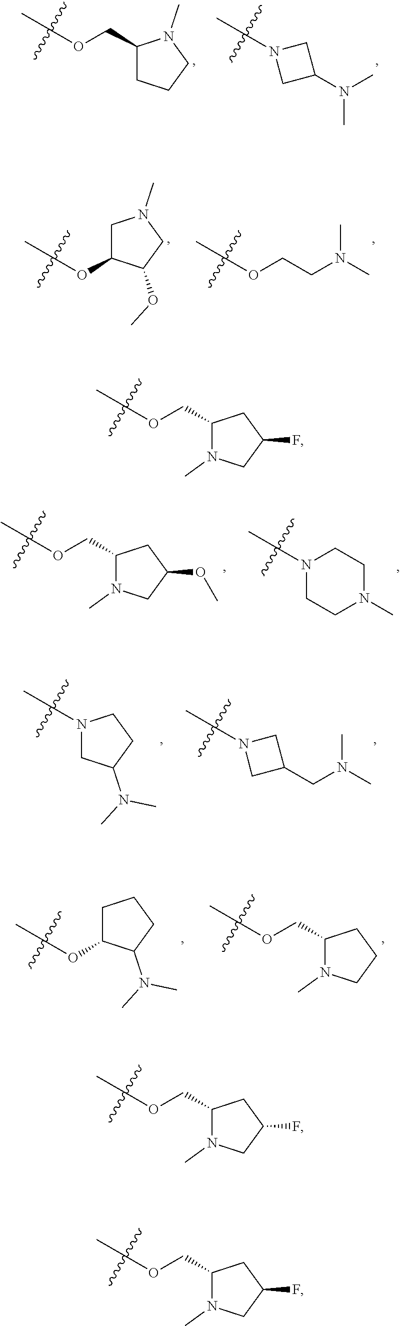

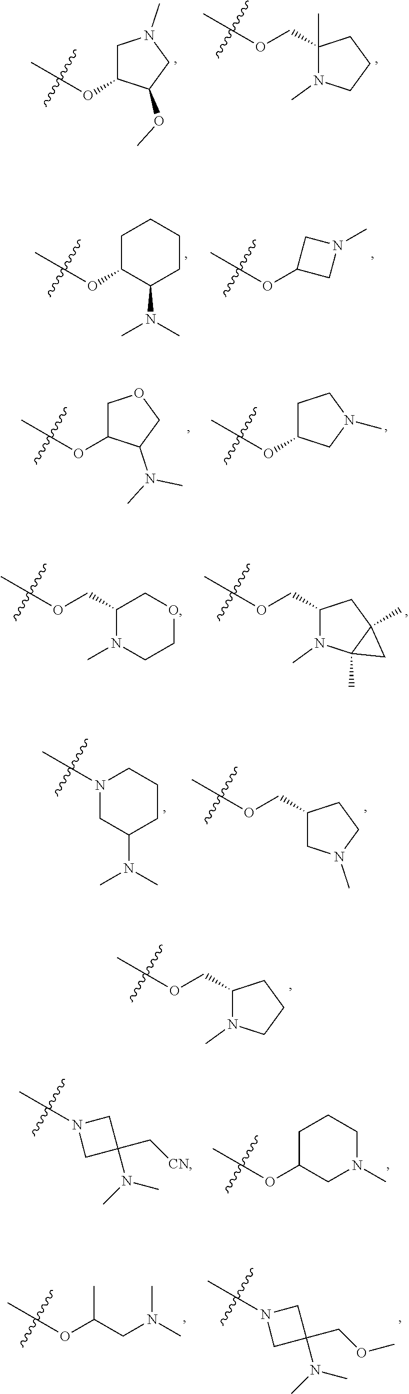

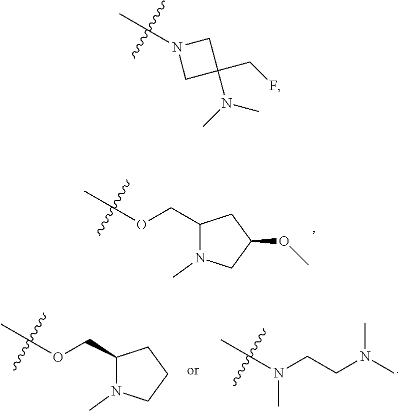

21. The compound according to claim 1, wherein r is 0, and [symbol] is a single bond or a double bond; or r is 1, and [symbol] is a single bond or a double bond.

22. A compound selected from the group consisting of: 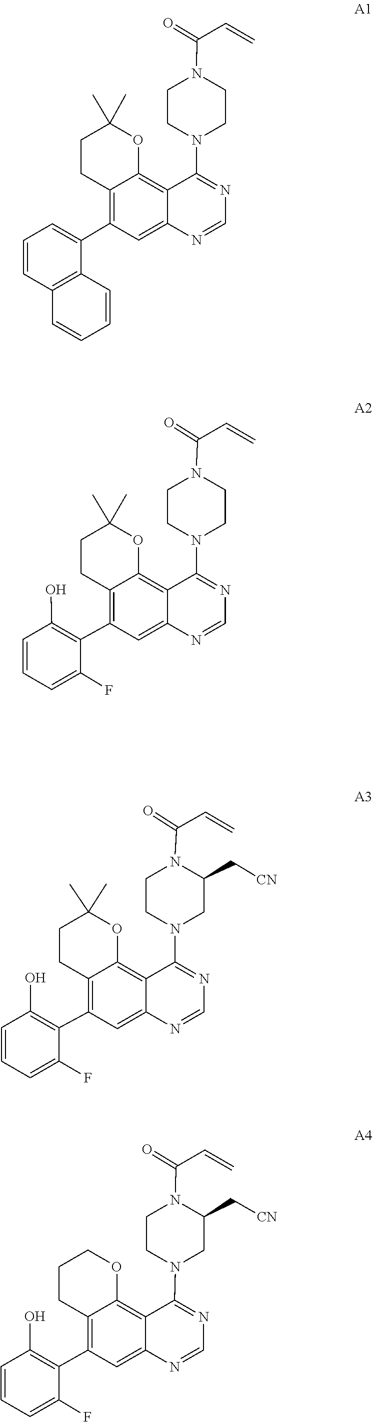 [SMILES: C=CC(=O)N1CCN(c2ncnc3cc(-c4cccc5ccccc45)c4c(c23)OC(C)(C)CC4)CC1 ; C=CC(=O)N1CCN(c2ncnc3cc(-c4c(O)cccc4F)c4c(c23)OC(C)(C)CC4)CC1 ; C=CC(=O)N1CCN(c2ncnc3cc(-c4c(O)cccc4F)c4c(c23)OC(C)(C)CC4)C[C@@H]1CC#N ; C=CC(=O)N1CCN(c2ncnc3cc(-c4c(O)cccc4F)c4c(c23)OCCC4)C[C@@H]1CC#N] 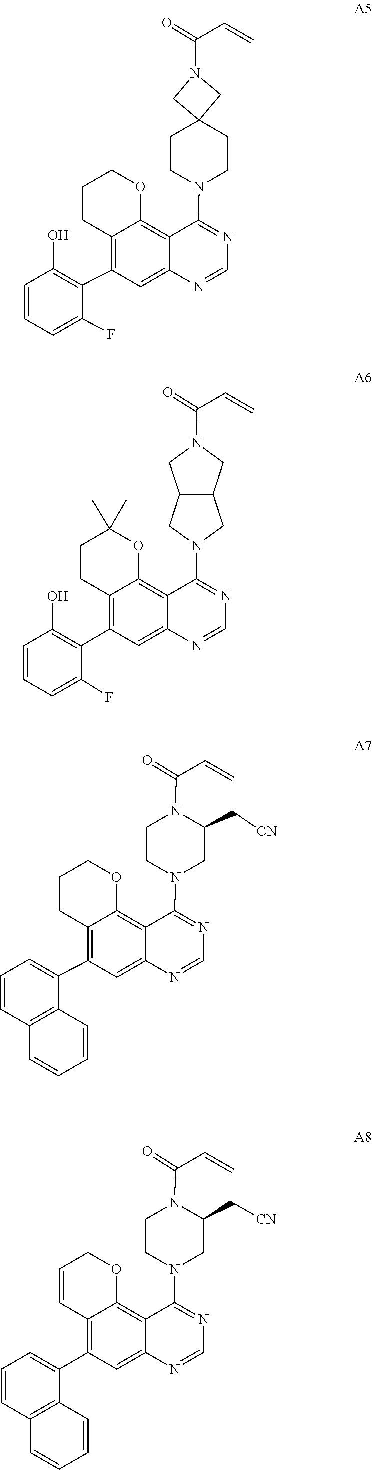 [SMILES: C=CC(=O)N1CC2(CCN(c3ncnc4cc(-c5c(O)cccc5F)c5c(c34)OCCC5)CC2)C1 ; C=CC(=O)N1CC2CN(c3ncnc4cc(-c5c(O)cccc5F)c5c(c34)OC(C)(C)CC5)CC2C1 ; C=CC(=O)N1CCN(c2ncnc3cc(-c4cccc5ccccc45)c4c(c23)OCCC4)C[C@@H]1CC#N ; C=CC(=O)N1CCN(c2ncnc3cc(-c4cccc5ccccc45)c4c(c23)OCC=C4)C[C@@H]1CC#N] 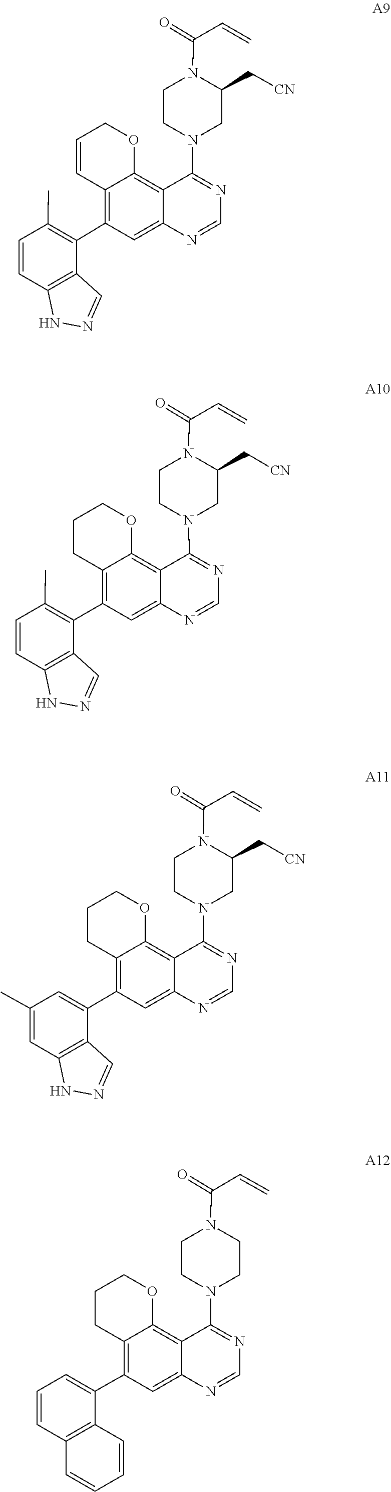 [SMILES: C=CC(=O)N1CCN(c2ncnc3cc(-c4c(C)ccc5[nH]ncc45)c4c(c23)OCC=C4)C[C@@H]1CC#N ; C=CC(=O)N1CCN(c2ncnc3cc(-c4c(C)ccc5[nH]ncc45)c4c(c23)OCCC4)C[C@@H]1CC#N ; C=CC(=O)N1CCN(c2ncnc3cc(-c4cc(C)cc5[nH]ncc45)c4c(c23)OCCC4)C[C@@H]1CC#N ; C=CC(=O)N1CCN(c2ncnc3cc(-c4cccc5ccccc45)c4c(c23)OCCC4)CC1] 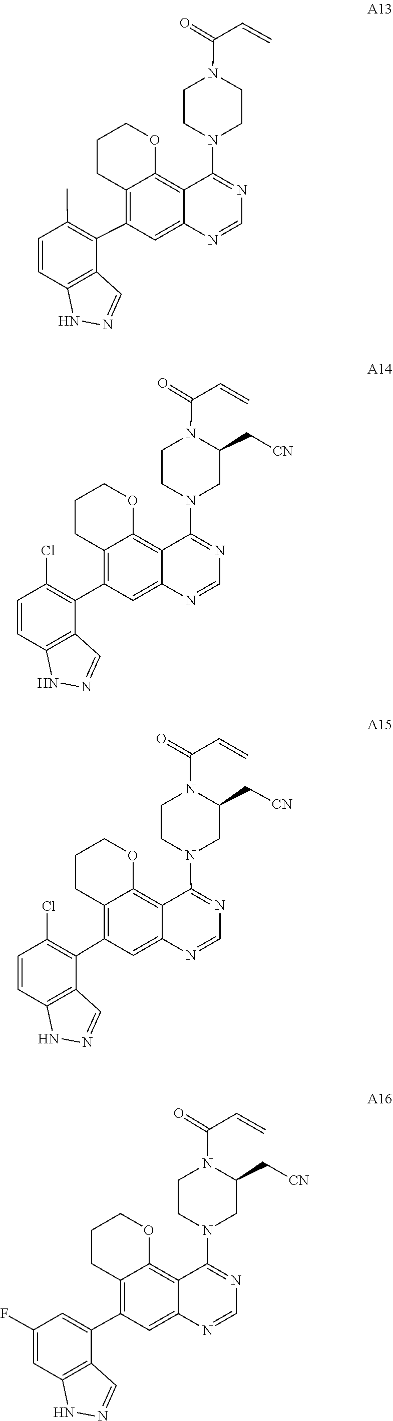 [SMILES: C=CC(=O)N1CCN(c2ncnc3cc(-c4c(C)ccc5[nH]ncc45)c4c(c23)OCCC4)CC1 ; C=CC(=O)N1CCN(c2ncnc3cc(-c4c(Cl)ccc5[nH]ncc45)c4c(c23)OCCC4)C[C@@H]1CC#N ; C=CC(=O)N1CCN(c2ncnc3cc(-c4c(Cl)ccc5[nH]ncc45)c4c(c23)OCCC4)C[C@@H]1CC#N ; C=CC(=O)N1CCN(c2ncnc3cc(-c4cc(F)cc5[nH]ncc45)c4c(c23)OCCC4)C[C@@H]1CC#N] 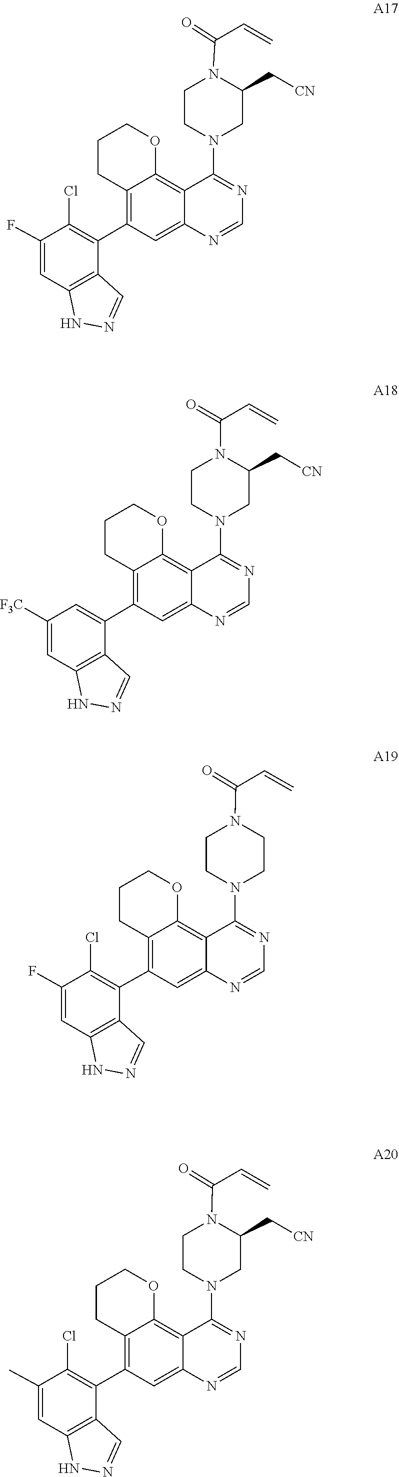 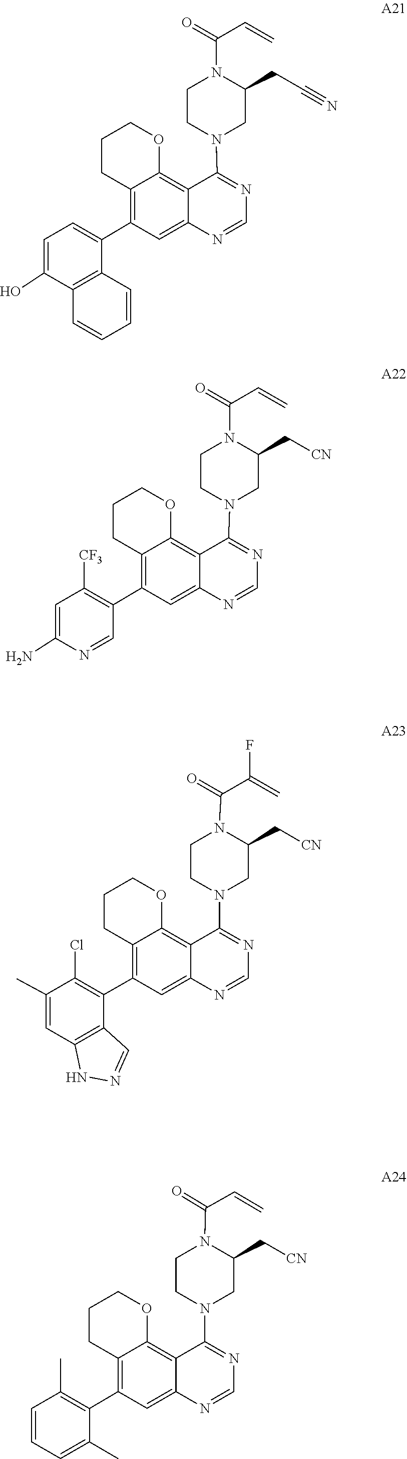 [SMILES: C=CC(=O)N1CCN(c2ncnc3cc(-c4ccc(O)c5ccccc45)c4c(c23)OCCC4)C[C@@H]1CC#N ; C=CC(=O)N1CCN(c2ncnc3cc(-c4cnc(N)cc4C(F)(F)F)c4c(c23)OCCC4)C[C@@H]1CC#N ; C=C(F)C(=O)N1CCN(c2ncnc3cc(-c4c(Cl)c(C)cc5[nH]ncc45)c4c(c23)OCCC4)C[C@@H]1CC#N ; C=CC(=O)N1CCN(c2ncnc3cc(-c4c(C)cccc4C)c4c(c23)OCCC4)C[C@@H]1CC#N] 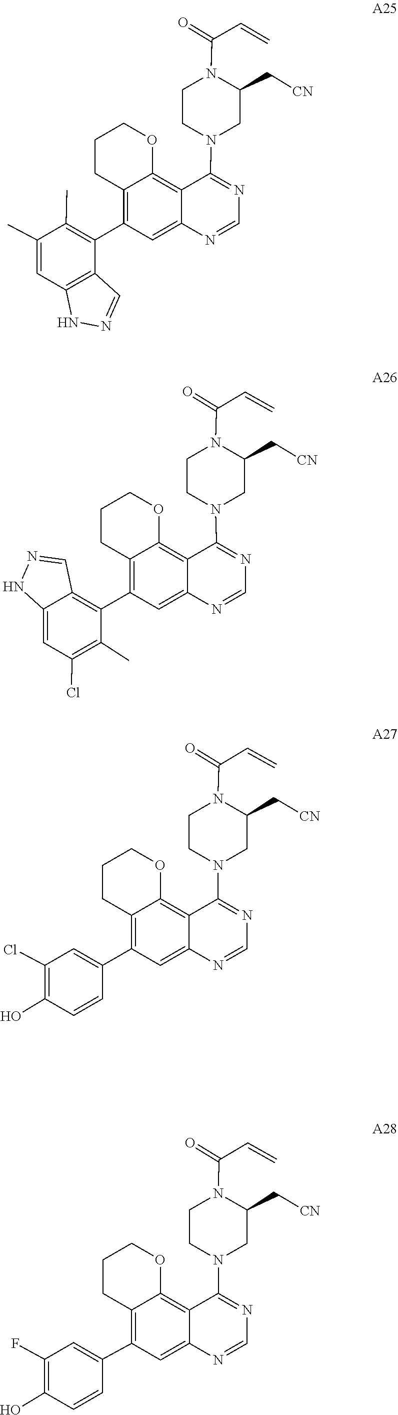 [SMILES: C=CC(=O)N1CCN(c2ncnc3cc(-c4c(C)c(C)cc5[nH]ncc45)c4c(c23)OCCC4)C[C@@H]1CC#N ; C=CC(=O)N1CCN(c2ncnc3cc(-c4c(C)c(Cl)cc5[nH]ncc45)c4c(c23)OCCC4)C[C@@H]1CC#N ; C=CC(=O)N1CCN(c2ncnc3cc(-c4ccc(O)c(Cl)c4)c4c(c23)OCCC4)C[C@@H]1CC#N ; C=CC(=O)N1CCN(c2ncnc3cc(-c4ccc(O)c(F)c4)c4c(c23)OCCC4)C[C@@H]1CC#N] 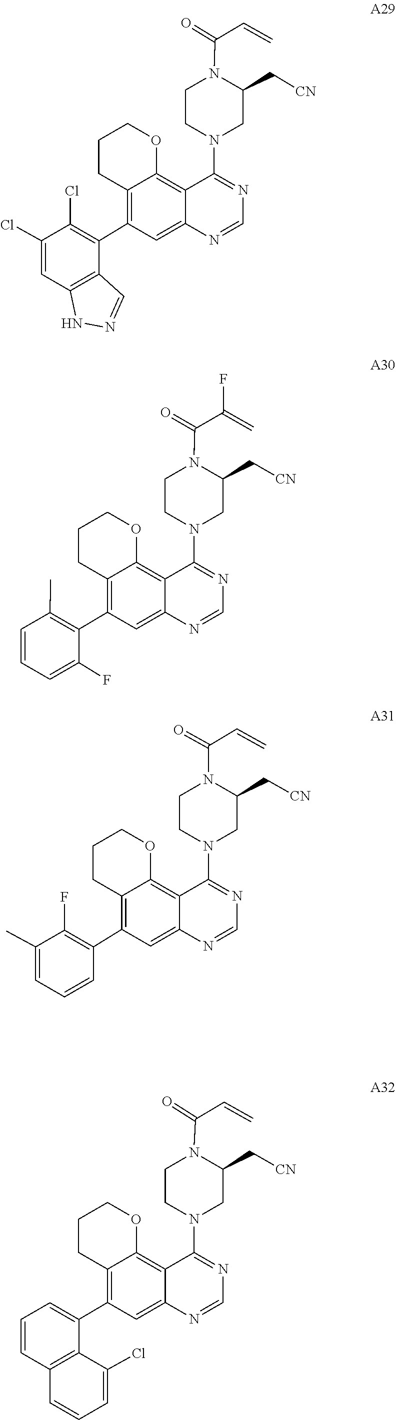 [SMILES: C=CC(=O)N1CCN(c2ncnc3cc(-c4c(Cl)c(Cl)cc5[nH]ncc45)c4c(c23)OCCC4)C[C@@H]1CC#N ; C=C(F)C(=O)N1CCN(c2ncnc3cc(-c4c(C)cccc4F)c4c(c23)OCCC4)C[C@@H]1CC#N ; C=CC(=O)N1CCN(c2ncnc3cc(-c4cccc(C)c4F)c4c(c23)OCCC4)C[C@@H]1CC#N ; C=CC(=O)N1CCN(c2ncnc3cc(-c4cccc5cccc(Cl)c45)c4c(c23)OCCC4)C[C@@H]1CC#N] 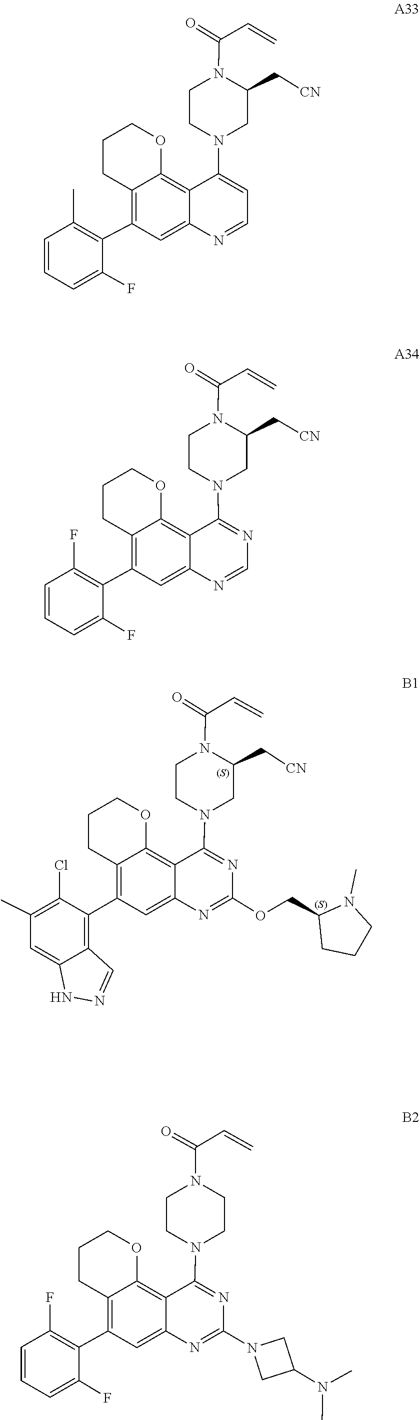 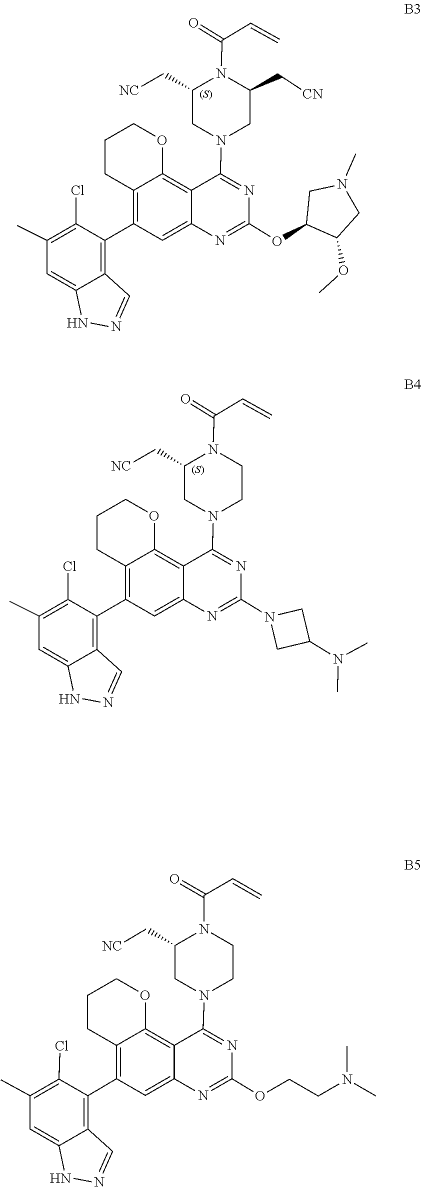 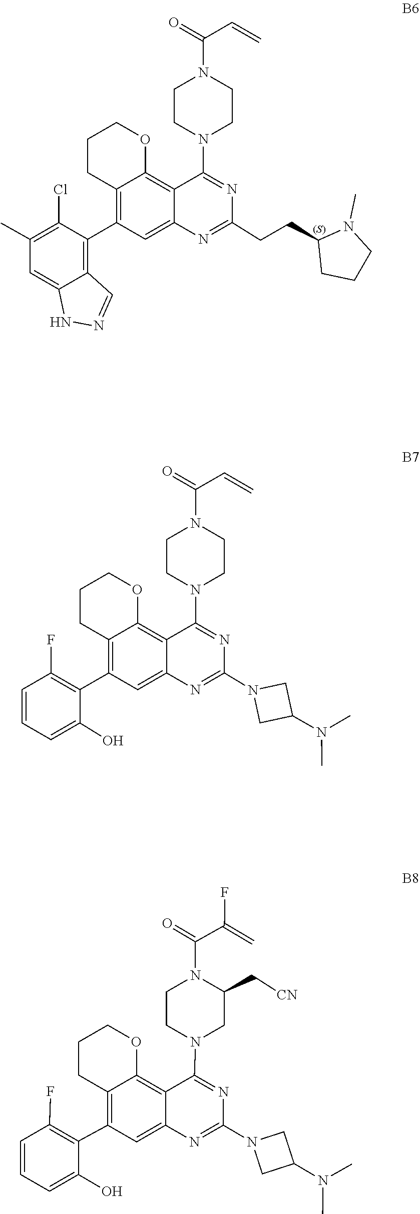 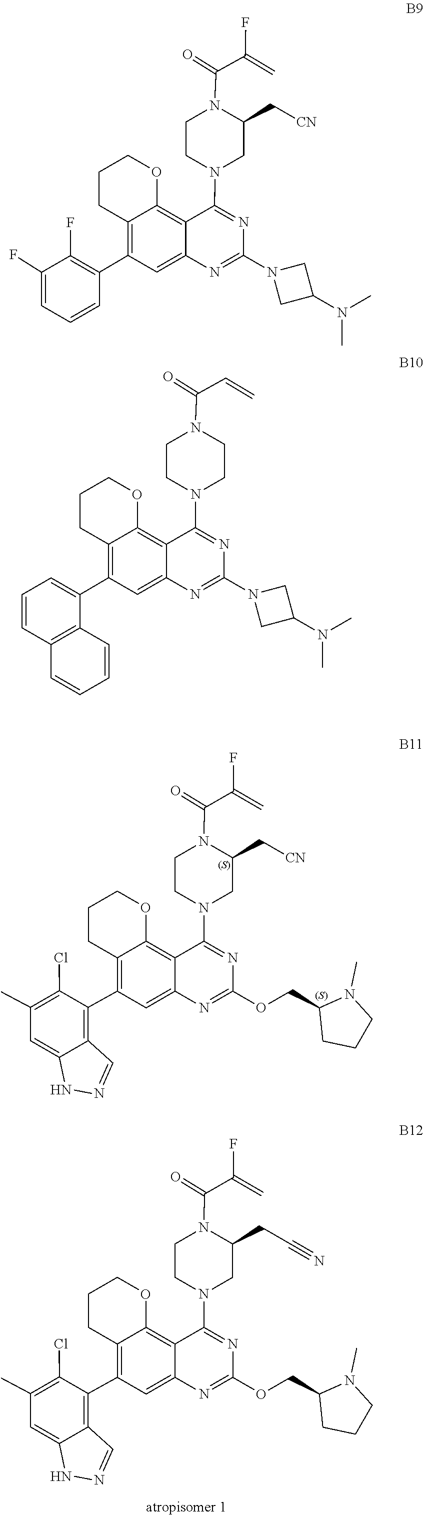 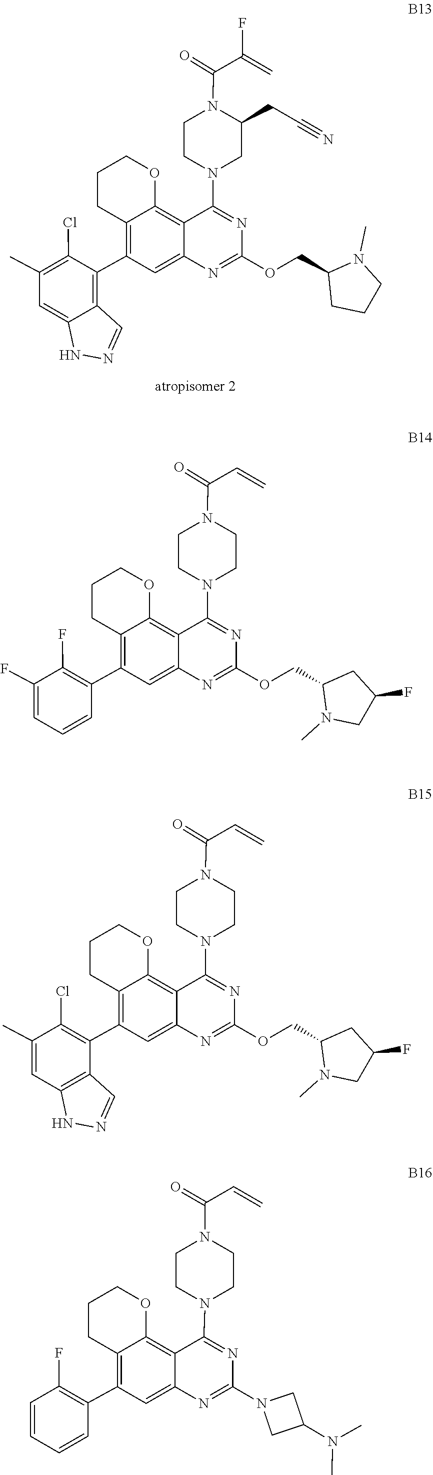 [SMILES: C=C(F)C(=O)N1CCN(c2nc(OC[C@@H]3CCCN3C)nc3cc(-c4c(Cl)c(C)cc5[nH]ncc45)c4c(c23)OCCC4)C[C@@H]1CC#N ; C=CC(=O)N1CCN(c2nc(OC[C@@H]3C[C@@H](F)CN3C)nc3cc(-c4cccc(F)c4F)c4c(c23)OCCC4)CC1 ; C=CC(=O)N1CCN(c2nc(OC[C@@H]3C[C@@H](F)CN3C)nc3cc(-c4c(Cl)c(C)cc5[nH]ncc45)c4c(c23)OCCC4)CC1 ; C=CC(=O)N1CCN(c2nc(N3CC(N(C)C)C3)nc3cc(-c4ccccc4F)c4c(c23)OCCC4)CC1] 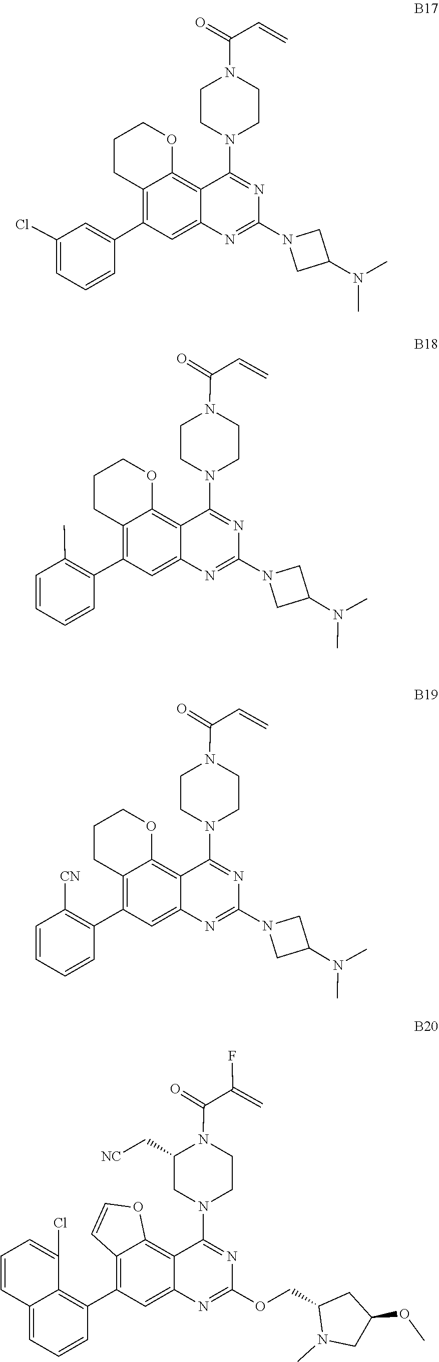 [SMILES: C=CC(=O)N1CCN(c2nc(N3CC(N(C)C)C3)nc3cc(-c4cccc(Cl)c4)c4c(c23)OCCC4)CC1 ; C=CC(=O)N1CCN(c2nc(N3CC(N(C)C)C3)nc3cc(-c4ccccc4C)c4c(c23)OCCC4)CC1 ; C=CC(=O)N1CCN(c2nc(N3CC(N(C)C)C3)nc3cc(-c4ccccc4C#N)c4c(c23)OCCC4)CC1 ; C=C(F)C(=O)N1CCN(c2nc(OC[C@@H]3C[C@@H](OC)CN3C)nc3cc(-c4cccc5cccc(Cl)c45)c4ccoc4c23)C[C@@H]1CC#N] 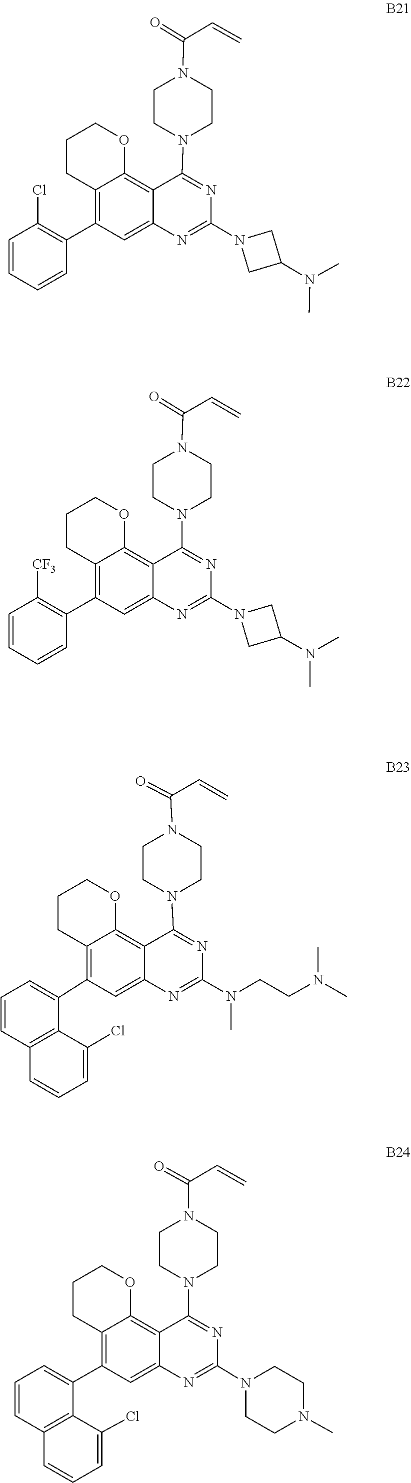 [SMILES: C=CC(=O)N1CCN(c2nc(N3CC(N(C)C)C3)nc3cc(-c4ccccc4Cl)c4c(c23)OCCC4)CC1 ; C=CC(=O)N1CCN(c2nc(N3CC(N(C)C)C3)nc3cc(-c4ccccc4C(F)(F)F)c4c(c23)OCCC4)CC1 ; C=CC(=O)N1CCN(c2nc(N(C)CCN(C)C)nc3cc(-c4cccc5cccc(Cl)c45)c4c(c23)OCCC4)CC1 ; C=CC(=O)N1CCN(c2nc(N3CCN(C)CC3)nc3cc(-c4cccc5cccc(Cl)c45)c4c(c23)OCCC4)CC1] 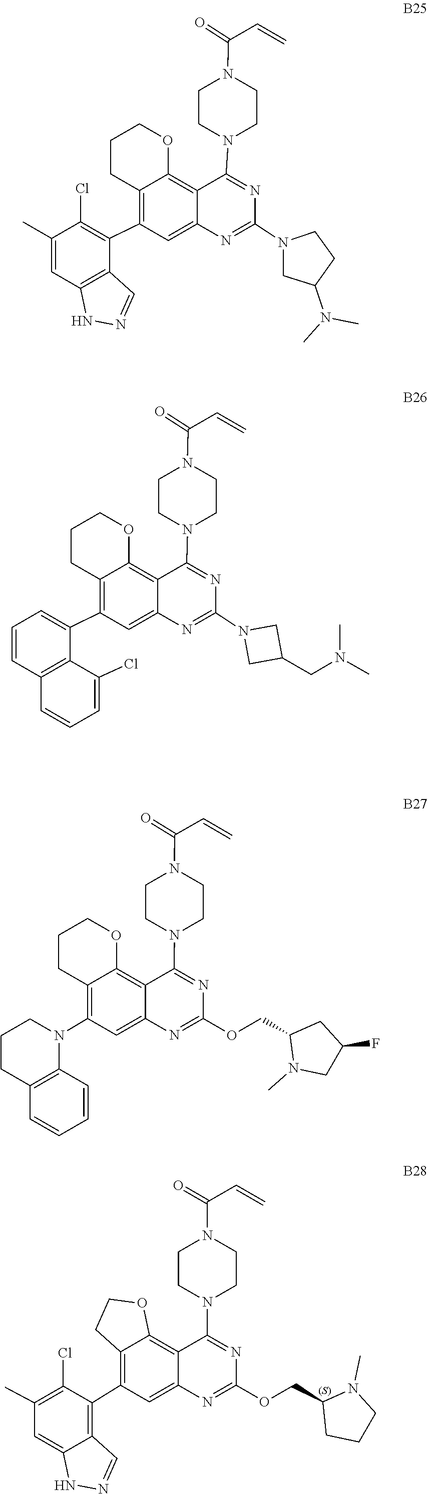 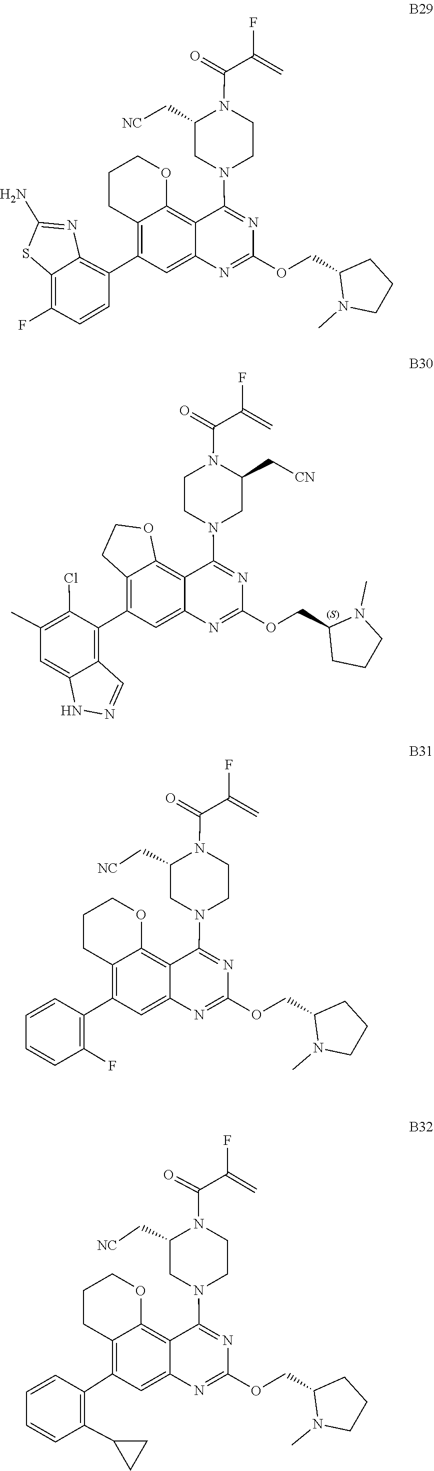 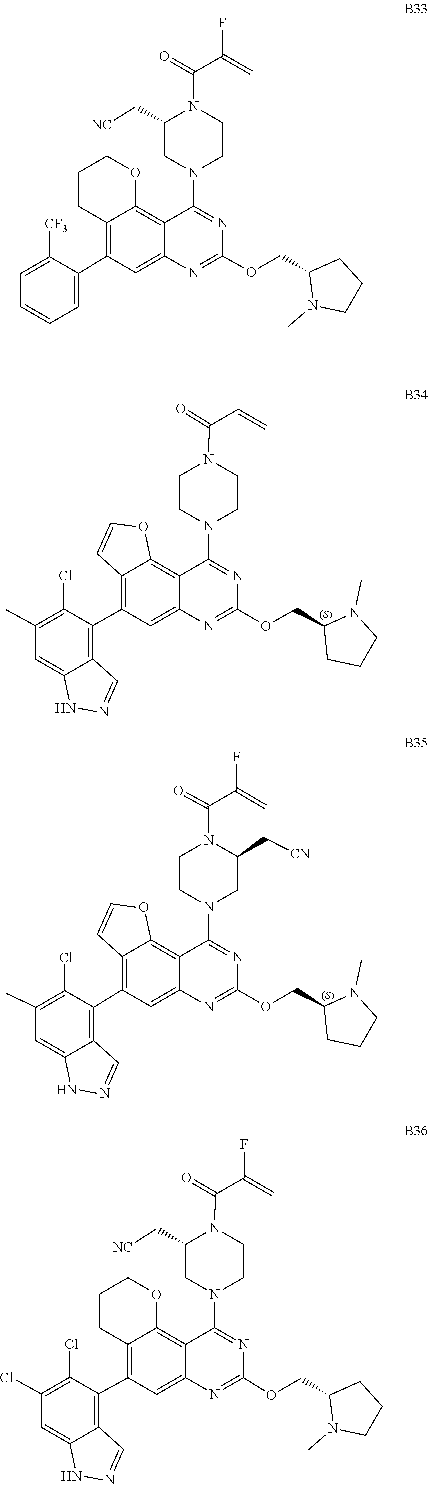 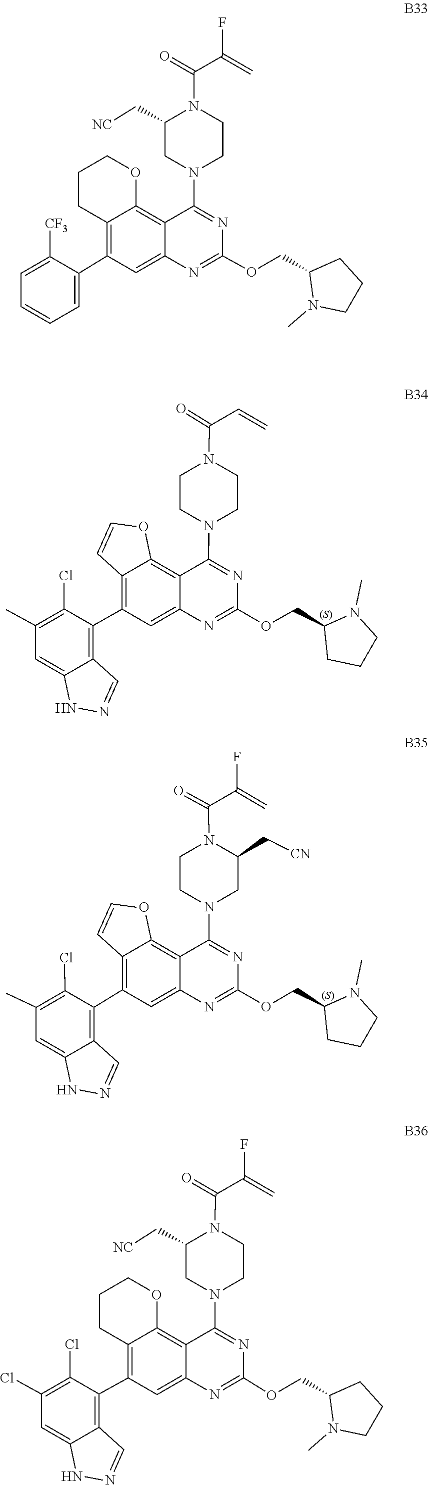 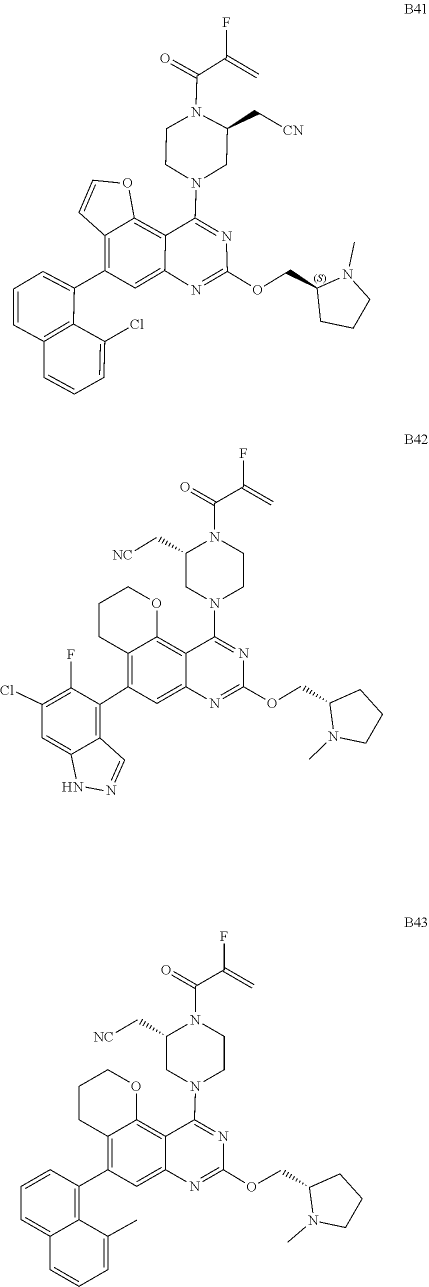 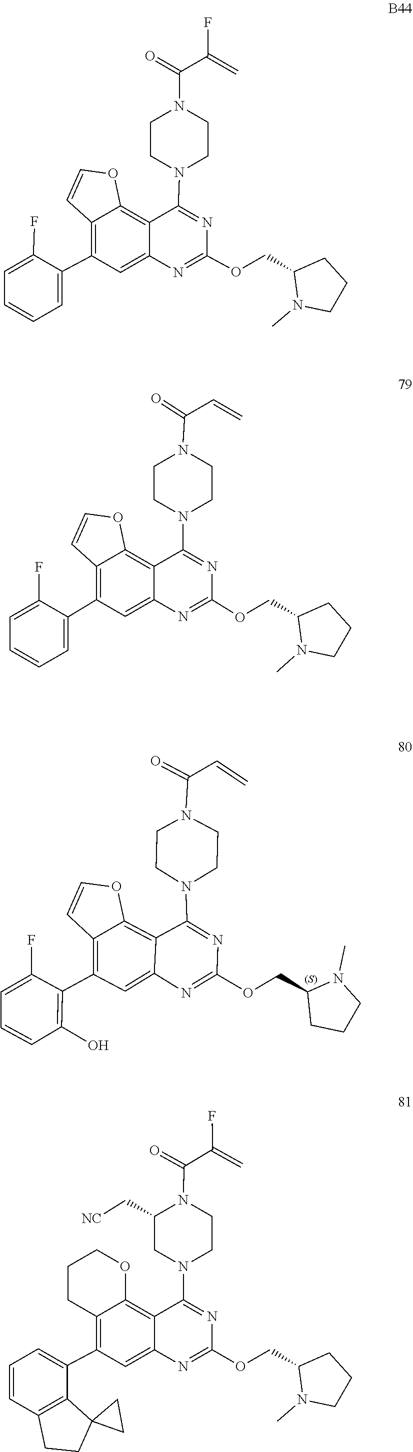 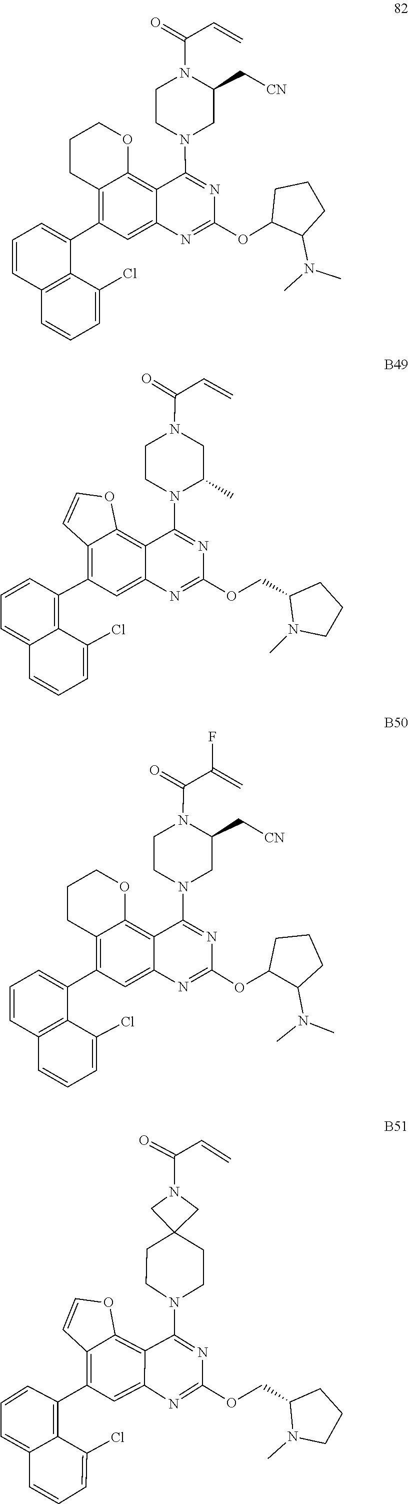 [SMILES: C=CC(=O)N1CCN(c2nc(OC3CCCC3N(C)C)nc3cc(-c4cccc5cccc(Cl)c45)c4c(c23)OCCC4)C[C@@H]1CC#N ; C=CC(=O)N1CCN(c2nc(OC[C@@H]3CCCN3C)nc3cc(-c4cccc5cccc(Cl)c45)c4ccoc4c23)[C@@H](C)C1 ; C=C(F)C(=O)N1CCN(c2nc(OC3CCCC3N(C)C)nc3cc(-c4cccc5cccc(Cl)c45)c4c(c23)OCCC4)C[C@@H]1CC#N ; C=CC(=O)N1CC2(CCN(c3nc(OC[C@@H]4CCCN4C)nc4cc(-c5cccc6cccc(Cl)c56)c5ccoc5c34)CC2)C1] 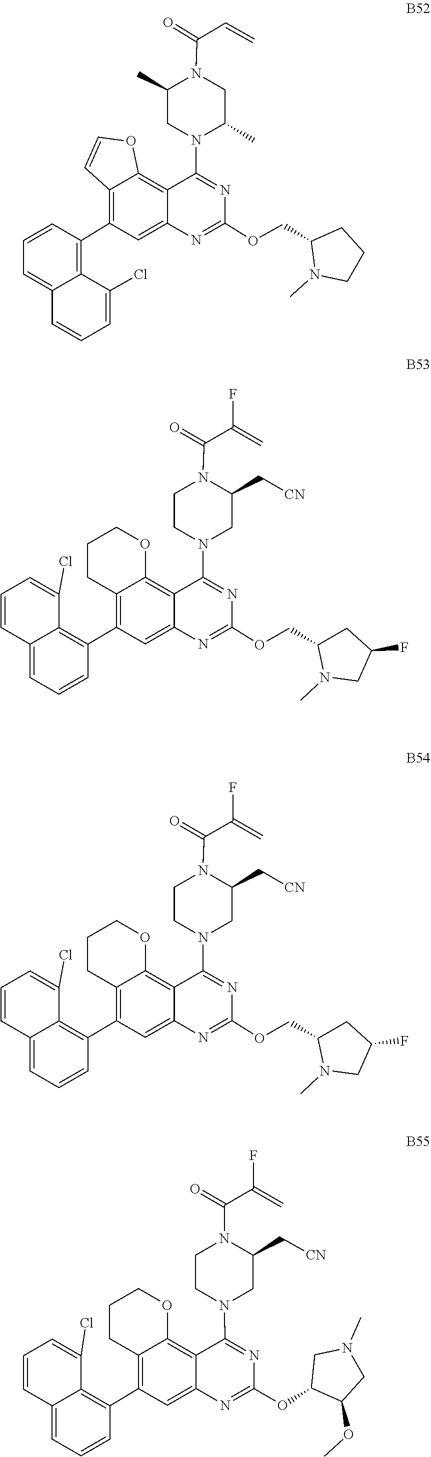 [SMILES: C=CC(=O)N1C[C@H](C)N(c2nc(OC[C@@H]3CCCN3C)nc3cc(-c4cccc5cccc(Cl)c45)c4ccoc4c23)C[C@H]1C ; C=C(F)C(=O)N1CCN(c2nc(OC[C@@H]3C[C@@H](F)CN3C)nc3cc(-c4cccc5cccc(Cl)c45)c4c(c23)OCCC4)C[C@@H]1CC#N ; C=C(F)C(=O)N1CCN(c2nc(OC[C@@H]3C[C@H](F)CN3C)nc3cc(-c4cccc5cccc(Cl)c45)c4c(c23)OCCC4)C[C@@H]1CC#N ; C=C(F)C(=O)N1CCN(c2nc(O[C@@H]3CN(C)C[C@H]3OC)nc3cc(-c4cccc5cccc(Cl)c45)c4c(c23)OCCC4)C[C@@H]1CC#N] 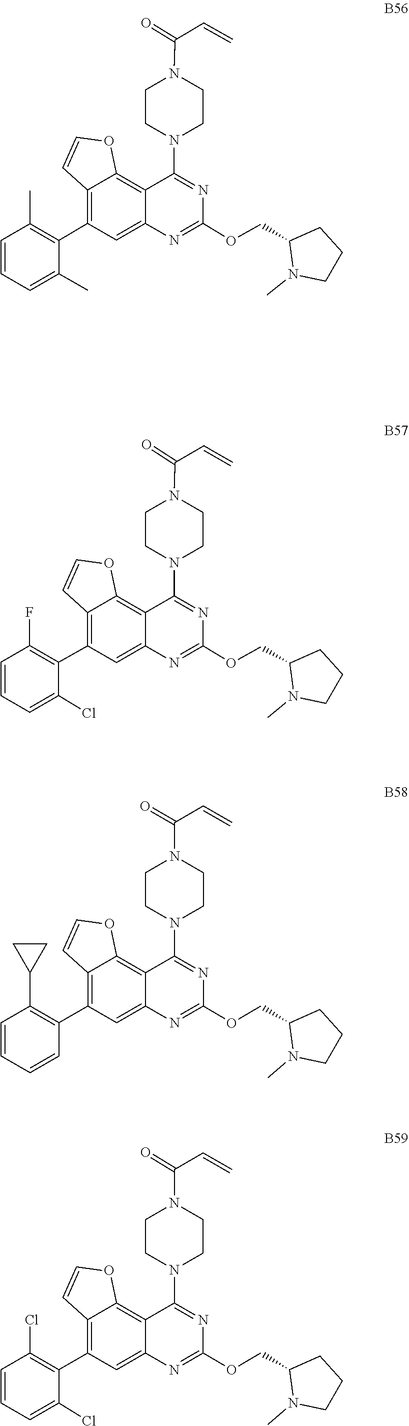 [SMILES: C=CC(=O)N1CCN(c2nc(OC[C@@H]3CCCN3C)nc3cc(-c4c(C)cccc4C)c4ccoc4c23)CC1 ; C=CC(=O)N1CCN(c2nc(OC[C@@H]3CCCN3C)nc3cc(-c4c(F)cccc4Cl)c4ccoc4c23)CC1 ; C=CC(=O)N1CCN(c2nc(OC[C@@H]3CCCN3C)nc3cc(-c4ccccc4C4CC4)c4ccoc4c23)CC1 ; C=CC(=O)N1CCN(c2nc(OC[C@@H]3CCCN3C)nc3cc(-c4c(Cl)cccc4Cl)c4ccoc4c23)CC1] 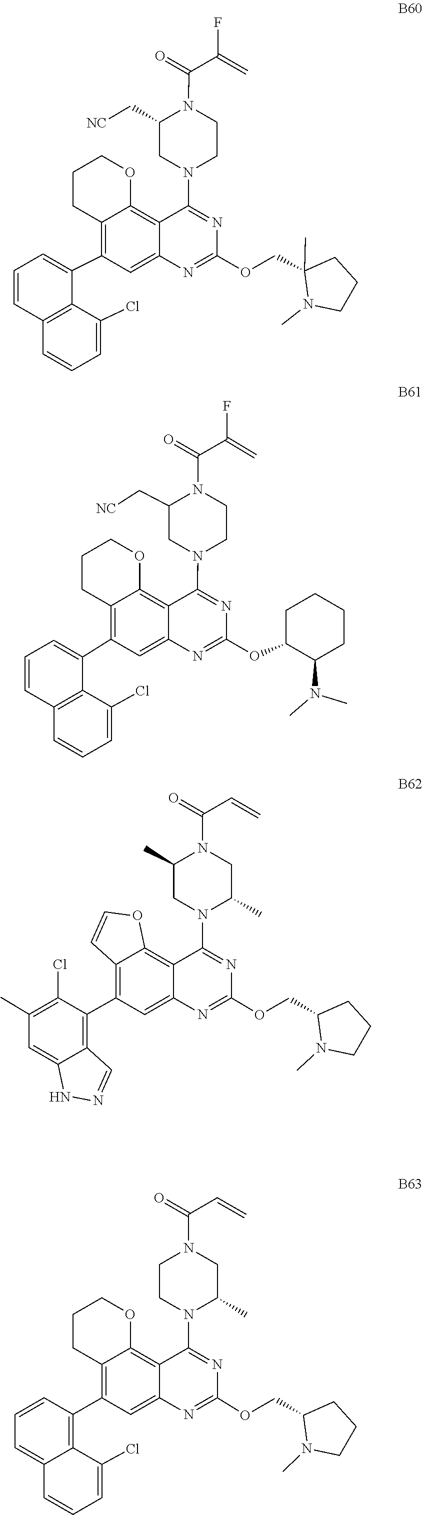 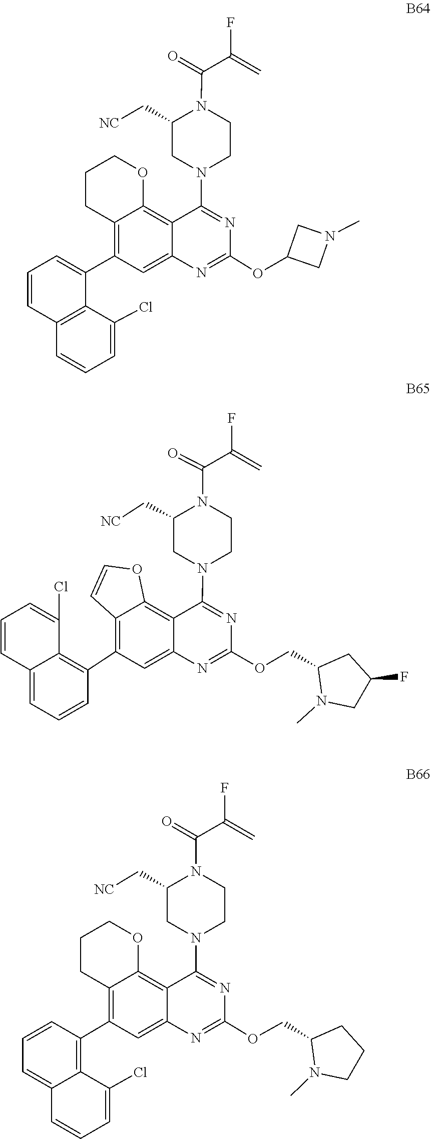 [SMILES: C=C(F)C(=O)N1CCN(c2nc(OC3CN(C)C3)nc3cc(-c4cccc5cccc(Cl)c45)c4c(c23)OCCC4)C[C@@H]1CC#N ; C=C(F)C(=O)N1CCN(c2nc(OC[C@@H]3C[C@@H](F)CN3C)nc3cc(-c4cccc5cccc(Cl)c45)c4ccoc4c23)C[C@@H]1CC#N ; C=C(F)C(=O)N1CCN(c2nc(OC[C@@H]3CCCN3C)nc3cc(-c4cccc5cccc(Cl)c45)c4c(c23)OCCC4)C[C@@H]1CC#N] 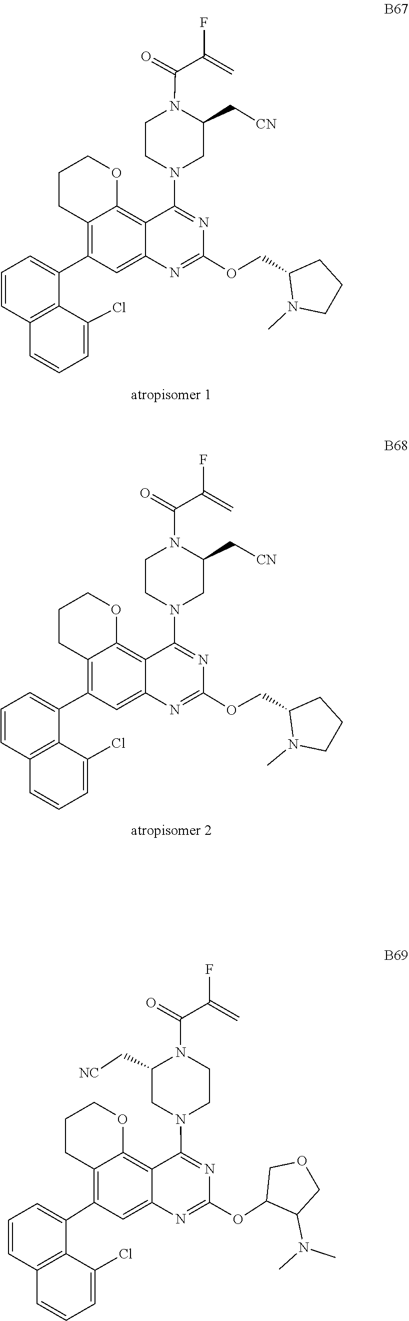 [SMILES: C=C(F)C(=O)N1CCN(c2nc(OC[C@@H]3CCCN3C)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc5cccc(Cl)c45)c4c(c23)OCCC4)C[C@@H]1CC#N ; C=C(F)C(=O)N1CCN(c2nc(OC3COCC3N(C)C)nc3cc(-c4cccc5cccc(Cl)c45)c4c(c23)OCCC4)C[C@@H]1CC#N] 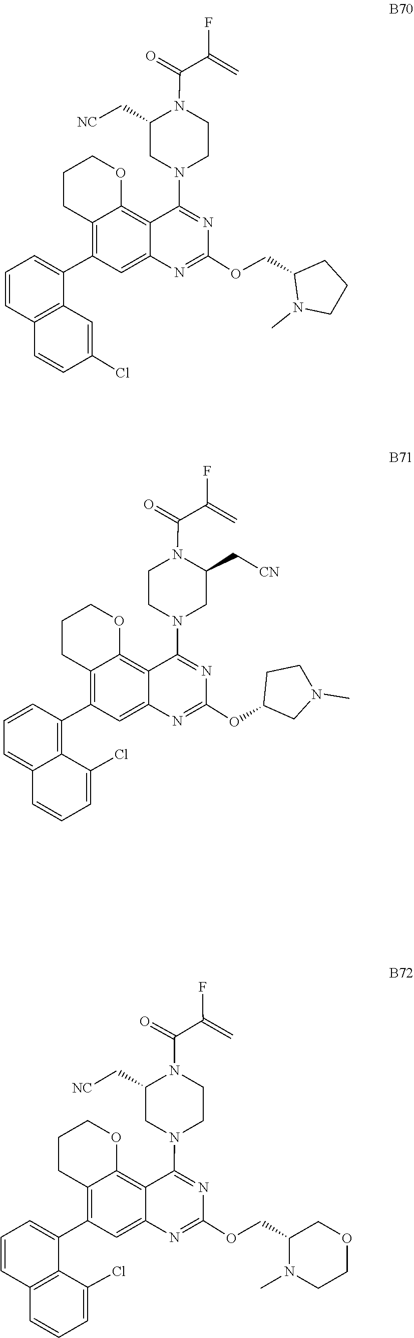 [SMILES: C=C(F)C(=O)N1CCN(c2nc(OC[C@@H]3CCCN3C)nc3cc(-c4cccc5ccc(Cl)cc45)c4c(c23)OCCC4)C[C@@H]1CC#N ; C=C(F)C(=O)N1CCN(c2nc(O[C@@H]3CCN(C)C3)nc3cc(-c4cccc5cccc(Cl)c45)c4c(c23)OCCC4)C[C@@H]1CC#N ; C=C(F)C(=O)N1CCN(c2nc(OC[C@@H]3COCCN3C)nc3cc(-c4cccc5cccc(Cl)c45)c4c(c23)OCCC4)C[C@@H]1CC#N] 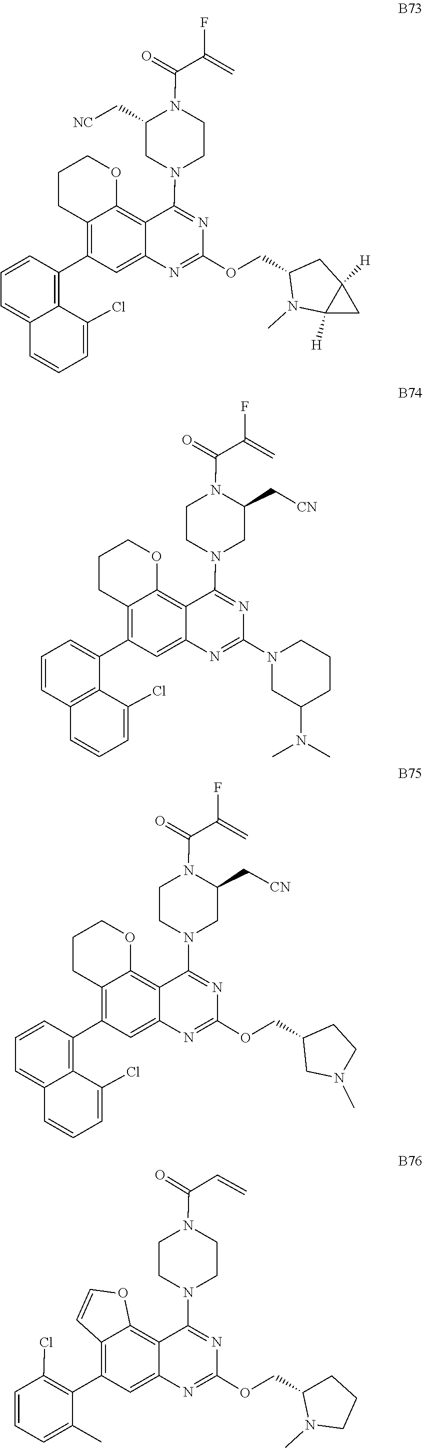 [SMILES: C=C(F)C(=O)N1CCN(c2nc(OC[C@@H]3C[C@H]4C[C@H]4N3C)nc3cc(-c4cccc5cccc(Cl)c45)c4c(c23)OCCC4)C[C@@H]1CC#N ; C=C(F)C(=O)N1CCN(c2nc(N3CCCC(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCN(C)C3)nc3cc(-c4cccc5cccc(Cl)c45)c4c(c23)OCCC4)C[C@@H]1CC#N ; C=CC(=O)N1CCN(c2nc(OC[C@@H]3CCCN3C)nc3cc(-c4c(C)cccc4Cl)c4ccoc4c23)CC1] 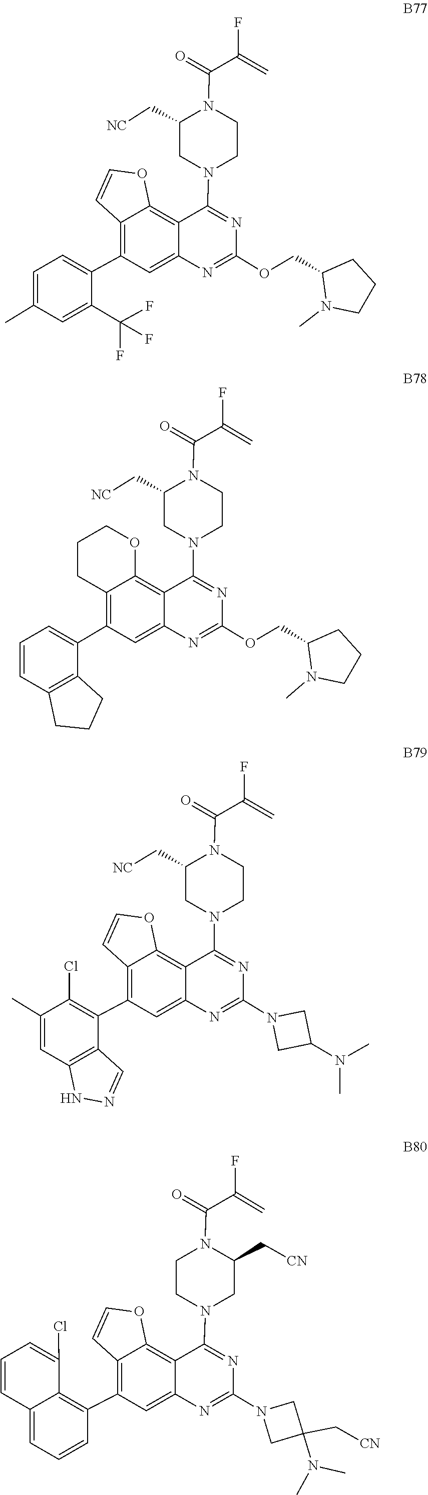 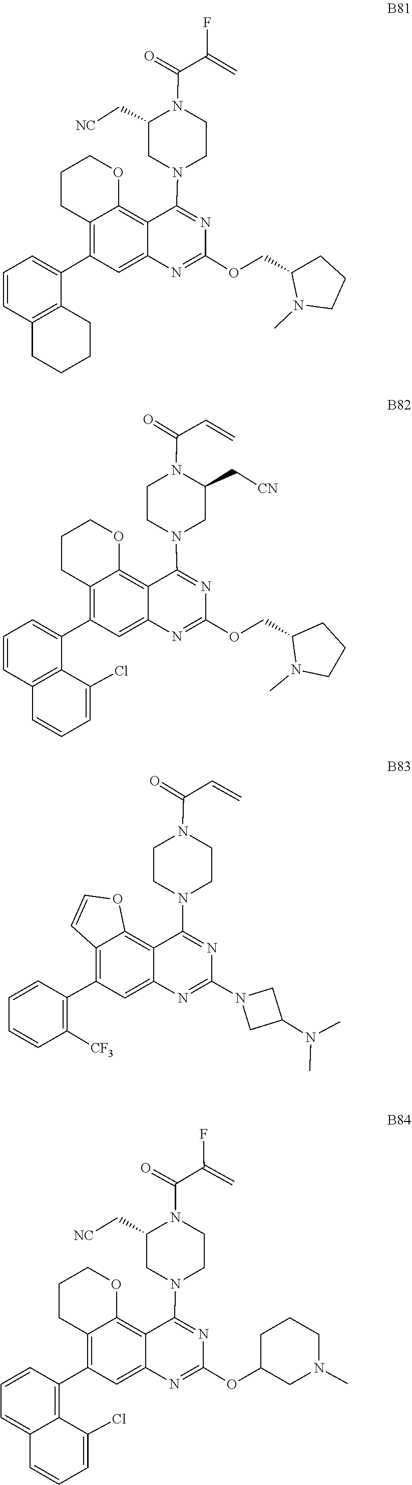 [SMILES: C=C(F)C(=O)N1CCN(c2nc(OC[C@@H]3CCCN3C)nc3cc(-c4cccc5c4CCCC5)c4c(c23)OCCC4)C[C@@H]1CC#N ; C=CC(=O)N1CCN(c2nc(OC[C@@H]3CCCN3C)nc3cc(-c4cccc5cccc(Cl)c45)c4c(c23)OCCC4)C[C@@H]1CC#N ; C=CC(=O)N1CCN(c2nc(N3CC(N(C)C)C3)nc3cc(-c4ccccc4C(F)(F)F)c4ccoc4c23)CC1 ; C=C(F)C(=O)N1CCN(c2nc(OC3CCCN(C)C3)nc3cc(-c4cccc5cccc(Cl)c45)c4c(c23)OCCC4)C[C@@H]1CC#N] 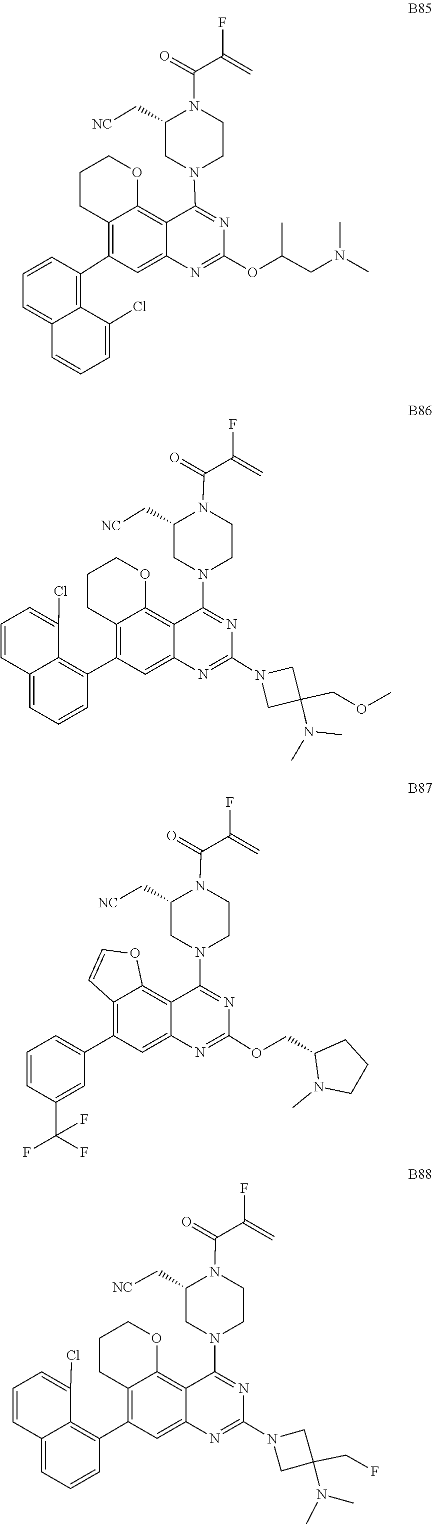 [SMILES: C=C(F)C(=O)N1CCN(c2nc(OC(C)CN(C)C)nc3cc(-c4cccc5cccc(Cl)c45)c4c(c23)OCCC4)C[C@@H]1CC#N ; C=C(F)C(=O)N1CCN(c2nc(N3CC(COC)(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc(C(F)(F)F)c4)c4ccoc4c23)C[C@@H]1CC#N ; C=C(F)C(=O)N1CCN(c2nc(N3CC(CF)(N(C)C)C3)nc3cc(-c4cccc5cccc(Cl)c45)c4c(c23)OCCC4)C[C@@H]1CC#N] 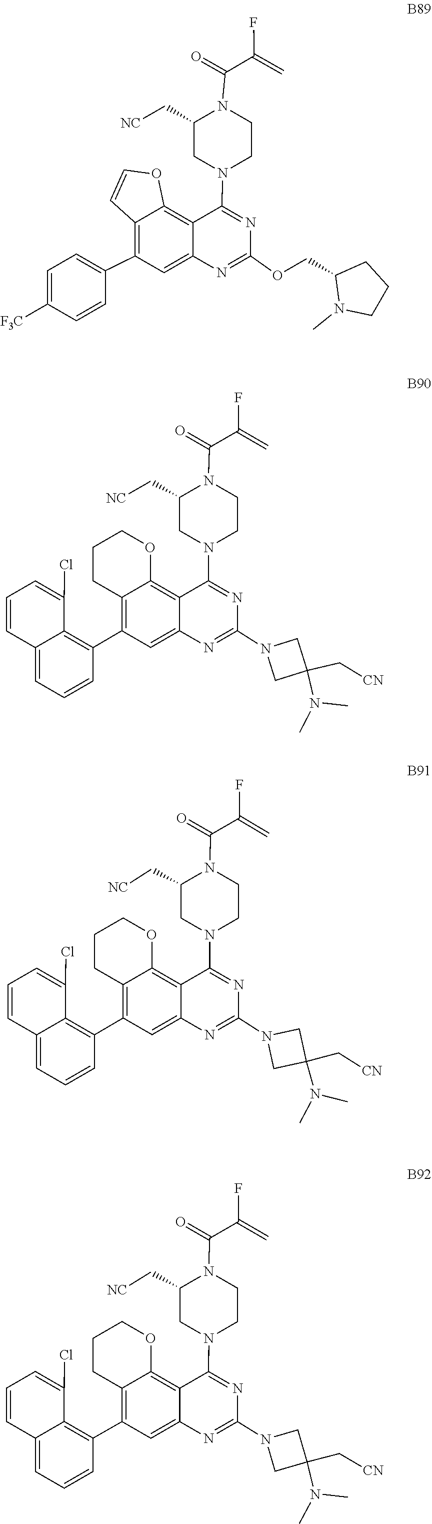 [SMILES: C=C(F)C(=O)N1CCN(c2nc(OC[C@@H]3CCCN3C)nc3cc(-c4ccc(C(F)(F)F)cc4)c4ccoc4c23)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N ; C=C(F)C(=O)N1CCN(c2nc(N3CC(CC#N)(N(C)C)C3)nc3cc(-c4cccc5cccc(Cl)c45)c4c(c23)OCCC4)C[C@@H]1CC#N] 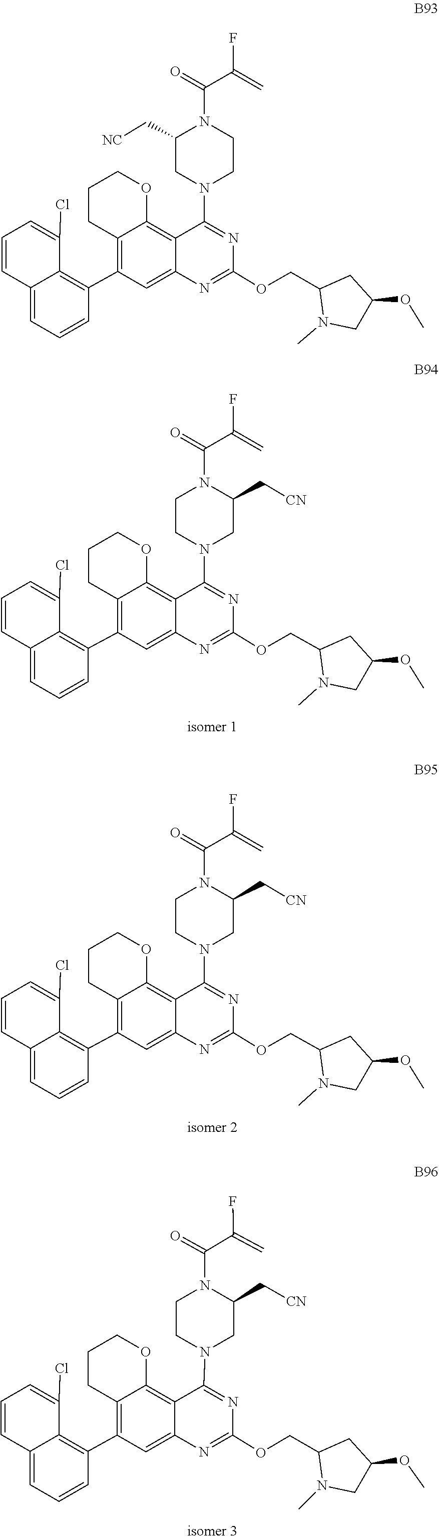 [SMILES: C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N] 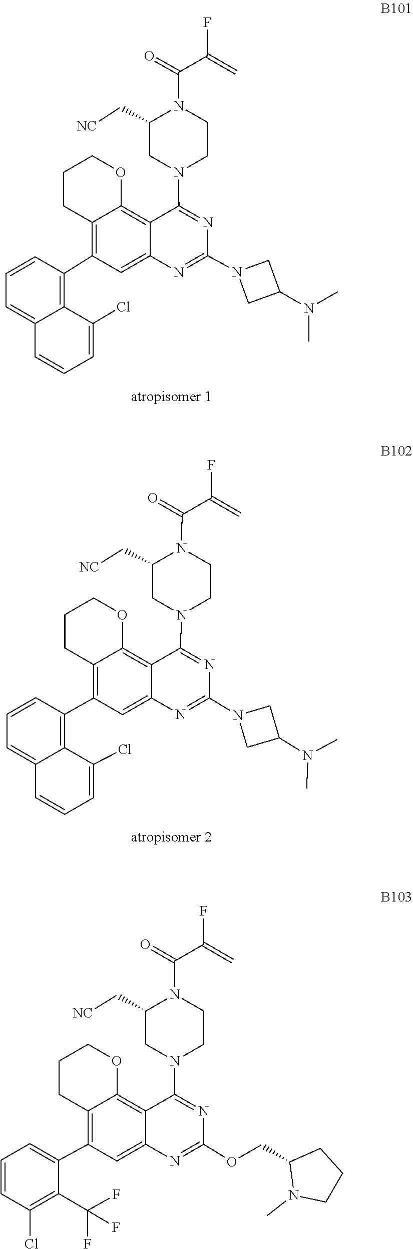 [SMILES: C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc(Cl)c4C(F)(F)F)c4c(c23)OCCC4)C[C@@H]1CC#N] 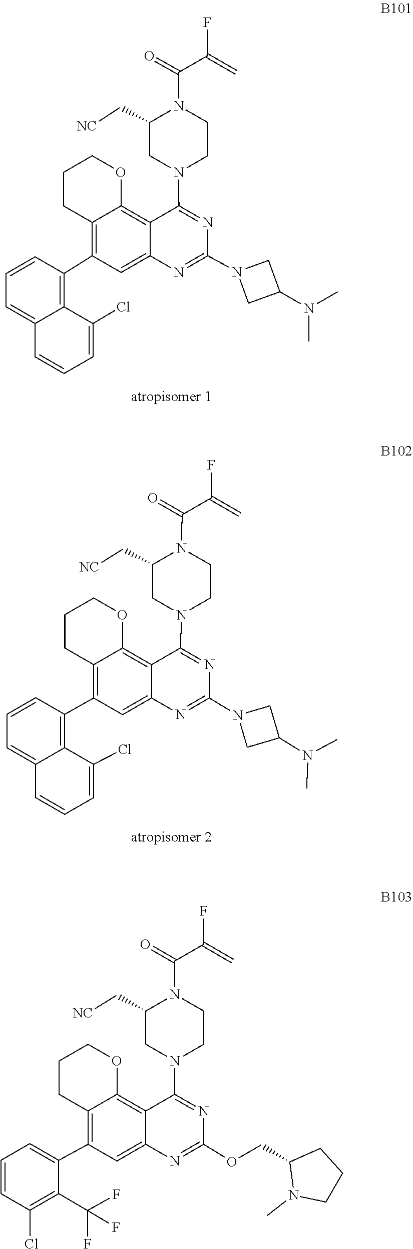 [SMILES: C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(N3CC(N(C)C)C3)nc3cc(-c4cccc5cccc(Cl)c45)c4c(c23)OCCC4)C[C@@H]1CC#N ; C=C(F)C(=O)N1CCN(c2nc(OC[C@@H]3CCCN3C)nc3cc(-c4cccc(Cl)c4C(F)(F)F)c4c(c23)OCCC4)C[C@@H]1CC#N] 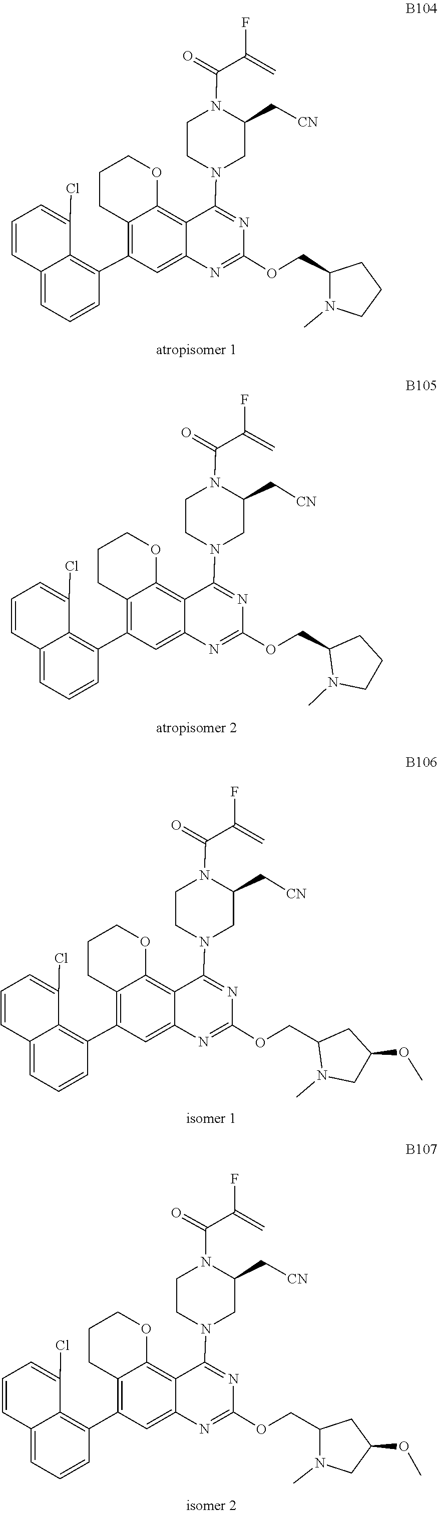 [SMILES: C=C(F)C(=O)N1CCN(c2nc(OC[C@H]3CCCN3C)nc3cc(-c4cccc5cccc(Cl)c45)c4c(c23)OCCC4)C[C@@H]1CC#N ; C=C(F)C(=O)N1CCN(c2nc(OC[C@H]3CCCN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N ; C=C(F)C(=O)N1CCN(c2nc(OCC3C[C@@H](OC)CN3C)nc3cc(-c4cccc5cccc(Cl)c45)c4c(c23)OCCC4)C[C@@H]1CC#N]
or a pharmaceutically acceptable salt thereof, or a stereoisomer thereof.

23. A pharmaceutical composition comprising the compound of claim 1 or a stereoisomer thereof, or a pharmaceutically acceptable salt thereof and a pharmaceutically acceptable excipient.

24. A method of treating cancer in subject in need thereof, comprising administering to the subject a therapeutically effective amount of the compound of claim 1 or a stereoisomer thereof, or a pharmaceutically acceptable salt thereof.
